(19) United States
(12) Patent Application Publication
(10) Pub. No.: US 2011/0269740 A1
(43) Pub. Date: Nov. 3, 2011

(54) JAK KINASE MODULATING COMPOUNDS AND METHODS OF USE THEREOF

(75) Inventors: Sunny Abraham, San Diego, CA (US); Qi Chao, San Diego, CA (US); Michael Hadd, San Diego, CA (US); Mark Holladay, San Diego, CA (US); Gang Liu, San Diego, CA (US); Mitchell Nambu, San Diego, CA (US); Eduardo Setti, San Mateo, CA (US)

(73) Assignee: Ambit Biosciences Corporation

(21) Appl. No.: 13/000,606

(22) PCT Filed: Jul. 2, 2009
(86) PCT No.: PCT/US2009/003938

§ 371 Date: Mar. 24, 2011

Related U.S. Application Data
(60) Provisional application No. 61/133,845, filed on Jul. 2, 2008.

(51) Int. Cl. A61K 31/53; A61K 31/5377; A61P 35/00; A61P 19/02; A61P 37/06; A61P 17/02; A61P 3/10; C07D 487/04; A61P 29/00

(52) U.S. Cl. 514/210.21; 544/183; 514/243; 544/112; 514/233.2

(57) ABSTRACT

Provided herein are pyrrolotriazine compounds for treatment of JAK kinase, including JAK2 kinase mediated diseases. Also provided are pharmaceutical compositions comprising the compounds and methods of using the compounds and compositions.

### RELATED APPLICATIONS

This application claims priority to U.S. provisional application No. 61/133,845, filed Jul. 2, 2008. The disclosure of the above referenced application is incorporated by reference herein in its entirety.

### FIELD

Provided herein are pyrrolotriazine compounds. In certain embodiments, the compounds are modulators of JAK kinases. Also provided are compositions comprising the compounds and methods of use thereof. The compounds provided are useful in the treatment, prevention, or amelioration of a disease or disorder related to JAK, including JAK2 activity or one or more symptoms associated with such diseases or disorders.

### BACKGROUND

Protein kinases (PKs) are enzymes that catalyze the phosphorylation of hydroxyl groups on tyrosine, serine or threonine residues of proteins. Protein kinases act primarily as growth factors or cytokine receptors and play a central role in signal transduction pathways regulating a number of cellular functions, such as cell cycle, cell growth, cell differentiation and cell death. One particular family of protein kinases that have been found to be the main mediator of signals from cytokines is the Janus kinase or JAK family of kinases.

The JAK kinase family is a cytoplasmic protein kinase family comprising the members JAK1, JAK2, JAK3 and TYK2. Various studies suggest that ligand binding to a receptor leads to receptor dimerization or oligomerization, which leads to JAK recruitment and activation either through autophosphorylation or phosphorylation by other JAK kinases or by other tyrosine kinases, which in turn leads to tyrosine phosphorylation of the receptors as well as down stream substrates of JAK. Growth factor or cytokine receptors that recruit JAK kinases include the interferon receptors, interleukin receptors (receptors for the cytokines IL-2 to IL-7, IL-9 to IL-13, IL-15, IL-23), various hormone receptors (erythropoietin (Epo) receptor, the thrombopoietin (Tpo) receptor, the leptin receptor, the insulin receptor, the prolactin (PRL) receptor, the Granulocyte Colony-Stimulating Factor (G-CSF) receptor and the growth hormone receptor), receptor protein tyrosine kinases (such as EGFR and PDGFR), and receptors for other growth factors such as leukemia inhibitory factor (LIF), Oncastatin M (OSM), IFNα/β/γ, Granulocyte-macrophage colony-stimulating factor (GM-CSF), Ciliary neurotrophic factor (CNTF), cardiotrophin-1 (CT-1) (See, Rane, S. G. and Reddy E. P., Oncogene 2000 19, 5662-5679).

Phosphorylated receptors serve as docking sites for other SH-2 domain containing signaling molecules that interact with JAKs such as the STAT family of transcription factors, Src family of kinases, MAP kinases PI3 kinase and protein tyrosine phosphatases (Rane S. G. and Reddy E. P., Oncogene 2000 19, 5662-5679). The family of latent cytoplasmic transcription factors, STATS, are the most well characterized downstream substrates for JAKs. The STAT proteins bind to phosphorylated cytokine receptors through their SH2 domains to become phosphorylated by JAKs, which event leads to their dimerization and release and eventual translocation to the nucleus where they activate gene transcription. The various members of STAT which have been identified thus far, are STAT1, STAT2, STAT3, STAT4, STAT5 (including STAT5a and STAT5b) and STAT6.

Since the JAK kinases may play an important signaling role via such receptors, disorders of fat metabolism, growth disorders and disorders of the immune system are all potential therapeutic targets.

The JAK kinases are implicated in myeloproliferative disorders which are comprised of several clonal hematologic diseases that are believed to arise from a transformation of a hematopoietic stem cell, and which include chronic myeloid leukemia (CML), polycythemia vera (PV), essential thrombocythemia (ET), primary myelofibrosis (PMF), chronic eosinophilic leukemia (CEL), chronic myelomonocytic leukemia (CMML) and systemic mastocytosis (SM). Myeloproliferative disorders are believed to arise from either gain-of-function mutations to JAK itself or from activation by the oncoprotein BCR-ABL, which specifically activates the JAK2 pathway.

The BCR ABL fusion gene caused by the Philadelphia chromosome translocation (Ph) is the molecular basis for most myeloproliferative disorders, and is the genetic basis for chronic myeloid leukemia (CML). BCR-ABL has been shown to activate the JAK2 pathway which in turn causes an increase in the expression of c-Myc, a transcription factor which is required for leukemic transformation (See Samanta et al. Cancer Res 2006, 66(13), 6468-647, Sawyers et al. Cell, 1992, 70, 901-910). Treatment of CML cell lines such as 32Dp210 and K562, as well as mouse Bcr-Abl+32D cells with a potent and specific inhibitor of the JAK2 kinase, AG490, lead to the downstream reduction of c-Myc, which suggests that JAK2 is an important therapeutic target for CML (Samanta et al. Cancer Res (2006) 66(13):6468-6472). AG490 was also found to induce apoptosis in BCR-Abl+BaF3 cells which carried the Abl T315I mutation and which therefore were resistant to imatinib treatment, which finding further indicates that JAK2 inhibition may be useful for the treatment of imatinib-resistant forms of CML (Samanta et al. Cancer Res (2006) 66(13):6468-6472).

The three main Ph-negative myeloproliferative disorders are polycythemia vera (PCV), essential thrombocythemia and idiopathic myelofibrosis (IMF). One particular JAK2 mutation, the JAK2 V617F mutation, renders the JAK2 enzyme constitutively active, and has been found in a high proportion of patients with polycythemia vera and related myeloproliferative disorders. 95% of patients with PCV carry the JAK2 V617F mutation (Tefferi N Eng J Med (2007) 356(5): 444-445) Baxter et al. Lancet (2005) 365:1054-1056, Levine et al. Blood (2006) 107:4139-4141), and 50-60% of patients with essential thrombocythemia or IMF carry this mutation (Jones et al. Blood (2005) 106:2162-2168. V617F mutation is associated with poorer survival IMF, which suggests that JAK2 would be a potentially important therapeutic target for IMF (Campbell et al. Blood (2006) 107(5): 2098-2100). Those patients with polycythemia vera who do not have the V617F mutation have other JAK2 mutations (Scott et al. N Eng J Med 2007 356(5): 459-468).

Other JAK2 mutations have been identified that are linked to disorders outside of MDS. A recently discovered constitutively active JAK2 mutation, JAK2 T875N mutation, has been recently identified and associated with acute megakaryoblastic leukemia (AMKL), a subtype of acute myeloid leukemia (AML) (See Mercher et al. Blood (2006) 108(8): 2770-2778.) Several fusions involving the JAK2 gene have been identified and reported to induce myeloid leukemias (See Lacronique et al. Science (1997) 278:1309-1312, Lacronique et al. Blood (2000) 95:2535-2540, Griesinger F. et al. Genes Chromosomes Cancer (2005) 44:329-333, Bousquet et al. Oncogene (2005) 24:7248-7252). For example, JAK2 rendered constitutively active by fusion with the oligomerization domain of the transcription factor TEL has been found to cause either myeloid or lymphoid leukemia in mice (See Scwhaller et al. 1998, Schwaller et al. Mol. Cell. 2000 6, 693-704, Zhao et al. EMBO 2002 21(9), 2159-2167).

Considering the pleiotropic effect of Jak2 signaling and its importance in erythropoiesis, one approach to developing a JAK2 inhibitor is to develop one that is selective for mutated forms of JAK2. However, in vitro studies indicate that hematopoietic colonies from JAK2V617F-positive patients are more sensitive to JAK2 activation than cells from normal subjects, which suggests that inhibitor of wild type JAK2 may also show efficacy in clinical trials (See Levine et al. Nature Reviews, 2007 7, 673-683).

Signaling by IL-6 generally induces phosphorylation of STAT3 mediated by JAK. STAT3 overactivation is reported in many types of malignancies including myeloma, head and neck cancers, prostate cancer, breast cancer, ovarian cancer, melanoma, lung cancers, brain tumors, pancreatic and renal carcinoma. STAT3 and STAT5 are overexpressed in some human malignancies such as head and neck and breast cancer (See Blume-Jensen (2001) review and Bromberg review (2002), Rane (2000) Oncogene). In one study, STAT3 phosphorylation was found to be mediated by JAK kinases in prostrate cancer cell lines DU145 and NRP-154, and although STAT3 was proposed as a good target to inhibit for prostate cancer, the study also showed that the JAK2 specific inhibitor AG490 induced apoptosis in prostate cancer cell DU145, and JAK1 specific inhibitor piceatannol induced apoptosis in prostate cancer cell NRP-154 (See Barton et al. Mol. Canc. Ther. 2004 3(1), 11-20), which suggests that JAK may also serve as a target for prostate cancer, including androgen-resistant prostate cancer.

Because cytokines play critical roles in regulating immunity and inflammation, JAKs as a prominent mediator of the cytokine signaling pathway, is considered to be a therapeutic target for inflammation and transplant rejections. An orally available, selective JAK3 antagonist, CP-690550 has been shown efficacy in preventing transplant rejection in two animal models, a murine heterotopic heart transplant model and a non-human primate renal transplant model (See O'Shea et al. Ann Rheum Dis 2004 63, 67-71).

Given the multitude of diseases attributed to the dysregulation of JAK signaling, there is an ever-existing need to provide novel classes of compounds that are useful as inhibitors of enzymes in the JAK signaling pathway.

### SUMMARY

Provided herein are compounds of formula (I) or pharmaceutically acceptable salts, solvates, hydrates or clathrates thereof. In certain embodiment, the compounds have activity as JAK kinase, including JAK2 kinase modulators. The compounds are useful in medical treatments, pharmaceutical compositions and methods for modulating the activity of JAK kinase, including wildtype and/or mutated forms of JAK kinase. In certain embodiments, the compounds provided herein have activity as JAK2 kinase modulators. In one embodiment, the compounds for use in the compositions and methods provided herein have formula (I).

In one embodiment, the compounds provided herein have formula (I):

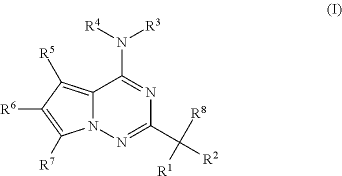

or pharmaceutically acceptable salts, solvates, hydrates or clathrates thereof, wherein:

R¹ and R² are selected from (i), (ii), (iii) and (iv) as follows:

(i) R¹ and R² together form ═O, ═S, ═NR⁹ or ═CR¹⁰R¹¹;

(ii) R¹ and R² together with the carbon atom to which they are attached, form optionally substituted cycloalkyl, oxacycloalkyl or heterocyclyl, wherein the substitutents, when present, are one, two or three groups selected from oxo, alkyl, and halo;

(iii) R¹ and R² are each independently hydrogen, halo, haloalkyl, alkyl, alkenyl, alkynyl, cycloalkyl, hydroxyalkyl, alkoxyalkyl, aminoalkyl, heterocyclylalkyl, heteroaryl, —OR¹² or —S(O)ₚR¹²; or

(iv) R¹ is —OR¹², —NR¹³R¹⁴, —S(O)ₚR¹², —N(R¹⁵)R¹⁶OR¹², —N(R¹⁵)R¹⁶NR¹³R¹⁴, —N(R¹⁵)R¹⁶S(O)ₚR¹², —NR¹⁵S(O)ₚR¹², —OR¹⁶OR¹², —OR¹⁶NR¹³R¹⁴, —OR¹⁶S(O)ₚR¹², —S(O)ₚR¹⁶OR¹², —S(O)ₚR¹⁶NR¹³R¹⁴, —S(O)ₚNR¹³R¹⁴, —S(CN), —OC(O)R¹², —NR¹⁵C(O)NR¹³R¹⁴, —NR¹⁵C(O)OR¹², —NR¹⁵C(O)R¹² or —R¹⁸C(O)OR¹², and R² is hydrogen, cyano, haloalkyl, alkyl, alkenyl, alkynyl, cycloalkyl, hydroxyalkyl or alkoxyalkyl;

R³ is cycloalkyl, aryl, heterocyclyl or heteroaryl;

R⁴ is hydrogen or alkyl;

R⁵, R⁶ and R⁷ are each independently selected from hydrogen, halo, nitro, hydroxy, alkyl, alkenyl, alkynyl, cycloalkyl, heterocyclyl, heterocyclylalkyl, —OR¹⁷, —NR¹⁵C(O)R¹⁷, —NR¹⁵C(O)OR¹⁷, —NR¹⁵C(O)NR¹⁹R²⁰, —NR¹⁵S(O)ₚR¹⁷, —C(O)NR¹⁹R²⁰, —S(O)ₚNR¹⁹R²⁰, —NR¹⁵C(O)OR¹⁷ or —NR¹⁵C(O)NR¹⁹R²⁰—C(S)NR¹⁹R²⁰, —C(O)OR¹⁷, —C(S)OR¹⁷ and —C(═NOR¹⁵)R²¹ wherein the alkyl, alkenyl, alkynyl, cycloalkyl, heterocyclyl and heterocyclylalkyl may each be optionally substituted with one, two, three or four groups selected from halo, alkyl, haloalkyl, hydroxyalkyl, cyano, oxo, —R^{w}—OR^{x}, —R^{w}—OR^{w}NR^{y}R^{z}, —R^{w}—NR^{y}R^{z}, —R^{w}—NR^{u}C(O)R^{v}, —R^{w}—NR^{u}S(O)ₚR^{v} and —S(O)ₚR^{v};

R⁸ is cycloalkyl, aryl, heteroaryl or heterocyclyl;

R⁹ is alkyl, —OR¹² or —NR¹³R¹⁴;

R¹⁰ and R¹¹ are each independently hydrogen, alkyl or —C(O)OR¹²;

R¹² is hydrogen, alkyl, cycloalkyl, cycloalkylalkyl, heterocyclyl or heterocyclylalkyl, wherein the alkyl, cycloalkyl, cycloalkylalkyl, heterocyclyl and heterocyclylalkyl may each be optionally substituted with one, two or three groups selected from halo, alkyl, hydroxy, alkoxy, amino, alkylthio, alkylsulfinyl and alkylsulfonyl;

each R¹³ and R¹⁴ is independently selected from (i) and (ii) below:

(i) R¹³ and R¹⁴ are each independently hydrogen, alkyl, cycloalkyl, cycloalkylalkyl, heterocyclyl or heterocyclylalkyl, wherein the alkyl, cycloalkyl, cycloalkylalkyl, heterocyclyl and heterocyclylalkyl may each be optionally substituted with one, two or three groups independently selected from halo, oxo, alkyl, hydroxy, alkoxy and amino, or

(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

each R¹⁵ is independently hydrogen or alkyl;

each R¹⁶ is independently alkylene or alkenylene;

each R¹⁷ is alkyl, alkenyl, alkynyl, cycloalkyl, cycloalkylalkyl, alkoxy, heterocyclyl, heterocyclylalkyl, aryl, aralkyl, heteroaryl or heteroaralkyl, any of which may be optionally substituted with one, two or three groups selected from halo, alkyl, hydroxy, alkoxy, amino, alkylthio and alkylsulfonyl;

each R¹⁸ is independently alkylene or a direct bond;

each R¹⁹ and R²⁰ is independently selected from (i) and (ii) below:

(i) R¹⁹ and R²⁰ are each independently hydrogen, alkyl, cycloalkyl, cycloalkylalkyl or heterocyclylalkyl; or

(ii) R¹⁹ and R²⁰, together with the nitrogen atom to which they are attached, form a heterocyclyl;

R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³, and —R¹⁸NR¹⁵C(O)R²¹; (wherein the alkyl, alkenyl, alkynyl, cycloalkyl, cycloalkylalkyl, heterocyclyl and heterocyclylalkyl may each be optionally substituted with one to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z}, and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

R²¹ is hydrogen, alkyl, haloalkyl or cycloalkyl;

R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, and —R¹⁸NR²²R²³; wherein the alkyl, alkenyl, alkynyl, cycloalkyl, cycloalkylalkyl, heterocyclyl and heterocyclylalkyl may each be optionally substituted with one to five groups independently selected from halo, cyano, oxo, alkyl, haloalkyl, hydroxyalkyl, alkoxyalkyl, cyanoalkyl, —R^{w}—OR^{x} and —R^{w}—NR^{y}r^{z}; and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z}; or two adjacent Q² groups on R⁸ may, together with the carbon atoms to which they are attached, form alkylenedioxy;

R²² and R²³ are independently selected from (i) or (ii) below:

(i) each R²² and R²³ is independently hydrogen or alkyl wherein each alkyl may each be optionally substituted by one or more substituents independently selected from the group consisting of halo, heterocyclyl, —OR^{x}, —NR^{y}R^{z} and —C(O)OR^{x}, or

(ii) R²² and R²³, together with the nitrogen atom to which they are attached, form a heterocyclyl which may optionally be substituted with one or more substituents independently selected from the group consisting of halo, oxo, alkyl, haloalkyl, hydroxyalkyl, —OR^{x}, —NR^{y}R^{z} and —C(O)OR^{x};

each R^{u} is independently hydrogen or alkyl;

each R^{v} is independently alkyl, haloalkyl, alkenyl, alkynyl or cycloalkyl;

each R^{w} is independently a direct bond or alkylene;

each R^{x} is independently hydrogen, alkyl, alkenyl, alkynyl or cycloalkyl;

each R^{y} and R^{z} is independently selected from (i) and (ii) below:

(i) R^{y} and R^{z} are each independently hydrogen, alkyl, cycloalkyl or cycloalkylalkyl, or

(ii) R^{y} and R^{z}, together with the nitrogen atom to which they are attached, form a heterocyclyl which may be optionally substituted with one or more substituents independently selected from the group consisting of halo, oxo, alkyl, haloalkyl, hydroxyalkyl, hydroxy and alkoxy; and

p is an integer selected from 0, 1 and 2.

In one embodiment, the compounds have formula (I), wherein

R¹ and R² are selected from (i), (ii), (iii) and (iv) as follows:

(i) R¹ and R² together form ═O, ═S, ═NR⁹ or ═CR¹⁰R¹¹;

(ii) R¹ and R² together with the carbon atom to which they are attached, form cycloalkyl or oxacycloalkyl;

(iii) R¹ and R² are each independently hydrogen, halo, haloalkyl, alkyl, alkenyl, alkynyl, cycloalkyl, hydroxyalkyl, alkoxyalkyl, —OR¹² or —SR¹²; or

(iv) R¹ is —OR¹², —NR¹³R¹⁴, —SR¹², —N(R¹⁵)R¹⁶OR¹² or —R¹⁸C(O)OR¹², and R² is hydrogen, cyano, haloalkyl, alkyl, alkenyl, alkynyl, cycloalkyl, hydroxyalkyl or alkoxyalkyl;

R³ is cycloalkyl, aryl, heterocyclyl or heteroaryl;

R⁴ is hydrogen or alkyl;

R⁵, R⁶ and R⁷ are each independently hydrogen, halo, nitro, alkyl, alkenyl, alkynyl or cycloalkyl, —OR^{x}, —NR¹⁵C(O)R¹⁷, —C(O)NR¹⁹R²⁰ wherein the alkyl, alkenyl, alkynyl and cycloalkyl may each be optionally substituted with one, two or three groups selected from halo, alkyl, haloalkyl, hydroxyalkyl, cyano, —R^{w}—OR^{x} and —R^{w}—NR^{y}R^{z};

R⁸ is cycloalkyl, aryl, heteroaryl or heterocyclyl;

R⁹ is alkyl, —OR¹² or —NR¹³R¹⁴;

R¹⁰ and R¹¹ are each independently hydrogen, alkyl or —C(O)OR¹²;

R¹² is hydrogen, alkyl, cycloalkyl, cycloalkylalkyl, heterocyclyl or heterocyclylalkyl wherein the alkyl, cycloalkyl, cycloalkylalkyl heterocyclyl or heterocyclylalkyl may each be optionally substituted with one, two or three groups selected from halo, alkyl, hydroxy, alkoxy and amino;

each R¹³ and R¹⁴ is independently selected from (i) or (ii) below:

(i) R¹³ and R¹⁴ are each independently hydrogen, alkyl, cycloalkyl, cycloalkylalkyl, heterocyclyl or heterocyclylalkyl wherein the alkyl, cycloalkyl, cycloalkylalkyl, heterocyclyl or heterocyclylalkyl may each be optionally substituted with one, two or three groups independently selected from halo, oxo, alkyl, hydroxy, alkoxy and amino, or

(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

each R¹⁵ is independently hydrogen or alkyl;

each R¹⁶ is independently alkylene or alkenylene;

each R¹⁷ is alkyl, alkenyl, alkynyl, cycloalkyl, cycloalkylalkyl, heterocyclyl, heterocyclylalkyl, aryl, aralkyl, heteroaryl or heteroaralkyl;

each R¹⁸ is independently alkylene or a direct bond;

each R¹⁹ and R²⁰ is independently selected from (i) or (ii) below:

(i) R¹⁹ and R²⁰ are each independently hydrogen, alkyl, cycloalkyl or

cycloalkylalkyl wherein the alkyl, cycloalkyl or cycloalkylalkyl may each be optionally substituted with one, two or three groups independently selected from halo, oxo, alkyl, haloalkyl, hydroxyalkyl, hydroxy, alkoxy and amino, or

(ii) R¹⁹ and R²⁰, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³, —R¹⁸NR¹⁵C(O)R²¹; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

each R²¹ is independently hydrogen, alkyl, haloalkyl, hydroxyalkyl, alkenyl, alkynyl, cycloalkyl, cycloalkylalkyl, heterocyclyl, heterocyclylalkyl, aryl, aralkyl, heteroaryl or heteroaralkyl;

R²² and R²³ are independently selected from (i) or (ii) below:

(i) each R²² and R²³ is independently hydrogen or alkyl wherein the alkyl may each be optionally substituted by one or more substituents independently selected from the group consisting of halo, heterocyclyl, —OR^{x}, —NR^{y}R^{z} and —C(O)OR^{x}, or

(ii) R²² and R²³, together with the nitrogen atom to which they are attached, form a heterocyclyl which may be optionally substituted by one or more substituents independently selected from the group consisting of halo, oxo, alkyl, haloalkyl, hydroxyalkyl, —OR^{x}, —NR^{y}R^{z} and —C(O)OR^{x};

each R^{w} is independently a direct bond or alkylene;

each R^{x} is independently hydrogen, alkyl, alkenyl, alkynyl or cycloalkyl; and

each R^{y} and R^{z} is independently selected from (i) or (ii) below:

(i) R^{y} and R^{z} are each independently hydrogen, alkyl, cycloalkyl or cycloalkylalkyl, or

(ii) R^{y} and R^{z}, together with the nitrogen atom to which they are attached, form a heterocyclyl which may be optionally substituted by one or more substituents independently selected from the group consisting of halo, oxo, alkyl, haloalkyl, hydroxyalkyl, hydroxy and alkoxy.

In one embodiment, the compound provided herein is a compound of formula (I). In one embodiment, the compound provided herein is a pharmaceutically acceptable salt of the compound of formula (I). In one embodiment, the compound provided herein is a solvate of the compound of formula (I). In one embodiment, the compound provided herein is a hydrate of compound of formula (I). In one embodiment, the compound provided herein is a prodrug of the compound of formula (I). In one embodiment, the compound provided herein is a clathrate of the compound of formula (I).

Also provided are pharmaceutical compositions formulated for administration by an appropriate route and means containing effective concentrations of one or more of the compounds provided herein, or pharmaceutically acceptable salts, solvates, hydrates and prodrugs thereof, and optionally comprising at least one pharmaceutical carrier.

Such pharmaceutical compositions deliver amounts effective for the treatment, prevention, or amelioration of diseases or disorders that are modulated or otherwise affected by JAK kinases, including JAK2 kinase, or one or more symptoms or causes thereof. Such diseases or disorders include without limitation, cancer, including myeloproliferative disorders such as polycythemia vera (PCV), essential thrombocythemia (ET), primary myelofibrosis (PMF), chronic eosinophilic leukemia (CEL), chronic myelomonocytic leukemia (CMML), systemic mastocytosis (SM) and idiopathic myelofibrosis (IMF), as well as myeloid leukemia including chronic myeloid leukemia (CML), imatinib-resistant forms of CML, acute myeloid leukemia (AML), and a subtype of AML, acute megakaryoblastic leukemia (AMKL); lymphoproliferative diseases such as myeloma; other cancers such as cancer of the head and neck, prostate cancer, breast cancer, ovarian cancer, melanoma, lung cancers, brain tumors, pancreatic cancer and renal cancer; and inflammatory diseases or disorders related to immune dysfunction, immunodeficiency, immunomodulation, autoimmune diseases, tissue transplant rejection, graft-versus-host disease, wound healing, kidney disease, multiple sclerosis, thyroiditis, type 1 diabetes, sarcoidosis, psoriasis, allergic rhinitis, inflammatory bowel disease including Crohn's disease and ulcerative colitis (UC), systemic lupus erythematosis (SLE), arthritis, osteoarthritis, rheumatoid arthritis, osteoporosis, asthma and chronic obstructive pulmonary disease (COPD).

Also provided herein are combination therapies using one or more compounds or compositions provided herein, or pharmaceutically acceptable derivatives thereof, in combination with other pharmaceutically active agents for the treatment of the diseases and disorders described herein.

In one embodiment, such additional pharmaceutical agents include one or more chemotherapeutic agents, anti-proliferative agents, anti-inflammatory agents, immunomodulatory agents or immunosuppressive agents.

The compounds or compositions provided herein, or pharmaceutically acceptable derivatives thereof, may be administered simultaneously with, prior to, or after administration of one or more of the above agents. Pharmaceutical compositions containing a compound provided herein and one or more of the above agents are also provided.

In certain embodiments, provided herein are methods of treating, preventing or ameliorating a disease or disorder that is modulated or otherwise affected by JAK kinases, including JAK2 kinase such as wild type and/or mutant JAK2 kinase, or one or more symptoms or causes thereof. In another embodiment, provided herein are methods of treating, preventing or ameliorating a disease or disorder by modulating the JAK2 kinase selectively over JAK3 kinase. In yet another embodiment, provided herein are methods of treating, preventing or ameliorating a disease or disorder by modulating the JAK3 kinase selectively over JAK2 kinase. In another embodiment, provided herein are methods of treating, preventing or amerliorating a disease or disorder by modulating both JAK2 and JAK3.

In practicing the methods, effective amounts of the compounds or compositions containing therapeutically effective concentrations of the compounds, which are formulated for systemic delivery, including parenteral, oral, or intravenous delivery, or for local or topical application are administered to an individual exhibiting the symptoms of the disease or disorder to be treated. The amounts are effective to ameliorate or eliminate one or more symptoms of the disease or disorder.

Further provided is a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like.

These and other aspects of the subject matter described herein will become evident upon reference to the following detailed description.

### DETAILED DESCRIPTION

Provided herein are compounds of formula (I) that have activity as JAK kinase, including JAK2 kinase, modulators. Further provided are methods of treating, preventing or ameliorating diseases that are modulated by JAK kinases, including JAK2 kinase, and pharmaceutical compositions and dosage forms useful for such methods. The methods and compositions are described in detail in the sections below.

In certain embodiments, the compounds provided herein are JAK2 selective, i.e., the compounds bind or interact with JAK2 at substantially lower concentrations than they bind or interact with other JAK receptors, including JAK3 receptor, at that same concentration. In certain embodiments, the compounds bind to JAK3 receptor at a binding constant at least about 3-fold higher, about 5-fold higher, about 10-fold higher, about 20-fold higher, about 25-fold higher, about 50-fold higher, about 75-fold higher, about 100-fold higher, about 200-fold higher, about 225-fold higher, about 250 fold higher, about 300 fold higher, than they bind JAK2 receptor.

In certain embodiments, the compounds provided herein are JAK3 selective, i.e., the compounds bind or interact with JAK3 at substantially lower concentrations than they bind or interact with other JAK receptors, including JAK2 receptor, at that same concentration. In certain embodiments, the compounds bind to JAK2 receptor at a binding constant at least about 3-fold higher, about 5-fold higher, about 10-fold higher, about 20-fold higher, about 25-fold higher, about 50-fold higher, about 75-fold higher, about 100-fold higher, about 200-fold higher, about 225-fold higher, about 250 fold higher, about 300 fold higher than they bind with JAK3 receptor.

### A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

“Alkyl” refers to a straight or branched hydrocarbon chain group consisting solely of carbon and hydrogen atoms, containing no unsaturation, having from one to ten, one to eight, one to six or one to four carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like.

“Alkenyl” refers to a straight or branched hydrocarbon chain group consisting solely of carbon and hydrogen atoms, containing at least one double bond, having from two to ten carbon atoms, and which is attached to the rest of the molecule by a single bond or a double bond, e.g., ethenyl, prop-1-enyl, but-1-enyl, pent-1-enyl, penta-1,4-dienyl, and the like.

“Alkynyl” refers to a straight or branched hydrocarbon chain group consisting solely of carbon and hydrogen atoms, containing at least one triple bond, having from two to ten carbon atoms, and which is attached to the rest of the molecule by a single bond or a triple bond, e.g., ethynyl, prop-1-ynyl, but-1-ynyl, pent-1-ynyl, pent-3-ynyl and the like.

“Alkylene” and “alkylene chain” refer to a straight or branched divalent hydrocarbon chain consisting solely of carbon and hydrogen, containing no unsaturation and having from one to eight carbon atoms, e.g., methylene, ethylene, propylene, n-butylene and the like. The alkylene chain may be attached to the rest of the molecule through any two carbons within the chain.

“Alkoxy” refers to the group having the formula —OR wherein R is alkyl or haloalkyl. An “optionally substituted alkoxy” refers to the group having the formula —OR wherein R is an optionally substituted alkyl as defined herein.

“Alkylsulfonyl” refers to the group having the formula —S(O)₂R wherein R is alkyl or haloalkyl.

“Alkylthio” refers to a group having the formula —SR wherein R is alkyl or haloalkyl.

“Amine” or “amino” refers to a group having the formula —NR′R″ wherein R′ and R″ are each independently hydrogen, alkyl, haloalkyl, hydroxyalkyl or alkoxyalkyl or wherein R′ and R″, together with the nitrogen atom to which they are attached form a heterocyclyl optionally substituted with halo, oxo, hydroxy or alkoxy.

“Aryl” refers to a group of carbocylic ring system, including monocyclic, bicyclic, tricyclic, tetracyclic C₆-C₁₈ ring systems, wherein at least one of the rings is aromatic. The aryl may be fully aromatic, examples of which are phenyl, naphthyl, anthracenyl, acenaphthylenyl, azulenyl, fluorenyl, indenyl and pyrenyl. The aryl may also contain an aromatic ring in combination with a non-aromatic ring, examples of which are acenaphene, indene, and fluorene.

“Cycloalkyl” refers to a stable monovalent monocyclic or bicyclic hydrocarbon group consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl, norbornane, norbornene, adamantyl, bicyclo[2.2.2]octane and the like.

“Cycloalkylalkyl” refers to a group of the formula —RₐR_{d} where Rₐ is an alkyl group as defined above and R_{d} is a cycloalkyl group as defined above. The alkyl group and the cylcoalkyl group may be optionally substituted as defined herein.

“Halo, “halogen” or “halide” refers to F, Cl, Br or I.

“Haloalkyl” refers to an alkyl group, in certain embodiments, C₁₋₆alkyl group in which one or more of the hydrogen atoms are replaced by halogen. Such groups include, but are not limited to, chloromethyl, trifluoromethyl 1-chloro-2-fluoroethyl, 2,2-difluoroethyl, 2-fluoropropyl, 2-fluoropropan-2-yl, 2,2,2-trifluoroethyl, 1,1-difluoroethyl, 1,3-difluoro-2-methylpropyl, 2,2-difluorocyclopropyl, (trifluoromethyl)cyclopropyl, 4,4-difluorocyclohexyl and 2,2,2-trifluoro-1,1-dimethyl-ethyl.

“Heterocyclyl” refers to a stable 3- to 15-membered non-aromatic ring radical which consists of carbon atoms and from one to five heteroatoms selected from a group consisting of nitrogen, oxygen and sulfur. In one embodiment, the heterocyclic ring system radical may be a monocyclic, bicyclic or tricyclic ring or tetracyclic ring system, which may include fused or bridged ring systems; and the nitrogen or sulfur atoms in the heterocyclic ring system radical may be optionally oxidized; the nitrogen atom may be optionally quaternized; and the heterocyclyl radical may be partially or fully saturated. The heterocyclic ring system may be attached to the main structure at any heteroatom or carbon atom which results in the creation of a stable compound. Exemplary heterocylic radicals include, azetidinyl, benzo[1,3]dioxol-5-yl, benzodioxolyl, 1,3-dioxolan-2-yl, dioxolanyl, morpholinyl, tetrahydrofuran, oxazolidin-2-onyl, oxazolidinonyl, piperidinyl, piperazinyl, pyranyl, tetrahydropyranyl, pyrrolidinonyl, oxathiolanyl, and pyrrolidinyl.

“Heteroaryl” refers to a heterocyclyl group as defined above which is aromatic. The heteroaryl group may be attached to the main structure at any heteroatom or carbon atom which results in the creation of a stable compound. Examples of such heteroaryl groups include, but are not limited to: acridinyl, benzimidazolyl, benzindolyl, benzisoxazinyl, benzo[4,6]imidazo[1,2-a]pyridinyl, benzofuranyl, benzonaphthofuranyl, benzothiadiazolyl, benzothiazolyl, benzothiophenyl, benzotriazolyl, benzothiopyranyl, benzoxazinyl, benzoxazolyl, benzothiazolyl, β-carbolinyl, carbazolyl, cinnolinyl, dibenzofuranyl, furanyl, imidazolyl, imidazopyridinyl, imidazothiazolyl, indazolyl, indolizinyl, indolyl, isobenzothienyl, isoindolinyl, isoquinolinyl, isothiazolidinyl, isothiazolyl, naphthyridinyl, octahydroindolyl, octahydroisoindolyl, oxazolidinonyl, oxazolidinyl, oxazolopyridinyl, oxazolyl, isoxazolyl, oxiranyl, perimidinyl, phenanthridinyl, phenathrolinyl, phenarsazinyl, phenazinyl, phenothiazinyl, phenoxazinyl, phthalazinyl, pteridinyl, purinyl, pyrazinyl, pyrazolyl, pyridazinyl, pyridinyl, pyridopyridinyl, pyrimidinyl, pyrrolyl, quinazolinyl, quinolinyl, quinoxalinyl, tetrazolyl, thiadiazolyl, thiazolyl, thienyl, triazinyl and triazolyl.

In certain embodiments, the heterocyclic or heteroaryl radicals include, but are not limited to: acridinyl, azepinyl, benzimidazolyl, benzindolyl, benzoisoxazolyl, benzisoxazinyl, benzo[4,6]imidazo[1,2-a]pyridinyl, benzodioxanyl, benzodioxolyl, benzofuranonyl, benzofuranyl, benzonaphthofuranyl, benzopyranonyl, benzopyranyl, benzotetrahydrofuranyl, benzotetrahydrothienyl, benzothiadiazolyl, benzothiazolyl, benzothiophenyl, benzotriazolyl, benzothiopyranyl, benzoxazinyl, benzoxazolyl, benzothiazolyl, β-carbolinyl, carbazolyl, chromanyl, chromonyl, cinnolinyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4-dithianyl, furanonyl, furanyl, imidazolidinyl, imidazolinyl, imidazolyl, imidazopyridinyl, imidazothiazolyl, indazolyl, indolinyl, indolizinyl, indolyl, isobenzotetrahydrofuranyl, isobenzotetrahydrothienyl, isobenzothienyl, isochromanyl, isocoumarinyl, isoindolinyl, isoindolyl, isoquinolinyl, isothiazolidinyl, isothiazolyl, isoxazolidinyl, isoxazolyl, morpholinyl, naphthyridinyl, octahydroindolyl, octahydroisoindolyl, oxadiazolyl, oxazolidinonyl, oxazolidinyl, oxazolopyridinyl, oxazolyl, oxiranyl, perimidinyl, phenanthridinyl, phenathrolinyl, phenarsazinyl, phenazinyl, phenothiazinyl, phenoxazinyl, phthalazinyl, piperazinyl, piperidinyl, 4-piperidonyl, pteridinyl, purinyl, pyrazinyl, pyrazolidinyl, pyrazolyl, pyridazinyl, pyridinyl, pyridopyridinyl, pyrimidinyl, pyrrolidinyl, pyrrolinyl, pyrrolyl, quinazolinyl, quinolinyl, quinoxalinyl, quinuclidinyl, tetrahydrofuryl, tetrahydrofuranyl, tetrahydroisoquinolinyl, tetrahydropyranyl, tetrahydrothienyl, tetrazolyl, thiadiazolopyrimidinyl, thiadiazolyl, thiamorpholinyl, thiazolidinyl, thiazolyl, thienyl, triazinyl, triazolyl and 1,3,5-trithianyl.

“Hydroxyalkyl” refers to a alkyl group, in certain embodiments, C₁₋₆alkyl group in which one or more of the hydrogen atoms are replaced by a hydroxy group.

“Alkylenedioxy” refers to a group of the formula —O—(CH₂)_{q}O— wherein q is an integer selected from 1, 2 and 3.

“Aminoalkyl” refers to a group of the formula —RₐR_{g} where Rₐ is an alkyl group as defined above, substituted by R_{g}, an amino group having the formula —NR′R″ also as defined above.

“Aralkyl” refers to a group of the formula —RₐR_{g} where Rₐ is an alkyl group as defined above, substituted by R_{g}, an aryl group, as defined above, e.g., benzyl. Both the alkyl and aryl groups may be optionally substituted as defined herein.

“Heteroaralkyl” refers to a group of the formula —RₐR_{f} where Rₐ is an alkyl group as defined above and R_{f} is a heteroaryl group as defined herein. The alkyl group and the heteroaryl group may be optionally substituted as defined herein.

“Heterocyclylalkyl” refers to a group of the formula —RₐR_{c} wherein Rₐ is an alkyl group as defined above and Rₑ is a heterocyclyl group as defined herein, where the alkyl groUp Rₐ may attach at either the carbon atom or the heteroatom of the heterocyclyl group Rₑ. The alkyl group and the heterocyclyl group may be optionally substituted as defined herein.

The term “azolyl” as used herein means a five-membered saturated or unsaturated heterocyclic group containing one or more hetero-atoms, as ring atoms, selected from the group consisting of nitrogen, sulfur, and oxygen atoms, wherein at least one of the hetero-atoms is a nitrogen atom. Examples of azolyl groups include, but are not limited to, imidazolyl, oxazolyl, isoxazolyl, pyrrolyl, tetrazolyl, thiadiazolyl, thiazolyl, and triazolyl.

The term “azinyl” as used herein means a six-membered saturated or unsaturated heterocyclic group containing one or more hetero-atoms, as ring atoms, selected from the group consisting of nitrogen, sulfur, and oxygen atoms, wherein at least one of the hetero-atoms is a nitrogen atom. Examples of azolyl groups include, but are not limited to, pyrazinyl, pyrazolyl, pyridazinyl, pyridinyl, pyrimidinyl, and triazinyl.

“IC₅₀” refers to an amount, concentration or dosage of a particular test compound that achieves a 50% inhibition of a maximal response, such as cell growth or proliferation measured via any the in vitro or cell based assay described herein.

“Imine” or “imino” refers to the group ═NR_{g} attached to a carbon atom where R_{g} may be hydrogen or optionally substituted alkyl.

The term “oxacycloalkyl” as used herein means a heterocyclic group containing one or two oxygen ring atom and two or more carbon ring atoms.

“Oxime” refers to the group ═N(OR^{j}) attached to a carbon atom wherein Rⱼ may be hydrogen or optionally substituted alkyl.

“Oxo” refers to the group ═O attached to a carbon atom.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

As used herein and unless otherwise indicated, the term “hydrate” means a compound provided herein or a salt thereof, that further includes a stoichiometric or non-stoichiometeric amount of water bound by non-covalent intermolecular forces.

As used herein and unless otherwise indicated, the term “solvate” means a solvate formed from the association of one or more solvent molecules to a compound provided herein. The term “solvate” includes hydrates (e.g., mono-hydrate, dihydrate, trihydrate, tetrahydrate and the like).

“Prodrug” is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (2005) Medicinal Chemistry A Biochemical Approach, Oxford University Press, New York).

As used herein, “substantially pure” means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

“Thioxo” refers to the group ═S attached to a carbon atom.

Unless stated otherwise specifically described in the specification, it is understood that the substitution can occur on any atom of the alkyl, alkenyl, alkynyl, cycloalkyl, heterocyclyl, aryl or heteroaryl group.

Unless specifically stated otherwise, where a compound may assume alternative tautomeric, regioisomeric and/or stereoisomeric forms, all alternative isomers are intended to be encompassed within the scope of the claimed subject matter. For example, where a compound is described as having one of two tautomeric forms, it is intended that the both tautomers be encompassed herein.

Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

It is to be understood that the compounds provided herein may contain chiral centers. Such chiral centers may be of either the (R) or (8) configuration, or may be a mixture thereof.

Optically active (+) and (−), (R)- and (S)-, or (D)- and (L)-isomers may be prepared using chiral synthons or chiral reagents, or resolved using conventional techniques, such as reverse phase HPLC.

As used herein, the term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the desired enantiomer.

Where the number of any given substituent is not specified (e.g., haloalkyl), there may be one or more substituents present. For example, “haloalkyl” may include one or more of the same or different halogens.

In the description herein, if there is any discrepancy between a chemical name and chemical structure, the structure preferably controls.

“Anti-cancer agents” refers to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxrubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent® and Bevacizumab) or any other cytotoxic agents, (estramustine phosphate, prednimustine), hormones or hormone agonists, antagonists, partial agonists or partial antagonists, kinase inhibitors, and radiation treatment.

“Anti-inflammatory agents” refers to matrix metalloproteinase inhibitors, inhibitors of pro-inflammatory cytokines (e.g., anti-TNF molecules, TNF soluble receptors, and IL1) non-steroidal anti-inflammatory drugs (NSAIDs) such as prostaglandin synthase inhibitors (e.g., choline magnesium salicylate, salicylsalicyclic acid), COX-1 or COX-2 inhibitors), or glucocorticoid receptor agonists such as corticosteroids, methylprednisone, prednisone, or cortisone.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 1972, 11:942-944).

### B. Compounds

In one embodiment, provided herein are compounds of formula (I), wherein:

R¹ and R² are selected from (i), (ii), (iii) and (iv) as follows:

(i) R¹ and R² together form ═O, ═S, or ═NR⁹;

(ii) R¹ and R² together with the carbon atom to which they are attached, form cycloalkyl;

(iii) R¹ and R² are each independently hydrogen, halo, haloalkyl, alkyl, alkenyl, alkynyl, cycloalkyl, hydroxyalkyl or alkoxyalkyl; or

(iv) R¹ is —OR¹² and R² is hydrogen, haloalkyl, alkyl, alkenyl, alkynyl, cycloalkyl, hydroxyalkyl or alkoxyalkyl;

R³ is cycloalkyl, aryl, heterocyclyl or heteroaryl;

R⁴ is hydrogen or alkyl;

R⁵, R⁶ and R⁷ are each independently hydrogen, halo, alkyl, haloalkyl, hydroxyalkyl or alkoxyalkyl;

R⁸ is cycloalkyl, aryl, heteroaryl or heterocyclyl;

R⁹ is alkyl or hydroxy;

R¹² is hydrogen or alkyl;

R³ and R⁸ are optionally substituted with one or more substituents independently selected from the group Q¹ consisting of nitro, halo, cyano, oxo, thioxo, imino, amino, alkyl, alkenyl, alkynyl or cycloalkyl; wherein the alkyl, alkenyl, alkynyl and cycloalkyl are each optionally substituted with one to five groups selected from halo, cyano, alkyl, haloalkyl, hydroxyalkyl, alkoxyalkyl, cyanoalkyl, alkoxy and hydroxyl.

In one embodiment, the compounds provided herein have formula (I), wherein:

R¹ and R² are selected from (i), (ii), (iii) and (iv) as follows:

(i) R¹ and R² together form ═O, ═S, ═NR⁹ or ═CR¹⁰R¹¹;

(ii) R¹ and R² together with the carbon atom to which they are attached, form oxacycloalkyl;

(iii) R¹ and R² are each independently hydrogen or halo; or

(iv) R¹ is —OR¹², —NR¹³R¹⁴, —SR¹²—N(R¹⁵)R¹⁶OR¹² or —R¹⁸C(O)OR¹², and R² is hydrogen, alkyl, haloalkyl, hydroxyalkyl, alkoxyalkyl or cycloalkyl;

R³ is cycloalkyl, aryl, heterocyclyl or heteroaryl;

R⁴ is hydrogen or alkyl;

R⁵, R⁶ and R⁷ are each independently hydrogen, halo, alkyl, —OR¹⁷, —NR¹⁵C(O)R¹⁷ or —C(O)NR¹⁹R²⁰ wherein the alkyl is optionally substituted with one, two or three groups selected from halo, alkyl, haloalkyl, hydroxyalkyl, cyano, —R^{w}—OR^{x} and —R^{w}—NR^{y}R^{z};

R⁸ is cycloalkyl, aryl, heteroaryl or heterocyclyl;

R⁹ is alkyl, —OR¹² or —NR¹³R¹⁴;

R¹⁰ and R¹¹ are each independently hydrogen, alkyl or —C(O)OR¹²;

R¹² is hydrogen or alkyl;

each R¹³ and R¹⁴ is independently selected from (i) or (ii) below:

(i) R¹³ and R¹⁴ are each independently hydrogen, alkyl, cycloalkyl, cycloalkylalkyl, heterocyclyl or heterocyclylalkyl wherein the alkyl, cycloalkyl, cycloalkylalkyl, heterocyclyl or heterocyclylalkyl may each be optionally substituted with one, two or three groups independently selected from halo, oxo, alkyl, hydroxy, alkoxy and amino, or

(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

each R¹⁵ is independently hydrogen or alkyl;

each R¹⁶ is independently alkylene or alkenylene;

each R¹⁷ is alkyl, alkenyl, alkynyl, cycloalkyl, cycloalkylalkyl, heterocyclyl, heterocyclylalkyl, aryl, aralkyl, heteroaryl or heteroaralkyl;

each R¹⁸ is independently alkylene or a direct bond;

each R¹⁹ and R²⁰ is independently selected from (i) or (ii) below:

(i) R¹⁹ and R²⁰ are each independently hydrogen, alkyl, cycloalkyl or cycloalkylalkyl wherein the alkyl, cycloalkyl or cycloalkylalkyl may each be optionally substituted with one, two or three groups independently selected from halo, oxo, alkyl, haloalkyl, hydroxyalkyl, hydroxy, alkoxy and amino, or

(ii) R¹⁹ and R²⁰, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³, —R¹⁸NR¹⁵C(O)R²¹; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

each R²¹ is independently hydrogen, alkyl, haloalkyl, hydroxyalkyl, alkenyl, alkynyl, cycloalkyl, cycloalkylalkyl, heterocyclyl, heterocyclylalkyl, aryl, aralkyl, heteroaryl or heteroaralkyl;

R²² and R²³ are independently selected from (i) or (ii) below:

(i) each R²² and R²³ is independently hydrogen or alkyl wherein the alkyl may each be optionally substituted with one or more substituents independently selected from the group consisting of halo, heterocyclyl, —OR^{x}, —NR^{y}R^{z} and —C(O)OR^{x}, or

(ii) R²² and R²³, together with the nitrogen atom to which they are attached, form a heterocyclyl which may be optionally substituted with one or more substituents independently selected from the group consisting of halo, oxo, alkyl, haloalkyl, hydroxyalkyl, —OR^{x}, —NR^{y}R^{z} and —C(O)OR^{x};

each R^{w} is independently a direct bond or alkylene;

each R^{x} is independently hydrogen, alkyl, alkenyl, alkynyl or cycloalkyl; and

each R^{y} and R^{z} is independently selected from (i) or (ii) below:

(i) R^{y} and R^{z} are each independently hydrogen, alkyl, cycloalkyl or cycloalkylalkyl, or

(ii) R^{y} and R^{z}, together with the nitrogen atom to which they are attached, form a heterocyclyl which may be optionally substituted with one or more substituents independently selected from the group consisting of halo, oxo, alkyl, haloalkyl, hydroxyalkyl, hydroxy and alkoxy.

In one embodiment, the compounds for use in the compositions and methods provided herein have formula (I), wherein:

R¹ and R² are selected from (i), (ii) or (iii) as follows:

(i) R¹ and R² together form ═O, ═S, ═NR⁹ or ═CR¹⁰R¹¹;

(ii) R¹ and R² are each independently hydrogen or halo; or

(iii) R¹ is —OR¹², —NR¹³R¹⁴, —SR¹²—N(R¹⁵)R¹⁶OR¹² or —R¹⁸C(O)OR¹², and R² is hydrogen, cyano, alkyl, haloalkyl, hydroxyalkyl or alkoxyalkyl or cycloalkyl;

R³ is cycloalkyl, aryl, heterocyclyl or heteroaryl;

R⁴ is hydrogen or alkyl;

R⁵, R⁶ and R⁷ are each independently hydrogen, halo, alkyl, —OR^{x}, —NR¹⁵C(O)R¹⁷ or —C(O)NR¹⁹R²⁰ wherein the alkyl is optionally substituted with one, two or three groups selected from halo, alkyl, haloalkyl, hydroxyalkyl, cyano, —R^{w}—OR^{x} and —R^{w}—NR^{y}R^{z};

R⁸ is cycloalkyl, aryl, heteroaryl or heterocyclyl;

R⁹ is alkyl, —OR¹² or —NR¹³R¹⁴;

R¹⁰ and R¹¹ are each independently hydrogen, alkyl or —C(O)OR¹²;

R¹² is hydrogen or alkyl;

each R¹³ and R¹⁴ is independently selected from (i) or (ii) below:

(i) R¹³ and R¹⁴ are each independently hydrogen, alkyl, cycloalkyl, cycloalkylalkyl, heterocyclyl or heterocyclylalkyl wherein the alkyl, cycloalkyl, cycloalkylalkyl, heterocyclyl or heterocyclylalkyl may each be optionally substituted with one, two or three groups independently selected from halo, oxo, alkyl, hydroxy, alkoxy and amino, or

(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

each R¹⁵ is independently hydrogen or alkyl;

each R¹⁶ is independently alkylene or alkenylene;

each R¹⁷ is alkyl, alkenyl, alkynyl, cycloalkyl, cycloalkylalkyl, heterocyclyl, heterocyclylalkyl, aryl, aralkyl, heteroaryl or heteroaralkyl;

each R¹⁸ is alkylene;

each R¹⁹ and R²⁰ is independently selected from (i) or (ii) below:

(i) R¹⁹ and R²⁰ are each independently hydrogen, alkyl, cycloalkyl or cycloalkylalkyl wherein the alkyl, cycloalkyl or cycloalkylalkyl may each be optionally substituted with one, two or three groups independently selected from halo, oxo, alkyl, haloalkyl, hydroxyalkyl, hydroxy, alkoxy and amino, or

(ii) R¹⁹ and R²⁰, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³, —R¹⁸NR¹⁵C(O)R²¹; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

each R²¹ is independently hydrogen, alkyl, haloalkyl, hydroxyalkyl, alkenyl, alkynyl, cycloalkyl, cycloalkylalkyl, heterocyclyl, heterocyclylalkyl, aryl, aralkyl, heteroaryl or heteroaralkyl;

R²² and R²³ are independently selected from (i) or (ii) below:

(iii) each R²² and R²³ is independently hydrogen or alkyl wherein the alkyl may each be optionally substituted with one or more substituents independently selected from the group consisting of halo, heterocyclyl, —OR^{x}, —NR^{y}r^{z} and —C(O)OR^{x}, or

(iv) R²² and R²³, together with the nitrogen atom to which they are attached, form a heterocyclyl which may be optionally substituted with one or more substituents independently selected from the group consisting of halo, oxo, alkyl, haloalkyl, hydroxyalkyl, —OR^{x}, —NR^{y}R^{z} and —C(O)OR^{x}; each R^{w} is independently a direct bond or alkylene;

each R^{x} is independently hydrogen, alkyl, alkenyl, alkynyl or cycloalkyl; and

each R^{y} and R^{z} is independently selected from (i) or (ii) below:

(i) R^{y} and R^{z} are each independently hydrogen, alkyl, cycloalkyl or cycloalkylalkyl, or

(ii) R^{y} and R^{z}, together with the nitrogen atom to which they are attached, form a heterocyclyl which may be optionally substituted with one or more substituents independently selected from the group consisting of halo, oxo, alkyl, haloalkyl, hydroxyalkyl, hydroxy and alkoxy.

In one embodiment, the compounds for use in the compositions and methods provided herein have formula (I), wherein:

R¹ and R² are selected from (i), (ii) and (iii) as follows:

(i) R¹ and R² together form ═O;

(ii) R¹ and R² are each independently hydrogen or halo; or

(iii) R¹ is —OR¹², —NR¹³R¹⁴, —SR¹², —N(R¹⁵)R¹⁶OR¹² or —R¹⁸C(O)OR¹², and R² is hydrogen, cyano, alkyl, haloalkyl, hydroxyalkyl or alkoxyalkyl or cycloalkyl;

R³ is cycloalkyl, aryl, heterocyclyl or heteroaryl;

R⁴ is hydrogen or alkyl;

R⁵, R⁶ and R⁷ are each independently hydrogen, halo, alkyl, —OR¹⁷, —NR¹⁵C(O)R¹⁷ or —C(O)NR¹⁹R²⁰ wherein the alkyl is optionally substituted with one, two or three groups selected from halo, alkyl, haloalkyl, hydroxyalkyl, cyano, —R^{w}—OR^{x} and —R^{w}—NR^{y}R^{z};

R¹² is hydrogen or alkyl;

each R¹³ and R¹⁴ is independently selected from (i) or (ii) below:

(i) R¹³ and R¹⁴ are each independently hydrogen, alkyl, cycloalkyl, cycloalkylalkyl, heterocyclyl or heterocyclylalkyl wherein the alkyl, cycloalkyl, cycloalkylalkyl, heterocyclyl or heterocyclylalkyl may each be optionally substituted with one, two or three groups independently selected from halo, oxo, alkyl, hydroxy, alkoxy and amino, or

(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

each R¹⁵ is independently hydrogen or alkyl;

each R¹⁶ is independently alkylene or alkenylene;

each R¹⁷ is alkyl, alkenyl, alkynyl, cycloalkyl, cycloalkylalkyl, heterocyclyl, heterocyclylalkyl, aryl, aralkyl, heteroaryl or heteroaralkyl;

each R¹⁸ is independently alkylene or a direct bond;

each R¹⁹ and R²⁰ is independently selected from (i) or (ii) below:

(i) R¹⁹ and R²⁰ are each independently hydrogen, alkyl, cycloalkyl or cycloalkylalkyl wherein the alkyl, cycloalkyl or cycloalkylalkyl may each be optionally substituted with one, two or three groups independently selected from halo, oxo, alkyl, haloalkyl, hydroxyalkyl, hydroxy, alkoxy and amino, or

(ii) R¹⁹ and R²⁰, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³, —R¹⁸NR¹⁵C(O)R²¹; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, and —R^{w}—NR^{y}R^{z};

R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z}; and R²¹, R²², R²³, R^{w}, R^{x}, R^{y} and R^{z} are as described elsewhere herein.

In one embodiment, the compounds for use in the compositions and methods provided herein have formula (I), wherein:

R¹ and R² are selected from (i), (ii) and (iii) as follows:

(i) R¹ and R² together form ═O;

(ii) R¹ and R² are each independently hydrogen or halo; or

(iii) R¹ is —OR¹², —NR¹³R¹⁴ or —N(R¹⁵)C(O)OR¹² and R² is hydrogen, cyano, alkyl, haloalkyl, hydroxyalkyl or alkoxyalkyl or cycloalkyl;

R³ is cycloalkyl, aryl, heterocyclyl or heteroaryl;

R⁴ is hydrogen or alkyl;

R⁵, R⁶ and R⁷ are each independently hydrogen, halo, alkyl, —OR¹⁷, —NR¹⁵C(O)R¹⁷ or —C(O)NR¹⁹R²⁰ wherein the alkyl is optionally substituted with one, two or three groups selected from halo, alkyl, haloalkyl, hydroxyalkyl, cyano, —R^{w}—OR^{x} and —R^{w}—NR^{y}R^{z};

R¹² is hydrogen or alkyl;

each R¹³ and R¹⁴ is independently selected from (i) or (ii) below:

(i) R¹³ and R¹⁴ are each independently hydrogen, alkyl, cycloalkyl, cycloalkylalkyl, heterocyclyl or heterocyclylalkyl wherein the alkyl, cycloalkyl, cycloalkylalkyl, heterocyclyl or heterocyclylalkyl may each be optionally substituted with one, two or three groups independently selected from halo, oxo, alkyl, hydroxy, alkoxy and amino, or

(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

each R¹⁵ is independently hydrogen or alkyl;

each R¹⁶ is independently alkylene or alkenylene;

each R¹⁷ is alkyl, alkenyl, alkynyl, cycloalkyl, cycloalkylalkyl, heterocyclyl, heterocyclylalkyl, aryl, aralkyl, heteroaryl or heteroaralkyl;

each R¹⁸ is independently alkylene or a direct bond;

each R¹⁹ and R²⁰ is independently selected from (i) or (ii) below:

(i) R¹⁹ and R²⁰ are each independently hydrogen, alkyl, cycloalkyl or

cycloalkylalkyl wherein the alkyl, cycloalkyl or cycloalkylalkyl may each be optionally substituted with one, two or three groups independently selected from halo, oxo, alkyl, haloalkyl, hydroxyalkyl, hydroxy, alkoxy and amino, or

(ii) R¹⁹ and R²⁰, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;

R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³, —R¹⁸NR¹⁵C(O)R²¹; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};

each R²¹ is independently hydrogen, alkyl, haloalkyl, hydroxyalkyl, alkenyl, alkynyl, cycloalkyl, cycloalkylalkyl, heterocyclyl, heterocyclylalkyl, aryl, aralkyl, heteroaryl or heteroaralkyl;

R²² and R²³ are independently selected from (i) or (ii) below:

(v) each R²² and R²³ is independently hydrogen or alkyl wherein the alkyl may each be optionally substituted with one or more substituents independently selected from the group consisting of halo, heterocyclyl, —OR^{x}, —NR^{y}R^{z} and —C(O)OR^{x}, or

(vi) R²² and R²³, together with the nitrogen atom to which they are attached, form a heterocyclyl which may be optionally substituted with one or more substituents independently selected from the group consisting of halo, oxo, alkyl, haloalkyl, hydroxyalkyl, —OR^{x}, —NR^{y}R^{z} and —C(O)OR^{x};

each R^{w} is independently a direct bond or alkylene;

each R^{x} is independently hydrogen, alkyl, alkenyl, alkynyl or cycloalkyl; and

each R^{y} and R^{z} is independently selected from (i) or (ii) below:

(i) R^{y} and R^{z} are each independently hydrogen, alkyl, cycloalkyl or cycloalkylalkyl, or

(ii) R^{y} and R^{z}, together with the nitrogen atom to which they are attached, form a heterocyclyl which may be optionally substituted with one or more substituents independently selected from the group consisting of halo, oxo, alkyl, haloalkyl, hydroxyalkyl, hydroxy and alkoxy.

In one embodiment, R¹ and R² are selected from (i), (ii) and (iii) as follows:

(i) R¹ and R² together form ═O;

(ii) R¹ and R² are each independently hydrogen or halo; and

(iii) R¹ is —OR¹² or —NR¹³R¹⁴ and R² is hydrogen, cyano, alkyl, haloalkyl, hydroxyalkyl or alkoxyalkyl or cycloalkyl;

In one embodiment, R¹ and R² together form ═O or ═S. In one embodiment, R¹ and R² together form ═O. In one embodiment, R¹ and R² together form ═NR⁹ and R⁹ is as described elsewhere herein. In one embodiment, R¹ and R² together from ═CR¹⁰R¹¹ and R¹⁰ and R¹¹ are as described elsewhere herein. In one embodiment, R¹ and R² together from K where R¹⁰ is —C(O)OR¹² and R¹¹ is as described elsewhere herein. In one embodiment, R¹ and R² together from ═CR¹⁰R¹¹, where R¹⁰ is —C(O)OR¹² and R¹¹ is hydrogen. In one embodiment, R¹ and R² together from ═CR¹⁰R¹¹, where R¹⁰ is —C(O)OR¹², R¹² is alkyl and R¹¹ is hydrogen. In one embodiment, R¹ and R² together from ═CR¹⁰R¹¹, where R¹⁰ is —C(O)OR¹², R¹² is ethyl and R¹¹ is hydrogen. In one embodiment, R¹ is —NR¹³R¹⁴, where R¹³ is hydroxyalkyl and R¹⁴ is hydrogen. In one embodiment, R¹ is —NR¹³R¹⁴, where R¹³ is hydroxyalkyl and R¹⁴ and R² are each hydrogen. In one embodiment, R¹ is —R¹⁸C(O)OR¹², and R² is as described elsewhere herein. In one embodiment, R¹ is —R¹⁸C(O)OR¹², and R² is hydrogen. In one embodiment, R¹ is —R¹⁸C(O)OR¹², where R¹⁸ is alkylene and R¹² is alkyl. In one embodiment, R¹ is —R¹⁸C(O)OR¹², where R¹⁸ is methylene and R¹² is ethyl.

In one embodiment, R¹ and R² are each independently hydrogen, hydroxyl, halo, cyano, cycloalkyl or alkyl. In one embodiment, R¹ and R² are each independently hydrogen, hydroxyl, halo, cycloalkyl or alkyl. In one embodiment, R¹ and R² are each independently hydrogen, hydroxyl, fluoro, isopropyl or cyclopropyl.

In one embodiment, R¹ and R² are each independently hydrogen or hydroxyl. In one embodiment, R¹ is hydrogen and R² is hydroxyl. In one embodiment, R¹ and R² are halo. In one embodiment, R¹ is hydrogen and R² is halo.

In one embodiment, R¹ and R² together with the carbon atom to which they are attached, form cycloalkyl or oxacycloalkyl. In one embodiment, the cycloalkyl or oxacycloalkyl comprises 3-7 carbon atoms, 3-6 carbon atoms or 3-5 carbon atoms.

In another embodiment, R³ is heteroaryl optionally substituted with one or more substituents selected from the group Q².

In another embodiment, R³ is 5-membered heteroaryl optionally substituted with one or more substituents selected from the group Q².

In another embodiment, R³ is optionally substituted with one, two or three Q² groups. In another embodiment, Q² is selected from halo, amino, alkyl and cycloalkyl, wherein the alkyl and cycloalkyl groups are optionally substituted with halo, alkyl, cyano, hydroxyl, haloalkyl, alkoxy or amino.

In another embodiment, R³ is optionally substituted azolyl. In another embodiment, R³ is azolyl optionally substituted with one to three substituents selected from the group consisting of halo, amino, alkyl, alkenyl, alkynyl, cycloalkyl, and cycloalkylalkyl. In another embodiment, R³ is azolyl optionally substituted with one to three substituents selected from the group consisting of halo, amino, alkyl and cycloalkyl. In another embodiment, R³ is optionally substituted 5-membered azolyl.

In another embodiment, the optionally substituted 5-membered azolyl is optionally substituted pyrazolyl, optionally substituted thiazolyl, optionally substituted isoxazolyl or optionally substituted imidazolyl. In another embodiment, R³ is optionally substituted 5-membered azolyl optionally substituted with one to three substituents selected from the group consisting of halo, amino, alkyl, alkenyl, alkynyl, cycloalkyl, and cycloalkylalkyl.

In another embodiment, R³ is azolyl, optionally substituted with one to three substituents selected from the group consisting of halo, amino, alkyl and cycloalkyl. In another embodiment, R³ is azolyl optionally substituted with alkyl. In another embodiment, R³ is azolyl substituted with alkyl. In another embodiment, R³ is azolyl substituted with methyl. In another embodiment, R³ is unsubstituted azolyl.

In another embodiment, R³ is pyrazolyl, optionally substituted with one to three substituents selected from halo, amino, alkyl and cycloalkyl. In another embodiment, R³ is pyrazolyl, optionally substituted with one to three substituents selected from fluoro, amino, methyl, cyclopropyl and cyclobutyl. In another embodiment, R³ is pyrazolyl optionally substituted with alkyl. In another embodiment, R³ is pyrazolyl substituted with alkyl. In another embodiment, R³ is pyrazolyl substituted with methyl. In another embodiment, R³ is unsubstituted pyrazolyl.

In another embodiment, R³ is thiazolyl, optionally substituted with one to three substituents selected from halo, amino, alkyl and cycloalkyl. In another embodiment, R³ is pyrazinyl. In another embodiment, R³ is pyrazinyl optionally substituted with alkyl. In another embodiment, R³ is pyrazinyl optionally substituted with methyl.

In another embodiment, R³ is optionally substituted azinyl. In another embodiment, R³ is azinyl optionally substituted with one to three substituents selected from the group consisting of halo, amino, alkyl, alkenyl, alkynyl, cycloalkyl, and cycloalkylalkyl. In another embodiment, R³ is azinyl optionally substituted with one to three substituents selected from the group consisting of halo, amino, alkyl and cycloalkyl.

In another embodiment, R³ is pyridinyl, optionally substituted with halo, amino, alkyl or cycloalkyl. In another embodiment, R³ is pyrimidinyl, optionally substituted with halo, amino, alkyl or cycloalkyl. In another embodiment, R³ is imidazolyl, optionally substituted with halo, amino, alkyl or cycloalkyl.

In one embodiment, R⁸ is optionally substituted cycloalkyl, aryl, heterocyclyl, or heteroaryl. In one embodiment, R⁸ is cycloalkyl, aryl, heterocyclyl, or heteroaryl, optionally substituted with one, two or three substituents selected from the group Q^{1a}, consisting of halo, cyano, alkyl, —R¹⁸OR²¹, —R¹⁸SR²¹ and —R¹⁸NR²²R²³ and R¹⁸, R²¹, R²² and R²³ are as described elsewhere herein. In one embodiment, R²¹, R²² and R²³ are each independently selected from hydrogen, methyl and ethyl. In another embodiment, R¹⁸ is a direct bond or alkylene. In one embodiment, R⁸ is cycloalkyl, aryl or heteroaryl, optionally substituted with one, two or three substituents selected from the group Q^{1a}, wherein Q^{1a} consists of halo, cyano, —R¹⁸OR²¹ and —R¹⁸SR²¹, and R¹⁸ and R²¹ are as described elsewhere herein. In one embodiment, R⁸ is cycloalkyl, aryl or heteroaryl, optionally substituted with one, two or three substituents selected from the group Q^{1a}, consisting of halo, cyano, alkyl and alkoxy.

In one embodiment, Q^{1a} is halo. In one embodiment, Q^{1a} is fluoro.

In certain embodiments, R⁸ is aryl optionally substituted with one or more substituents selected from the group Q^{1a}. In certain embodiments, R⁸ is phenyl optionally substituted with one or more substituents selected from the group Q^{1a} at the para position. In one embodiment, R⁸ is phenyl optionally substituted with one, two or three substituents selected from halo, cyano, alkyl, —OR¹² and —SR¹² wherein R¹² is alkyl. In one embodiment, R⁸ is phenyl optionally substituted with one, two or three substituents selected from fluoro, chloro, cyano, methyl, —OR¹² and —SR¹² wherein R¹² is methyl. In another embodiment, R⁸ is phenyl substituted with halo, methoxy or cyano at the para position. In another embodiment, R⁸ is phenyl substituted with halo, or cyano at the para position.

In another embodiment, R⁸ is thienyl optionally substituted with one or more substituents selected from the group Q^{1a}.

In one embodiment, Q^{1a}, consists of halo, cyano, alkyl, —R¹⁸OR²¹, —R¹⁸SR²¹ and —R¹⁸NR²²R²³ and R¹⁸, R²¹, R²² and R²³ are as described elsewhere herein.

In another embodiment, R¹ and R² are selected from (i) and (ii) as follows:

(i) R¹ and R² together form oxo; or

(ii) R¹ is OR¹² and R² is hydrogen, halo, alkyl, cycloalkyl, heterocyclyl, aryl, or heteroaryl and R¹² is as described elsewhere herein.

In another embodiment, R¹ and R² are selected from (i) or (ii) as follows:

(i) R¹ and R² together form oxo or thioxo, or

(ii) R¹ is —OR¹² and R² is hydrogen, halo or alkyl; and R¹² is as described elsewhere herein.

In another embodiment, R¹ and R² together form oxo.

In another embodiment, R¹ and R² together form thioxo.

In another embodiment, R¹ is —OR¹² and R² is hydrogen, halo, alkyl, cycloalkyl, or haloalkyl; and R¹⁰ is as described elsewhere herein.

In another embodiment, R¹ is —OH and R² is hydrogen or alkyl. In another embodiment, R¹ is —OH and R² is hydrogen.

In another embodiment, R¹ and R² are both —OR¹² and R¹² is as defined elsewhere herein. In another embodiment, R¹ and R² are both —SR¹² and R¹² is as described elsewhere herein. In another embodiment, R¹ is —NR¹³R¹⁴ or —N(R¹⁵)R¹⁶OR¹², R² is hydrogen or alkyl and R¹², R¹³, R¹⁴, R¹⁵ and R¹⁶ are as described elsewhere herein.

In another embodiment, R¹ and R² are selected from (i) or (ii) as follows:

(i) R¹ and R² together form ═CR¹⁰R¹¹, or

(ii) R¹ is —R¹⁸C(O)OR¹² and R² is hydrogen, halo or alkyl; R¹⁸ is alkylene and R¹⁰, R¹¹ and R¹² are as described elsewhere herein.

In one embodiment, R⁵, R⁶ and R⁷ are each independently hydrogen, halo, nitro, hydroxy, alkyl, alkenyl, alkynyl, cycloalkyl, heterocyclyl, heterocyclylalkyl, —OR¹⁷, —C(O)NR¹⁹R²⁰, —C(S)NR¹⁹R²⁰—C(O)OR¹⁷, —C(S)OR¹⁷ and —C(═NOR¹⁵)R²¹ wherein the alkyl, alkenyl, alkynyl, cycloalkyl, heterocyclyl and heterocyclylalkyl may each be optionally substituted with one, two, three or four groups selected from halo, alkyl, haloalkyl, hydroxyalkyl, cyano, oxo, —R^{w}—OR^{x}, —R^{w}—OR^{w}NR^{Y}R^{z}, —R^{w}—NR^{y}r^{z}, —R^{w}—NR^{u}C(O)R^{v} and —S(O)ₚR^{v} or R⁵ is halo, R⁶ is —NR¹⁵C(O)R¹⁷ and R⁷ is hydrogen.

In another embodiment, R⁵, R⁶ and R⁷ are each independently hydrogen, halo, nitro, alkyl, —OR¹⁷, —NR¹⁵C(O)R¹⁷ or —C(O)NR¹⁹R²⁰ wherein the alkyl may be optionally substituted with one, two or three groups selected from halo, alkyl, haloalkyl, hydroxyalkyl, cyano, and —R^{w}—NR^{y}R^{z}, and R¹⁵, R¹⁷, R¹⁹, R²⁰, R^{w}, R^{x}, R^{y} and R^{z} are as described elsewhere herein. In another embodiment, R⁵, R⁶ and R⁷ are each independently hydrogen, halo, hydroxyl, alkyl, alkoxy, haloalkyl, hydroxyalkyl or cycloalkyl. In another embodiment, R⁵, R⁶ and R⁷ are each independently hydrogen, halo or alkyl. In another embodiment, R⁵, R⁶ and R⁷ are each independently hydrogen, halo, alkyl, —OR¹⁷ or —NR¹⁵C(O)R¹⁷ wherein the alkyl may be optionally substituted with one, two or three groups selected from halo, alkyl, haloalkyl, hydroxyalkyl, cyano, —R^{w}—OR^{x} and —R^{w}—NR^{y}R^{z} and R^{w}, R^{x}, R^{y}, R^{x}, R¹⁵ and R¹⁷ are as described elsewhere herein. In another embodiment, R⁵, R⁶ and R⁷ are each independently hydrogen, halo, alkyl or —NR¹⁵C(O)R¹⁷ wherein the alkyl may be optionally substituted with one, two or three groups selected from halo, alkyl, haloalkyl, hydroxyalkyl, cyano, —R^{w}—OR^{x} and —R^{w}—NRYle and R^{w}, R^{x}, R^{y}, R^{x}, R¹⁵ and R¹⁷ are as described elsewhere herein. In another embodiment, R⁵, R⁶ and R⁷ are each independently hydrogen or halo. In another embodiment, R⁵ is hydrogen or halo. In another embodiment, R⁵ is hydrogen. In another embodiment, R⁶ is hydrogen or halo. In another embodiment, R⁶ is hydrogen. In another embodiment, R⁶ is halo. In another embodiment, R⁷ is hydrogen or halo. In another embodiment, R⁷ is hydrogen. In another embodiment, R⁷ is halo. In another embodiment, R⁵, R⁶ and R⁷ are each independently hydrogen, chloro or fluoro. In another embodiment, R⁵ is halo and R⁶ is —NR¹⁵C(O)R¹⁷. In another embodiment, R⁵ is halo, R⁶ is —NR¹⁵C(O)R¹⁷ and R⁷ is hydrogen. In another embodiment, R⁷ is hydrogen and R⁵ and R⁶ are each independently halo, nitro, hydroxy, alkyl, alkenyl, alkynyl, cycloalkyl, heterocyclyl, heterocyclylalkyl, —OR¹⁷, —NR¹⁵C(O)R¹⁷, —C(O)NR¹⁹R²⁰, —C(S)NR¹⁹R²⁰, —C(O)OR¹⁷, —C(S)OR¹⁷ and —C(═NOR¹⁵)R²¹ wherein the alkyl, alkenyl, alkynyl, cycloalkyl, heterocyclyl and heterocyclylalkyl may each be optionally substituted with one, two, three or four groups selected from halo, alkyl, haloalkyl, hydroxyalkyl, cyano, —R^{w}—OR^{x}, —R^{w}—OR^{w}NR^{y}R^{z}, —R^{w}—NR^{y}r^{z}, —R^{w}—NR^{u}C(O)R^{v} and —S(O)_{P}R^{v}. In another embodiment, R⁵ is halo, R⁷ is hydrogen and R⁶ is halo, nitro, hydroxy, alkyl, alkenyl, alkynyl, cycloalkyl, heterocyclyl, heterocyclylalkyl, —OR¹⁷, —NR¹⁵C(O)R¹⁷, —C(O)NR¹⁹R²⁰, —C(S)NR¹⁹R²⁰, —C(O)OR¹⁷, —C(S)OR¹⁷ and —C(═NOR¹⁵)R²¹ wherein the alkyl, alkenyl, alkynyl, cycloalkyl, heterocyclyl and heterocyclylalkyl may each be optionally substituted with one, two, three or four groups selected from halo, alkyl, haloalkyl, hydroxyalkyl, cyano, —R^{w}—OR^{w}NR^{y}R^{z}, —R^{w}—NR^{y}R^{z}, —R^{w}—NR^{u}C(O)R^{v} and —S(O)_{P}R^{v}.

In one embodiment, the compounds provided are of formula (II)

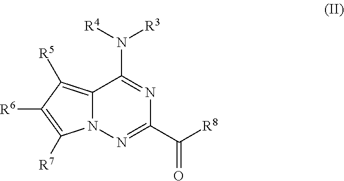

or a pharmaceutically acceptable salt, solvate, clathrate or hydrate thereof, wherein the variables are as described elsewhere herein.

In another embodiment, the compounds provided are of formula (III)

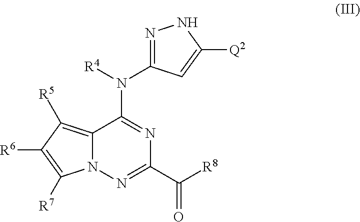

or a pharmaceutically acceptable salt, solvate, clathrate or hydrate thereof, wherein the variables are as described elsewhere herein.

In one embodiment, the compounds provided are of formula (IV)

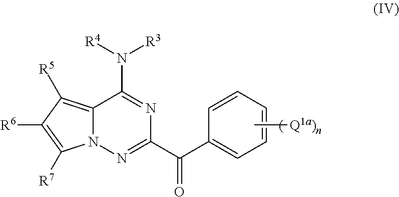

or a pharmaceutically acceptable salt, solvate, clathrate or hydrate thereof, where n is 0-5 and the other variables are as described elsewhere herein.

In one embodiment, the compounds provided are of formula (V)

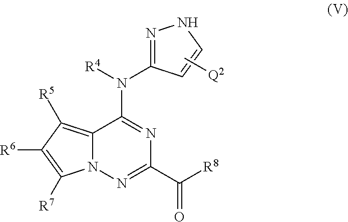

or a pharmaceutically acceptable salt, solvate, clathrate or hydrate thereof, wherein n is an integer from 0 to 5 and the other variables are as described elsewhere herein.

In another embodiment, the compounds provided are of formula (VI)

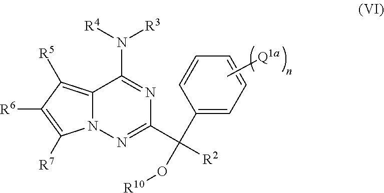

or a pharmaceutically acceptable salt, solvate, clathrate or hydrate thereof, wherein n is an integer from 0 to 5, and the other variables are as described elsewhere herein.

In another embodiment, the compounds provided are of formula (VIIa) or (VIIb)

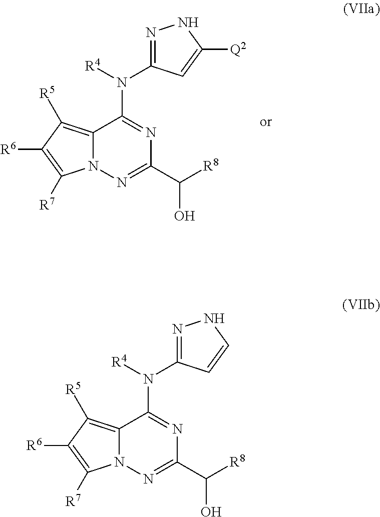

or a pharmaceutically acceptable salt, solvate, clathrate or hydrate thereof, wherein the variables are as described elsewhere herein.

In another embodiment, the compounds provided are of formula (VIII)

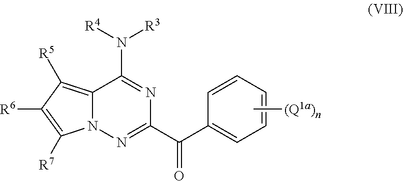

or a pharmaceutically acceptable salt, solvate, clathrate or hydrate thereof, where n is 0 to 5 and the other variables are as described elsewhere herein.

In another embodiment, the compounds provided are of formula (IXa), (IXb) or (IXc)

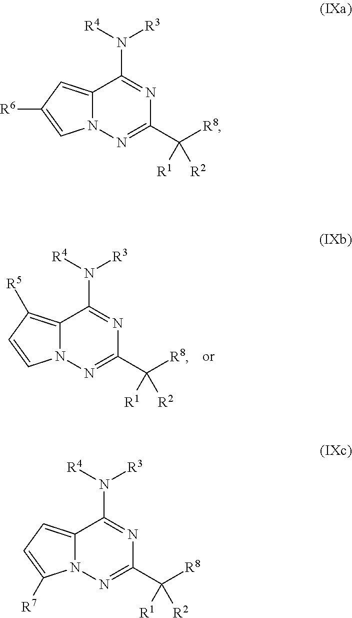

or a pharmaceutically acceptable salt, solvate, clathrate or hydrate thereof, where the variables are as described elsewhere herein.

In another embodiment, the compounds provided are of formula (IXa) or (IXb)

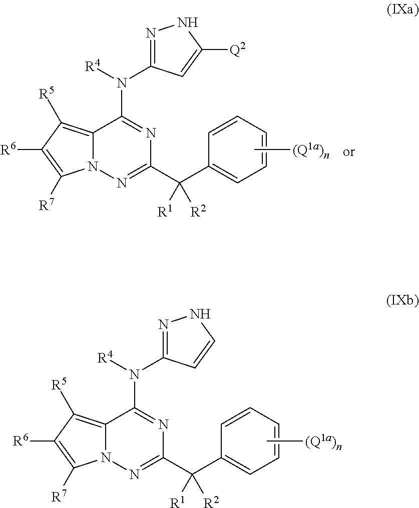

or a pharmaceutically acceptable salt, solvate, clathrate or hydrate thereof, wherein n is an integer from 0 to 5, and the other variables are as described elsewhere herein.

In another embodiment, the compounds provided are of formula (Xa), (Xb), (Xc) or (Xd)

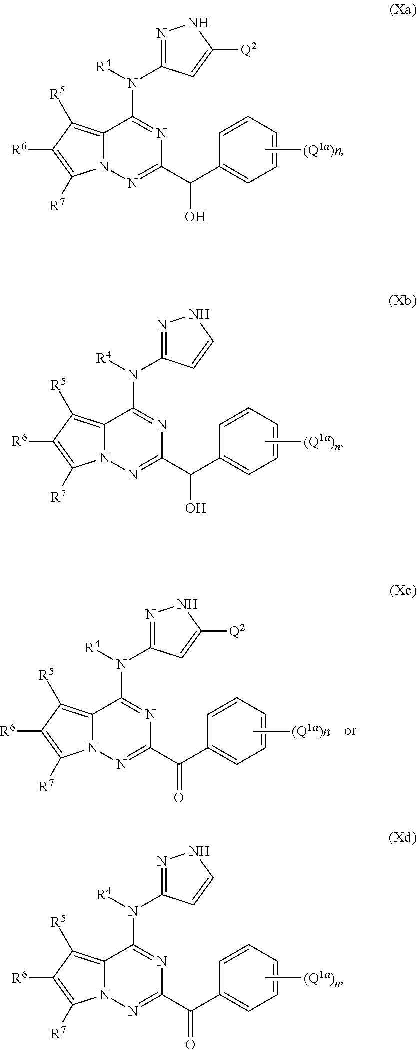

or a pharmaceutically acceptable salt, solvate, clathrate or hydrate thereof, wherein n is an integer from 0 to 5, and the other variables are as described elsewhere herein. In one embodiment, n is 0, 1 or 2. In one embodiment, n is 0 or 1.

In another embodiment, the compounds provided are of formula (Xe):

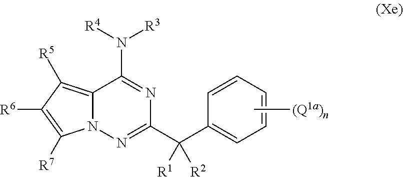

or a pharmaceutically acceptable salt, solvate, clathrate or hydrate thereof, wherein n is an integer from 0 to 5 and wherein R¹, R², R³, R⁴, R⁵, R⁶, R⁷, and Q^{1a} are as described elsewhere herein. In one embodiment, n is 0, 1 or 2. In one embodiment, n is 0 or 1.

In another embodiment, the compounds provided are of formula (Xf):

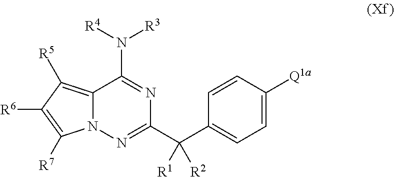

or a pharmaceutically acceptable salt, solvate, clathrate or hydrate thereof, wherein R¹, R², R³, R⁴, R⁵, R⁶, R⁷ and Q^{1a} are as described elsewhere herein.

In one embodiment, the compounds provided are of formula (XI)

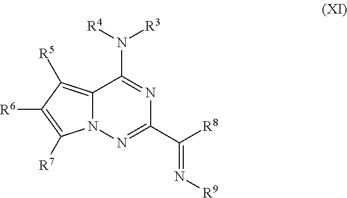

or a pharmaceutically acceptable salt, solvate, clathrate or hydrate thereof, wherein the variables are as described elsewhere herein.

In one embodiment, the compounds provided are of formula (XII)

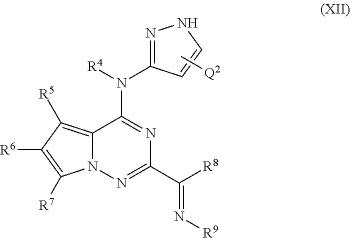

or a pharmaceutically acceptable salt, solvate, clathrate or hydrate thereof, wherein the variables are as described elsewhere herein.

In one embodiment, the compounds provided are of formula (XIII)

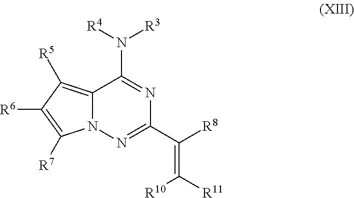

or a pharmaceutically acceptable salt, solvate, clathrate or hydrate thereof, wherein the variables are as described elsewhere herein.

In one embodiment, the compounds provided are of formula (XIV)

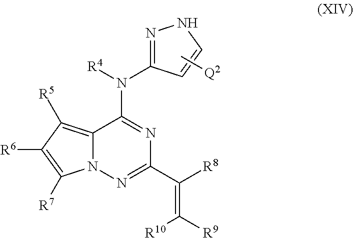

or a pharmaceutically acceptable salt, solvate, clathrate or hydrate thereof, wherein the variables are as described elsewhere herein.

In one embodiment, the compounds provided are of formula (XVa) or (XVb)

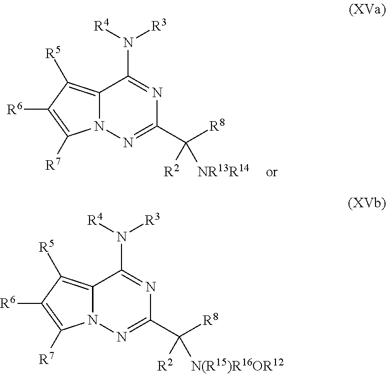

or a pharmaceutically acceptable salt, solvate, clathrate or hydrate thereof, wherein the variables are as described elsewhere herein. In one embodiment, the compound has formula XVa, where R¹³ is hydroxyalkyl, R¹⁴ is hydrogen and other variables are as described elsewhere herein.

In one embodiment, the compounds provided are of formula (XVI) or (XVII):

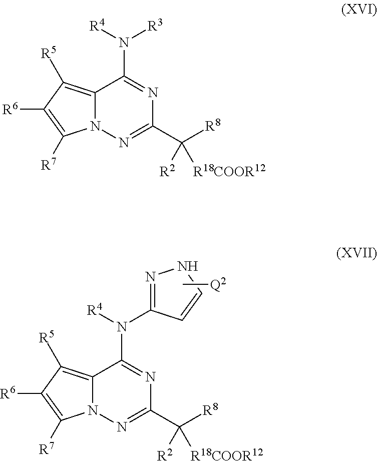

or a pharmaceutically acceptable salt, solvate, clathrate or hydrate thereof, wherein the variables are as described elsewhere herein. In another embodiment, R² is hydrogen. In one embodiment, R¹⁸ is alkylene and R¹² is alkyl. In one embodiment, R¹⁸ is methylene and R¹² is ethyl.

In another embodiment, the compound provided is selected from
(4-fluorophenyl)(4-(5-methyl-1H-pyrazol-3-ylamino)pyrrolo[1,2,4][1,2,4]triazin-2-yl)methanone;
(4-fluorophenyl)(4-(5-methyl-1H-pyrazol-3-ylamino)pyrrolo[1,2-f][1,2,4]triazin-2-yl)methanol;
2-(fluoro(4-fluorophenyl)methyl)-N-(5-methyl-1H-pyrazol-3-yl)pyrrolo[1,2-f][1,2,4]triazin-4-amine;
2-(difluoro(4-fluorophenyl)methyl)-N-(5-methyl-1H-pyrazol-3-yl)pyrrolo[1,2-f][1,2,4]triazin-4-amine;
(4-(1H-pyrazol-3-ylamino)pyrrolo[1,2-f][1,2,4]triazin-2-yl)(4-fluorophenyl)methanone;
(4-(1H-pyrazol-3-ylamino)pyrrolo[1,2-f][1,2,4]triazin-2-yl) (4-fluorophenyl)methanol;
2-(fluoro(4-fluorophenyl)methyl)-N-(1H-pyrazol-3-yl)pyrrolo[1,2-f][1,2,4]triazin-4-amine;
2-(difluoro(4-fluorophenyl)methyl)-N-(1H-pyrazol-3-yl)pyrrolo[1,2-f][1,2,4]triazin-4-amine;
(4-(1H-pyrazol-3-ylamino)-7-chloropyrrolo[1,2,4][1,2,4]triazin-2-yl)(4-fluorophenyl)methanone;
2-(4-fluorobenzyl)-N-(1H-pyrazol-3-yl)pyrrolo[1,2,4][1,2,4]triazin-4-amine;
(3-fluorophenyl)(4-(5-methyl-1H-pyrazol-3-ylamino)pyrrolo[1,2,4][1,2,4]triazin-2-yl)methanone;
(3-fluorophenyl)(4-(5-methyl-1H-pyrazol-3-ylamino)pyrrolo[1,2-f][1,2,4]triazin-2-yl)methanol;
(4-(1H-pyrazol-3-ylamino)pyrrolo[1,2-f][1,2,4]triazin-2-yl)(3-fluorophenyl)methanone;
(4-(1H-pyrazol-3-ylamino)pyrrolo[1,2-f][1,2,4]triazin-2-yl)(3-fluorophenyl)methanol;
(4-(1H-pyrazol-3-ylamino)pyrrolo[1,2-f][1,2,4]triazin-2-yl)(2-methoxyphenyl)methanone;
(4-(1H-pyrazol-3-ylamino)pyrrolo[1,2-f][1,2,4]triazin-2-yl)(2-methoxyphenyl)methanol;
(5-fluoro-2-methoxyphenyl)(4-(5-methyl-1H-pyrazol-3-ylamino)pyrrolo-[1,2-f][1,2,4]triazin-2-yl)methanone;
(5-fluoro-2-methoxyphenyl)(4-(5-methyl-1H-pyrazol-3-ylamino)pyrrolo[1,2-f][1,2,4]triazin-2-yl)methanol;
(4-(1H-pyrazol-3-ylamino)pyrrolo[1,2-f][1,2,4]triazin-2-yl)(5-fluoro-2-methoxyphenyl)methanone;
4-(4-(1H-pyrazol-3-ylamino)pyrrolo[1,2-f][1,2,4]triazine-2-carbonyl)benzonitrile;
4-((4-(1H-pyrazol-3-ylamino)pyrrolo[1,2-f][1,2,4]triazin-2-yl)(hydroxy)methyl)benzonitrile;
(4-(1H-pyrazol-3-ylamino)pyrrolo[1,2-f][1,2,4]triazin-2-yl)(2,4-difluorophenyl)methanone;
5-(4-(1H-pyrazol-3-ylamino)pyrrolo[1,2-f][1,2,4]triazine-2-carbonyl)-2-fluorobenzonitrile;
3-(4-(1H-pyrazol-3-ylamino)pyrrolo[1,2-f][1,2,4]triazine-2-carbonyl)benzonitrile;
(4-(5-cyclopropyl-1H-pyrazol-3-ylamino)pyrrolo[1,2-f][1,2,4]triazin-2-yl)(4-fluorophenyl)methanone;
(4-(5-cyclopropyl-1H-pyrazol-3-ylamino)pyrrolo[1,2-f][1,2,4]triazin-2-yl)(4-fluorophenyl)methanol;
(4-(5-cyclobutyl-1H-pyrazol-3-ylamino)pyrrolo[1,2-f][1,2,4]triazin-2-yl)(4-fluorophenyl)methanone;
(4-(5-cyclobutyl-1H-pyrazol-3-ylamino)pyrrolo[1,2-f][1,2,4]triazin-2-yl)(4-fluorophenyl)methanol;
(4-fluorophenyl)(4-(thiazol-2-ylamino)pyrrolo[1,2-f][1,2,4]triazin-2-yl)methanone;
(4-fluorophenyl)(4-(thiazol-2-ylamino)pyrrolo[1,2-f][1,2,4]triazin-2-yl)methanol;
(4-fluorophenyl)(4-(5-fluorothiazol-2-ylamino)pyrrolo[1,2-f][1,2,4]triazin-2-yl)methanone;
(4-fluorophenyl)(4-(pyrazin-2-ylamino)pyrrolo[1,2-f][1,2,4]triazin-2-yl)methanone;
(4-(1H-pyrazol-3-ylamino)pyrrolo[1,2-f][1,2,4]triazin-2-yl)(cyclopropyl)methanone;
(4-(1H-pyrazol-3-ylamino)pyrrolo[1,2-f][1,2,4]triazin-2-yl)(cyclohexyl)methanone;
(4-(5-methyl-1H-pyrazol-3-ylamino)pyrrolo[1,2-f][1,2,4]triazin-2-yl) (thiophen-2-yl)methanone;
(4-(1H-pyrazol-3-ylamino)pyrrolo[1,2-f][1,2,4]triazin-2-yl)(thiophen-2-yl)methanone;
(4-(1H-pyrazol-3-ylamino)pyrrolo[1,2-f][1,2,4]triazin-2-yl) (thiophen-2-yl)methanol;
N-((4-(1H-pyrazol-3-ylamino)pyrrolo[1,2-f][1,2,4]triazin-2-yl)(4-fluorophenyl)methyl)-2-hydroxyethanamonium acetate;
2-(1-(4-fluorophenyl)vinyl)-N-(1H-pyrazol-5-yl)pyrrolo[1,2-f][1,2,4]triazin-4-amine;
ethyl 3-(4-(1H-pyrazol-3-ylamino)pyrrolo[1,2-f][1,2,4]triazin-2-yl)-3-(4-fluorophenyl)acrylate;
ethyl 3-(4-(1H-pyrazol-3-ylamino)pyrrolo[1,2-f][1,2,4]triazin-2-yl)-3-(4-fluorophenyl)propanoate;
(4-(5-methyl-1H-pyrazol-3-ylamino)pyrrolo[1,2-f][1,2,4]triazin-2-yl)(quinolin-8-yl)methanone;
(4-(5-methyl-1H-pyrazol-3-ylamino)pyrrolo[1,2-f][1,2,4]triazin-2-yl)(quinolin-8-yl)methanol;
2-((4-(1H-pyrazol-3-ylamino)pyrrolo[1,2-f][1,2,4]triazin-2-yl)(4-fluorophenyl)methylamino)ethanol;
2-(difluoro(4-fluorophenyl)methyl)-4-(5-methyl-1H-pyrazol-3-ylamino)-N-(2-morpholinoethyl)pyrrolo[1,2-f][1,2,4]triazine-6-carboxamide;
2-[difluoro-(4-fluoro-phenyl)-methyl]-4-(5-methyl-1H-pyrazol-3-ylamino)pyrrolo[2,1-f][1,2,4]triazine-6-carboxylic acid (2-dimethylamino-ethyl)-amide;
2-[difluoro-(4-fluoro-phenyl)-methyl]-4-(5-methyl-1H-pyrazol-3-ylamino)pyrrolo[2,1-f][1,2,4]triazine-6-carboxylic acid (3-pyrrolidin-1-yl-propyl)-amide;
[2-[difluoro-(4-fluoro-phenyl)-methyl]-4-(5-methyl-1H-pyrazol-3-ylamino)pyrrolo[2,1-f][1,2,4]triazin-6-yl]-(4-methyl-piperazin-1-yl)-methanone;
[1,4′]Bipiperidinyl-1′-yl-[2-[difluoro-(4-fluoro-phenyl)-methyl]-4-(5-methyl-1H-pyrazol-3-ylamino)-pyrrolo[2,1-f][1,2,4]triazin-6-yl]-methanone;
2-[Difluoro-(4-fluoro-phenyl)-methyl]-4-(5-methyl-1H-pyrazol-3-ylamino)pyrrolo[2,1-f][1,2,4]triazine-6-carboxylic acid (3-morpholin-4-yl-propyl)-amide;
2-[Difluoro-(4-fluoro-phenyl)-methyl]-4-(5-methyl-1H-pyrazol-3-ylamino)pyrrolo[2,1-f][1,2,4]triazine-6-carboxylic acid (3-piperidin-1-yl-propyl)-amide;
2-[Difluoro-(4-fluoro-phenyl)-methyl]-4-(5-methyl-1H-pyrazol-3-ylamino)pyrrolo[2,1-f][1,2,4]triazine-6-carboxylic acid [2-(4-methyl-piperazin-1-yl)-ethyl]-amide;
3-(Diethylamino)-N-(2-(difluoro(4-fluorophenyl)methyl)-4-(5-methyl-1H-pyrazol-3-ylamino)pyrrolo[1,2-f][1,2,4]triazin-6-yl)propanamide;
N-[2-[Difluoro-(4-fluoro-phenyl)-methyl]-4-(5-methyl-1H-pyrazol-3-ylamino)pyrrolo[2,1-f][1,2,4]triazin-6-yl]-3-(4,4-difluoro-piperidin-1-yl)-propionamide;
N-[2-[Difluoro-(4-fluoro-phenyl)-methyl]-4-(5-methyl-1H-pyrazol-3-ylamino)pyrrolo[2,1-f][1,2,4]triazin-6-yl]-3-(4-methyl-piperazin-1-yl)-propionamide;
(2-(difluoro(4-fluorophenyl)methyl)-4-(5-methyl-1H-pyrazol-3-ylamino) pyrrolo[1,2-f][1,2,4]triazin-6-yl)methanol;
6-(aminomethyl)-2-(difluoro(4-fluorophenyl)methyl)-N-(5-methyl-1H-pyrazol-3-yl)pyrrolo[1,2-f][1,2,4]triazin-4-amine;
N-((2-(difluoro(4-fluorophenyl)methyl)-4-(5-methyl-1H-pyrazol-3-ylamino)pyrrolo[1,2-f][1,2,4]triazin-6-yl)methyl)acetamide;
[2-[Difluoro-(4-fluoro-phenyl)-methyl]-4-(5-methyl-1H-pyrazol-3-ylamino)pyrrolo[2,1-f][1,2,4]triazin-6-ylmethyl]-carbamic acid methyl ester;

N-[2-[Difluoro-(4-fluoro-phenyl)-methyl]-4-(5-methyl-1H-pyrazol-3-ylamino)pyrrolo[2,1-f][1,2,4]triazin-6-ylmethyl]-methanesulfonamide;
N-[2-[Difluoro-(4-fluoro-phenyl)-methyl]-4-(5-methyl-1H-pyrazol-3-ylamino)pyrrolo[2,1-f][1,2,4]triazin-6-ylmethyl]-isobutyramide;
N-[2-[Difluoro-(4-fluoro-phenyl)-methyl]-4-(5-methyl-1H-pyrazol-3-ylamino)pyrrolo[2,1-f][1,2,4]triazin-6-ylmethyl]-2,2-dimethyl-propionamide;
1-(2-(difluoro(4-fluorophenyl)methyl)-4-(5-methyl-1H-pyrazol-3-ylamino)pyrrolo[1,2-f][1,2,4]triazin-6-yl)ethanone;
1-(2-(difluoro(4-fluorophenyl)methyl)-4-(5-methyl-1H-pyrazol-3-ylamino)pyrrolo[1,2-f][1,2,4]triazin-6-yl)ethanol;
2-(difluoro(4-fluorophenyl)methyl)-6-(ethoxymethyl)-N-(5-methyl-1H-pyrazol-3-yl)pyrrolo[1,2-f][1,2,4]triazin-4-amine;
2-(difluoro(4-fluorophenyl)methyl)-6-(isopropoxymethyl)-N-(5-methyl-1H-pyrazol-3-yl)pyrrolo[1,2-f][1,2,4]triazin-4-amine;
N-(2-(Difluoro(4-fluorophenyl)methyl)-4-(5-methyl-1H-pyrazol-3-ylamino)pyrrolo[1,2-f][1,2,4]triazin-6-yl)-2-morpholinoacetamide;
N-[2-[Difluoro-(4-fluoro-phenyl)-methyl]-4-(5-methyl-1H-pyrazol-3-ylamino)pyrrolo[2,1-f][1,2,4]triazin-6-yl]-difluoro-piperidin-1-yl)-acetamide
N-[2-[Difluoro-(4-fluoro-phenyl)-methyl]-4-(5-methyl-1H-pyrazol-3-ylamino)pyrrolo[2,1-f][1,2,4]triazin-6-yl]-2-(3,3-difluoro-pyrrolidin-1-yl)-acetamide
2-(3,3-Difluoro-azetidin-1-yl)-N-[2-[difluoro-(4-fluoro-phenyl)-methyl]-4-(5-methyl-1H-pyrazol-3-ylamino)-pyrrolo[2,1-f][1,2,4]triazin-6-yl]-acetamide
N-[2-[Difluoro-(4-fluoro-phenyl)-methyl]-4-(5-methyl-1H-pyrazol-3-ylamino)pyrrolo[2,1-f][1,2,4]triazin-6-yl]-2-(4,4-difluoro-piperidin-1-yl)-acetamide
N-[2-[Difluoro-(4-fluoro-phenyl)-methyl]-4-(5-methyl-1H-pyrazol-3-ylamino)pyrrolo[2,1-f][1,2,4]triazin-6-yl]-2-(4-methyl-piperazin-1-yl)-acetamide
2-(difluoro(4-fluorophenyl)methyl)-N-(5-methyl-1H-pyrazol-3-yl)-6-(morpholinomethyl)pyrrolo[1,2-f][1,2,4]triazin-4-amine;
6-((cyclopropylamino)methyl)-2-(difluoro(4-fluorophenyl)methyl)-N-(5-methyl-1H-pyrazol-3-yl)pyrrolo[1,2-f][1,2,4]triazin-4-amine;
2-(difluoro(4-fluorophenyl)methyl)-6-((3,3-difluoropyrrolidin-1-yl)methyl)-N-(5-methyl-1H-pyrazol-3-yl)pyrrolo[1,2-f][1,2,4]triazin-4-amine;
2-(difluoro(4-fluorophenyl)methyl)-6-((3,3-difluoroazetidin-1-yl)methyl)-N-(5-methyl-1H-pyrazol-3-yl)pyrrolo[1,2-f][1,2,4]triazin-4-amine;
(Z)-1-(2-(difluoro(4-fluorophenyl)methyl)-4-(5-methyl-1H-pyrazol-3-ylamino)pyrrolo[1,2-f][1,2,4]triazin-6-yl)ethanone oxime;
2-(difluoro(4-fluorophenyl)methyl)-4-(5-methyl-1H-pyrazol-3-ylamino)pyrrolo[1,2f][1,2,4]triazine-6-carbaldehyde oxime;
(4-fluorophenyl)(6-(hydroxymethyl)-4-(5-methyl-1H-pyrazol-3-ylamino)pyrrolo[1,2-f][1,2,4]triazin-2-yl)methanol;
2-(2((4-fluorophenyl)(hydroxy)methyl)-4-(5-methyl-1H-pyrazol-3-ylamino)pyrrolo[1,2-f][1,2,4]triazin-6-yl)propan-2-ol;
2-(2-(1-(4-fluorophenyl)-1-hydroxyethyl)-4-(5-methyl-1H-pyrazol-3-ylamino)pyrrolo[1,2-f][1,2,4]triazin-6-yl)propan-2-ol;
2-(difluoro(4-fluorophenyl)methyl)-4-(5-methyl-1H-pyrazol-3-ylamino)pyrrolo[1,2-f][1,2,4]triazin-6-ol;
2-(difluoro(4-fluorophenyl)methyl)-N-(5-methyl-1H-pyrazol-3-yl)-6-(3-morpholinopropoxy)pyrrolo[1,2-f][1,2,4]triazin-4-amine;
2-(2-((4-fluorophenyl)(methoxy)methyl)-4-(5-methyl-1H-pyrazol-3-ylamino)pyrrolo[1,2-f][1,2,4]triazin-6-yl)propan-2-ol;
methyl (4-fluorophenyl)(6-(2-hydroxypropan-2-yl)-4-(5-methyl-1H-pyrazol-3-ylamino)pyrrolo[1,2-f][1,2,4]triazin-2-yl)methylcarbamate;
2-(difluoro(4-fluorophenyl)methyl)-N-(5-methyl-1H-pyrazol-3-yl)-6-((2-(piperidin-1-yl)ethoxy)methyl)pyrrolo[1,2-f][1,2,4]triazin-4-amine acetate;
2-(difluoro(4-fluorophenyl)methyl)-N-(5-methyl-1H-pyrazol-3-yl)-6-(((1-methylpiperidin-3-yl)methoxy)methyl)pyrrolo[1,2-f][1,2,4]triazin-4-amine;
2-(difluoro(4-fluorophenyl)methyl)-N-(5-methyl-1H-pyrazol-3-yl)-6-((2-(pyrrolidin-1-yl)ethoxy)methyl)pyrrolo[1,2-f][1,2,4]triazin-4-amine;
2-(difluoro(4-fluorophenyl)methyl)-N-(5-methyl-1,1-pyrazol-3-yl)-6-((2-morpholinoethoxy)methyl)pyrrolo[1,2-f][1,2,4]triazin-4-amine;
2-(difluoro(4-fluorophenyl)methyl)-N-(5-methyl-1H-pyrazol-3-yl)-6-((3-morpholinopropoxy)methyl)pyrrolo[1,2-f][1,2,4]triazin-4-amine;
(4-fluorophenyl)(4-(5-methyl-1H-pyrazol-3-ylamino)pyrrolo[1,2-f][1,2,4]triazin-2-yl)methanone oxime;
1-(4-fluorophenyl)-1-(4-(5-methyl-1H-pyrazol-3-ylamino)pyrrolo[1,2-f][1,2,4]triazin-2-yl)ethanol;
1-(4-Fluoro-phenyl)-2-methyl-1-[4-(5-methyl-1H-pyrazol-3-ylamino)-pyrrolo[2,1-f][1,2,4]triazin-2-yl]-propan-1-ol;
Cyclopropyl-(4-fluoro-phenyl)-[4-(5-methyl-1H-pyrazol-3-ylamino)-pyrrolo[2,1-f][1,2,4]triazin-2-yl]-methanol;
1-[6-Fluoro-4-(5-methyl-1H-pyrazol-3-ylamino)-pyrrolo[2,1-f][1,2,4]triazin-2-yl]-1-(4-fluoro-phenyl)-ethanol;
3-(4-(1H-pyrazol-3-ylamino)pyrrolo[1,2-f][1,2,4]triazin-2-yl)-3-(4-fluorophenyl)propan-1-ol;
2-(1-(4-fluorophenyl)-3-morpholinopropyl)-N-(1H-pyrazol-3-yl)pyrrolo[1,2-f][1,2,4]triazin-4-amine;
{2-[1-(4-Fluoro-phenyl)-3-piperidin-1-yl-propyl]-pyrrolo[2,1-f][1,2,4]triazin-4-yl}-(1H-pyrazol-3-yl)-amine;
{2-[1-(4-Fluoro-phenyl)-3-pyrrolidin-1-yl-propyl]-pyrrolo[2,1-f][1,2,4]triazin-4-yl}-(1H-pyrazol-3-yl)-amine;
{2-[3-Dimethylamino-1-(4-fluoro-phenyl)-propyl]-pyrrolo[2,1-f][1,2,4]triazin-4-yl}-(1H-pyrazol-3-yl)-amine;
2-(amino(4-fluorophenyl)methyl)-N-(5-methyl-1H-pyrazol-3-yl)pyrrolo[1,2-f][1,2,4]triazin-4-amine;
N-((4-fluorophenyl)(4-(5-methyl-1H-pyrazol-3-ylamino)pyrrolo[1,2-f][1,2,4]triazin-2-yl)methyl)-2-morpholinoacetamide;
N-{(4-Fluoro-phenyl)-[4-(5-methyl-1-pyrazol-3-ylamino)-pyrrolo[2,1-f][1,2,4]triazin-2-yl]-methyl}-2-(4-methyl-piperazin-1-yl)-acetamide;
2-Dimethylamino-N-{(4-fluoro-phenyl)-[4-(5-methyl-1H-pyrazol-3-ylamino)pyrrolo[2,1-f][1,2,4]triazin-2-yl]-methyl}-acetamide;
2-(Difluoro-piperidin-1-yl)-N-{(4-fluoro-phenyl)-[4-(5-methyl-1H-pyrazol-3-ylamino)-pyrrolo[2,1-f][1,2,4]triazin-2-yl]-methyl}-acetamide;
N-{(4-Fluoro-phenyl)-[4-(5-methyl-1-pyrazol-3-ylamino)-pyrrolo[2,1-f][1,2,4]triazin-2-yl]-methyl}-2-(4-methanesulfonyl-piperazin-1-yl)-acetamide;
{(4-Fluoro-phenyl)-[4-(5-methyl-1H-pyrazol-3-ylamino)-pyrrolo[2,1-f][1,2,4]triazin-2-yl]-methyl}-carbamic acid 3-morpholin-4-yl-propyl ester;
{(4-Fluoro-phenyl)-[4-(5-methyl-1H-pyrazol-3-ylamino)-pyrrolo[2,1-f][1,2,4]triazin-2-yl]-methyl}-carbamic acid 3-(3,3-difluoro-azetidin-1-yl)-propyl ester;
{(4-Fluoro-phenyl)-[4-(5-methyl-1H-pyrazol-3-ylamino)-pyrrolo[2,1-f][1,2,4]triazin-2-yl]-methyl}-carbamic acid 2-morpholin-4-yl-ethyl ester;
N-((4-fluorophenyl)(4-(5-methyl-1H-pyrazol-3-ylamino)pyrrolo[1,2-f][1,2,4]triazin-2-yl)methyl)cyclopropanecarboxamide;
2-((4-fluorophenyl)(2-morpholinoethoxy)methyl)-N-(5-methyl-1H-pyrazol-3-yl)pyrrolo[1,2-f][1,2,4]triazin-4-amine;
{2-[(4-Fluoro-phenyl)-(3-pyrrolidin-1-yl-propoxy)-methyl]-pyrrolo[2,1-f][1,2,4]triazin-4-yl}-(5-methyl-1H-pyrazol-3-yl)-amine;
{2-[(4-Fluoro-phenyl)-(3-morpholin-4-yl-propoxy)-methyl]-pyrrolo[2,1-f][1,2,4]triazin-4-yl}-(5-methyl-1H-pyrazol-3-yl)-amine;
(2-{(4-Fluoro-phenyl)-[3-(4-methyl-piperazin-1-yl)-propoxy]-methyl}-pyrrolo[2,1-f][1,2,4]triazin-4-yl)-(5-methyl-1H-pyrazol-3-yl)-amine;
{2-[(4-Fluoro-phenyl)-(2-pyrrolidin-1-yl-ethoxy)-methyl]-pyrrolo[2,1-f][1,2,4]triazin-4-yl}-(5-methyl-1H-pyrazol-3-yl)-amine;
(2-{(4-Fluoro-phenyl)-[2-(4-methyl-piperazin-1-yl)-ethoxy]-methyl}-pyrrolo[2,1-f][1,2,4]triazin-4-yl)-(5-methyl-1H-pyrazol-3-yl)-amine;
{2-[(2-Dimethylamino-ethoxy)-(4-fluoro-phenyl)-methyl]-pyrrolo[2,1-f][1,2,4]triazin-4-yl}-(5-methyl-1H-pyrazol-3-yl)-amine;
2-((4-fluorophenyl)(methylamino)methyl)-N-(5-methyl-1H-pyrazol-3-yl)pyrrolo[1,2-f][1,2,4]triazin-4-amine;
{2-[(4-Fluoro-phenyl)-pyrrolidin-1-yl-methyl]-pyrrolo[2,1-f][1,2,4]triazin-4-yl}-(5-methyl-1H-pyrazol-3-yl)-amine;
{2-[(4-Fluoro-phenyl)-morpholin-4-yl-methyl]-pyrrolo[2,1-f][1,2,4]triazin-4-yl}-(5-methyl-1H-pyrazol-3-yl)-amine;
N¹-((4-fluorophenyl)(4-(5-methyl-1H-pyrazol-3-ylamino)pyrrolo[1,2-f][1,2,4]triazin-2-yl)methyl)-N²,N²-dimethylethane-1,2-diamine;
{2-[(4-Fluoro-phenyl)-(2-methoxy-ethylamino)-methyl]-pyrrolo[2,1-f][1,2,4]triazin-4-yl}-(5-methyl-1H-pyrazol-3-yl)-amine;
{2-[(4-Fluoro-phenyl)-imidazol-1-yl-methyl]-pyrrolo[2,1-f][1,2,4]triazin-4-yl}-(5-methyl-1H-pyrazol-3-yl)-amine;
N-{(4-Fluoro-phenyl)-[4-(5-methyl-1-pyrazol-3-ylamino)-pyrrolo[2,1-f][1,2,4]triazin-2-yl]-methyl}-N,N′,N′-trimethyl-ethane-1,2-diamine;
{2-[Dimethylamino-(4-fluoro-phenyl)-methyl]-pyrrolo[2,1-f][1,2,4]triazin-4-yl}-(5-methyl-1H-pyrazol-3-yl)-amine;
{2-[(4-Fluoro-phenyl)-(tetrahydro-pyran-4-yloxy)-methyl]-pyrrolo[2,1-f][1,2,4]triazin-4-yl}-(5-methyl-1H-pyrazol-3-yl)-amine;
{2-[(4-Fluoro-phenyl)-(tetrahydro-furan-3-yloxy)-methyl]-pyrrolo[2,1-f][1,2,4]triazin-4-yl}-(5-methyl-1H-pyrazol-3-yl)-amine;
2-((R,S)-(4-fluorophenyl)((R)-tetrahydrofuran-3-yloxy)methyl)-N-(5-methyl-1H-pyrazol-3-yl)pyrrolo[1,2-f][1,2,4]triazin-4-amine;
{2-[(4-Fluoro-phenyl)-(2-pyrrolidin-1-yl-ethylamino)-methyl]-pyrrolo[2,1-f][1,2,4]triazin-4-yl}-(5-methyl-1H-pyrazol-3-yl)-amine;
{2-[(4-Fluoro-phenyl)-(2-pyridin-2-yl-ethylamino)-methyl]-pyrrolo[2,1-f][1,2,4]triazin-4-yl}-(5-methyl-1H-pyrazol-3-yl)-amine;
{2-[(2-Dimethylamino-ethylsulfanyl)-(4-fluoro-phenyl)-methyl]-pyrrolo[2,1-f][1,2,4]triazin-4-yl}-(5-methyl-1H-pyrazol-3-yl)-amine;
4-{(4-Fluoro-phenyl)-[4-(5-methyl-1H-pyrazol-3-ylamino)-pyrrolo[2,1-f][1,2,4]triazin-2-yl]-methoxy}-2-methyl-butan-2-ol;
1-(2-{(4-Fluoro-phenyl)-[4-(5-methyl-1H-pyrazol-3-ylamino)-pyrrolo[2,1-f][1,2,4]triazin-2-yl]-methoxy}-ethyl)-pyrrolidin-2-one;
{2-[Ethylamino-(4-fluoro-phenyl)-methyl]-pyrrolo[2,1-f][1,2,4]triazin-4-yl}-(5-methyl-1H-pyrazol-3-yl)-amine;
{2-[(4-Fluoro-phenyl)-isopropylamino-methyl]-pyrrolo[2,1-f][1,2,4]triazin-4-yl}-(5-methyl-1H-pyrazol-3-yl)-amine;
{2-[(4-Fluoro-phenyl)-isobutylamino-methyl]-pyrrolo[2,1-f][1,2,4]triazin-4-yl}-(5-methyl-1H-pyrazol-3-yl)-amine;
{2-[tert-Butylamino-(4-fluoro-phenyl)-methyl]-pyrrolo[2,1-f][1,2,4]triazin-4-yl}-(5-methyl-1H-pyrazol-3-yl)-amine;
{2-[Cyclobutylamino-(4-fluoro-phenyl)-methyl]-pyrrolo[2,1-f][1,2,4]triazin-4-yl}-(5-methyl-1H-pyrazol-3-yl)-amine;
{2-[Cyclopropylamino-(4-fluoro-phenyl)-methyl]-pyrrolo[2,1-f][1,2,4]triazin-4-yl}-(5-methyl-1H-pyrazol-3-yl)-amine;
{2-[(2-tert-Butoxy-ethoxy)-(4-fluoro-phenyl)-methyl]-pyrrolo[2,1-f][1,2,4]triazin-4-yl}-(5-methyl-1H-pyrazol-3-yl)-amine;
2-{(4-Fluoro-phenyl)-[4-(5-methyl-1-pyrazol-3-ylamino)-pyrrolo[2,1-f][1,2,4]triazin-2-yl]-methylsulfanyl}-ethanol;
{2-[(Cyclopropylmethyl-amino)-(4-fluoro-phenyl)-methyl]-pyrrolo[2,1-f][1,2,4]triazin-4-yl}-(5-methyl-1H-pyrazol-3-yl)-amine;
2-((R,S)-(4-fluorophenyl)(((S)-tetrahydrofuran-2-yl)methylamino)methyl)-N-(5-methyl-1H-pyrazol-3-yl)pyrrolo[1,2-f][1,2,4]triazin-4-amine;
2-((R,S)-(4-fluorophenyl)(((R)-tetrahydrofuran-2-yl)methylamino)methyl)-N-(5-methyl-1H-pyrazol-3-yl)pyrrolo[1,2-f][1,2,4]triazin-4-amine;
{2-[Ethoxy-(4-fluoro-phenyl)-methyl]-pyrrolo[2,1-f][1,2,4]triazin-4-yl}-(5-methyl-1H-pyrazol-3-yl)-amine;
{2-[(4-Fluoro-phenyl)-(2,2,2-trifluoro-ethoxy)-methyl]-pyrrolo[2,1-f][1,2,4]triazin-4-yl}-(5-methyl-1H-pyrazol-3-yl)-amine;
{2-[(2,2-Difluoro-ethoxy)-(4-fluoro-phenyl)-methyl]-pyrrolo[2,1-f][1,2,4]triazin-4-yl}-(5-methyl-1H-pyrazol-3-yl)-amine;
2-[(4-Fluoro-phenyl)-thiocyanato-methyl]-pyrrolo[2,1-f][1,2,4]triazin-4-yl}-(5-methyl-1H-pyrazol-3-yl)-amine;
{2-[(4-Fluoro-phenyl)-methylsulfanyl-methyl]-pyrrolo[2,1-f][1,2,4]triazin-4-yl}-(5-methyl-1H-pyrazol-3-yl)-amine;
2-(difluoro(4-fluorophenyl)methyl)-6-((2-methoxyethoxy)methyl)-N-(5-methyl-1H-pyrazol-3-yl)pyrrolo[1,2-f][1,2,4]triazin-4-amine;
3-(2-(4-fluorobenzoyl)pyrrolo[1,2-f][1,2,4]triazin-4-ylamino)-1H-pyrazole-5-carbonitrile;
5-{2-[(4-Fluoro-phenyl)-hydroxy-methyl]-pyrrolo[2,1-f][1,2,4]triazin-4-ylamino}-21H-pyrazole-35-carbonitrile;
(6-chloro-4-(5-methyl-1H-pyrazol-3-ylamino)pyrrolo[1,2-f][1,2,4]triazin-2-yl)(4-fluorophenyl)methanone;
[6-Chloro-4-(5-methyl-1H-pyrazol-3-ylamino)-pyrrolo[2,1-f][1,2,4]triazin-2-yl]-(4-fluoro-phenyl)-methanol;
2-((4-fluorophenyl)(((R)-tetrahydrofuran-2-yl)methoxy)methyl)-N-(5-methyl-1H-pyrazol-3-yl)pyrrolo[1,2-f][1,2,4]triazin-4-amine;
2-((2-(tert-butylamino)ethoxy)(4-fluorophenyl)methyl)-N-(5-methyl-1H-pyrazol-3-yl)pyrrolo[1,2-f][1,2,4]triazin-4-amine;
2-((4-fluorophenyl)(2-morpholinoethylthio)methyl)-N-(5-methyl-1H-pyrazol-3-yl)pyrrolo[1,2-f][1,2,4]triazin-4-amine;
2-((4-fluorophenyl)(3-(methylthio)propylamino)methyl)-N-(5-methyl-1H-pyrazol-3-yl)pyrrolo[1,2-f][1,2,4]triazin-4-amine;
N-((2-(difluoro(4-fluorophenyl)methyl)-4-(5-methyl-1,4-pyrazol-3-ylamino)pyrrolo[1,2-f][1,2,4]triazin-6-yl)methyl)-2-morpholinoacetamide;
1-(2-(difluoro(4-fluorophenyl)methyl)-4-(5-methyl-1H-pyrazol-3-ylamino)pyrrolo[1,2-f][1,2,4]triazin-6-yl)-2,2,2-trifluoroethanol;
2-amino-1-(2-(difluoro(4-fluorophenyl)methyl)-4-(5-methyl-1H-pyrazol-3-ylamino)pyrrolo[1,2-f][1,2,4]triazin-6-yl)ethanol;
N-(2-(2-(difluoro(4-fluorophenyl)methyl)-4-(5-methyl-1H-pyrazol-3-ylamino)pyrrolo[1,2-f][1,2,4]triazin-6-yl)-2-hydroxyethyl)acetamide;
1-(2-(difluoro(4-fluorophenyl)methyl)-4-(5-methyl-1H-pyrazol-3-ylamino)pyrrolo[1,2-f][1,2,4]triazin-6-yl)-2-morpholinoethanol;
5-(2-(difluoro(4-fluorophenyl)methyl)-4-(5-methyl-1H-pyrazol-3-ylamino)pyrrolo[1,2-f][1,2,4]triazin-6-yl)oxazolidin-2-one;
2-(1-(4-fluorophenyl)ethyl)-N-(1H-pyrazol-3-yl)pyrrolo[1,2-f][1,2,4]triazin-4-amine;
4-(4-(1H-pyrazol-3-ylamino)pyrrolo[1,2-f][1,2,4]triazin-2-yl)-4-(4-fluorophenyl)butan-1-ol;
2-(2-(difluoro(4-fluorophenyl)methyl)-4-(5-methyl-1H-pyrazol-3-ylamino) pyrrolo[1,2-f][1,2,4]triazin-6-yl)propan-2-ol;
[2-(4-Fluoro-benzyl)-pyrrolo[2,1-f][1,2,4]triazin-4-yl]-(5-methyl-1H-pyrazol-3-yl)amine;
(6-fluoro-4-(5-methyl-1H-pyrazol-3-ylamino)pyrrolo[1,2-f][1,2,4]triazin-2-yl)(4-fluorophenyl)methanone;
(6-fluoro-4-(5-methyl-1H-pyrazol-3-ylamino)pyrrolo[1,2-f][1,2,4]triazin-2-yl)(4-fluorophenyl)methanol;
6-fluoro-2-((4-fluorophenyl)(methoxy)methyl)-N-(5-methyl-1H-pyrazol-3-yl)pyrrolo[1,2-f][1,2,4]triazin-4-amine;
(4-(1H-pyrazol-3-ylamino)-6-fluoropyrrolo[1,2-f][1,2,4]triazin-2-yl)(4-fluorophenyl)methanone;
2-(difluoro(4-fluorophenyl)methyl)-6-fluoro-N-(5-methyl-1H-pyrazol-3-yl)pyrrolo[1,2-f][1,2,4]triazin-4-amine;
2-(difluoro(4-fluorophenyl)methyl)-6-fluoro-N-(1H-pyrazol-3-yl)pyrrolo[1,2-f][1,2,4]triazin-4-amine;
(2-(difluoro(4-fluorophenyl)methyl)-4-(5-methyl-1H-pyrazol-3-ylamino)pyrrolo[1,2-f][1,2,4]triazin-6-yl)(morpholino) methanethione;
2-(difluoro(4-fluorophenyl)methyl)-N-(5-methyl-1H-pyrazol-3-yl)-6-(methylsulfonylmethyl)pyrrolo[1,2-f][1,2,4]triazin-4-amine;
N-(2-(difluoro(4-fluorophenyl)methyl)-4-(5-methyl-1H-pyrazol-3-ylamino)pyrrolo[1,2-f][1,2,4]triazin-6-yl)-3-morpholinopropanamide;
N-(5-chloro-2-(difluoro(4-fluorophenyl)methyl)-4-(5-methyl-1H-pyrazol-3-ylamino)pyrrolo[1,2-f][1,2,4]triazin-6-yl)-3-morpholinopropanamide;
N-(2-(difluoro(4-fluorophenyl)methyl)-4-(5-methyl-1H-pyrazol-3-ylamino)pyrrolo[1,2-f][1,2,4]triazin-6-yl)acrylamide;
N-(2-(difluoro(4-fluorophenyl)methyl)-4-(5-methyl-1H-pyrazol-3-ylamino)pyrrolo[1,2-f][1,2,4]triazin-6-yl)-2-(4,4-difluoropiperidin-1-yl)ethanesulfonamide;
(7-bromo-4-(5-methyl-1H-pyrazol-3-ylamino)pyrrolo[1,2-f][1,2,4]triazin-2-yl)(4-fluorophenyl)methanone;
2-(4-fluorophenyl)-2-(4-(5-methyl-1H-pyrazol-3-ylamino)pyrrolo[1,2-f][1,2,4]triazin-2-yl)ethanol;
1-(4-fluorophenyl)-1-(4-(5-methyl-1H-pyrazol-3-ylamino)pyrrolo[1,2-f][1,2,4]triazin-2-yl)ethane-1,2-diol;
2-fluoro-2-(4-fluorophenyl)-2-(4-(5-methyl-1H-pyrazol-3-ylamino)pyrrolo[1,2-f][1,2,4]triazin-2-yl)ethanol;
2-(1-fluoro-1-(4-fluorophenyl)-2-methoxyethyl)-N-(5-methyl-1H-pyrazol-3-yl)pyrrolo[1,2-f][1,2,4]triazin-4-amine;
2-(2-amino-1-fluoro-1-(4-fluorophenyl)ethyl)-N-(5-methyl-1H-pyrazol-3-yl)pyrrolo[1,2-f][1,2,4]triazin-4-amine;
5-(4-fluorophenyl)-5-(4-(5-methyl-1H-pyrazol-3-ylamino)pyrrolo[1,2-f][1,2,4]triazin-2-yl)oxazolidin-2-one;
2-((4-fluorophenyl)(methoxyamino)methyl)-N-(5-methyl-1H-pyrazol-3-yl)pyrrolo[1,2-f][1,2,4]triazin-4-amine;
2-(2-(4-fluorophenyl)-1,3-dioxolan-2-yl)-N-(5-methyl-1H-pyrazol-3-yl)pyrrolo[1,2-f][1,2,4]triazin-4-amine;
2-(2-(4-fluorophenyl)-1,3-oxathiolan-2-yl)-N-(5-methyl-1H-pyrazol-3-yl)pyrrolo[1,2-f][1,2,4]triazin-4-amine;
3-(4-fluorophenyl)-3-(4-(5-methyl-1H-pyrazol-3-ylamino)pyrrolo[1,2-f][1,2,4]triazin-2-yl)propan-1-ol;
1-((4-fluorophenyl)(4-(5-methyl-1H-pyrazol-3-ylamino)pyrrolo[1,2-f][1,2,4]triazin-2-yl)methyl)-3-methylurea;
2-(1-amino-1-(4-fluorophenyl)ethyl)-N-(5-methyl-1H-pyrazol-3-yl)pyrrolo[1,2-f][1,2,4]triazin-4-amine;
2-((4-fluorophenyl)(methylsulfinyl)methyl)-N-(5-methyl-1H-pyrazol-3-yl)pyrrolo[1,2-f][1,2,4]triazin-4-amine;
2((4-fluorophenyl)(methylsulfonyl)methyl)-N-(5-methyl-1H-pyrazol-3-yl)pyrrolo[1,2-f][1,2,4]triazin-4-amine;
(4-(1H-pyrazol-3-ylamino)pyrrolo[1,2-f][1,2,4]triazin-2-yl)(2-(methylthio)phenyl)methanone;
(4-chlorophenyl)(4-(5-methyl-1H-pyrazol-3-ylamino)pyrrolo[1,2-f][1,2,4]triazin-2-yl)methanone;
(4-Methoxy-phenyl)-[4-(5-methyl-1H-pyrazol-3-ylamino)-pyrrolo[2,1-f][1,2,4]triazin-2-yl]-methanone;
(4-Fluoro-3-methyl-phenyl)-[4-(5-methyl-1H-pyrazol-3-ylamino)-pyrrolo[2,1-f][1,2,4]triazin-2-yl]-methanone;
Benzo[1,3]dioxol-5-yl-[4-(5-methyl-1H-pyrazol-3-ylamino)-pyrrolo[2,1-f][1,2,4]triazin-2-yl]-methanone;
(3,4-Difluoro-phenyl)-[4-(5-methyl-1H-pyrazol-3-ylamino)-pyrrolo[2,1-f][1,2,4]triazin-2-yl]-methanone;
(4-Methoxy-phenyl)-[4-(5-methyl-1H-pyrazol-3-ylamino)-pyrrolo[2,1-f][1,2,4]triazin-2-yl]-methanol;
Benzo[1,3]dioxol-5-yl-[4-(5-methyl-1H-pyrazol-3-ylamino)-pyrrolo[2,1-f][1,2,4]triazin-2-yl]-methanol;
(3,4-Difluoro-phenyl)-[4-(5-methyl-1H-pyrazol-3-ylamino)-pyrrolo[2,1-f][1,2,4]triazin-2-yl]-methanol;
(4-Fluoro-3-methyl-phenyl)-[4-(5-methyl-1H-pyrazol-3-ylamino)-pyrrolo[2,1-f][1,2,4]triazin-2-yl]-methanol;
(4-Chloro-phenyl)-[4-(5-methyl-1H-pyrazol-3-ylamino)-pyrrolo[2,1-f][1,2,4]triazin-2-yl]-methanol;
methyl (4-fluorophenyl)(4-(5-methyl-1H-pyrazol-3-ylamino)pyrrolo[1,2f][1,2,4]triazin-2-yl)methylcarbamate;
{(4-Fluoro-phenyl)-[4-(5-methyl-1H-pyrazol-3-ylamino)-pyrrolo[2,1-f][1,2,4]triazin-2-yl]-methyl}-carbamic acid ethyl ester;
2-Amino-N-{(4-fluoro-phenyl)-[4-(5-methyl-1H-pyrazol-3-ylamino)-pyrrolo[2,1-f][1,2,4]triazin-2-yl]-methyl}-propionamide;
(R)-2-amino-N—((R,S)-(4-fluorophenyl)(4-(5-methyl-1H-pyrazol-3-ylamino)pyrrolo[1,2-f][1,2,4]triazin-2-yl)methyl)propanamide;
2-Amino-N-{(4-fluoro-phenyl)-[4-(5-methyl-1H-pyrazol-3-ylamino)-pyrrolo[2,1-f][1,2,4]triazin-2-yl]-methyl}-acetamide;
3-{(4-Fluoro-phenyl)-[4-(5-methyl-1H-pyrazol-3-ylamino)-pyrrolo[2,1-f][1,2,4]triazin-2-yl]-methyl}-1,1-dimethyl-urea;
N-{(4-Fluoro-phenyl)-[4-(5-methyl-1-pyrazol-3-ylamino)-pyrrolo[2,1-f][1,2,4]triazin-2-yl]-methyl}-formamide;
N-{(4-Fluoro-phenyl)-[4-(5-methyl-1-pyrazol-3-ylamino)-pyrrolo[2,1-f][1,2,4]triazin-2-yl]-methyl}-methanesulfonamide;
N-{(4-Fluoro-phenyl)-[4-(5-methyl-1H-pyrazol-3-ylamino)-pyrrolo[2,1-f][1,2,4]triazin-2-yl]-methyl}-acetamide;
N-(4-Fluoro-phenyl)-[4-(5-methyl-1-pyrazol-3-ylamino)-pyrrolo[2,1-f][1,2,4]triazin-2-yl]-methyl}-isobutyramide;
2,2,2-Trifluoro-N-{(4-fluoro-phenyl)-[4-(5-methyl-1H-pyrazol-3-ylamino)pyrrolo[2,1-f][1,2,4]triazin-2-yl]-methyl}-acetamide;
2-((4-fluorophenyl)(2-methoxyethoxy)methyl)-N-(5-methyl-1H-pyrazol-3-yl)pyrrolo[1,2-f][1,2,4]triazin-4-amine;
{2-[(2-Ethoxy-ethoxy)-(4-fluoro-phenyl)-methyl]pyrrolo[2,1-f][1,2,4]triazin-4-yl}-(5-methyl-1H-pyrazol-3-yl)-amine;
2-{(4-Fluoro-phenyl)-[4-(5-methyl-1H-pyrazol-3-ylamino)-pyrrolo[2,1-f][1,2,4]triazin-2-yl]-methoxy}-ethanol;
3-{(4-Fluoro-phenyl)-[4-(5-methyl-1-pyrazol-3-ylamino)-pyrrolo[2,1-f][1,2,4]triazin-2-yl]-methoxy}-propan-1-ol;
2-((4-fluorophenyl)(3-morpholinopropylthio)methyl)-N-(5-methyl-1H-pyrazol-3-yl)pyrrolo[1,2-f][1,2,4]triazin-4-amine;
(2-{(4-Fluoro-phenyl)-[2-(4-methyl-piperazin-1-yl)-ethylsulfanyl]-methyl}-pyrrolo[2,1-f][1,2,4]triazin-4-yl)-(5-methyl-1H-pyrazol-3-yl)-amine;
(2-{(4-Fluoro-phenyl)-[3-(4-methyl-piperazin-1-yl)-propylsulfanyl]-methyl}-pyrrolo[2,1-f][1,2,4]triazin-4-yl)-(5-methyl-1H-pyrazol-3-yl)-amine;
{2-[(3-Dimethylamino-propylsulfanyl)-(4-fluoro-phenyl)-methyl]-pyrrolo[2,1-f][1,2,4]triazin-4-yl}-(5-methyl-1H-pyrazol-3-yl)-amine;
{2-[(2-Amino-ethylsulfanyl)-(4-fluoro-phenyl)-methyl]-pyrrolo[2,1-f][1,2,4]triazin-4-yl}-(5-methyl-1H-pyrazol-3-yl)-amine;
{2-[[2-(3,3-Difluoro-pyrrolidin-1-yl)-ethylsulfanyl]-(4-fluoro-phenyl)-methyl]-pyrrolo[2,1-f][1,2,4]triazin-4-yl}-(5-methyl-1H-pyrazol-3-yl)-amine;
(2S)-((R,S)(4-fluorophenyl) (4-(5-methyl-1H-pyrazol-3-ylamino)pyrrolo[1,2-f][1,2,4]triazin-2-yl)methyl) 2-aminopropanoate;
Amino-acetic acid (4-fluoro-phenyl)-[4-(5-methyl-1H-pyrazol-3-ylamino)pyrrolo[2,1-f][1,2,4]triazin-2-yl]-methyl ester;
(R)-((R,S)-(4-fluorophenyl)(4-(5-methyl-1H-pyrazol-3-ylamino)pyrrolo[1,2-f][1,2,4]triazin-2-yl)methyl) 2-aminopropanoate;
Morpholin-4-yl-acetic acid (4-fluoro-phenyl)-[4-(5-methyl-1H-pyrazol-3-ylamino)pyrrolo[2,1-f][1,2,4]triazin-2-yl]-methyl ester;
2-Amino-2-methyl-propionic acid (4-fluoro-phenyl)-[4-(5-methyl-1H-pyrazol-3-ylamino)-pyrrolo[2,1-f][1,2,4]triazin-2-yl]-methyl ester;
2-Methyl-2-methylamino-propionic acid (4-fluoro-phenyl)-[4-(5-methyl-1H-pyrazol-3-ylamino)-pyrrolo[2,1-f][1,2,4]triazin-2-yl]-methyl ester;
methyl 2-((4-fluorophenyl)(methoxy)methyl)-N-(5-methyl-1H-pyrazol-3-yl)pyrrolo[1,2-f][1,2,4]triazin-4-amine; and
N-(4-bromo-5-methyl-1H-pyrazol-3-yl)-2-(difluoro(4-fluorophenyl)methyl)-6-(methylsulfonylmethyl)pyrrolo[1,2-f][1,2,4]triazin-4-amine.

### C. Formulation of Pharmaceutical Compositions

The pharmaceutical compositions provided herein contain therapeutically effective amounts of one or more of compounds provided herein that are useful in the prevention, treatment, or amelioration of JAK kinase, including JAK2 kinase, mediated diseases or one or more of the symptoms thereof.

The compositions contain one or more compounds provided herein. The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art.

In the compositions, effective concentrations of one or more compounds or pharmaceutically acceptable salt, solvate, hydrate or prodrug is (are) mixed with a suitable pharmaceutical carrier or vehicle. The concentrations of the compounds in the compositions are effective for delivery of an amount, upon administration, that treats, prevents, or ameliorates one or more of the symptoms of JAK kinase, including JAK2 kinase, mediated diseases.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of JAK kinase mediated diseases.

Typically a therapeutically effective dosage should produce a serum concentration of active ingredient of from about 1 ng/ml to about 50-100 μg/ml. The pharmaceutical compositions typically should provide a dosage of from about 10 mg to about 4000 mg of compound per kilogram of body weight per day. Pharmaceutical dosage unit forms are prepared to provide from about 10 mg to about 1000 mg and in certain embodiments, from about 10 mg to about 500 mg, from about 20 mg to about 250 mg or from about 25 mg to about 100 mg of the essential active ingredient or a combination of essential ingredients per dosage unit form. In certain embodiments, the pharmaceutical dosage unit forms are prepared to provide about 10 mg, 20 mg, 25 mg, 50 mg, 100 mg, 250 mg, 500 mg, 1000 mg or 2000 mg of the essential active ingredient.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing JAK kinase, including JAK2 kinase, mediated diseases. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including, but not limited to, orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. In one embodiment, the effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001%-100% active ingredient, in certain embodiments, about 0.1-85%, typically about 75-95%.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as JAK kinase, including JAK2 kinase mediated diseases. It is to be understood that such combination therapy constitutes a further aspect of the compositions and methods of treatment provided herein.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin; methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. In one embodiment, the composition is administered as an aqueous solution with hydroxypropyl-beta-cyclodextrin (HPBCD) as an excipient. In one embodiment, the aqueous solution contains about 1% to about 50% HPBCD. In one embodiment, the aqueous solution contains about 1%, 3%, 5%, 10% or about 20% HPBCD.

Implantation of a slow-release or sustained-release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate.

Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose, hydroxypropyl-beta-cyclodextrin (HPBCD) or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, 100-500 mg, 10-500 mg, 50-250 mg or 25-100 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation. These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity of the therapeutic target, i.e., thus requiring only a fraction of the systemic dose. In some embodiments, a controlled release device is introduced into a subject in proximity of the site of inappropriate immune activation or a tumor. The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

7. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### D. Evaluation of the Activity of the Compounds

Standard physiological, pharmacological and biochemical procedures are available for testing the compounds to identify those that possess biological activities that modulate the activity of JAK kinases, including wild type and mutant JAK kinases.

Such assays include, for example, biochemical assays such as binding assays, radioactivity incorporation assays, as well as a variety of cell based assays.

Exemplary cell based assay methodologies include measurement of STAT5A phosphorylation or proliferation in leukemic cell lines such as TF-1 or HEL-2.

Cells useful in the assays include cells with wildtype or mutated forms. Suitable cells include those derived through cell culture from patient samples as well as cells derived using routine molecular biology techniques, e.g., retroviral transduction, transfection, mutagenesis, etc.

### E. Methods of Use of the Compounds and Compositions

Also provided herein are methods of using the disclosed compounds and compositions, or pharmaceutically acceptable salts, solvates, hydrates or prodrugs thereof, for the treatment, prevention, or amelioration of a disease or disorder that is mediated or otherwise affected via JAK kinase, including JAK2 kinase activity or one or more symptoms of diseases or disorders that are mediated or otherwise affected via JAK kinase, including JAK2 kinase, activity. JAK kinase can be wild type and/or mutant form of JAK2 kinase. Consistent with the description above, such diseases or disorders include without limitation: cancers, including myeloproliferative disorders such as polycythemia vera (PCV), essential thrombocythemia and idiopathic myelofibrosis (IMF); myeloid leukemia including chronic myeloid leukemia (CML), imatinib-resistant forms of CML, acute myeloid leukemia (AML), and a subtype of AML, acute megakaryoblastic leukemia (AMKL); lymphoproliferative diseases such as myeloma, head and neck cancers, prostate cancer, breast cancer, ovarian cancer, melanoma, lung cancers, brain tumors, pancreatic and renal carcinoma; and inflammatory diseases or disorders related to immune dysfunction, immunodeficiency, immunomodulation, autoimmune diseases, tissue transplant rejection, graft-versus-host disease, wound healing, kidney disease, multiple sclerosis, thyroiditis, type 1 diabetes, sarcoidosis, psoriasis, allergic rhinitis, inflammatory bowel disease including Crohn's disease and ulcerative colitis (UC), systemic lupus erythematosis (SLE), arthritis, osteoarthritis, rheumatoid arthritis, osteoporosis, asthma and chronic obstructive pulmonary disease (COPD).

### F. Combination Therapy

Furthermore, it will be understood by those skilled in the art that the compounds, isomers, prodrugs and pharmaceutically acceptable derivatives provided herein, including pharmaceutical compositions and formulations containing these compounds, can be used in a wide variety of combination therapies to treat the conditions and diseases described above. Thus, also contemplated herein is the use of compounds, isomers, prodrugs and pharmaceutically acceptable derivatives provided herein in combination with other active pharmaceutical agents for the treatment of the disease/conditions described herein.

In one embodiment, such additional pharmaceutical agents include without limitation anti-cancer agents, including chemotherapeutic agents and anti-proliferative agents; anti-inflammatory agents and immunomodulatory agents or immunosuppressive agents.

In certain embodiments, the anti-cancer agents include anti-metabolites (e.g., 5-fluoro-uracil, cytarabine, methotrexate, fludarabine and others), antimicrotubule agents (e.g., vinca alkaloids such as vincristine, vinblastine; taxanes such as paclitaxel and docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, satraplatin and CI-973), anthracyclines (e.g., doxrubicin and daunorubicin), antitumor antibiotics (e.g., mitomycin, idarubicin, adriamycin and daunomycin), topoisomerase inhibitors (e.g., etoposide and camptothecins), anti-angiogenesis agents (e.g. Sutent®, sorafenib and Bevacizumab) or any other cytotoxic agents, (e.g. estramustine phosphate, prednimustine), hormones or hormone agonists, antagonists, partial agonists or partial antagonists, kinase inhibitors (such as imatinib), and radiation treatment.

In certain embodiments, the anti-inflammatory agents include matrix metalloproteinase inhibitors, inhibitors of pro-inflammatory cytokines (e.g., anti-TNF molecules, TNF soluble receptors, and IL1) non-steroidal anti-inflammatory drugs (NSAIDs) such as prostaglandin synthase inhibitors (e.g., choline magnesium salicylate and salicylsalicyclic acid), COX-1 or COX-2 inhibitors, or glucocorticoid receptor agonists such as corticosteroids, methylprednisone, prednisone, or cortisone.

The compound or composition provided herein, or pharmaceutically acceptable derivative thereof, may be administered simultaneously with, prior to, or after administration of one or more of the above agents.

Pharmaceutical compositions containing a compound provided herein or pharmaceutically acceptable derivative thereof, and one or more of the above agents are also provided.

Also provided is a combination therapy that treats or prevents the onset of the symptoms, or associated complications of cancer and related diseases and disorders comprising the administration to a subject in need thereof, of one of the compounds or compositions disclosed herein, or pharmaceutically acceptable derivatives thereof, with one or more anti-cancer agents.

### G. Preparation of Compounds

Starting materials in the synthesis examples provided herein are either available from commercial sources or via literature procedures either as cited or as found, for example in March Advanced Organic Chemistry: Reactions, Mechanisms, and Structure, (1992) 4th Ed.; Wiley Interscience, New York. All commercially available compounds were used without further purification unless otherwise indicated. Proton (¹H) nuclear magnetic resonance (NMR) spectra were recorded on a Bruker Avance 300 MHz NMR spectrometer. Significant peaks are tabulated and typically include: number of protons, and multiplicity (s, singlet; d, double; t, triplet; q, quartet; m, multiplet; br s, broad singlet). Chemical shifts are reported as parts per million (8) downfield relative to tetramethylsilane. Low resolution mass spectra (MS) were obtained as electrospray ionization (ESI) mass spectra, which were recorded on a Shimadzu HPLC/MS instrument using reverse-phase conditions (acetonitrile/water, 0.05% acetic acid). Preparative HPLC was performed using Varian HPLC systems and Phenomenex columns.

It is understood that in the following description, combinations of substituents and/or variables of the depicted formulae are permissible only if such contributions result in stable compounds under standard conditions.

It will also be appreciated by those skilled in the art that in the process described below, the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein. The use of protecting groups is described in detail in Green, T. W. and P. G. M. Wutz, Protective Groups in Organic Synthesis (1991), 2nd Ed., Wiley-Interscience.

One of ordinary skill in the art could easily ascertain which choices for each substituent are possible for the reaction conditions of each Scheme. Moreover, the substituents are selected from components as indicated in the specification heretofore, and may be attached to starting materials, intermediates, and/or final products according to schemes known to those of ordinary skill in the art.

Also it will be apparent that the compounds provided herein could exist as one or more isomers, that is, E/Z isomers, enantiomers and/or diastereomers. Compounds of formula (I) may be generally prepared as depicted in the following schemes, unless otherwise noted, the various substituents are as defined elsewhere herein.

Standard abbreviations and acronyms as defined in J. Org. Chem. 2007 72(1): 23A-24A are used herein. Other abbreviations and acronyms used herein are as follows:

DAST (diethylamino)sulfur trifluoride

### DIPEA N,N-diisopropylethylamine

DCM dichloromethane
EtOAc—ethyl acetate
EtOH—ethanol
FBS fetal bovine serum
HOAc—acetic acid
MeOH—methanol
min minute(s)
NEAA nonessential amino acids
Na pyr/P/S sodium pyruvate and penicillin/streptomycin.
TEA triethylamine
TMSCl chlorotrimethylsilane

In the following schemes, R¹-R⁸, R¹⁰, R¹¹, R¹³ and R¹⁴ are as described herein for compounds of Formulae (I)-(XIV). Scheme 1 depicts preparation of a carboxylate intermediate which is a precursor to the 2-keto pyrrolotriazine analogues. The pyrrole carbonitrile (1) can be reacted with NaH, potassium t-butoxide or other strong base, in a suitable solvent such as DMF, THF, water, toluene, NMP or DME. The addition of a chloroamine yields the aminated pyrrole (2). The pyrrole carbonitrile is converted to the pyrrole carboxamide (3) under alkaline conditions, for example, by treatment with NaOH, KOH, Ca(OH)₂. The addition of a chlorooxoacetate (where R is alkyl) in a suitable solvent such as TEA and DCE yields a pyrrole-1-ylamino-2-oxoacetate intermediate (4). Cyclization occurs in the presence of TMSCl in a suitable solvent such as TEA and DCE which is allowed to reflux to afford the 4-hydroxy pyrrolotriazine carboxylate intermediate (5). A suitable chlorinating agent such as POCl₃ affords the 4-chloro pyrrolotriazine carboxylate intermediate (6) which may undergo further reactions such as described in Scheme 3 to afford the 2-keto pyrrolotriazine. Preparation of compound 3 may also be effected through an analogous sequence wherein (1) is replaced by a pyrrole-2-carboxylic acid alkyl ester to give the corresponding 1-amino-pyrrole-carboxylic ester in place of (2), which is treated with ammonia to give amide (3).

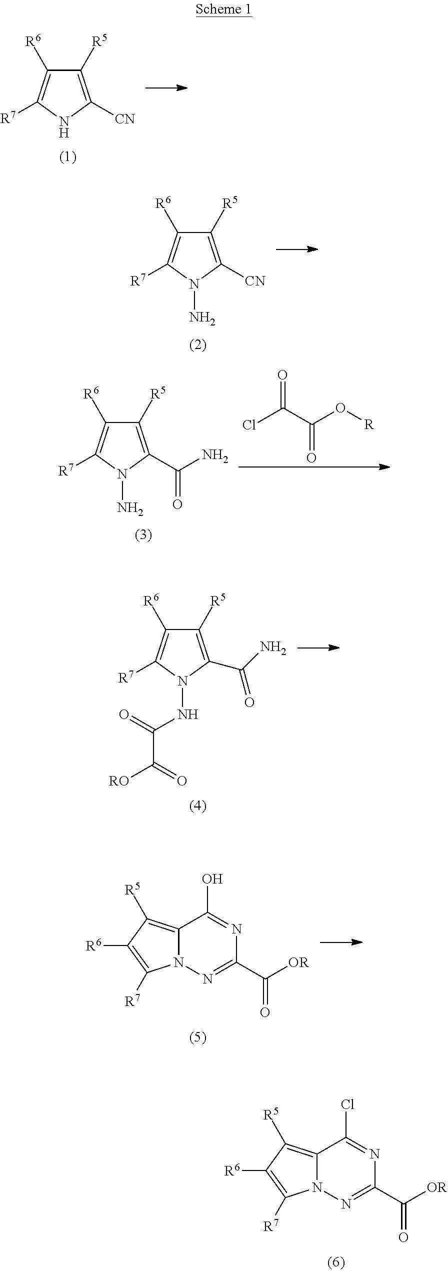

Scheme 2 shows an alternative method of forming the pyrrolotriazine, which introduces a secondary, tertiary or quaternary carbon substituent at the 2-position of the pyrrolotriazine. The pyrrole carbonitrile (1) is aminated with a chloroamine in the presence of a strong base such as NaH in a suitable solvent such as DMF. The pyrrole carbonitrile is converted to the pyrrole carboxamide (3 under aqueous alkaline conditions, for example, by treatment with NaOH, KOH, or Ca(OH)₂. Reaction with the R¹-, R²-, R⁸-substituted acetyl chloride (which may be prepared by the reaction of the appropriately substituted acetic acid with oxalyl chloride) in a suitable organic solvent such as DCM affords the 1-acetamido pyrrole-2-carboxamide intermediate (7). Cyclization occurs in the presence of TMSCl in a suitable solvent such as DCE and TEA, with heating to about 85° C. to yield the 4-hydroxy pyrrolotriazine (8). Alternatively, cyclization may occur in the presence of acetic acid heated to 120° C. for about 2-4 hours. A suitable chlorinating agent such as POCl₃ affords the 4-chloro pyrrolotriazine carboxylate intermediate (9). Treatment of (9) with the appropriately substituted amine (for example, an arylamine or heteroarylamine) in the presence of potassium iodide and DIPEA in DMF at room temperature yields the corresponding 4-amino-pyrrolotriazine derivative (10).

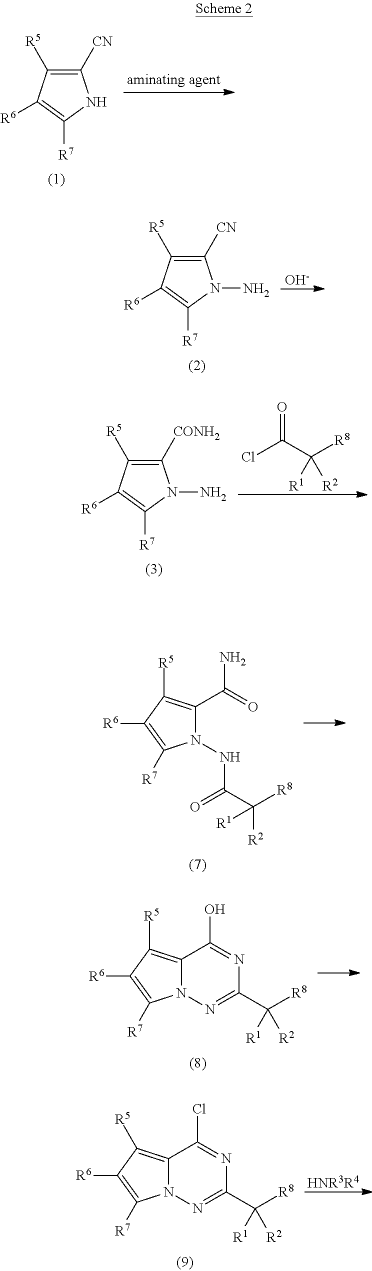

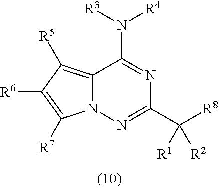

Scheme 3 depicts the synthesis of the 2-keto analogues. The treatment of intermediate (6) with an organomagnesium or organolithium compound in THF at low temperature affords the keto-intermediate (11). Treatment of (11) with the appropriately substituted amine (for example an aryl amine or heteroaryl amine) in the presence of potassium iodide and DIPEA in DMF at room temperature yields the corresponding 4-amino-pyrrolotriazine derivative (12).

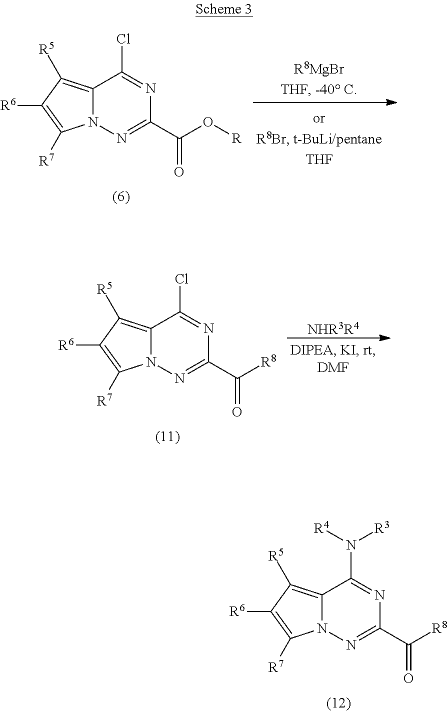

Scheme 4 shows the preparation of hydroxy derivatives (13) which are prepared by treatment of corresponding keto-analogue (12) with a hydride reducing agent such as sodium borohydride under suitable conditions such as in MeOH at 0° C. Treatment of the hydroxy derivative with DAST or derivatives of DAST in a mixture of DCM/THF at room temperature affords the corresponding fluoro derivative having the general Formula (14).

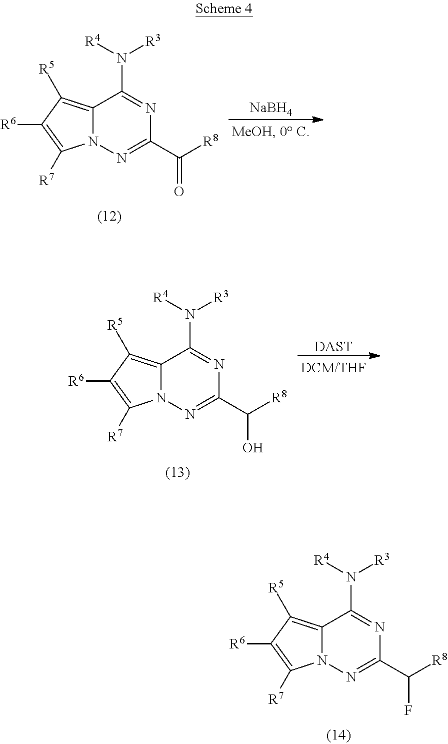

Scheme 5 depicts a method wherein hydroxy derivatives (15) (where R² is an alkyl group) may be prepared by reacting the keto-derivative with an excess of the appropriate organomagnesium or organolithium compound in THF at room temperature.

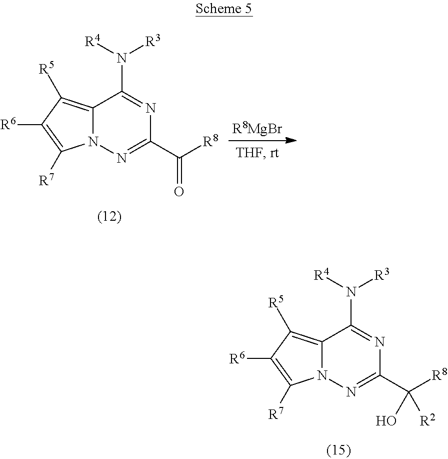

Scheme 6 depicts a way in which compounds of formula (17) may be obtained from compounds having the formula (13) via a two-step sequence. Activation of the alcohol by conversion to a suitable leaving group X and then displacement by an amine yields compounds of the formula (17). A typical activation would be formation of a sulfonate ester (X═—OS(O)₂-alkyl or —OS(O)₂-aryl) by reaction of the alcohol with the corresponding sulfonyl chloride or sulfonic anhydride in an aprotic solvent, together with a suitable base (triethylamine, pyridine, or N,N-4-dimethylaminopyridine or the like). Another typical activation would be the transformation of the hydroxy group into a halide (X═F, Cl, Br, I). Some typical methods of achieving this transformation are by reaction of the alcohol with thionyl chloride, phosphorous pentachloride, phosphorous tribromide, triphenylphosphine-iodine, triphenylphosphine-carbon tetrachloride, DAST, or similar reagents, in an aprotic solvent to produce the corresponding halide (16). After activation, displacement by an amine, typically in a polar, aprotic solvent (for example DMF, dioxane, THF, NMP, DMSO, or the like) affords compounds of formula (17). In the case that one or both of R¹³ and R¹⁴ is hydrogen, the amino group may be further functionalized, for example by acylation, carbamoylation, alkoxycarbonylation, or sulfonation, under conditions well known in the art, to form additional embodiments.

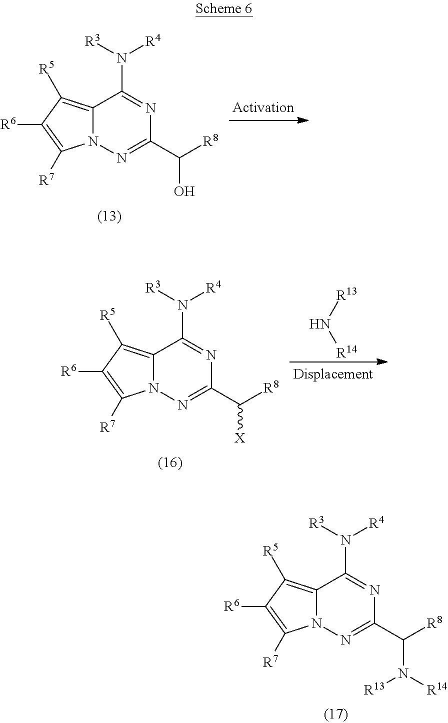

Alternatively, Scheme 7 depicts a way in which the hydroxyl group in compound (13) can be directly replaced by an alkoxyl group by treatment of (13) with an alcohol in the presence of acid under anhydrous conditions to form additional embodiments. When an R group that has been introduced by this procedure contains a leaving group such as halide as a substitutent, the leaving group may subsequently be displaced by another nucleophile such as an amine to produce additional embodiments.

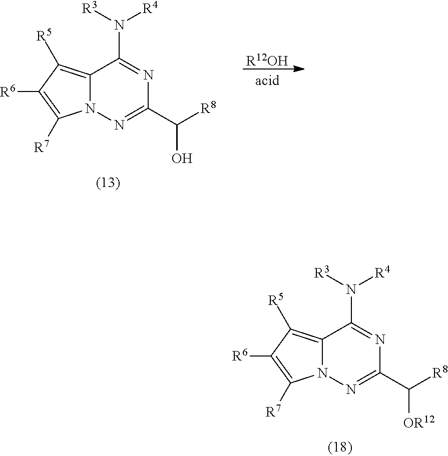

Leaving group X in intermediate (16) may furthermore be displaced by other nucleophiles such as alcoholates or thiolates to produce compounds of formulae (18) and (19), as depicted in Scheme 8. Thiolates may be oxidized to sulfoxides (n=1) or sulfones (n=2) to produce compounds of formula (20).

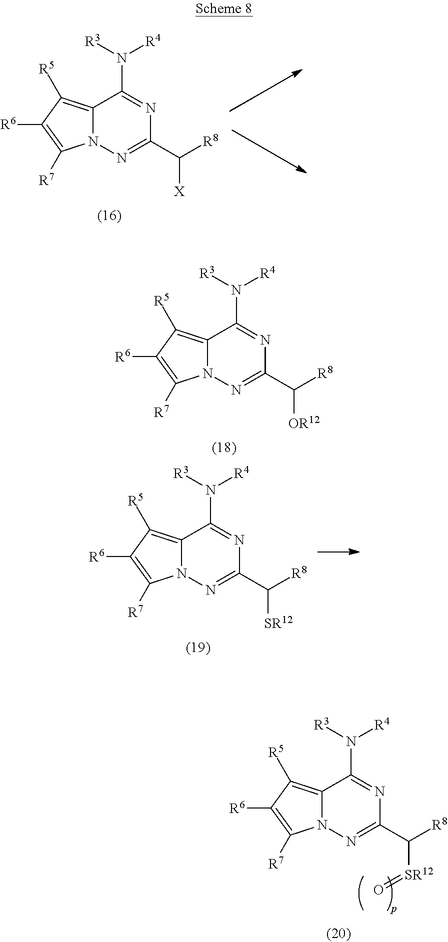

Alternatively, leaving group X in intermediate (16) may be substituted with a mercapto group —SH (19b) through the intermediacy of an S-acyl precursor (19a), as depicted in Scheme 9. Upon treatment with a suitable dihaloalkane, a monohalo thioether is obtained (19c). The halide may be displaced by an amine or an alcoholate to form additional embodiments, such as compounds of formulae (19d) and (19e).

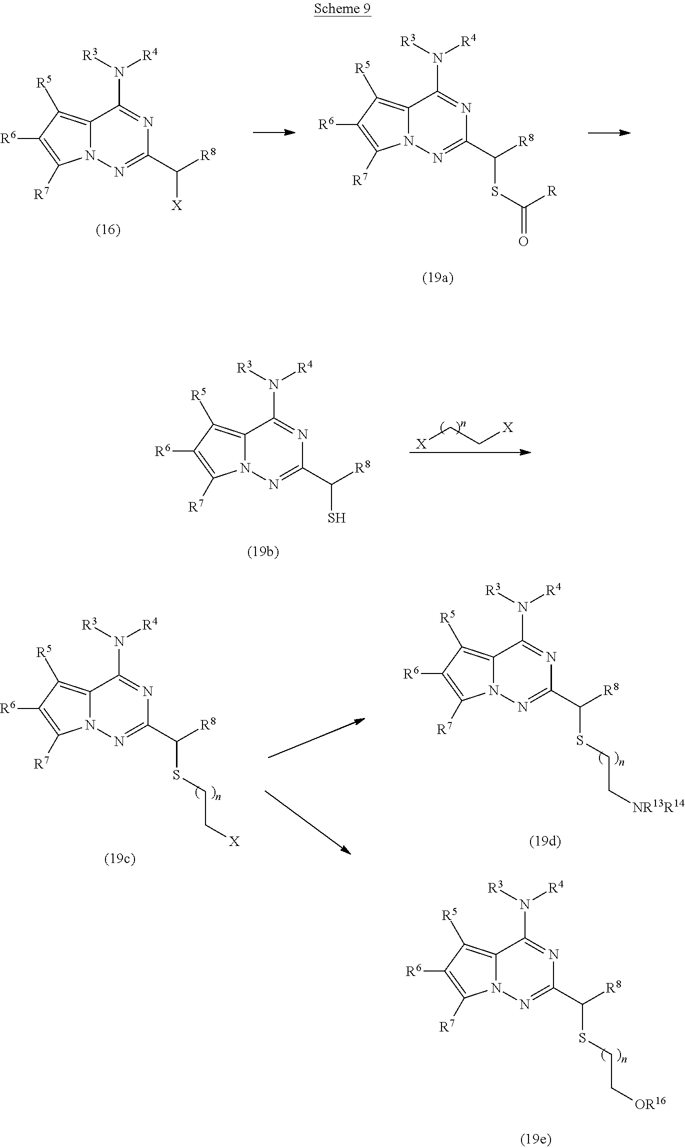

Scheme 10 depicts a way in which a leaving group X in intermediate (16) may furthermore be displaced by nucleophilic carboxylates to form ester products, which constitute additional embodiments. As previously described in general terms, R may be introduced in protected form, for example as a nitrogen-protected aminoalkyl group, which is N-deprotected to form additional embodiments.

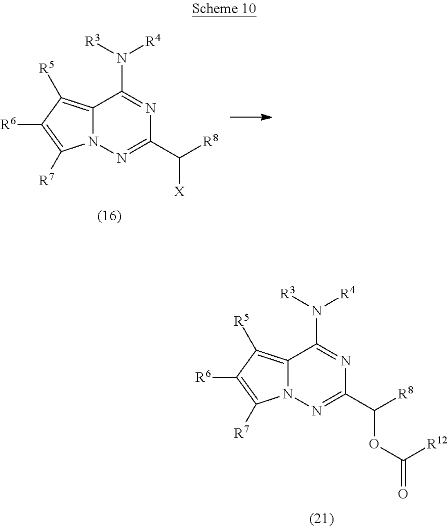

Compounds of formula (22), (23) and (24) may be generally prepared as depicted in the Scheme 11 unless otherwise noted and the various substituents are as defined elsewhere herein. The 2-keto pyrrolotriazine (12) can be reacted with a carbon nucleophile to form the corresponding carbinol (22). Some examples of this chemistry include carbonyl attack by organometallic compounds, for example, where M is a metal not limited to Mg, Li, Zn, Na, K or Cu (Hartley, F. R.; Patai, S. The Chemistry of the Metal-Carbon Bond, vols 2, 3, and 4; Wiley: N.Y, 1985-1987). A typical example is the addition of an excess of an organolithium or organomagnesium compound (Grignard reagent) to a solution of the ketone in an appropriate solvent such as an ether.

Dehydration of the carbinol (22) to form olefinic compounds of formula (23) can be accomplished by various dehydrating methodologies including, but not limited to, heating at 65° C. for 20 hours with a mixture of TFA and TFAA. Alternatively, the olefin (23) can be directly synthesized from ketone (12) via a Wittig, Horner-Emmons or similar type of reaction, one example of which is shown in Example 40. Reduction of the corresponding olefin (23) to form benzylic substituted compound (24) can be accomplished by various reducing methodologies including, but not limited to, catalytic hydrogenolysis, dissolving metal reductions, zinc in acetic acid, diimide, and others (March, Jerry, Advanced Organic Chemistry, 5^{th} ed., Wiley, N.Y., 2001, pp. 1002-1008). Catalytic hydrogenation can be promoted by various metal catalysts, such as palladium on carbon, in a suitable solvent such as an alcohol in the presence of a hydrogen source such as hydrogen gas or any of the known transfer hydrogenation reagents such as formic acid, ammonium formate, cyclohexadiene or cyclohexene.

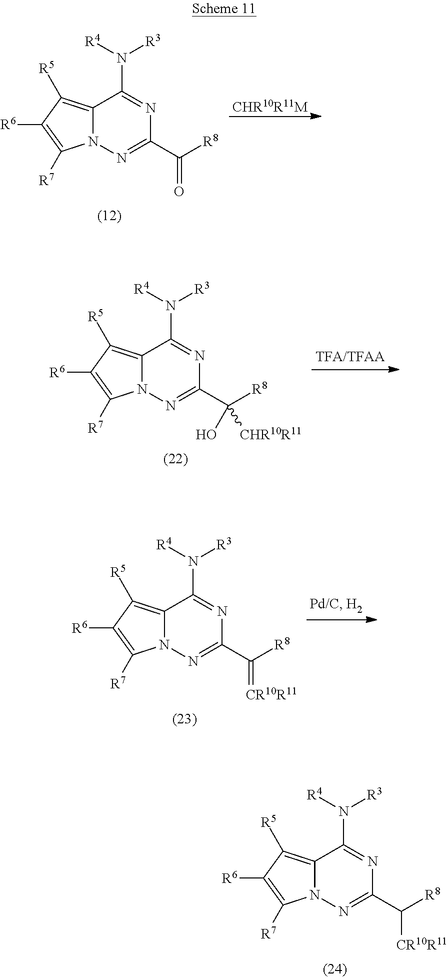

The 2-keto pyrrolotriazine analogs having the formulae (II) or (12) may be modified into the corresponding thioxo using thionating reagents such as P₄S₁₀ or Lawesson's reagent, in a suitable solvent including, but not limited to, toluene, xylene, carbon disulfide, or acetonitrile, optionally with heating. The 2-keto pyrrolotriazine derivatives of formulae (11) or (12) may undergo condensation with amines, hydroxylamines or hydrazines to yield the corresponding imine, oxime or hydrazone. Condensation reactions may be carried out in the presence of trimethyl orthoformate or under conditions of azeotropic water removal, or using 4 A molecular sieves, and may optionally be carried out in the presence of an acid catalyst.

It is understood that in preparing the compounds disclosed herein, some substituents R¹, R², R³, R⁴, R⁵, R⁶, R⁷ and R⁸ may be incorporated by initially incorporating a precursor form of the respective R groups using the methods described herein. Such precursor forms, which may themselves be an embodiment of the invention, may be further transformed at a later stage to new variants of R¹, R², R³, R⁴, R⁵, R⁶, R⁷ and R⁸, respectively. For example, R⁶ may initially be incorporated as a nitro group (25), which subsequently is converted to an acylamido substituent by reduction (for example by catalytic hydrogenation) followed by acylation (for example with an acyl chloride or other activated carboxylic, carbonic, or carbamic acid) to obtain compounds of formulae (27), (28) and (29). When a newly introduced R group contains a leaving group such as halide as a substitutent, the leaving group may subsequently be displaced by another nucleophile such as an amine to produce additional embodiments.

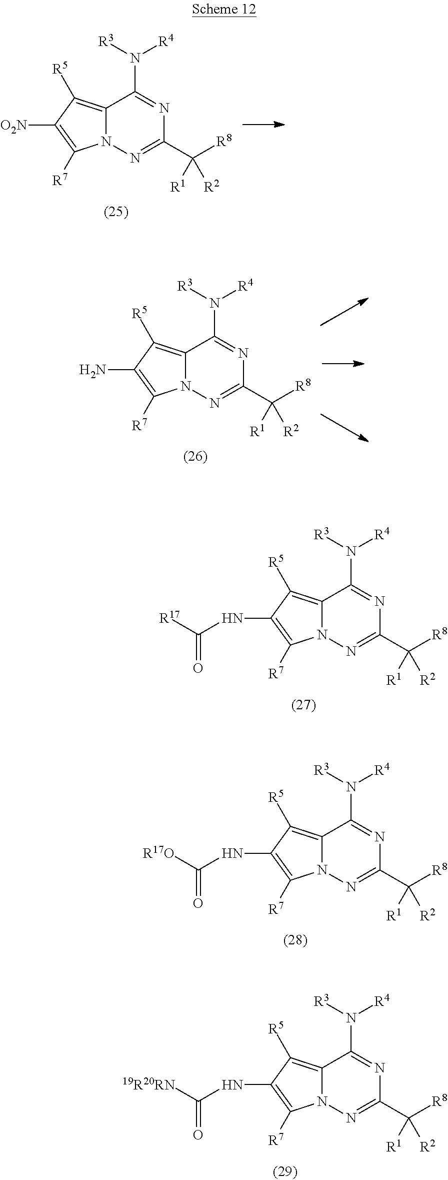

As another example, R⁶ may be incorporated as an alkoxycarbonyl substituent (30), which is converted at a late stage of synthesis into a substituted alkyl group by the following sequence: ester reduction to a corresponding alcohol (31), for example, by using a hydride reducing agent such as lithium borohydride or lithium aluminum hydride in an ether solvent; conversion of the resulting hydroxyl to a leaving group X (32), for example by conversion to a halide using a reagent such as thionyl chloride or triphenylphosphine dibromide, or by conversion to a sulfonate using a reagent such as methanesulfonyl chloride or an arylsulfonyl chloride; nucleophilic displacement of the leaving group by treatment with a primary or secondary amine to form, respectively, a secondary or tertiary amine (33) or with an alcoholate to form an ether (34), or with a thiolate to form a thioether (35), or with azide followed by azide reduction using triphenylphosphine to form a primary amine (33). When the product is a primary or secondary amine, the primary or secondary amino group may be further functionalized, for example by acylation, carbamoylation, alkoxycarbonylation, or sulfonation, under conditions well known in the art, to form additional embodiments.

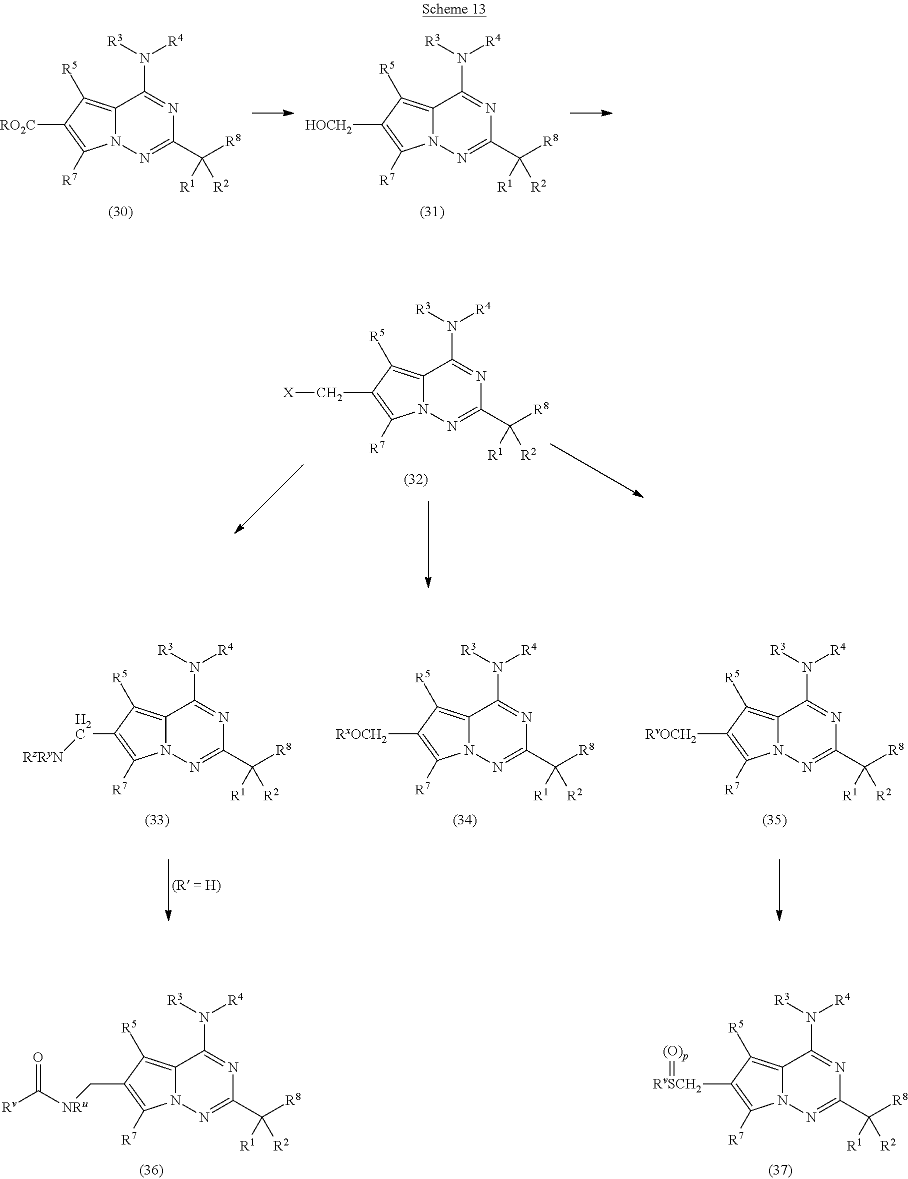

Alternatively, ester reduction to the corresponding alcohol (as above) followed by oxidation to the aldehyde, followed by reductive amination, that is, by treatment with a primary or secondary amine under reducing conditions, for example, sodium cyanoborohydride or sodium triacetoxyborohydride in a suitable solvent system such as methanol and acetic acid, forms, respectively, a secondary or tertiary amino substituent as additional embodiments. Alternatively, treatment of the intermediate aldehyde compound with alkyllithium or alkylmagnesium halide reagents yields secondary carbinols (40) as additional embodiments.

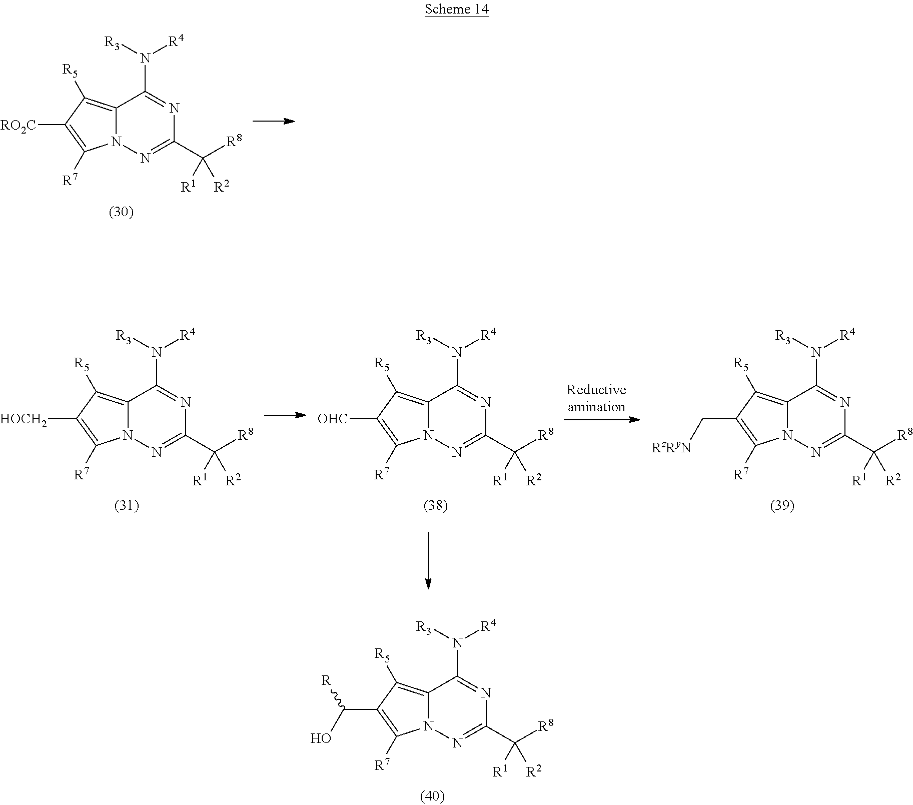

Alternatively, the above ester compound may be treated with excess methyl magnesium halide to form the dimethylcarbinol (41) as another embodiment. Subsequent treatment with hydrogen peroxide effects Baeyer-Villiger-type oxidation such that the alkoxycarbonyl group of the ester is replaced by a hydroxyl group (42), which represents another embodiment. This hydroxyl group can optionally be further substituted to form further embodiments, for example, compounds of formula (43) by alkylation with an alkyl halide in a suitable solvent such as DMF or THF and promoted as necessary by the presence of a suitable base such as sodium hydride or DIPEA or by elevated temperature.

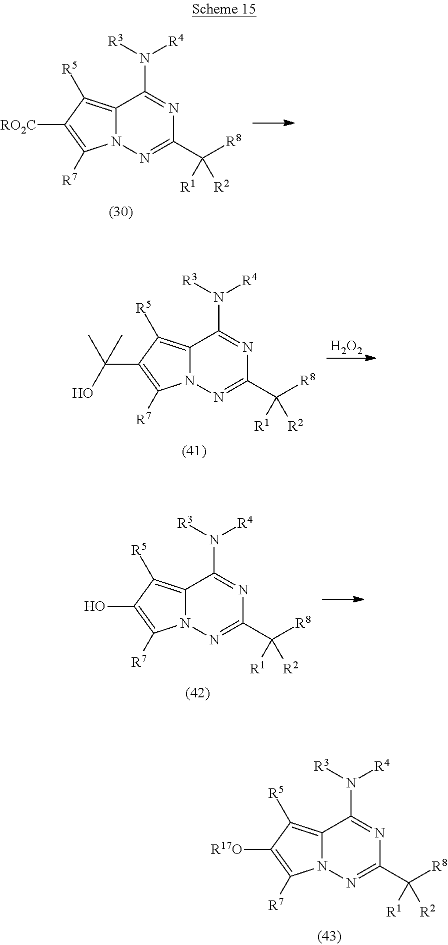

As another example, R¹ may be initially incorporated as an alkoxycarbonylalkyl substituent (44), which is converted at a late stage of synthesis into a substituted alkyl group by the following sequence: ester reduction to a corresponding alcohol (45) (for example using a hydride reducing agent such as lithium borohydride or lithium aluminum hydride in an ether solvent); conversion of the resulting hydroxyl to a leaving group X (46) (for example by conversion to a halide using a reagent such as thionyl chloride or triphenylphosphine dibromide, or by conversion to a sulfonate using a reagent such as methanesulfonyl chloride or an arylsulfonyl chloride); nucleophilic displacement of the leaving group by treatment with a primary or secondary amine to form, respectively, a secondary or tertiary amine (47), or with an alcoholate to form an ether (48), or with a thiolate to form a thioether (49), or with azide followed by azide reduction using triphenylphosphine to form a primary amine (47)

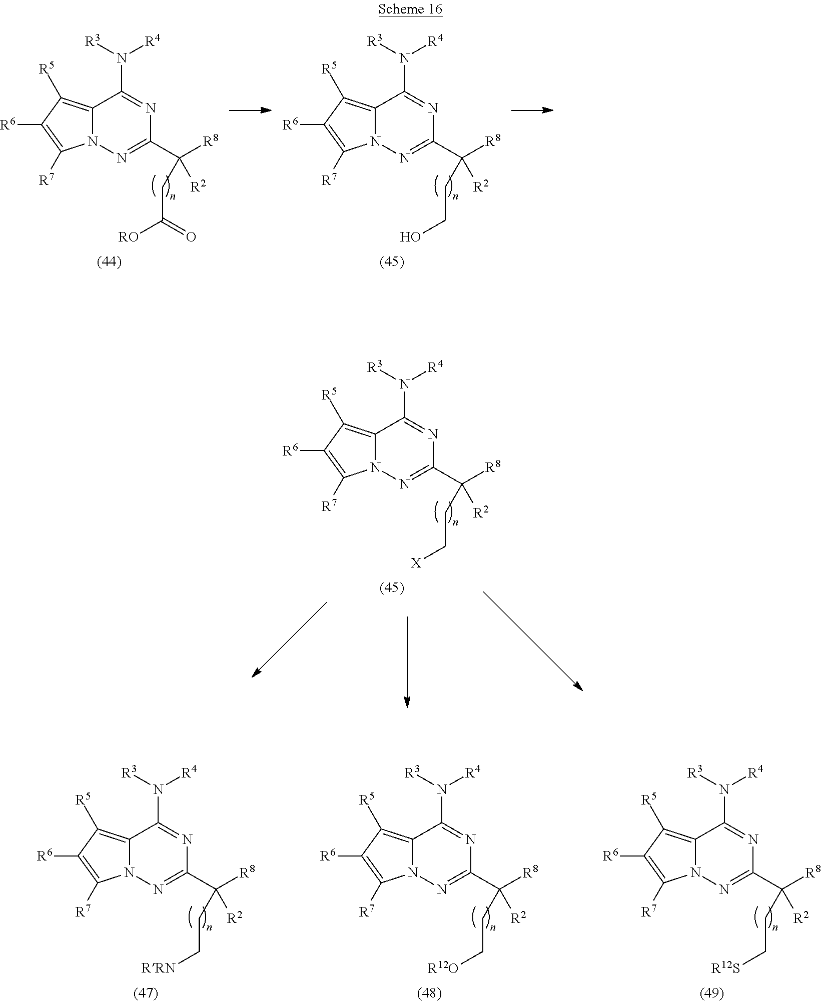

The subject matter has been described in an illustrative manner, and it is to be understood that the terminology used is intended to be in the nature of description rather than of limitation. Thus, it will be appreciated by those of skill in the art that conditions such as choice of solvent, temperature of reaction, volumes, reaction time may vary while still producing the desired compounds. In addition, one of skill in the art will also appreciate that many of the reagents provided in the following examples may be substituted with other suitable reagents. See, e.g., Smith & March, Advanced Organic Chemistry, 5^{th} ed. (2001). Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

### General Procedure A

### Synthesis of 4-chloropyrrolo[1,2-f][1,2,4]triazin-2-carboxylate

Step A. Synthesis of 1-amino-1H-pyrrole-2-carboxamide: To a solution of 1H-pyrrole-2-carbonitrile (10 g, 0.108 mol) in DMF (100 mL) at 10° C., was slowly added NaH (60% in oil, 5.2 g, 0.13 mol). The reaction mixture was left stirring at rt under argon for 1 h. An ethereal solution of NH₂Cl was prepared by suspending NH₄Cl (17.41 g, 0.325 mol, 1.5 equiv) in ether (400 mL). After cooling to −5° C., a concentrated solution of NH₄OH (30%, 28 mL) was added slowly. To this reaction was added sodium hypochlorite (424 mL) at −5° C. and the mixture was vigorously stirred for 20-25 min. The organic layer was separated, washed with brine and dried over CaCl₂ for 90 min. After filtering, 500 mL of ethereal NH₂Cl was obtained. The ethereal solution of NH₂Cl (250 mL) was added at rt to the 1H-pyrrole-2-carbonitrile reaction mixture and the resulting mixture was left stirring vigorously overnight under argon.

The reaction mixture was diluted with water (100 mL) and extracted with ether (2×200 mL). The combined organic layers were washed with brine and dried over MgSO₄. The solvent was evaporated under vacuum to yield a brown residue. The brown residue (1-amino-1H-pyrrole-2-carbonitrile) was taken in a solution of KOH (130 g in 300 mL of water) and stirred overnight at rt. The suspension was left stirring at 0° C. for 1 h, then the solid was filtered and washed with fresh water. The solid was taken in a mixture of 2:1 of isopropanol/water. The solid was filtered and washed with isopropanol (7.1 g, 52%). ¹HNMR (300 MHz, DMSO-d₆) δ 7.9 (1H, bs), 7.10 (1H, bs), 6.85 (1H, m), 6.75 (1H, m), 6.6 (2H, s), 5.9 (1H, m).

### Step B. Synthesis of ethyl 2-(2-carbamoyl-1H-pyrrol-1-ylamino)-2-oxoacetate

To a stirred solution of 1-amino-1H-pyrrole-2-carboxamide (9.0 g, 0.072 mol) in dry DCE (200 mL) at 0° C., triethylamine (10.0 mL, 0.072 mol) and ethyl chlorooxoacetate (8.0 mL, 0.072 mol) were added. The reaction mixture was left stirring overnight at rt. The mixture was diluted with ethyl acetate and washed with water, brine and dried over MgSO₄. The crude product was purified on silica gel column, using a mixture of ethyl acetate/hexane as eluant (5.3 g, 32%). ¹HNMR (300 MHz, DMSO-d₆) δ 12.10 (s, 1H), 7.52 (bs, 1H), 6.92 (s, 1H), 6.90 (bs, 1H, 6.82 (m, 1H), 6.06 (m, 1H), 4.27 (q, 2H), 1.24 (t, 3H).

### Step C. Synthesis of ethyl 4-hydroxypyrrolo[1,2-f][1,2,4]triazine-2-carboxylate

To a stirred solution of ethyl 2-(2-carbamoyl-1H-pyrrol-1-ylamino)-2-oxoacetate (5.5 g, 0.0244 mol) in dry DCE (150 mL), TEA (136 mL, 0.976 mol) and TMSCl (46.5 mL, 0.366 mol) were added. The reaction mixture was refluxed for 24 h. The solid was separated by filtration and the filtrate was evaporated to dryness. The crude material was loaded in a column of silica gel and purified using a mixture of ethyl acetate/hexane (7:3) as eluant to afford ethyl 4-hydroxypyrrolo[1,2-f][1,2,4]triazine-2-carboxylate (2.2 g, 40%). ¹HNMR (300 MHz, DMSO-d₆) δ 12.00 (s, 1H), 7.71 (d, 1H), 6.94 (t, 1H), 6.60 (d, 1H0, 4.30 (q, 2H), 1.24 (t, 3H).

### Step D. Synthesis of ethyl 4-chloropyrrolo[1,2-f][1,2,4]triazine-2-carboxylate

A solution of ethyl 4-hydroxypyrrolo[1,2-f][1,2,4]triazine-2-carboxylate (2.0 g, 0.0096 mol) in POCl₃ (20 mL) was refluxed for 20 h under argon. POCl₃ was removed under vacuum. The resulting residue was taken in toluene and evaporated to dryness to afford ethyl 4-chloropyrrolo[1,2-f][1,2,4]triazine-2-carboxylate as a brown solid (2.2 g, 100%). ¹HNMR (300 MHz, DMSO-d₆) δ 8.46 (d, 1H), 6.94 (t, 1H), 6.60 (d, 1H), 4.30 (q, 2H), 1.24 (t, 3H).

### General Procedure B

### Synthesis of (4-chloropyrrolo[1,2-f][1,2,4]triazin-2-yl)(4-fluorophenyl)methanone

To a solution of ethyl 4-chloropyrrolo[1,2-f][1,2,4]triazine-2-carboxylate (500 mg, 2.22 mmol) in THF (6 mL) at −40° C., a 2.0 M solution of 4-fluorophenylmagnesium bromide in ether (1.3 mL, 2.66 mmol) was added dropwise. After 4 hours of stirring at −40° C., a 0.5 N solution of HCl (6 mL) and ethyl acetate (6 mL) were added. The organic layer was separated, washed with brine and dried over MgSO₄. The crude product was purified by silica gel, using a mixture of ethyl acetate:hexane to afford (4-chloropyrrolo[1,2-f][1,2,4]triazin-2-yl)(4-fluorophenyl)methanone (527 mg, 86%). ¹H NMR (300 MHz, DMSO-d₆) δ 8.43 (s, 1H), 8.19 (2H, m), 7.38 (2H, m), 7.30 (2H, m); LC-MS (ESI) m/z 276 (M+H)⁺.

### Example 1

### General Procedure C

### Preparation of H-pyrazol-3-ylamino)pyrrolo[1,2-f][1,2,4]triazin-2-yl)methanone

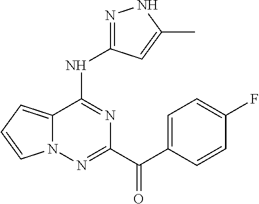

To a solution of (4-chloropyrrolo[1,2-f][1,2,4]triazin-2-yl)(4-fluorophenyl)methanone (200 mg, 0.72 mmol) in DMF (3 mL) were added KI (119 mg, 0.72), 5-methyl-1H-pyrazol-3-amine (77 mg, 0.79 mmol) and DIPEA (0.150 mL, 0.86 mmol). The reaction mixture was stirred at rt overnight. The mixture was diluted with EtOAc (15 mL) and washed with water, brine and dried over MgSO₄. The crude product was purified on silica gel, using DCM/MeOH as eluant. ¹H NMR (300 MHz, DMSO-d₆) 8$ 12.23 (s, 1H), 10.87 (s, 1H), 8.14 (m, 2H), 7.87 (s, 1H), 7.40 (m, 3H), 6.84 (m, 1H), 6.54 (s, 1H), 1.99 (s, 3H). LC-MS (ESI) m/z 337 (M+H)⁺.

### Example 2

### General Procedure D

### Preparation of (4-fluorophenyl)(4-(5-methyl-1H-pyrazol-3-ylamino)pyrrolo[1,2-f][1,2,4]triazin-2-yl)methanol

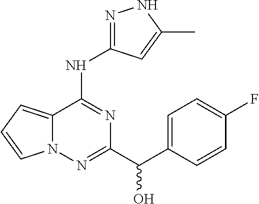

To a solution of (4-fluorophenyl)(4-(5-methyl-1H-pyrazol-3-ylamino)pyrrolo[1,2-f][1,2,4]triazin-2-yl)methanone (150 mg, 0.446 mmol) in a 1:1 mixture of methanol and THF (4 mL) at 0° C., was added sodium borohydride (NaBH₄) (25 mg, 0.669 mmol). After 2 h of stirring at 0° C., acetone (5 drops) was added and the mixture was purified by preparative HPLC to afford 27.1 mg of (4-fluorophenyl)(4-(5-methyl-1H-pyrazol-3-ylamino)pyrrolo[1,2-f][1,2,4]triazin-2-yl)methanol. LC-MS (ESI) m/z 339 (M+H)⁺.

In an alternative preparation, (4-fluorophenyl)(4-(5-methyl-1H-pyrazol-3-ylamino)pyrrolo[1,2-f][1,2,4]triazin-2-yl)methanone (40 mg, 0.119 mmol) in MeOH (3 mL) was treated with sodium borohydride (6.8 mg, 0.178 mmol). After stirring at rt for 2 h, 0.5 M HCl (ca. 0.2 mL) was added. The solvents were removed under vacuum and the residue dissolved in a mixture of 1:1 of ethyl acetate and THF (15 mL), washed with brine and dried over MgSO₄. The crude product was purified by preparative TLC to afford (4-fluorophenyl)(4-(5-methyl-1H-pyrazol-3-ylamino)pyrrolo[1,2-f][1,2,4]triazin-2-yl)methanol (4.5 mg). ¹H NMR (300 MHz, DMSO-d₆) δ 12.15 (s, 1H), 10.56 (s, 1H), 7.71 (s, 1H), 7.54 (m, 2H), 7.15 (m, 4H), 6.66 (m, 1H), 6.43 (s, 1H), 5.55 (s, 1H), 2.20 (s, 3H). LC-MS (ESI) m/z 339 (M+H)⁺.

### Example 3

### Preparation of 2-(fluoro(4-fluorophenyl)methyl)-N-(5-methyl-1H-pyrazol-3-yl)pyrrolo[1,2-f][1,2,4]triazin-4-amine

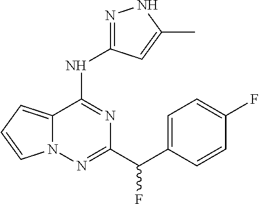

To a solution of (4-fluorophenyl)(4-(5-methyl-1H-pyrazol-3-ylamino)pyrrolo[1,2-f][1,2,4]triazin-2-yl)methanol (50 mg, 0.148 mmol) in a mixture of DCM/THF (2:1, 15 mL), bis(2-methoxyethyl-amino)sulfur trifluoride (41 ul, 0.22 mmol) was added at room temperature. The mixture was left stirring overnight. The reaction mixture was evaporated and the residue was dissolved in DMF (1 mL) and purified on reverse phase HPLC. ¹H NMR (300 MHz, DMSO-d₆) δ 12.2 (s, 1H), 10.70 (1, 1H), 7.77 (m, 1H), 7.63 (m, 2H), 7.27 (m, 3H), 6.42 (d, 1H), 6.39 (s, 1H), 6.35 (s, 1H), 2.24 (s, 3H). LC-MS (ESI) m/z 341(M+H)⁺.

### Example 4

### Preparation of 2-(difluoro(4-fluorophenyl)methyl)-N-(5-methyl-1H-pyrazol-3-yl)pyrrolo[1,2-f][1,2,4]triazin-4-amine

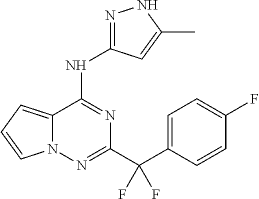

Step A: To a solution of 2,2-difluoro-2-(4-fluorophenyl)acetic acid (obtained according to Middleton et al., J. Org. Chem., 1980, 45(14): 2883-2887, from ethyl 2-(4-fluorophenyl)-2-oxoacetate and subsequent basic hydrolysis) (5.31 g, 0.0279 mol) in DCM (20 mL) were added oxalyl chloride (2.83 mL, 0.033 mol) and 4 drops of DMF at room temperature. The reaction mixture was stirred for 3 h. The solvent was evaporated under vacuum. Additional DCM was added and the solvent evaporated again to remove traces of oxalyl chloride. The residue was taken in DCE (20 mL). This solution was added slowly to a mixture of 1-amino-1H-pyrrole-2-carboxamide (5.31 g, 0.0279 mol) and TEA (4.27 mL, 0.0307 mol) at room temperature. After stirring overnight, EtOAc (50 mL) was added and the solution was washed with a saturated solution of NaHCO₃ and brine. After drying over MgSO₄, the solvents were evaporated and the crude product was purified on silica gel to afford partially purified 1-(2,2-difluoro-2-(4-fluorophenyl)acetamido)-1H-pyrrole-2-carboxamide (Yield: 1.77 g, not completely pure). LC-MS (ESI) m/z 298 (M+H)⁺.

Step B: To a solution of 1-(2,2-difluoro-2-(4-fluorophenyl)acetamido)-1H-pyrrole-2-carboxamide (˜1.70 g, ˜0.0058 mol.) in 1,2-dichloroethane (50 mL) were added TEA (32 mL, 0.234 mol) and TMSCl (11.1 mL, 0.0877 mol). The reaction mixture was stirred at 85° C. for 20 h. The solid was filtered and the filtrate was evaporated to dryness. The residue was purified on silica gel, using DCM/MeOH as eluant to afford 2-(difluoro(4-fluorophenyl)methyl)pyrrolo[1,2-f][1,2,4]triazin-4-ol as a white solid (0.442 g, 27%). ¹H NMR (300 MHz, DMSO-d₆) δ 12.61 (s, 1H), 7.76 (m, 2H), 7.65 (m 1H), 7.40 (t, 2H), 6.98 (m, 1H), 6.61 (m, 1H). LC-MS (ESI) m/z 280 (M+H)⁺.

Step C: A solution of 2-(difluoro(4-fluorophenyl)methyl)pyrrolo[1,2-f][1,2,4]triazin-4-ol (0.442 g, 1.58 mmol) in POCl₃ (5 mL) was heated at 110° C. for 6 h. POCl₃ was evaporated under vacuum. The residue was dissolved in toluene and the solvent was evaporated again. The residue was purified on silica gel, using DCM as eluant to afford 4-chloro-2-(difluoro(4-fluorophenyl)methyl)pyrrolo[1,2-f][1,2,4]triazine. NMR (300 MHz, DMSO-d₆) δ 8.42 (m, 1H), 7.73 (m, 2H), 7.37 (t, 2H), 7.25 (m, 2H). LC-MS (ESI) m/z 298 (M+H)⁺.

Step D: General Procedure C was followed using 4-chloro-2-(difluoro(4-fluorophenyl)methyl)pyrrolo[1,2-f][1,2,4]triazine in place of (4-chloropyrrolo[1,2-f][1,2,4]triazin-2-yl)(4-fluorophenyl)methanone. The reaction mixture was diluted with water and the solid was collected by filtration. After washing with fresh water, the solid was triturated with small amount of MeOH to afford the title compound (yield: 54%). ¹H NMR (300 MHz, DMSO-d₆) δ 12.21 (s, 1H), 10.87 (s, 1H), 7.84 (m, 1H), 7.71 (m, 2H), 7.36 (t, 3H), 6.77 (m, 1H), 6.25 (s, 1H), 2.22 (s, 3H). LC/MS, M+1: 359.

### Example 5

### Preparation of (4-(1H-pyrazol-3-ylamino)pyrrolo[1,2-f][1,2,4]triazin-2-yl)(4-fluorophenyl)methanone

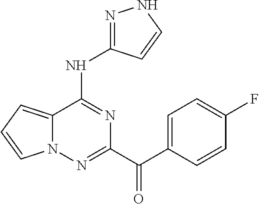

General Procedure C was followed using 1H-pyrazol-3-amine in place of 5-methyl-1H-pyrazol-3-amine. (Yield: 32%). ¹H NMR (300 MHz, DMSO-d₆) δ 12.55 (s, 1H), 11.99 (s, 1H), 8.15 (m, 2H), 7.99 (s, 1H), 7.88 (s, 1H), 7.38 (m, 3H), 6.85 (s, 1H), 6.78 (s, 1H). LC-MS (ESI) m/z 323 (M+H)⁺.

### Example 6

### Preparation of (4-(1H-pyrazol-3-ylamino)pyrrolo[1,2-f][1,2,4]triazin-2-yl) (4-fluorophenyl)methanol

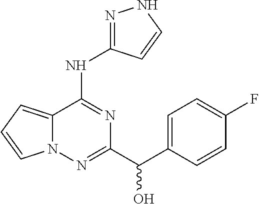

General Procedure D was followed using (4-(1H-pyrazol-3-ylamino)pyrrolo[1,2-f][1,2,4]triazin-2-yl)(4-fluorophenyl)methanone in place of (4-fluorophenyl)(4-(5-methyl-1H-pyrazol-3-ylamino)pyrrolo[1,2-f][1,2,4]triazin-2-yl)methanone. Yield: 48%. ¹H NMR (300 MHz, DMSO-d₆) δ 12.46 (s, 1H), 10.62 (s, 1H), 7.60 (m, 4H), 7.16 (m, 3H), 6.83 (s, 1H), 6.66 (s, 1H), 5.94 (s, 1H), 5.54 (d, 1H). LC-MS (ESI) m/z 325 (M+H)⁺.

### Example 7

### Preparation of 2-(fluoro(4-fluorophenyl)methyl)-N-(1H-pyrazol-3-yl)pyrrolo[1,2-f][1,2,4]triazin-4-amine

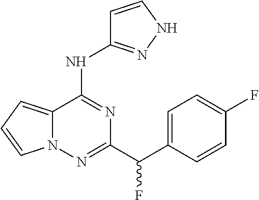

To a solution of (4-(1H-pyrazol-3-ylamino)pyrrolo[1,2-f][1,2,4]triazin-2-yl)(4-fluorophenyl)methanol (0.139 g, 0.428 mmol) in a mixture of DCM (12 mL) and THF (6 mL) under argon at rt was added DAST (0.084 mL, 0.642 mmol). The mixture was stirred at rt for 5 h and quenched with H₂O (2 mL) and concentrated in reduced pressure. The residue wad purified on preparative HPLC (Phenomenex phenylhexyl reverse phase column, eluted with gradient of solvent B=0.05% HOAc/CH₃CN and solvent A=0.05% HOAc/H₂O) to afford 2-(fluoro(4-fluorophenyl)methyl)-N—OH-pyrazol-3-yl)pyrrolo[1,2-f][1,2,4]triazin-4-amine as a white solid (0.012 g, 9%). NMR (300 MHz, DMSO-d₆) δ 12.52 (s, 1H), 10.78 (s, 1H), 7.80 (s, 1H), 7.68 (s, 1H), 7.62 (m, 2H), 7.25 (m, 2H), 6.72 (bs, 2H), 6.52 (s, 0.5H), 6.36 (s, 0.5H); LC-MS (ESI) m/z 327 (M+H)⁺.

### Example 8

### Preparation of 2-(difluoro(4-fluorophenyl)methyl)-N-(1H-pyrazol-3-yl)pyrrolo[1,2-f][1,2,4]triazin-4-amine

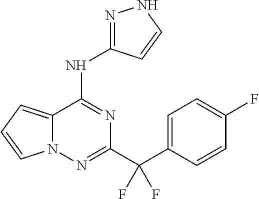

General Procedure C was followed using 4-chloro-2-(difluoro(4-fluorophenyl)methyl)pyrrolo[1,2-f][1,2,4]triazine and 1H-pyrazol-3-amine. The reaction mixture was diluted with water and the solid was collected by filtration. After washing with fresh water, the solid was triturated with small amount of MeOH to afford the title compound (yield: 60.8%). ¹H NMR (300 MHz, DMSO-d₆) δ 12.55 (s, 1H), 11.00 (s, 1H), 7.85 (m, 1H), 7.70 (m, 3H), 7.34 (t, 3H), 6.78 (m, 1H), 6.65 (s, 1H). LC-MS (ESI) m/z 345(M+H)⁺.

### Example 9

### Preparation of (4-(1H-pyrazol-3-ylamino)-7-chloropyrrolo[1,2-f][1,2,4]triazin-2-yl)(4-fluorophenyl)methanone

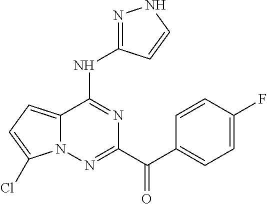

To (4-chloropyrrolo[1,2-f][1,2,4]triazin-2-yl)(4-fluorophenyl)methanone (50 mg, 0.18 mmol) in DMF (1 mL) was added N-chlorosuccinimide (25 mg, 0.18 mmol) and the mixture was stirred for 30 min at rt. The mixture was then heated to 70° C. for 2 h and then cooled to rt. 3-aminopyrazole (23 mg, 0.27 mmol) and diisopropylethylamine (0.047 mL, 0.27 mmol) were then added and the mixture was stirred at rt overnight. DMSO (2 mL) was added and the mixture was purified by reverse phase HPLC (Varian diphenyl reverse phase column, eluted with gradient of solvent B=0.05% HOAc/CH₃CN and solvent A=0.05% HOAc/H₂O). Fractions that contained solid after standing for 3 h were filtered and the solid dried to afford (4-(1H-pyrazol-3-ylamino)-7-chloropyrrolo[1,2-f][1,2,4]triazin-2-yl)(4-fluorophenyl)methanone (8.5 mg, 13%). ¹H NMR (300 MHz, DMSO-d₆) δ 6.75 (s, 1H), 6.97 (d, 1H), 7.40 (t, 2H), 7.52 (bs, 1H), 7.67 (s, 1H), 8.18 (m, 2H), 11.15 (s, 1H), 12.58 (s, 1H); LC-MS (ESI) m/z 357 (M+H)⁺.

### Example 10

### General Procedure E

### Preparation of 2-(4-fluorobenzyl)-N-(1H-pyrazol-3-yl)pyrrolo[1,2,4]triazin-4-amine

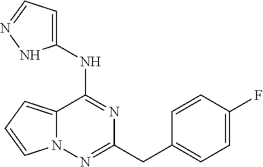

To (4-(1H-pyrazol-5-ylamino)pyrrolo[1,2-f][1,2,4]triazin-2-yl)(4-fluorophenyl)methanol (80 mg, 0.25 mmol) in trifluoroacetic acid (3 mL) at 5° C. was added triethylsilane (2 mL). After stirring for 10 min DCM (1 mL) was added. After stirring for 20 h, 3 drops of methanesulfonic acid was added and the solution was heated to 35° C. After five h the solvent was removed, and the residue was diluted with ethyl acetate. The solution was washed with a saturated aqueous solution of sodium bicarbonate and brine. After drying over sodium sulfate the solution was concentrated and the residue chromatographed on silica gel using a Biotage 25S, eluting with a gradient of 1-6% MeOH/DCM over 25 min to give a white solid (60 mg, 79% yield). ¹H NMR (300 MHz, DMSO-d₆) δ 12.45 (bs, 1H), 10.55 (bs, 1H), 7.66 (bs, 2H), 7.40 (m, 2H), 7.19 (bs, 1H), 7.12 (m, 2H), 6.74 (bs, 1H), 6.63 (m, 1H), 3.91 (s, 2H)). LC-MS (ESI) m/z 309 (M+H)⁺.

### Example 11

### Preparation of (3-fluorophenyl)(4-(5-methyl-1H-pyrazol-3-ylamino)pyrrolo[1,2-f][1,2,4]triazin-2-yl)methanone

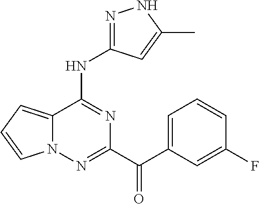

Step 11A: General Procedure B was followed using 3-fluorophenylmagnesium bromide in place of 4-fluorophenyl magnesium bromide to afford (4-chloropyrrolo[1,2-f][1,2,4]triazin-2-yl)(3-fluorophenyl)methanone (Yield: 52%). ¹H NMR (300 MHz, DMSO-d₆) δ 8.45 (m, 1H), 7.90 (m, 2H), 7.62 (m, 2H), 7.29 (m, 2H).

Step 11B: General Procedure C was followed using (4-chloropyrrolo[1,2-f][1,2,4]triazin-2-yl)(3-fluorophenyl)methanone in place of (4-chloropyrrolo[1,2-f][1,2,4]triazin-2-yl)(4-fluorophenyl)methanone to afford the title compound (Yield: 55%). ¹H NMR (300 MHz, DMSO-d₆) δ 12.24 (s, 1H), 10.88 (s, 1H), 7.86 (m, 3H), 7.60 (m, 2H), 7.40 (s, 1H), 6.85 (m, 1H), 6.56 (s, 1H), 2.19 (s, 3H). LC-MS (ESI) m/z 337 (M+H)⁺.

### Example 12

### Preparation of (3-fluorophenyl)(4-(5-methyl-1H-pyrazol-3-ylamino)pyrrolo[1,2-f][1,2,4]triazin-2-yl)methanol

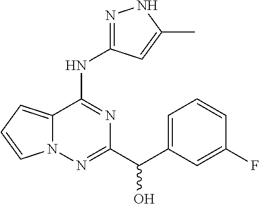

General Procedure D was followed using (3-fluorophenyl)(4-(5-methyl-1H-pyrazol-3-ylamino)pyrrolo[1,2-f][1,2,4]triazin-2-yl)methanone in place of (4-fluorophenyl)(4-(5-methyl-1H-pyrazol-3-ylamino)pyrrolo[1,2-f][1,2,4]triazin-2-yl)methanone to afford the title compound (Yield: 56%). ¹H NMR (300 MHz, DMSO-d₆) δ 12.13 (s, 114), 10.49 (s, 1H), 7.71 (s, 1H), 7.30 (m, 3H), 7.20 (m, 1H), 7.08 (m, 1H), 6.66 (m, 1H), 6.46 (s, 1H), 6.02 (d, 1H), 5.55 (d, 1H), 2.24 (s, 3H). LC-MS (ESI) m/z 339 (M+H)⁺.

### Example 13

### Preparation of (4-(1H-pyrazol-3-ylamino)pyrrolo[1,2-f][1,2,4]triazin-2-yl)(3-fluorophenyl)methanone

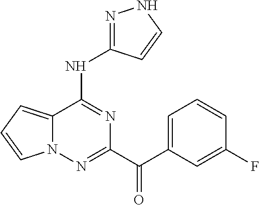

General Procedure C was followed using (4-chloropyrrolo[1,2-f][1,2,4]triazin-2-yl)(3-fluorophenyl)methanone and 1H-pyrazol-3-amine. (Yield: 33%). ¹H NMR (300 MHz, DMSO-d₆) δ 12.56 (s, 1H), 11.01 (s, 1H), 7.86 (m, 3H), 7.60 (m, 3H), 7.40 (bs, 1H), 6.86 (m, 1H), 6.81 (s, 1H). LC/MS, M+1: 323 (M+H)⁺.

### Example 14

### Preparation of (4-(1H-pyrazol-3-ylamino)pyrrolo[1,2-f][1,2,4]triazin-2-yl)(3-fluorophenyl)methanol

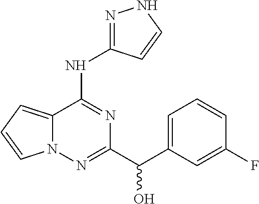

General Procedure D was followed using (4-(1H-pyrazol-3-ylamino)pyrrolo[1,2-f][1,2,4]triazin-2-yl)(3-fluorophenyl)methanone in place of (4-fluorophenyl)(4-(5-methyl-1H-pyrazol-3-ylamino)pyrrolo[1,2-f][1,2,4]triazin-2-yl)methanone to afford the title compound (Yield: 33%). ¹H NMR (300 MHz, DMSO-d₆) δ 12.47 (s, 1H), 10.63 (s, 1H), 7.72 (s, 1H), 7.68 (s, 1H), 7.33 (m, 3H), 7.22 (m, 1), 7.06 (m, 1H), 6.82 (s, 1H), 6.67 (m, 1H), 6.04 (s, 1H), 5.55 (s, 1H). LC-MS (ESI) m/z 325 (M+H)⁺.

### Example 15

### Preparation of (4-(1H-pyrazol-3-ylamino)pyrrolo[1,2-f][1,2,4]triazin-2-yl)(2-methoxyphenyl)methanone

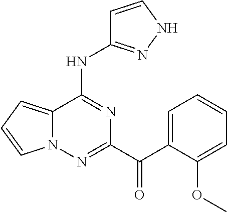

Step A: To a solution of ethyl 4-chloropyrrolo[1,2-f][1,2,4]triazine-2-carboxylate from General Procedure A step D (0.250 g, 1.10 mmol) in THF (10 mL) at −40° C. under argon was added (2-methoxyphenyl)magnesium bromide (0.5 M solution in THF, 3.10 mL, 1.54 mmol). The mixture was stirred at −40° C. for 4 h and quenched with 1.0 N HCl (2.5 mL). The organic layer was washed with brine (25 mL×2), dried over anhydrous Na₂SO₄, filtered, and concentrated under reduced pressure. The residue was purified on silica gel eluting with 30% EtOAc/hexanes to afford (4-chloropyrrolo[1,2-f][1,2,4]triazin-2-yl)(2-methoxyphenyl)methanone as a yellow solid (0.260 g, 84%). ¹H NMR (300 MHz, DMSO-d₆) δ 8.41 (s, 1H), 7.63 (s, 2H), 7.19 (m, 4H), 3.48 (s, 3H); LC-MS (ESI) m/z 288 (M+H)⁺.

Step B: To a solution of (4-chloropyrrolo[1,2-f][1,2,4]triazin-2-yl)(2-methoxyphenyl)methanone (0.130 g, 0.45 mmol) in DMF (2 mL) at rt under argon was added potassium iodide (0.082 g, 0.49 mmol), DIPEA (0.086 mL, 0.49 mmol), and 1H-pyrazol-3-amine (0.041 g, 0.49 mmol). The mixture was stirred at rt for 2 h then heated at 60° C. for 2 h. The mixture was cooled to rt and purified on preparative HPLC (Phenomenex phenylhexyl reverse phase column, eluted with gradient of solvent B=0.05% HOAc/CH₃CN and solvent A=0.05% HOAc/H₂O) to afford (4-(1H-pyrazol-3-ylamino)pyrrolo[1,2-f][1,2,4]triazin-2-yl)(2-methoxyphenyl)methanone as a tan solid (0.066 g, 44%). ¹HNMR (300 MHz, DMSO-d₆) δ 12.58 (s, 1H), 10.98 (s, 1H), 7.92 (s, 1H), 7.61 (m, 2H), 7.47 (s, 1H), 7.21 (m, 2H), 6.90 (t, 1H), 3.69 (s, 3H); LC-MS (ESI) m/z 335 (M+H)⁺.

### Example 16

### Preparation of (4-(1H-pyrazol-3-ylamino)pyrrolo[1,2-f][1,2,4]triazin-2-yl)(2-methoxyphenyl)methanol

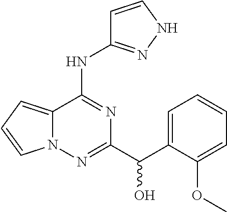

To a suspension of (4-(1H-pyrazol-3-ylamino)pyrrolo[1,2-f][1,2,4]triazin-2-yl)(2-methoxyphenyl)methanone (0.050 g, 0.149 mmol) in a mixture of THF:methanol (1 mL, 1:1) at 0° C. under argon was added NaBH₄ (0.008 g, 0.22 mmol). The mixture was stirred at 0° C. for 1.5 h and quenched with two drops of acetone and concentrated under reduced pressure. The residue was purified on reverse phase HPLC (Phenomenex phenylhexyl reverse phase column, eluted with gradient of solvent B=0.05% HOAc/CH₃CN and solvent A=0.05% HOAc/H₂O) to afford (4-(1H-pyrazol-3-ylamino)pyrrolo[1,2-f][1,2,4]triazin-2-yl)(2-methoxyphenyl)methanol as a white solid (0.031 g, 62%). ¹H NMR (300 MHz, DMSO-d₆) δ 12.39 (s, 1H), 10.52 (s, 1H), 7.75 (m, 2H), 7.58 (s, 1H), 7.25 (m, 2H), 6.95 (m, 2H), 6.75 (s, 1H), 6.65 (s, 1H), 5.88 (d, 1H), 5.68 (s, 1H), 3.72 (s, 3H); LC-MS (ESI) m/z 337 (M+H)⁺.

### Example 17

### Preparation of (5-fluoro-2-methoxyphenyl)(4-(5-methyl-1H-pyrazol-3-ylamino)pyrrolo-[1,2-f][1,2,4]triazin-2-yl)methanone

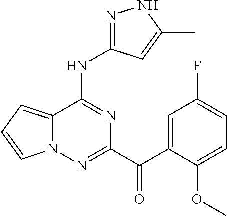

Step A: General Procedure B was followed, using (5-fluoro-2-methoxyphenyl)magnesium bromide in place of 4-fluoro-phenylmagnesium bromide to afford (4-chloropyrrolo[1,2-f][1,2,4]triazin-2-yl)(5-fluoro-2-methoxyphenyl)methanone (Yield: 75%). NMR (300 MHz, DMSO-d₆) δ 8.40 (s, 1H), 7.45 (m, 2H), 7.25 (m, 3H), 3.60 (s, 3H). LC-MS (ESI) m/z 306 (M+Step B: General Procedure C was followed using (4-chloropyrrolo[1,2-f][1,2,4]triazin-2-yl)(5-fluoro-2-methoxyphenyl)methanone in place of (4-chloropyrrolo[1,2-f][1,2,4]triazin-2-yl)(4-fluorophenyl)methanone to afford the title compound (Yield: 15%). ¹H NMR (300 MHz, DMSO-d₆) δ 12.15 (s, 1H), 10.79 (s, 1H), 7.86 (m, 1H), 7.40 (m, 3H), 7.19 (m, 1H), 6.82 (m, 1H), 6.23 (s, 1H), 3.60 (s, 3H), 2.15 (s, 3H). LC-MS (ESI) m/z 366 (M+H)⁺.

### Example 18

### Preparation of (5-fluoro-2-methoxyphenyl)(4-(5-methyl-1H-pyrazol-3-ylamino)pyrrolo[1,2-f][1,2,4]triazin-2-yl)methanol

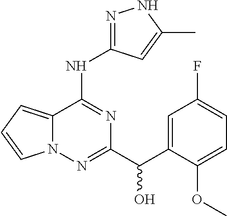

General Procedure D was followed using (5-fluoro-2-methoxyphenyl)(4-(5-methyl-1H-pyrazol-3-ylamino)pyrrolo-[1,2-f][1,2,4]triazin-2-yl)methanone in place of (4-fluorophenyl)(4-(5-methyl-1H-pyrazol-3-ylamino)pyrrolo[1,2-f][1,2,4]triazin-2-yl)methanone to afford the title compound(Yield: 37.9%). ¹H NMR (300 MHz, DMSO-d₆) δ 12.06 (s, 1H), 10.42 (s, 1H), 7.69 (m, 1H), 7.47 (dd, 1H), 7.17 (bs, 1H), 7.10 (d, 1H), 7.06 (dd, 1H), 6.94 (dd, 1H), 6.64 (m, 1H), 6.23 (bs, 1H), 5.94 (m, 1H), 5.78 (d, 1H), 3.67 (s, 3H), 2.19 (s, 3H). LC-MS (ESI) m/z 369 (M+

### Example 19

### Preparation of (4-(1H-pyrazol-3-ylamino)pyrrolo[1,2-f][1,2,4]triazin-2-yl)(5-fluoro-2-methoxyphenyl)methanone

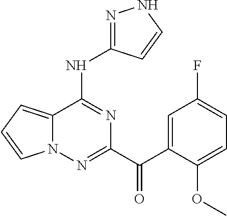

General Procedure C was followed using (4-chloropyrrolo[1,2-f][1,2,4]triazin-2-yl)(5-fluoro-2-methoxyphenyl)methanone in place of (4-chloropyrrolo[1,2-f][1,2,4]triazin-2-yl)(4-fluorophenyl)methanone and 1H-pyrazol-3-amine to afford the title compound (Yield: 37%). ¹H NMR (300 MHz, DMSO-d₆) δ 12.49 (s, 1H), 10.91 (s, 1H), 7.85 (m, 1H), 7.60 (s, 1H), 7.40 (m, 3H), 7.18 (m, 1H), 6.83 (m, 1H), 6.63 (s, 1H), 3.60 (s, 3H). LC-MS (ESI) m/z 353 (M+H)⁺.

### Example 20

### Preparation of 4-(4-(1H-pyrazol-3-ylamino)pyrrolo[1,2-f][1,2,4]triazine-2-carbonyl)benzonitrile

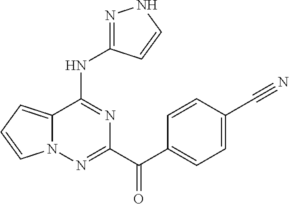

Step A (General Procedure F): To 4-cyano-iodobenzene (183 mg, 0.80 mmol) in THF (4 mL) at −40° C. was added dropwise 2 N isopropyl magnesium bromide in THF (465 μL, 0.93 mmol). The solution was allowed to stir between −40 and −50° C. for 5 h. To this solution was added via cannula ethyl 4-chloropyrrolo[1,2-f][1,2,4]triazine-2-carboxylate (150 mg, 0.66 mmol) in THF (1 mL) at −40° C. After stirring for 4 h at −30° C., the solution was allowed to slowly warm to rt. After 18 h the solution was quenched by addition of a saturated aqueous solution of ammonium chloride. After removal of the THF, the residue was diluted with ethyl acetate and washed with aqueous ammonium chloride, water, and brine. After concentration, the residue was purified on reverse phase HPLC column to afford 4-(4-chloropyrrolo[1,2-f][1,2,4]triazine-2-carbonyl)benzonitrile (49 mg, 26%) as a yellow solid. ¹H NMR (300 MHz, CDCl₃) δ 8.22 (m, 1H), 8.21 (m, 1H), 8.01 (dd, J=2.04 Hz, 2.04 Hz), 7.84 (m, 1H), 7.81 (m, 1H), 7.18 (m, 2H). LC-MS (ESI) m/z 283 (M+H)⁺.

Step B: General Procedure C was followed using 4-(4-chloropyrrolo[1,2-f][1,2,4]triazine-2-carbonyl)benzonitrile in place of (4-chloropyrrolo[1,2-f][1,2,4]triazin-2-yl)(4-fluorophenyl)methanone to obtain the title compound. ¹H NMR (300 MHz, DMSO-d₆) δ 12.56 (bs, 1H), 11.03 (bs, 1H), 8.17 (m, 2H), 8.03 (m, 2H), 7.89 (m, 1H), 7.68 (s, 1H), 7.44 (bs, 1H), 6.88 (m, 1H), 6.82 (bs, 1H). LC-MS (ESI) m/z 330 (M+H)⁺.

### Example 21

### Preparation of 4-((4-(1H-pyrazol-3-ylamino)pyrrolo[1,2-f][1,2,4]triazin-2-yl)(hydroxy)methyl)benzonitrile

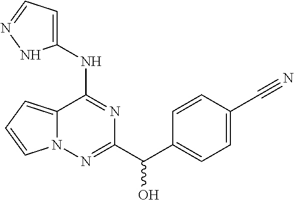

General Procedure D was followed using 4-(4-(1H-pyrazol-3-ylamino)pyrrolo[1,2-f][1,2,4]triazine-2-carbonyl)benzonitrile in place of (4-fluorophenyl)(4-(5-methyl-1H-pyrazol-3-ylamino)pyrrolo[1,2-f][1,2,4]triazin-2-yl)methanone to afford the title compound. ¹H NMR (300 MHz, DMSO-d₆) δ 12.45 (bs, 1H), 10.62 (bs, 1H), 7.78 (m, 2H), 7.69 (m, 4H), 7.22 (bs, 1H), 6.74 (bs, 1H), 6.70 (m, 1H), 6.16 (d, J=4.7 Hz, 1H), 5.62 (d, J=4.6 Hz, 1H)). LC-MS (ESI) m/z 333 (M+H)⁺.

### Examples 22-24

The following compounds were made in a similar manner following General Procedure F using the appropriately substituted phenylmagnesium bromide and then following General Procedure C using 1H-pyrazol-3-amine in place of 5-methyl-1H-pyrazol-3-amine.

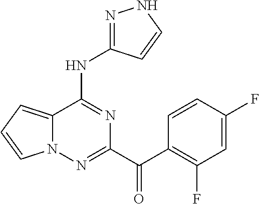

### Example 22

(4-(1H-pyrazol-3-ylamino)pyrrolo[1,2-f][1,2,4]triazin-2-yl)(2,4-difluorophenyl)methanone. NMR (300 MHz, DMSO-d₆) δ 12.55 (bs, 1H), 11.03 (bs, 1H), 7.90 (m, 2H), 7.66 (bs, 1H), 7.44 (m, 2H), 7.29 (m, 1H), 6.87 (m, 1H), 6.73 (s, 1H) LC-MS (ESI) m/z 341 (M+H)⁺.

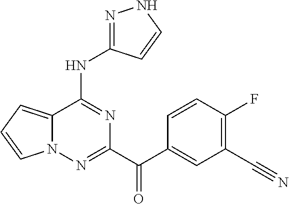

### Example 23

5-(4-(1H-pyrazol-3-ylamino)pyrrolo[1,2-f][1,2,4]triazine-2-carbonyl)-2-fluorobenzonitrile. ¹H NMR (300 MHz, DMSO-d₆) δ 12.58 (bs, 1H), 11.03 (bs, 1H), 8.69 (bs, 1H), 8.46 (bs, 1H), 7.93 (bs, 1H), 7.71 (m, 2H), 7.45 (bs, 1H), 6.88 (bs, 1H), 6.82 (bs, 1H). LC-MS (ESI) m/z 348 (M+H)⁺.

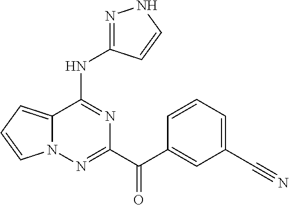

### Example 24

3-(4-(1H-pyrazol-3-ylamino)pyrrolo[1,2-f][1,2,4]triazine-2-carbonyl)benzonitrile. NMR (300 MHz, DMSO-d₆) δ 12.56 (bs, 1H), 11.02 (bs, 1H), 8.51 (bs, 1H), 8.32 (m, 1H), 8.15 (m, 1H), 7.91 (m, 1H), 7.77 (m, 1H), 7.67 (s, 1H), 7.44 (bs, 1H), 6.88 (m, 1H, 6.80 (s, 1H). LC-MS (ESI) m/z 330 (M+H)⁺.

### Example 25

### Preparation of (4-(5-cyclopropyl-1H-pyrazol-3-ylamino)pyrrolo[1,2-f][1,2,4]triazin-2-yl)(4-fluorophenyl)methanone

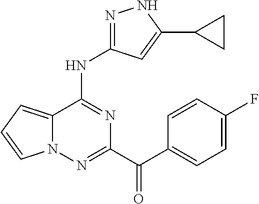

To (4-chloropyrrolo[1,2-f][1,2,4]triazin-2-yl)(4-fluorophenyl)methanone (100 mg, 0.36 mmol) in DMF (2 mL) was added 5-cyclopropyl-1H-pyrazol-3-amine (57 mg, 0.46 mmol), DIPEA (0.080 mL, 0.46 mmol) and potassium iodide (60 mg, 0.36 mmol) and the mixture was stirred for 4.5 h at rt. Acetic acid (0.2 mL) was added and then the mixture was purified by reverse phase HPLC (Varian diphenyl column, eluted with gradient of solvent B=0.05% HOAc/CH₃CN and solvent A=0.05% HOAc/H₂O) to afford (4-(5-cyclopropyl-1H-pyrazol-3-ylamino)pyrrolo[1,2-f][1,2,4]triazin-2-yl)(4-fluorophenyl)methanone (14 mg, 11%). Impure fractions were collected and evaporated, then dissolved in a minimum volume of ethyl acetate. Upon the addition of hexanes, a solid formed which was filtered to give an additional quantity of the title compound (65 mg, 50%). ¹H NMR (300 MHz, DMSO-d₆) δ 0.52 (m, 2H), 0.88 (m, 2H), 1.82 (m, 1H), 6.32 (s, 1H), 6.84 (m, 1H), 7.37-7.42 (m, 3H), 7.87 (s, 1H), 8.14 (m, 2H), 10.86 (s, 1H), 12.19 (s, 1H); LC-MS (ESI) m/z 363 (M+H)⁺.

### Example 26

### Preparation of (4-(5-cyclopropyl-1H-pyrazol-3-ylamino)pyrrolo[1,2-f][1,2,4]triazin-2-yl)(4-fluorophenyl)methanol

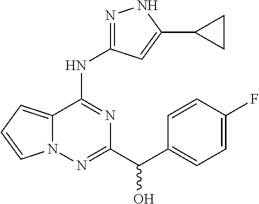

To (4-(5-cyclopropyl-1H-pyrazol-3-ylamino)pyrrolo[1,2-f][1,2,4]triazin-2-yl)(4-fluorophenyl)methanone (30 mg, 0.08 mmol), in THF (1 mL) and MeOH (0.1 mL) was added sodium borohydride (20 mg) and the mixture was stirred for 1 h at rt. Water (0.5 mL) was added followed by 1 mL of dimethylsulfoxide, and the mixture was purified by reverse phase HPLC (Varian diphenyl column, eluted with gradient of solvent B=0.05% HOAC/CH₃CN and solvent A=0.05% HOAc/H₂O) to afford (4-(5-cyclopropyl-1H-pyrazol-3-ylamino)pyrrolo[1,2-f][1,2,4]triazin-2-yl)(4-fluorophenyl)methanol (17 mg, 58%). ¹H NMR (300 MHz, DMSO-d₆) δ 0.67 (m, 2H), 0.97 (m, 2H), 1.86 (m, 1H), 5.54 (s, 1H), 5.94 (s, 1H), 6.28 (s, 1H), 6.66 (m, 1H), 7.11-7.17 (m, 2H), 7.53 (m, 2H), 7.70 (s, 1H), 10.5 (s, 1H), 12.2 (bs, 1H); LC-MS (ESI) m/z 365 (M+H)⁺.

### Example 27

### Preparation of (4-(5-cyclobutyl-1H-pyrazol-3-ylamino)pyrrolo[1,2-f][1,2,4]triazin-2-yl)(4-fluorophenyl)methanone

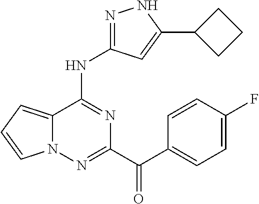

To (4-chloropyrrolo[1,2-f][1,2,4]triazin-2-yl)(4-fluorophenyl)methanone (69 mg, 0.25 mmol), in DMF (2 mL) was added 5-cyclobutyl-1H-pyrazol-3-amine (34 mg, 0.25 mmol), diisopropylethylamine (0.050 mL, 0.29 mmol) and potassium iodide (41 mg, 0.25 mmol) and the mixture was stirred for 4 h at rt. Acetic acid (0.2 mL) was added followed by 1 mL of dimethylsulfoxide and the mixture was purified by preparative HPLC (Varian diphenyl reverse phase column, eluted with gradient of solvent B=0.05% HOAC/CH₃CN and solvent A=0.05% HOAc/H₂O). Fractions containing the desired material were combined and concentrated and then the residue was dissolved in a minimum volume of ethyl acetate. Upon the addition of hexanes, a solid formed which was filtered and dried to afford (4-(5-cyclobutyl-1H-pyrazol-3-ylamino)pyrrolo[1,2-f][1,2,4]triazin-2-yl)(4-fluorophenyl)methanone (20 mg, 21%). ¹H NMR (300 MHz, DMSO-d₆) δ 1.74-2.47 (m, 6H), 3.4 (m, 1H), 6.54 (s, 1H), 6.85 (m, 1H), 7.30-7.43 (m, 3H), 7.88 (s, 1H), 8.13 (m, 2H), 10.88 (s, 1H), 12.25 (s, 1H); LC-MS (ESI) m/z 377 (M+H)⁺.

### Example 28

### Preparation of (4-(5-cyclobutyl-1H-pyrazol-3-ylamino)pyrrolo[1,2-f][1,2,4]triazin-2-yl)(4-fluorophenyl)methanol

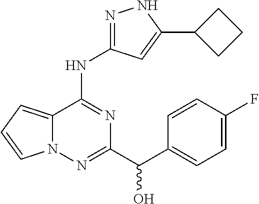

To (4-(5-cyclobutyl-1H-pyrazol-3-ylamino)pyrrolo[1,2-f][1,2,4]triazin-2-yl)(4-fluorophenyl)methanone (7 mg, 0.02 mmol), in THF (0.5 mL) and MeOH (0.05 mL) was added sodium borohydride (15 mg) and the mixture was stirred for 2 h at rt. Water (0.5 mL) was added followed by DMSO (0.5 mL) and the mixture was purified by preparative HPLC (Phenomenex C-18 reverse phase column, eluted with gradient of solvent B=0.05% HOAc/CH₃CN and solvent A=0.05% HOAc/H₂O) to afford (4-(5-cyclobutyl-1H-pyrazol-3-ylamino)pyrrolo[1,2-f][1,2,4]triazin-2-yl)(4-fluorophenyl)methanol (2.2 mg, 29%). ¹H NMR (300 MHz, DMSO-d₆) δ 1.78-2.33 (m, 6H), 3.47 (m, 1H), 5.55 (s, 1H), 5.95 (s, 1H), 6.48 (s, 1H), 6.67 (m, 1H), 7.10-7.18 (m, 2H), 7.56 (m, 2H), 7.71 (s, 1H), 10.55 (s, 1H), 12.20 (bs, 1H); LC-MS (ESI) m/z 379 (M+H)⁺.

### Example 29

### Preparation of (4-fluorophenyl)(4-(thiazol-2-ylamino)pyrrolo[1,2-f][1,2,4]triazin-2-yl)methanone

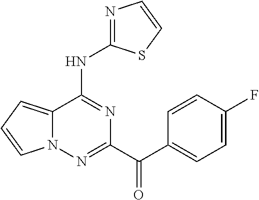

To a mixture of (4-chloropyrrolo[1,2-f][1,2,4]triazin-2-yl)(4-fluorophenyl)methanone (75 mg, 0.27 mmol), tris(dibenzylideneacetone)dipalladium (10 mg, 0.01 mmol), 4,5-bis(diphenylphosphino)-9,9-dimethylxanthene (19 mg, 0.03 mmol), 2-aminothiazole (33 mg, 0.33 mmol), and sodium carbonate (40 mg, 0.37 mmol) was added toluene (2.0 mL) and water (0.01 mL). The vial was evacuated and purged with argon three times and then heated to 100° C. overnight. The crude mixture was filtered, rinsed with ethyl acetate, and the filtrate was concentrated under reduced pressure. DMSO (2 mL) and MeOH (1 mL) were added and the mixture was purified by preparative HPLC (Varian diphenyl reverse phase column, eluted with gradient of solvent B=0.05% HOAc/CH₃CN and solvent A=0.05% HOAc/H₂O) to afford (4-fluorophenyl)(4-(thiazol-2-ylamino)pyrrolo[1,2-f][1,2,4]triazin-2-yl)methanone (5.2 mg, 6%). ¹H NMR (300 MHz, DMSO-d₆) δ 6.96 (s, 1H), 7.27 (s, 1H), 7.37-7.55 (m, 4H), 8.02 (s, 1H), 8.15-8.20 (m, 2H), 12.80 (s, 114); LC-MS (ESI) m/z 340 (M+H)⁺.

### Example 30

### Preparation of (4-fluorophenyl)(4-(thiazol-2-ylamino)pyrrolo[1,2-f][1,2,4]triazin-2-yl)methanol

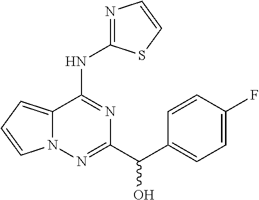

To (4-fluorophenyl)(4-(thiazol-2-ylamino)pyrrolo[1,2-f][1,2,4]triazin-2-yl)methanone (10 mg, 0.03 mmol), in 5 mL of MeOH cooled to 0° C. was added sodium borohydride (20 mg). After 10 min, acetic acid (0.1 mL) was added and the solvents removed under reduced pressure. DMSO (2.5 mL) was added and the mixture was purified by preparative HPLC (Varian diphenyl reverse phase column, eluted with gradient of solvent B=0.05% HOAc/CH₃CN and solvent A=0.05% HOAc/H₂O) to afford (4-fluorophenyl)(4-(thiazol-2-ylamino)pyrrolo[1,2-f][1,2,4]triazin-2-yl)methanol (4.5 mg, 45%). ¹H NMR (300 MHz, MeOD-d₄) δ 5.80 (s, 1H), 6.80 (s, 1H), 7.0-7.20 (m, 4H), 7.50 (s, 1H), 7.60-7.80 (m, 3H); LC-MS (ESI) m/z 342 (M+H)⁺.

### Example 31

### Preparation of (4-fluorophenyl)(4-(5-fluorothiazol-2-ylamino)pyrrolo[1,2-f][1,2,4]triazin-2-yl)methanone

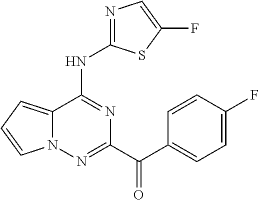

To a mixture of (4-chloropyrrolo[1,2-f][1,2,4]triazin-2-yl)(4-fluorophenyl)methanone (100 mg, 0.36 mmol), tris(dibenzylideneacetone)dipalladium (13 mg, 0.01 mmol), 4,5-bis(diphenylphosphino)-9,9-dimethylxanthene (25 mg, 0.04 mmol), 5-fluorothiazol-2-amine hydrochloride (67 mg, 0.43 mmol), and sodium carbonate (92 mg, 0.86 mmol) was added toluene (2.0 mL) and water (0.01 mL). The vial was evacuated and purged with argon three times and then heated to 100° C. overnight. The crude mixture was filtered, rinsed with ethyl acetate, and the solvents removed under reduced pressure. DMSO (2 mL) was added and the mixture was purified by preparative HPLC (Varian diphenyl reverse phase column, eluted with gradient of solvent B=0.05% HOAc/CH₃CN and solvent A=0.05% HOAc/H₂O) followed by additional purification by preparative HPLC (Phenomenex C-18 reverse phase column, eluted with gradient of solvent B=0.05% HOAc/CH₃CN and solvent A=0.05% HOAc/H₂O) to afford (4-fluorophenyl)(4-(5-fluorothiazol-2-ylamino)pyrrolo[1,2-f][1,2,4]triazin-2-yl)methanone (1.79 mg, 1.4%). ¹H NMR (300 MHz, DMSO-d₆) δ 6.92 (m, 1H), 7.38-7.44 (m, 4H), 8.03 (s, 1H), 8.14-8.19 (m, 2H), 12.5 (bs, 1H); LC-MS (ESI) m/z 380 (M+Na)⁺.

### Example 32

### Preparation of (4-fluoronhenyl)(4-(pyrazin-2-ylamino)pyrrolo[1,2-f][1,2,4]triazin-2-yl)methanone

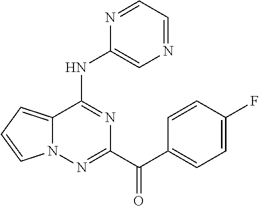

To a mixture of (4-chloropyrrolo[1,2-f][1,2,4]triazin-2-yl)(4-fluorophenyl)methanone (100 mg, 0.36 mmol), tris(dibenzylideneacetone)dipalladium (13 mg, 0.01 mmol), 4,5-bis(diphenylphosphino)-9,9-dimethylxanthene (25 mg, 0.04 mmol), pyrazin-2-amine (42 mg, 0.43 mmol), and sodium carbonate (54 mg, 0.504 mmol) were added toluene (2.0 mL) and water (0.01 mL). The vial was evacuated and purged with argon three times and then heated to 100° C. overnight. The crude mixture was filtered, rinsing with ethyl acetate, and the solvents were removed under reduced pressure. DMSO (2 mL) was added and the mixture was purified by preparative HPLC (Varian diphenyl reverse phase column, eluted with gradient of solvent B=0.05% HOAC/CH₃CN and solvent A=0.05% HOAc/H₂O) to afford (4-fluorophenyl)(4-(pyrazin-2-ylamino)pyrrolo[1,2-f][1,2,4]triazin-2-yl)methanone (17.3 mg, 14%). ¹H NMR (300 MHz, DMSO-d₆) δ 6.97 (m, 1H), 7.40 (t, 2H), 7.59 (d, 1H), 8.03 (s, 1H), 8.17 (m, 2H), 8.38 (s, 1H), 8.48 (s, 1H), 9.74 (s, 1H), 11.38 (s, 1H); LC-MS (ESI) m/z 335 (M+H)⁺.

### Example 33

### Preparation of (4-(1H-pyrazol-3-ylamino)pyrrolo[1,2-f][1,2,4]triazin-2-yl)(cyclopropyl)methanone

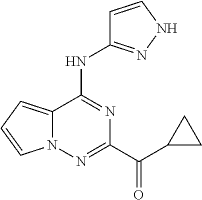

To a suspension of ethyl 4-(1H-pyrazol-3-ylamino)pyrrolo[1,2-f][1,2,4]triazine-2-carboxylate (0.100 g, 0.37 mmol) in THF (5 mL) under argon at −40° C. was added cyclopropylmagnesium bromide (0.5 M solution in THF, 4.40 mL, 2.20 mmol). The mixture was stirred for 5 h at −40° C. and quenched with 1.0 N HCl (5 mL). The organic layer was separated and the aqueous layer was washed with 10% MeOH/DCM (25 mL×2). The combined organic layers were washed with saturated aq NaCl (25 mL), dried over Na₂SO₄, filtered, and concentrated under reduced pressure. The residue wad purified on preparative HPLC (Phenomenex phenylhexyl reverse phase column, eluted with gradient of solvent B=0.05% HOAc/CH₃CN and solvent A=0.05% HOAc/H₂O) to afford (4-(1H-pyrazol-3-ylamino)pyrrolo[1,2-f][1,2,4]triazin-2-yl)(cyclopropypmethanone as a tan solid (0.019 g, 19%). ¹H NMR (300 MHz, DMSO-d₆) δ 12.52 (s, 1H), 10.92 (s, 1H), 7.92 (s, 1H), 7.70 (s, 1H), 7.38 (s, 1H), 7.08 (s, 1H), 6.85 (d, 1H), 3.20 (m, 1H), 1.10 (m, 4H); LC-MS (ESI) m/z 269 (M+H)⁺.

### Example 34

### Preparation of (4-(1H-pyrazol-3-ylamino)pyrrolo[1,2-f][1,2,4]triazin-2-yl)(cyclohexyl)methanone

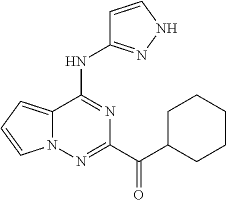

To a suspension of ethyl 4-(1H-pyrazol-3-ylamino)pyrrolo[1,2-f][1,2,4]triazine-2-carboxylate (0.100 g, 0.367 mmol) in THF (5 mL) under argon at −40° C. was added cyclohexylmagnesium bromide (1.0 M solution in THF, 6.60 mL, 6.60 mmol). The mixture was allowed to warm to −30° C. and then stirred at −30° C. overnight. The mixture was quenched with 1.0 N HCl until the pH was ˜7. The mixture was extracted with EtOAc (25 mL×2). The combined organic layers were dried over anhydrous Na₂SO₄, filtered, and concentrated under reduced pressure. The residue was purified on preparative HPLC (Phenomenex phenylhexyl reverse phase column, eluted with gradient of solvent B=0.05% HOAc/CH₃CN and solvent A=0.05% HOAc/H₂O) to afford (4-(1H-pyrazol-3-ylamino)pyrrolo[1,2-f][1,2,4]triazin-2-yl)(cyclohexyl)methanone as a white solid (0.027 g, 16%). ¹H NMR (300 MHz, DMSO-d₆) δ 12.52 (s, 1H), 10.92 (s, 1H), 7.90 (s, 1H); 7.75 (s, 1H), 7.48 (s, 1H), 7.05 (s, 1H), 6.85 (s, 1H), 3.60 (m, 1H), 1.80 (m, 5H), 1.35 (m, 5H); LC-MS (ESI) m/z 311 (M+H)⁺.

### Example 35

### Preparation of (4-(5-methyl-1H-pyrazol-3-ylamino)pyrrolo[1,2-f][1,2,4]triazin-2-yl)(thiophen-2-yl)methanone

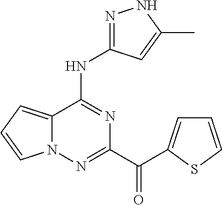

Step A: General Procedure B was followed using 1.0 M solution thiophen-2-ylmagnesium bromide in place of 4-fluoro-phenylmagnesium bromide to yield (4-chloropyrrolo[1,2-f][1,2,4]triazin-2-yl)(thiophen-2-yl)methanone (Yield: 33%). LC-MS (ESI) m/z 264 (M+H)⁺.

Step B: General Procedure C was followed using (4-chloropyrrolo[1,2-f][1,2,4]triazin-2-yl)(thiophen-2-yl)methanone in place of (4-chloropyrrolo[1,2-f][1,2,4]triazin-2-yl)(4-fluorophenyl)methanone to afford the title compound (Yield: 76%). ¹H NMR (300 MHz, DMSO-d₆) δ 12.27 (s, 1H), 10.87 (s, 1H), 8.27 (m, 1H), 8.14 (d, 1H), 7.92 (s, 1H), 7.41 (bs, 1H), 7.30 (m, 1H), 6.86 (m, 1H), 6.73 (s, 1H), 2.25 (s, 3H). LC-MS (ESI) m/z 325 (M+H)⁺.

### Example 36

### Preparation of (4-(1H-pyrazol-3-ylamino)pyrrolo[1,2-f][1,2,4]triazin-2-yl)(thiophen-2-yl)methanone

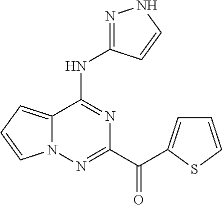

General Procedure C was followed using (4-chloropyrrolo[1,2-f][1,2,4]triazin-2-yl)(thiophen-2-yl)methanone (4-chloropyrrolo[1,2-f][1,2,4]triazin-2-yl)(4-fluorophenyl)methanone and 1H-pyrazol-3-amine in place of 5-methyl-1H-pyrazol-3-amine to afford the title compound (Yield: 100%). NMR (300 MHz, DMSO-d₆) δ12.58 (s, 1H), 10.99 (s, 1H), 8.25 (d, 1H), 8.13 (d, 1H), 7.94 (s, 1H), 7.72 (s, 1H), 7.41 (bs, 1H), 7.30 (m, 1H), 6.97 (s, 1H), 6.87 (m, 1H). LC-MS (ESI) m/z 311 (M+H)⁺.

### Example 37

### Preparation of (4-(1H-pyrazol-3-ylamino)pyrrolo[1,2-f][1,2,4]triazin-2-yl) (thiophen-2-yl)methanol

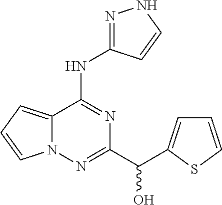

General Procedure D was followed using (4-(1H-pyrazol-3-ylamino)pyrrolo[1,2-f][1,2,4]triazin-2-yl)(thiophen-2-yl)methanone in place of (4-fluorophenyl)(4-(5-methyl-1H-pyrazol-3-ylamino)pyrrolo[1,2-f][1,2,4]triazin-2-yl)methanone to afford the title compound (Yield: 29%). ¹HNMR (300 MHz, DMSO-d₆) δ 12.47 (s, 1H), 10.67 (s, 1H), 7.71 (s, 2H), 7.41 (1H, d), 7.25 (bs, 1H), 7.08 (m, 2H), 6.94 (m, 1H), 6.67 (m, 1H), 6.10 (d, 1H), 5.75 (d, 1H). LC-MS (ESI) m/z 313 (M+H)⁺.

### Example 38

### Preparation of N-((4-(1H-pyrazol-3-ylamino)pyrrolo[1,2-f][1,2,4]triazin-2-yl)(4-fluorophenyl)methyl)-2-hydroxyethanamonium acetate

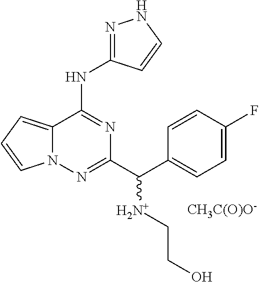

To (4-(1H-pyrazol-5-ylamino)pyrrolo[1,2-f][1,2,4]triazin-2-yl)(4-fluorophenyl)methanol (100 mg, 0.31 mmol) cooled to 0° C. was added thionyl chloride (4 ml) and pyridine (100 uL). After 10 min the solvent was removed and then DMF (2 ml) was added followed by cesium carbonate (201 mg, 0.62 mmol) and sodium iodide (46 mg, 0.31 mmol). After stirring at 80° C. for 20 h, the solution was cooled and diluted with water. The precipitate was collected by filtration. The precipitate was chromatographed on preparative thin layer chromatography followed by reverse phase HPLC purification to give 14 mg of pure product as the formic acid salt. ¹HNMR (300 MHz, DMSO-d₆) δ 10.61 (s, 1H), 7.70 (m, 1H), 7.66 (m, 1H), 7.52 (m, 2H), 7.21 (bs, 1H), 7.12 (m, 2H), 6.76 (bs, 1H), 6.65 (m, 1H), 4.72 (bs, 1H), 4.55 (bs, 1H), 3.50 (bs, 2H), 2.51 (m, 2H), 1.91 (s, 3H). LC-MS (ESI) m/z 368 (M+H)⁺.

### Example 39

### Preparation of 2-(1-(4-fluorophenyl)vinyl)-N-(1H-pyrazol-5-yl)pyrrolo[1,2-f][1,2,4]triazin-4-amine

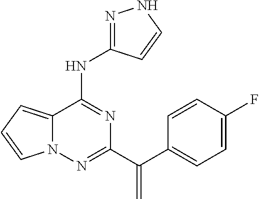

Step A. Synthesis of 1-(4-(1H-pyrazol-3-ylamino)pyrrolo[1,2-f][1,2,4]triazin-2-yl)-1-(4-fluorophenyl)ethanol: To (4-(1H-pyrazol-3-ylamino)pyrrolo[1,2-f][1,2,4]triazin-2-yl)(4-fluorophenyl)methanone (600 mg, 1.86 mmol) in THF (20 ml) cooled to −20° C. was added 3.0 N methylmagnesium bromide in THF (1.98 ml, 5.96 mmol, 3.2 equiv.) dropwise over two minutes. After the addition was complete the solution was placed in an ice bath and stirred under argon for 20 hours. The reaction was incomplete so the solution was recooled to −20° C. and an additional equivalent of 3.0 N methylmagnesium bromide in THF (0.620 ml, 1.86 mmol) was added dropwise. After 3 hours of stirring at 0° C. the solution was quenched by addition of a saturated aqueous ammonium chloride solution. After concentration the residue was diluted with water, and the solids were collected to give crude product, 1-(4-(1H-pyrazol-3-ylamino)pyrrolo[1,2-f][1,2,4]triazin-2-yl)-1-(4-fluorophenyl)ethanol (600 mg) which was used directly in the next step. LC/MS (ESI) m/z 339 (M+H)⁺.

Step B. To 1-(4-(1H-pyrazol-5-ylamino)pyrrolo[1,2-f][1,2,4]triazin-2-yl)-1-(4-fluorophenyl)ethanol (100 mg, 0.30 mmol) at room temperature was added trifluoroacetic acid (2 ml) and trifluoroacetic anhydride (2 mL) in a sealed tube. The mixture was stirred at 65° C. for 20 h. Following removal of the volatile compounds, the remaining residue was purified on a one millimeter preparative thin layer chromatography plate eluting with 50% ethyl acetate/DCM to give the title compound as a tan solid (48 mg, 51%) ¹H NMR (300 MHz, DMSO-d₆) δ 12.47 (bs, 1H), 10.64 (bs, 1H), 7.66 (m, 2H), 7.47 (m, 2H), 7.27-7.17 (m, 3H), 6.71-6.68 (m, 2H), 6.32 (m, 1H), 5.68 (m, 1H); LC-MS (ESI) m/z 321 (M+H)⁺.

### Example 40

### Preparation of ethyl 3-(4-(1H-pyrazol-3-ylamino)pyrrolo[1,2-f][1,2,4]triazin-2-yl)-3-(4-fluorophenyl)acrylate

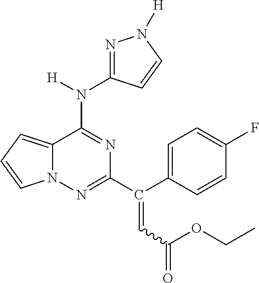

To a suspension of NaH (60% in oil, ˜87 mg, ˜1 mmol) in THF (10 mL) at 0° C. was added triethyl phosphonoacetate (431 μL, 2.17 mmol). After stirring for 30 min at 0° C., (4-(1H-pyrazol-3-ylamino)pyrrolo[1,2-f][1,2,4]triazin-2-yl)(4-fluorophenyl)methanone (140 mg, 0.434 mmol) was added. The reaction mixture was heated at 50° C. overnight. After diluting with ethyl acetate (10 mL), the solution was washed with water and brine, then dried over MgSO₄. The crude product was purified on reverse phase HPLC to afford a mixture of E- and Z-isomers of the title compound (35 mg, 20%). LC-MS (ESI) m/z 393 (M+H)⁺.

### Example 41

### Preparation of ethyl 3-(4-(1H-pyrazol-3-ylamino)pyrrolo[1,2-f][1,2,4]triazin-2-yl)-3-(4-fluorophenyl)propanoate

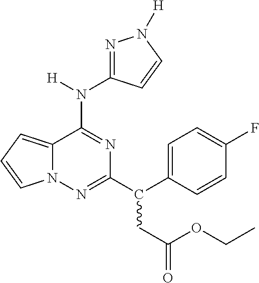

To ethyl 3-(4-(1H-pyrazol-3-ylamino)pyrrolo[1,2-f][1,2,4]triazin-2-yl)-3-(4-fluorophenyl)acrylate (25 mg, 0.063 mmol) in MeOH (2 mL), was added Pd/C (10%) as catalyst in the presence of 1 atmosphere of hydrogen overnight. The reaction mixture was filtered through Celite and the filtrate was concentrated to dryness. The crude product was purified on reverse phase HPLC to afford the title compound as a solid (11 mg, 44%). ¹H NMR (300 MHz, DMSO-d₆) δ 12.40 (bs, 1H), 10.60 (s, 1H), 7.70 (d, 1H), 7.65 (m, 1H), 7.43-7.39 (m, 2H), 7.19 (s, 1H), 7.10 (t, 2H), 6.77 (s, 1H), 6.76-6.62 (m, 1H), 4.41-4.36 (m, 1H), 3.99 (q, 2H), 3.33-3.25 (m, 1H), 2.92 (dd, 1H), 1.05 (t, 3H). LC-MS (ESI) m/z 395 (M+H)⁺.

### Example 42

### Preparation of (4-(5-methyl-1H-pyrazol-3-ylamino)pyrrolo[1,2-f][1,2,4]triazin-2-yl)(quinolin-8-yl)methanone

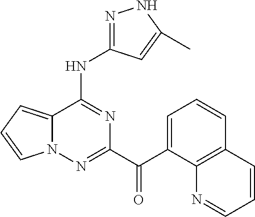

Step A: To a solution of ethyl 4-chloropyrrolo[1,2-f][1,2,4]triazine-2-carboxylate (338 mg, 1.5 mmol) in THF (8 mL) at −78° C. was added dropwise 1.7 M solution of t-BuLi in pentane (1 mL, 1.7 mmol). After stirring at −78° C. for 20 min, a solution of 8-bromoquinoline in THF (4 mL) was added and stirring continued at −78° C. for 1 h. The reaction was quenched with water and the mixture extracted with DCM. The extracts were dried over MgSO₄ and concentrated under reduced pressure. The residue was purified by silica gel chromatography with 10-35% ethyl acetate/hexane as eluant to afford (4-chloropyrrolo[1,2-f][1,2,4]triazin-2-yl)(quinolin-8-yl)methanone as solid (145 mg, 31%). ¹H NMR (300 MHz, CDCl₃) δ 8.66 (dd, 1H), 8.20 (dd, 1H), 8.07 (dd, 1H), 8.04 (dd, 1H), 7.80 (dd, 1H), 7.68 (dd, 1H), 7.36 (dd, 1H), 7.02-7.06 (m, 2H); LC-MS (ESI) m/z 309 (M+H)⁺.

Step B: A mixture of LI-chloropyrrolo[1,2-f][1,2,4]triazin-2-yl)(quinolin-8-yl)methanone (145 mg, 0.47 mmol) and KI (83 mg, 0.5 mmol) in DMF (6 mL) was stirred at room temperature for 20 minutes, then 3-methyl-1H-pyrazol-5-amine (49 mg, 0.5 mmol) and diisopropylethylamine (129 mg, 1 mmol) were added. The reaction mixture was stirred at room temperature overnight. After DMF was removed under reduced pressure, the residue was diluted with water and extracted with DCM. The extracts were dried over MgSO₄ and concentrated under reduced pressure and the residue was purified by preparative HPLC (diphenyl reverse phase column eluted with acetonitrile/water with 0.05% HOAc) to afford (4-(5-methyl-1H-pyrazol-3-ylamino)pyrrolo[1,2-f][1,2,4]triazin-2-yl)(quinolin-8-yl)methanone as a solid (56 mg, 32%). ¹H NMR (300 MHz, DMSO-d₆) δ 12.01 (br, 1H), 10.65 (br, 1H), 8.72 (dd, 1H), 8.48 (dd, 1H), 8.21 (dd, 1H), 7.91 (dd, 1H), 7.82 (dd, 1H), 7.76 (dd, 1H), 7.52 (dd, 1H), 7.30 (m, 1H), 6.80 (dd, 1H), 5.44 (s, 1H) 2.00 (s, 3H); LC-MS (ESI) m/z 370 (M+H)⁺.

### Example 43

### Preparation of (4-(5-methyl-1H-pyrazol-3-ylamino)pyrrolo[1,2-f][1,2,4]triazin-2-yl)(quinolin-8-yl)methanol

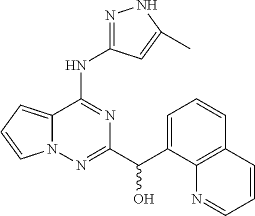

To a solution of (4-(5-methyl-1H-pyrazol-3-ylamino)pyrrolo[1,2-f][1,2,4]triazin-2-yl)(quinolin-8-yl)methanone (310 mg, 0.84 mmol) in THF (5 mL) and MeOH (5 mL) at 0° C. was added NaBH₄ (50 mg, 1.3 mmol). The reaction mixture was stirred at room temperature for 2 h. It was quenched with 10% NH₄Cl solution and extracted with DCM. The extracts were dried over MgSO₄ and concentrated under reduced pressure. The residue was purified by preparative HPLC (diphenyl column, acetonitrile/water with 0.05% acetic acid) to afford as solid (82 mg, 26%). ¹H NMR (300 MHz, DMSO-d₆) δ 11.95 (br, 1H), 10.30 (br, 1H), 8.87 (t, 1H), 8.36 (dd, 1H), 8.10 (d, 1H), 7.94 (t, 1H), 7.69 (t, 2H), 7.51 (dd, 1H), 7.14 (m, 1H), 6.73 (d, 1H), 6.33 (d, 1H), 6.15 (d, 1H), 5.91 (s, 1H) 2.12 (s, 3H); LC-MS (ESI) m/z 372 (M+H)⁺.

### Example 44

### Preparation of methyl 2-(difluoro(4-fluorophenyl)methyl)-4-(5-methyl-1H-pyrazol-3-ylamino)pyrrolo[1,2-f][1,2,4]triazine-6-carboxylate

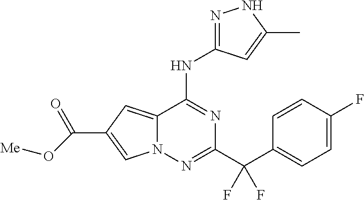

Step A: To a solution of cuprous oxide (832 mg, 5.9 mmol) and 1,10-phenanthroline (2.14 g, 11.89 mmol) in dioxane (200 mL) were added methyl propiolate (10 g, 118.9 mmol) and methyl isocyanoacetate (8.95 g, 98.6 mmol). The reaction mixture was heated at 100° C. for 2 h. The mixture was filtered through Celite, solvents were evaporated, and the residue was purified by silica gel chromatography using a mixture of EtOAc-hexanes as eluent. The isolated product was sonicated with DCM to afford dimethyl 1H-pyrrole-2,4-dicarboxylate (8.6 gm, 48%) as a solid. ¹H NMR: (300 MHz, DMSO-d₆) δ 12.55 (s, 1H), 7.60 (s, 1H), 7.10 (s, 1H), 3.92 (s, 3H), 3.90 (s, 3H); LC-MS (ESI) m/z 184 (M+H)⁺.

Step B: To a solution of dimethyl 1H-pyrrole-2,4-dicarboxylate (4.29 g, 23.4 mmol) in THF (40 mL) was added NaH (60%, 1.41 g, 35.1 mmol) at 0° C. The reaction mixture was stirred for 30 min at rt, then cooled to 0° C. and 0.3 M chloramine in ether (117 mL, 35.1 mmol) was added. The reaction mixture was stirred at rt overnight. The reaction was quenched with aq ammonium chloride and the mixture was extracted with ethyl acetate. The organic layer was separated, dried and concentrated to give dimethyl 1-amino-1H-pyrrole-2,4-dicarboxylate (4.40 g, 95%) containing a small amount of dimethyl 1H-pyrrole-2,4-dicarboxylate. ¹H NMR (300 MHz, DMSO-d₆) δ 7.55 (s, 1H), 7.04 (s, 1H), 6.51 (s, 2H), 3.78 (s, 3H), 3.71 (s, 3H); LC-MS (ESI) m/z 199 (M+H)⁺.

Step C: A solution of dimethyl 1-amino-1H-pyrrole-2,4-dicarboxylate (1 g, 5.05 mmol) in 7N NH₃ in MeOH (15 mL) was heated at 110° C. in a sealed tube for 36 h. The solvents were evaporated and the residue was used for the next step without further purification. LC-MS (ESI) m/z 184 (M+H)⁺.

Step D: To a solution of 2,2-difluoro-2-(4-fluorophenyl)acetic acid (683 mg, 3.60 mmol) in DMF (7 mL) was added HATU (1.37 g, 3.60 mmol) and the mixture was stirred for 10 min at rt. Methyl 1-amino-5-carbamoyl-1H-pyrrole-3-carboxylate (600 mg, 3.27 mmol) was added followed by DIPEA (0.684 mL, 3.93 mmol). The reaction mixture was stirred overnight at rt. The reaction mixture was diluted with 10% MeOH/EtOAc and the organic layer was separated, dried and concentrated. The residue was sonicated in DCM and the solid was collected by filtration and dried to give methyl 5-carbamoyl-1-(2,2-difluoro-2-(4-fluorophenyl)acetamido)-1H-pyrrole-3-carboxylate (480 mg, 42%). ¹H NMR (300 MHz, DMSO-d₆) δ 13.90 (s, 1H), 12.60 (s, 2H), 7.85-7.75 (m, 3H), 7.45-7.35 (m, 3H), 3.70 (s, 3H); LC-MS (ESI) m/z 356 (M+H)⁺.

Step E: To a solution of methyl 5-carbamoyl-1-(2,2-difluoro-2-(4-fluorophenyl)acetamido) 1H-pyrrole-3-carboxylate (3.5 g, 9.85 mmol) in dichloroethane (100 mL) were added triethylamine (54.92 mL, 394 mmol) and TMSCl (18.88 mL, 147.75 mmol) and the mixture was heated at 85° C. for 12 h. The solvents were evaporated and the residue was diluted with ethyl acetate and washed with aq ammonium chloride, water and brine. The organic layer was concentrated and the residue was purified by silica gel chromatography using DCM/MeOH as eluent to afford methyl 2-(difluoro(4-fluorophenyl)methyl)-4-oxo-3,4-dihydropyrrolo[1,2-f][1,2,4]triazine-6-carboxylate (2.1 g, 64%) as a white solid. ¹H NMR: (300 MHz, DMSO-d₆) δ 12.90 (s, 1H), 8.17 (s, 1H), 7.77 (m, 2H), 7.40 (m, 2H), 7.23 (s, 1H), 3.78 (s, 3H); LC-MS (ESI) m/z 338 (M+H)⁺.

Step F: To a solution of methyl 2-(difluoro(4-fluorophenyl)methyl)-4-oxo-3,4-dihydropyrrolo[1,2-f][1,2,4]triazine-6-carboxylate (1.1 g, 3.26 mmol) in POCl₃ (16 mL) was added DMA (8 drops) and the mixture was heated at 125° C. for 8 h in a sealed tube. The solvents were evaporated and the residue was used directly in the next step.

Step G: To a solution of methyl 4-chloro-2-(difluoro(4-fluorophenyl)methyl)pyrrolo[1,2-f][1,2,4]triazine-6-carboxylate (1.15 g, 3.26 mmol, crude) in DMF (12 mL) was added potassium iodide (541 mg, 3.26 mmol) and the mixture was stirred at rt for 10 min. Then 3-amino-5-methylpyrazole (496 mg, 5.11 mmol) was added followed by DIPEA (0.890 mL, 5.11 mmol) and the mixture was stirred for 2 days. The reaction mixture was diluted with ethyl acetate and washed with water and brine. The solvents were evaporated and dried to give crude methyl 2-(difluoro(4-fluorophenyl)methyl)-4-(5-Methyl-1H-pyrazol-3-ylamino)pyrrolo[1,2-f][1,2,4]triazine-6-carboxylate (1.3 g, 95%) LC-MS (ESI) m/z 417 (M+H)⁺.

### Example 45

### Preparation of 2-(difluoro(4-fluorophenyl)methyl)-4-(5-methyl-1H-pyrazol-3-ylamino)-N-(2-morpholinoethyl)pyrrolo[1,2-f][1,2,4]triazine-6-carboxamide

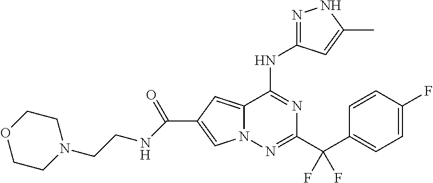

Step A: To a solution of 2-(difluoro(4-fluorophenyl)methyl)-4-(5-methyl-1H-pyrazol-3-ylamino)pyrrolo[1,2-f][1,2,4]triazine-6-carboxylate from Example 44 (600 mg, 1.44 mmol) in 1:1 THF/MeOH (12 mL) at 0° C. was slowly added 3N NaOH (6 mL). The reaction mixture was allowed to warm to rt and stir overnight. The solvents were evaporated and the residue was dissolved in water, then cooled to 0° C. and acidified with 6N HCl. The precipitated solid was collected by filtration and dried to give 2-(difluoro(4-fluorophenyl)methyl)-4-(5-methyl-1H-pyrazol-3-ylamino)pyrrolo[1,2-f][1,2,4]triazine-6-carboxylic acid (545 mg, 94%) as a white solid. ¹H NMR: (300 MHz, DMSO-d₆) δ 11.23 (s, 1H), 8.25 (s, 1H), 7.75-7.70 (m, 3H), 7.41-7.35 (m, 2H), 6.24 (s, 1H), 2.23 (s, 3H); LC-MS (ESI) m/z 403 (M+H)⁺.

Step B: To a solution of 2-(difluoro(4-fluorophenyl)methyl)-4-(5-methyl-1H-pyrazol-3-ylamino)pyrrolo[1,2-f][1,2,4]triazine-6-carboxylic acid (73 mg, 0.182 mmol) in DMF (3 mL) was added HATU (69.01 mg, 0.182 mmol) and the mixture was stirred at rt for 10 min. Then 2-morpholinoethanamine (23.6 mg, 0.182 mmol) was added followed by DIPEA (37.9 uL, 0.217 mmol) and the mixture was stirred at rt for 2 h. The reaction mixture was purified by preparative HPLC (Phenomenex phenylhexyl reverse phase column eluted with gradient of solvent A=0.05% HOAc/H₂O and solvent B=0.05% HOAc/CH₃CN) to afford 2-(difluoro(4-fluorophenyl)methyl)-4-(5-methyl-1H-pyrazol-3-ylamino)-N-(2-morpholinoethyl)pyrrolo[1,2-f][1,2,4]triazine-6-carboxamide (33.19 mg, 36%) as a white solid. ¹H NMR (300 MHz, DMSO-d₆) δ 12.24 (s, 1H), 11.14 (s, 1H), 8.27 (m, 2H), 7.73-7.66 (m, 3H), 7.43-7.33 (m, 2H), 6.23 (s, 1H), 3.63 (s, 4H), 3.43-3.25 (m, 6H), 2.50 (m, 2H), 2.27 (s, 3H); LC-MS (ESI) m/z 515 (M+H)⁺.

The following compounds were prepared using a similar procedure, but replacing the 2-morpholinoethanamine with the appropriately substituted amine:

| Example | | | Analytical |
|---|---|---|---|
| Number | Structure | Name | Data |
| 45a | 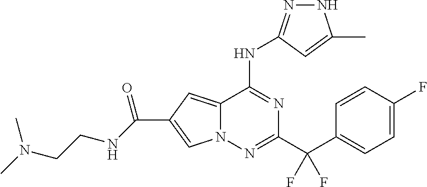 | 2-[Difluoro-(4-fluoro- phenyl)-methyl]-4-(5- methyl-1H-pyrazol-3- ylamino)-pyrrolo[2,1- f][1,2,4]triazine-6- carboxylic acid (2- dimethylamino-ethyl)- amide | LC-MS (ESI) m/z 473 (M + H)⁺; 9.41 min |
| 45b | 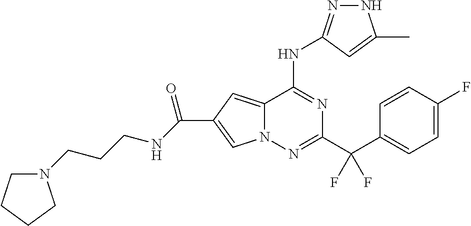 | 2-[Difluoro-(4-fluoro- phenyl)-methyl]-4-(5- methyl-1H-pyrazol-3- ylamino)-pyrrolo[2,1- f][1,2,4]triazine-6- carboxylic acid (3- pyrrolidin-1-yl-propyl)- amide | LC-MS (ESI) m/z 513 (M + H)⁺; 8.61 min |
| 45c | 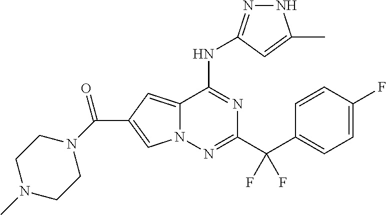 | [2-[Difluoro-(4-fluoro- phenyl)-methyl]-4-(5- methyl-1H-pyrazol-3- ylamino)-pyrrolo[2,1- f][1,2,4]triazin-6-yl]- (4-methyl-piperazin-1- yl)-methanone | LC-MS (ESI) m/z 485 (M + H)⁺; 9.41 min |
| 45d | 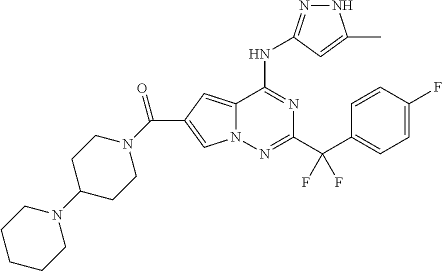 | [1,4′]Bipiperidinyl-1′- yl-[2-[difluoro-(4- fluoro-phenyl)- methyl]-4-(5-methyl- 1H-pyrazol-3- ylamino)-pyrrolo[2,1- f][1,2,4]triazin-6-yl]- methanone | LC-MS (ESI) m/z 553 (M + H)⁺; 8.51 min |
| 45e | 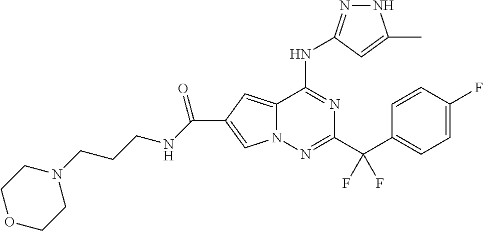 | 2-[Difluoro-(4-fluoro- phenyl)-methyl]-4-(5- methyl-1H-pyrazol-3- ylamino)-pyrrolo[2,1- f][1,2,4]triazine-6- carboxylic acid (3- morpholin-4-yl- propyl)-amide | LC-MS (ESI) m/z 529 (M + H)⁺; 9.41 min |
| 45f | 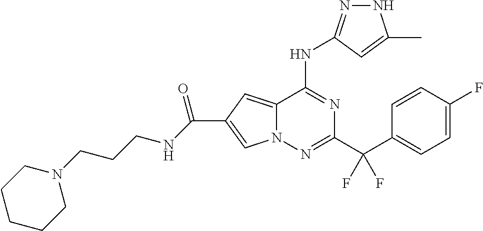 | 2-[Difluoro-(4-fluoro- phenyl)-methyl]-4-(5- methyl-1H-pyrazol-3- ylamino)-pyrrolo[2,1- f][1,2,4]triazine-6- carboxylic acid (3- piperidin-1-yl-propyl)- amide | LC-MS (ESI) m/z 527 (M + H)⁺; 8.61 min |
| 45g | 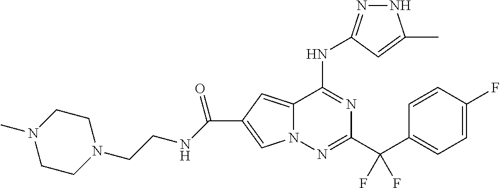 | 2-[Difluoro-(4-fluoro- phenyl)-methyl]-4-(5- methyl-1H-pyrazol-3- ylamino)-pyrrolo[2,1- f][1,2,4]triazine-6- carboxylic acid [2-(4- methyl-piperazin-1-yl)- ethyl]-amide | LC-MS (ESI) m/z 528 (M + H)⁺; 9.48 min |

### Example 46

### Preparation of 3-(Diethylamino)-N-(2-(difluoro(4-fluorophenyl)methyl)-4-(5-methyl-1H-pyrazol-3-ylamino)pyrrolo[1,2-f][1,2,4]triazin-6-yl)propanamide

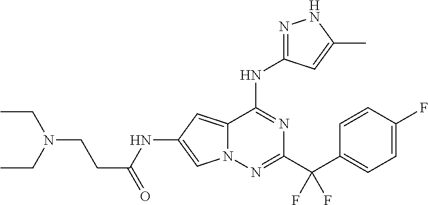

Step A: To a suspension of sodium hydride (2.84 g, 118.6 mmol) in DMF (20 mL) was added ethyl 4-nitro-1H-pyrrole-2-carboxylate (18.2 g, 98.8 mmol) in DMF (20 mL). The mixture was stirred at room temperature for 1 h. Chloramine, prepared as described in General Procedure A, (395 mL, 118.6 mmol) was added and the mixture was stirred at rt overnight. The crude mixture was partitioned between ether (600 mL) and water (700 mL), and the ether layer was dried over magnesium sulfate and concentrated. Trituration of the residue with ethyl acetate gave ethyl 1-amino-4-nitro-1H-pyrrole-2-carboxylate (7.0 g, 36%).

Step B: Methanol was added to a pressure vessel, cooled to −10° C., and ammonia gas bubbled through the solution for 30 min. Ethyl 1-amino-4-nitro-1H-pyrrole-2-carboxylate (7.0 g, 35 mmol) was added and the vessel was sealed and heated at 80° C. overnight. The mixture was cooled and a solid was formed, which was collected by filtration to afford 1-amino-4-nitro-1H-pyrrole-2-carboxamide (6.0 g, 100%).

Step C: To a solution of 2,2-difluoro-2-(4-fluorophenyl)acetic acid prepared as described in Example 4 (7.605 g, 40 mmol) in DMF (50 mL) was added HATU (18.25 g, 48 mmol). After stirring at rt for 30 minutes, 1-amino-4-nitro-1H-pyrrole-2-carboxamide (5.71 g, 33.56 mmol) was added, followed by DIPEA (10.34 g, 80 mmol). The mixture was stirred at room temperature overnight, then the reaction was quenched by addition of water. The mixture was extracted with EtOAc, and the combined organic layers were dried over MgSO₄. The crude product was purified on a silica gel column using a mixture of EtOAc-hexane as eluent to give 1-(2,2-difluoro-2-(4-fluorophenyl)acetamido)-4-nitro-1H-pyrrole-2-carboxamide as a solid (10.885 g, 95%). NMR (300 MHz, DMSO-d₆) δ 12.89 (br, 1H), 8.45 (d, 1H), 7.92 (br, 1H), 7.78 (dd, 2H), 7.55 (d, 1H), 7.44 (br, 1H), 7.39 (t, 2H).

Step D: To a suspension of 1-(2,2-difluoro-2-(4-fluorophenyl)acetamido)-4-nitro-1H-pyrrole-2-carboxamide (5.75 g, 16.8 mmol) in 1,2-dichloroethane (90 mL) was added chlorotrimethylsilane (21.73 g, 11.9 mmol), followed by Et₃N (40.48 g, 23.8 mmol). The mixture was heated at 85° C. overnight, then the reaction was quenched by adding ice and the solution was extracted with DCM. The combined organic layers were dried over MgSO₄, and the crude product was purified on a silica gel column using a mixture of MeOH— DCM as eluent to give 2-(difluoro(4-fluorophenyl)methyl)-6-nitropyrrolo[1,2-f][1,2,4]triazin-4-ol as a solid (2.966 g, 54%). LC-MS (ESI) m/z 322 (M−H)⁻.

Step E: To a solution of 2-(difluoro(4-fluorophenyl)methyl)-6-nitropyrrolo[1,2-f][1,2,4]triazin-4-ol (270 mg, 0.83 mmol) in POCl₃ (10 mL) was added N,N-dimethylaniline (5 drops). The mixture was heated at 120° C. overnight, then POCl₃ was evaporated under reduced pressure. Toluene was added and concentrated under reduced pressure three times. The residue was then extracted with diethyl ether three times. The extracts were combined, concentrated under reduced pressure, and dried under vacuum to give 4-chloro-2-(difluoro(4-fluorophenyl)methyl)-6-nitropyrrolo[1,2-f][1,2,4]triazine as a solid (285 mg, >99%). ¹H NMR (300 MHz, CDCl₃) δ 8.08 (d, 1H), 7.62-7.67 (m, 3H), 7.22 (t, 2H).

Step F: General Procedure C was followed using 4-chloro-2-(difluoro(4-fluorophenyl)methyl)-6-nitropyrrolo[1,2-f][1,2,4]triazine in place of (4-chloropyrrolo[1,2-f][1,2,4]triazin-2-yl)(4-fluorophenyl)methanone. The reaction mixture was diluted with water and the solid was collected by filtration. After washing with water, the solid was dried under vacuum over P₂O₅ to give 2-(difluoro(4-fluorophenyl)methyl)-N-(5-methyl-1H-pyrazol-3-yl)-6-nitropyrrolo[1,2-f][1,2,4]triazin-4-amine (92%). ¹HNMR (300 MHz, DMSO-d₆) δ 12.39 (br, 1H), 11.52 (br, 1H), 8.88 (d, 1H), 8.02 (d, 1H), 7.74 (dd, 2H), 7.39 (t, 2H), 6.25 (s, 1H), 2.23 (s, 3H); LC-MS (ESI) m/z 404 (M+H)⁺.

Step G: A mixture of 2-(difluoro(4-fluorophenyl)methyl)-N-(5-methyl-1,1-pyrazol-3-yl)-6-nitropyrrolo[1,2-f][1,2,4]triazin-4-amine (2.75 g, 6.8 mmol) and 10% palladium on carbon (350 mg) in MeOH (50 mL) and 10% HCl (5 mL) was shaken under a hydrogen atmosphere (35 psi) for 18 h. The mixture was filtered through Celite and washed with MeOH. The filtrate was concentrated under reduced pressure, and diethyl ether was added to form a solid. The solid was collected by filtration.

To the above solid were added MeOH (115 mL) and 10% palladium on active carbon (430 mg). The mixture was stirred at room temperature under an atmosphere of hydrogen overnight. The mixture was filtered through Celite, washing with MeOH. The filtrate was concentrated to give 2-(difluoro(4-fluorophenyl)methyl)-N⁴-(5-methyl-1H-pyrazol-3-yl)pyrrolo[1,2-f][1,2,4]triazine-4,6-diamine hydrochloride as a solid (73%). ¹HNMR (300 MHz, DMSO-d₆) δ 11.17 (br, 1H), 10.46 (br, 2H), 7.98 (d, 1H), 7.71 (dd, 2H), 7.35-7.44 (m, 3H), 6.23 (s, 1H), 5.28 (br, 2H), 2.2 (s, 3H); LC-MS (ESI) m/z 374 (M+H)⁺.

Step H: To a mixture of 2-(difluoro(4-fluorophenyl)methyl)-N⁴-(5-methyl-1H-pyrazol-3-yl)pyrrolo[1,2-f][1,2,4]triazine-4,6-diamine hydrochloride (164 mg, 0.4 mmol), 3-(diethylamino)propanoic acid hydrochloride (91 mg, 0.5 mmol), and HATU (228 mg, 0.6 mmol) in DMF (8 mL) was added Et₃N (303 mg, 3 mmol). The mixture was stirred at rt overnight. After DMF was evaporated under reduced pressure, the residue was purified by preparative reverse phase HPLC (diphenyl column) using MeCN/water (0.05% AcOH) as eluents to give 3-(diethylamino)-N-(2-(difluoro(4-fluorophenyl)methyl)-4-(5-methyl-1H-pyrazol-3-ylamino)pyrrolo[1,2-f][1,2,4]triazin-6-yl)propanamide as a solid (17 mg, 9%). ¹H NMR (300 MHz, DMSO-d₆) δ 12.21 (br, 1H), 10.88 (br, 1H), 10.65 (br, 1H), 8.06 (d, 1H), 7.69 (dd, 2H), 7.37 (t, 2H), 7.25 (s, 1H), 6.20 (s, 1H), 3.39 (t, 2H), 3.12-3.19 (m, 4H), 2.82 (t, 2H), 1.23 (m, 6H); LC-MS (ESI) m/z 501 (M+H)⁺.

The following compounds were made using a similar procedure, replacing 3-(diethylamino)propanoic acid hydrochloride with the appropriately substituted propanoic acid hydrochloride:

| Example | | | |
|---|---|---|---|
| Number | Structure | Name | Analytical Data |
| 46a | 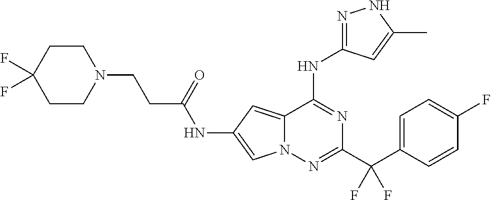 | N-[2- [Difluoro-(4- fluoro- phenyl)- methyl]-4-(5- methyl-1H- pyrazol-3- ylamino)- pyrrolo[2,1- f][1,2,4]tria- zin-6-yl]-3- (4,4-difluoro- piperidin-1- yl)- propionamide | ¹H NMR (300 MHz, DMSO- d₆) δ 1.99 (m, 4H), 2.21 (s, 3H), 2.50 (overlapping with solvent, 6H), 2.73 (t, 2H), 6.20 (s, 1H), 7.36 (t and s, 3H), 7.69 (dd, 2H), 8.05 (d, 1H), 10.5 (s, 1H), 10.81 (s, 1H), 12.19 (s, 1H); LC-MS (ESI) m/z 549 (M + H)⁺; 9.27 min. |
| 46b | 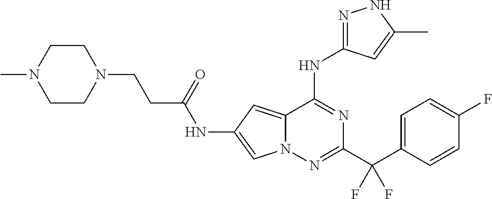 | N-[2- [Difluoro-(4- fluoro- phenyl)- methyl]-4-(5- methyl-1H- pyrazol-3- ylamino)- pyrrolo[2,1- f][1,2,4]tria- zin-6-yl]-3-(4- methyl- piperazin-1- yl)- propionamide | ¹H NMR (300 MHz, CD₃OD) δ 2.24 (s, 3H), 2.29 (s, 3H), 2.59 (t, 2H), 2.65 (m, 4H), 2.81 (t, 2H), 3.26 (overlapping with solvent, 4H) 6.35 (s, 1H), 7.00 (s, 1H), 7.21 (t, 2H), 7.70 (dd, 2H), 8.07 (d, 1H); LC-MS (ESI) m/z 528 (M + H)⁺; 8.48 min. |

### Example 47

### Preparation of (2-(difluoro(4-fluorophenyl)methyl)-4-(5-methyl-1H-pyrazol-3-ylamino)pyrrolo[1,2-f][1,2,4]triazin-6-yl)methanol

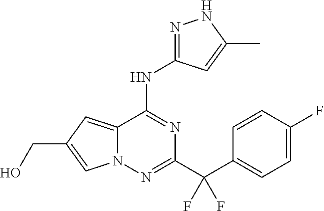

To a suspension of methyl 2-(difluoro(4-fluorophenyl)methyl)-4-(5-methyl-1H-pyrazol-3-ylamino)pyrrolo[1,2-f][1,2,4]triazine-6-carboxylate from Example 44 (3.5 g, 0.0084 mol) in THF (50 mL), was added DIBALH (1M in THF) at rt. After stirring for 3 h at room temperature, the mixture was cooled at 0° C. and the reaction was quenched by slow addition of MeOH (100 mL). The mixture was stirred for 30 min and THF (100 mL) was added. The solid was filtered and washed with a mixture of THF/MeOH (1/1). The filtrate was concentrated to dryness to afford (2-(difluoro(4-fluorophenyl)methyl)-4-(5-methyl-1H-pyrazol-3-ylamino)pyrrolo[1,2-f][1,2,4]triazin-6-yl)methanol as a white solid (98%). ¹H NMR (DMSO-d₆): δ 12.20 (s, 1H), 10.82 (s, 1H), 7.74 (s, 1H), 7.69 (m, 2H), 7.36 (t, 2H), 7.26 (s, 1H), 6.22 (s, 1H), 5.09 (t, 1H), 4.53 (d, 2H), 2.21 (s, 3H). LC-MS (ESI) m/z 389 (M+H)⁺.

### Example 48

### Preparation of 6-(aminomethyl)-2-(difluoro(4-fluorophenyl)methyl)-N-(5-methyl-1H-pyrazol-3-yl)pyrrolo[1,2-f][1,2,4]triazin-4-amine

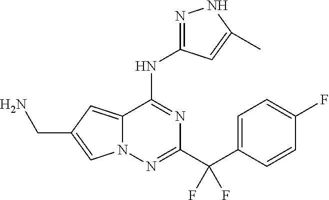

To the oxime from Example 55 (240 mg, 0.60 mmol) in 20 mL of HOAc was added activated zinc powder (200 mg, 3.0 mmol). After 1 h, additional zinc powder (100 mg) was added. After an additional 2 h at room temperature, LC-MS indicated the reaction was complete. The reaction mixture was filtered through Celite, the solvent was removed in vacuo, and the residue was purified by reverse-phase preparative HPLC (eluting with 10-80% acetonitrile). Lyophilization of the pure fractions afforded 6-(aminomethyl)-2-(difluoro(4-fluorophenyl)methyl)-N-(5-methyl-1H-pyrazol-3-yl)pyrrolo[1,2-f][1,2,4]triazin-4-amine. ¹H NMR (300 MHz, DMSO-d₆) δ 7.99 (s, 2H), 7.71 (m, 2H), 7.38 (m, 3H), 6.24 (s, 2H), 4.01 (s, 2H), 2.22 (s, 3H); LC-MS (ESI) m/z 388 (M+H)⁺.

### Example 49

### Preparation of N((2-(difluoro(4-fluorophenyl)methyl)-4-(5-methyl-1H-pyrazol-3-ylamino)pyrrolo[1,2-f][1,2,4]triazin-6-yl)methyl)acetamide

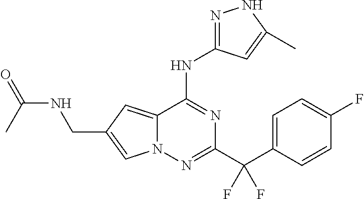

To 6-(aminomethyl)-2-(difluoro(4-fluorophenyl)methyl)-N-(5-methyl-1H-pyrazol-3-yl)pyrrolo[1,2-f][1,2,4]triazin-4-amine from Example 48 (150 mg, 0.38 mmol) and DIPEA (100 μL, 0.57 mmol) in DMF (5 mL) at 0° C. was added dropwise acetyl chloride (32 μL, 0.46 mmol) in DMF (0.5 mL). The reaction mixture was stirred at room temperature overnight, then purified by reverse-phase preparative HPLC (eluting with 10-80% acetonitrile). Lyophilization of the pure fractions gave N-((2-(difluoro(4-fluorophenyl)methyl)-4-(5-methyl-1H-pyrazol-3-ylamino)pyrrolo[1,2-f][1,2,4]triazin-6-yl)methyl)acetamide. ¹HNMR (300 MHz, MeOH-d₄) δ 7.70 (dd, 2H), 7.64 (d, 1H), 7.21 (t, 2H), 6.95 (s, 1H), 6.31 (s, 1H), 4.41 (s, 2H), 2.28 (s, 3H), 1.99 (s, 3H); LC-MS (ESI) m/z 430 (M+H)⁺.

The following compounds were made using a similar procedure, replacing the acetyl chloride with an appropriately substituted acyl halide or sulfonyl halide.

| Example | | | Analytical |
|---|---|---|---|
| Number | Structure | Name | Data |
| 49a | 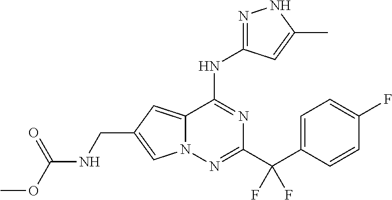 | [2-[Difluoro-(4-fluoro- phenyl)-methyl]-4-(5- methyl-1H-pyrazol-3- ylamino)-pyrrolo[2,1- f][1,2,4]triazin-6- ylmethyl]-carbamic acid methyl ester | LC-MS (ESI) m/z 446 (M + H)⁺; 11.9 min |
| 49b | 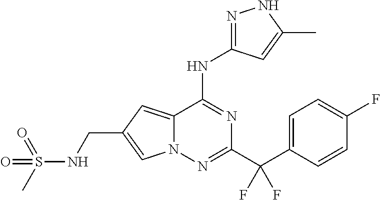 | N-[2-[Difluoro-(4-fluoro- phenyl)-methyl]-4-(5- methyl-1H-pyrazol-3- ylamino)-pyrrolo[2,1- f][1,2,4]triazin-6- ylmethyl]- methanesulfonamide | LC-MS (ESI) m/z 466 (M + H)⁺; 11.7 min |
| 49c | 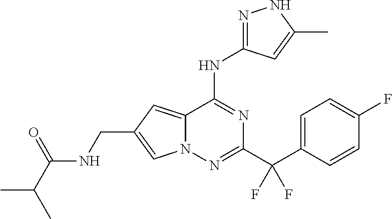 | N-[2-[Difluoro-(4-fluoro- phenyl)-methyl]-4-(5- methyl-1H-pyrazol-3- ylamino)-pyrrolo[2,1- f][1,2,4]triazin-6- ylmethyl]-isobutyramide | LC-MS (ESI) m/z 458 (M + H)⁺; 11.9 min |
| 49d | 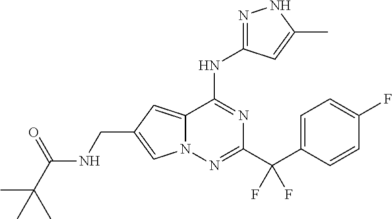 | N-[2-[Difluoro-(4-fluoro- phenyl)-methyl]-4-(5- methyl-1H-pyrazol-3- ylamino)-pyrrolo[2,1- f][1,2,4]triazin-6- ylmethyl]-2,2-dimethyl- propionamide | LC-MS (ESI) m/z 472 (M + H)⁺; 12.5 min |

### Example 50

### Preparation of 1-(2-(difluoro(4-fluorophenyl)methyl)-4-(5-methyl-1H-pyrazol-3-ylamino)pyrrolo[1,2-f][1,2,4]triazin-6-yl)ethanone

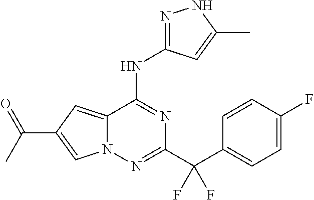

Step A: To a solution of 2-(difluoro(4-fluorophenyl)methyl)-4-(5-methyl-1H-pyrazol-3-ylamino)pyrrolo[1,2-f][1,2,4]triazine-6-carboxylic acid from Example 45 Step H (2.04 g, 5.07 mmol) in DMF (20 mL) was added HATU (2.12 g, 5.57 mmol) and the reaction mixture was stirred for 10 min at rt. Then N,O-dimethylhydroxylamine hydrochloride (494.5 mg, 5.07 mmol) was added followed by DIPEA (1.766 mL, 11.14 mmol), and the reaction mixture was stirred for 4 h. The reaction mixture was diluted with water resulting in precipitation of a solid. The solid was collected by filtration and dried to give 2-(difluoro(4-fluorophenyl)methyl)-N-methoxy-N-methyl-4-(5-methyl-1H-pyrazol-3-ylamino)pyrrolo[1,2-f][1,2,4]triazine-6-carboxamide (1.9 g, 84%) as a solid. ¹H NMR: (300 MHz, DMSO-d₆) δ 12.35 (s, 1H), 11.25 (s, 1H), 8.24 (s, 1H), 7.90 (s, 1H), 7.79-7.75 (m, 2H), 7.45-7.39 (m, 2H), 6.28 (s, 1H), 3.81 (s, 3H), 3.33 (s, 3H), 2.27 (s, 3H); LC-MS (ESI) m/z 446 (M+H)⁺.

Step B: To a solution of 2-(difluoro(4-fluorophenyl)methyl)-N-methoxy-N-methyl-4-(5-methyl-1H-pyrazol-3-ylamino)pyrrolo[1,2-f][1,2,4]triazine-6-carboxamide (1 g, 2.24 mmol) in THF (10 mL) was added methylmagnesium bromide (3 M in ether, 3.73 mL, 11.2 mmol) at 0° C. and the reaction mixture was allowed to warm to rt and stir for 2 h. Additional methylmagnesium bromide (5.22 mL, 15.68 mmol) was added and the mixture was stirred for another 1 h, then portioned between DCM and aqueous ammonium chloride. The emulsion formed was removed by passing through a celite pad. The organic layer from the filtrate was separated, washed with brine, dried (Na₂SO₄) and concentrated. The residue was sonicated with DCM to afford 1-(2-(difluoro(4-fluorophenyl)methyl)-4-(5-methyl-1H-pyrazol-3-ylamino)pyrrolo[1,2-f][1,2,4]triazin-6-yl)ethanone (573 mg, 64%) as a solid. ¹H NMR: (300 MHz, DMSO-d₆) δ 12.22 (s, 1H), 11.25 (s, 1H), 8.54 (s, 1H), 7.75-7.69 (m, 3H), 7.41-7.35 (m, 2H), 6.27 (s, 1H), 2.23 (s, 3H), 1.91 (s, 3H); LC-MS (ESI) m/z 401 (M+H)⁺.

### Example 51

### Preparation of 1-(2-(difluoro(4-fluorophenyl)methyl)-4-(5-methyl-1H-pyrazol-3-ylamino)pyrrolo[1,2-f][1,2,4]triazin-6-yl)ethanol

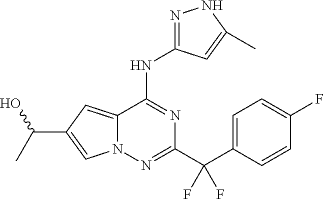

To a stirred solution of ketone from Example 50 (85 mg, 0.21 mmol) in EtOH/THF (3:1) at rt was added 50 mg of NaBH₄. The resulting mixture was stirred at rt overnight, whereupon LC-MS indicated that the reaction was complete. The crude product was purified by reverse-phase preparative HPLC (eluting with 10-80% acetonitrile). Lyophilization of the pure fractions gave 1-(2-(difluoro(4-fluorophenyl)methyl)-4-(5-methyl-1H-pyrazol-3-ylamino)pyrrolo[1,2-f][1,2,4]triazin-6-yl)ethanol. ¹H NMR (300 MHz, DMSO-d₆) δ 12.19 (s, 1H), 10.79 (s, 1H), 7.72 (s, 1H), 7.68 (m, 2H), 7.36 (t, 2H), 7.28 (s, 1H), 6.24 (s, 1H), 5.12 (d, 1H), 4.83 (t, 1H), 2.22 (s, 3H), 1.40 (d, 3H); LC-MS (ESI) m/z 403 (M+H)⁺.

### Example 52

### Preparation of 2-(difluoro(4-fluorophenyl)methyl)-6-(ethoxymethyl)-N-(5-methyl-1H-pyrazol-3-yl)pyrrolo[1,2-f][1,2,4]triazin-4-amine

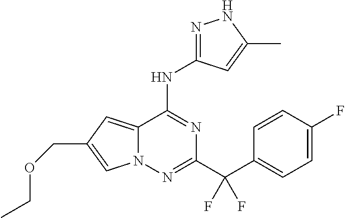

Step A. To a stirred suspension of (2-(difluoro(4-fluorophenyl)methyl)-4-(5-methyl-1H-pyrazol-3-ylamino)pyrrolo[1,2-f][1,2,4]triazin-6-yl)methanol from Example 47 (900 mg, 0.0023 mol) in DCM (20 mL), was added PBr₃ (0.44 mL, 0.0046 mol). The reaction mixture was heated at 60° C. for 10 min, then diluted with DCM (20 mL), cooled to 0° C., treated with saturated aq NaHCO₃ (20 mL). After the mixture was stirred for 15 min, the organic layer was washed with brine and dried over MgSO₄. Crude 6-(bromomethyl)-2-(difluoro(4-fluorophenyl)methyl)-N-(5-methyl-1H-pyrazol-3-yl)pyrrolo[1,2-f][1,2,4]triazin-4-amine was obtained as an off-white solid (800 mg, 77%) and used without further purification. LC-MS (ESI) m/z 452 (M+H)⁺.

Step B. A solution of 6-(bromomethyl)-2-(difluoro(4-fluorophenyl)methyl)-N-(5-methyl-1H-pyrazol-3-yl)pyrrolo[1,2-f][1,2,4]triazin-4-amine (266 mg, 0.59 mmol) in EtOH (15 mL) was heated at 80° C. for 3 h. The solvent was evaporated and the residue was dissolved in DMF and purified on reverse phase HPLC to afford 2-(difluoro(4-fluorophenyl)methyl)-6-(ethoxymethyl)-N-(5-methyl-1H-pyrazol-3-yl)pyrrolo[1,2-f][1,2,4]triazin-4-amine as a white solid (33 mg, 13%). ¹HNMR (DMSO-d₆): δ 12.18 (s, 1H), 10.85 (s, 1H), 7.81 (s, 1H), 7.69 (m, 2H), 7.36 (t, 2H), 7.30 (s, 1H), 6.22 (s, 1H), 4.49 (s, 2H), 3.46 (q, 2H), 2.21 (s, 3H), 1.14 (t, 3H). LC-MS (ESI) m/z 417 (M+H)⁺.

The following compound was made using a similar procedure but replacing the ethanol with isopropyl alcohol:

| Example | | | |
|---|---|---|---|
| Number | Structure | Name | Analytical Data |
| 52a | 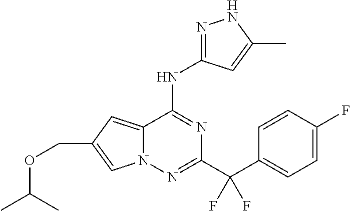 | 2-(difluoro(4- fluorophenyl)methyl)- 6- (isopropoxymethyl)- N-5-methyl-1H- pyrazol-3- yl)pyrrolo[1,2- f][1,2,4]triazin-4- amine | ¹HNMR (DMSO-d6): δ12.21 (s, 1H), 10.84 (s, 1H), 7.79 (s, 1H), 7.70 (m, 2H), 7.36 (t, 2H), 7.30 (s, 1H), 6.23 (s, 1H), 4.50 (s, 2H), 3.65 (q, 1H), 2.21 (s, 3H), 1.13 (d, 6H). LC- MS (ESI) m/z 431 (M + H)⁺ |

### Example 53

### Preparation of N-(2-(Difluoro(4-fluorophenyl)methyl)-4-(5-methyl-1H-pyrazol-3-ylamino)pyrrolo[1,2-f][1,2,4]triazin-6-yl)-2-morpholinoacetamide

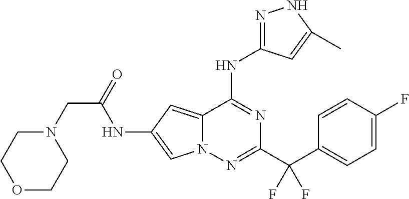

Step A: To a suspension of 2-(difluoro(4-fluorophenyl)methyl)-N⁴-(5-methyl-1H-pyrazol-3-yl)pyrrolo[1,2-f][1,2,4]triazine-4,6-diamine hydrochloride from Example 46, Step G (615 mg, 1.5 mmol) in DCM (15 mL) was added bromoacetyl chloride (354 mg, 2.25 mmol), followed by saturated aq NaHCO₃. After stirring at rt overnight, additional saturated aq NaHCO₃ was added and the mixture was extracted with DCM. The combined organic layers were dried over MgSO₄ and concentrated under reduced pressure. To the residue was added EtOAc (20 mL) and 10% NaOH solution (8 mL) and the mixture was stirred at rt for 30 minutes, then H₂O was added and the mixture was extracted with EtOAc. The combined organic layers were dried over MgSO₄ and concentrated under reduced pressure to afford 2-bromo-N-(2-(difluoro(4-fluorophenyl)methyl)-4-(5-methyl-1H-pyrazol-3-ylamino)pyrrolo[1,2-f][1,2,4]triazin-6-yl)acetamide as a solid (560 mg, 76%). ¹H NMR (300 MHz, DMSO-d₆) δ 12.21 (br, 1H), 10.86 (br, 2H), 8.04 (d, 1H), 7.70 (dd, 2H), 7.30-7.39 (m, 3H), 6.21 (s, 1H), 4.06 (s, 2H), 2.22 (s, 3H); LC-MS (ESI) m/z 494 (M+H)⁺.

Step B: A mixture of 2-bromo-N-(2-(difluoro(4-fluorophenyl)methyl)-4-(5-methyl-1H-pyrazol-3-ylamino)pyrrolo[1,2-f][1,2,4]triazin-6-yl)acetamide (186 mg, 0.38 mmol), morpholine (44 mg, 0.5 mmol), KI (66 mg, 0.4 mmol), and N,N-diisopropylethylamine (0.5 mL) in DMF (7 mL) was heated at 60° C. overnight. The mixture was purified on a silica gel column using a mixture of MeOH-DCM as eluent to give N-(2-(difluoro(4-fluorophenyl)methyl)-4-(5-methyl-1H-pyrazol-3-ylamino)pyrrolo[1,2-f][1,2,4]triazin-6-yl)-2-morpholinoacetamide as a solid (36 mg, 19%). ¹H NMR (300 MHz, DMSO-d₆) δ 12.19 (br, 1H), 10.83 (br, 1H), 10.23 (br, 1H), 8.05 (s, 1H), 7.79 (dd, 2H), 7.33-7.39 (m, 3H), 6.20 (s, 1H), 3.63 (t, 4H), 3.15 (s, 2H), 2.51 (t, 4H, overlapping with solvent), 2.27 (s, 3H); LC-MS (ESI) m/z 501 (M+H)⁺.

The following compounds were prepared using a similar procedure, replacing the morpholine in step B with the appropriate amine.

| Example | | | Analytical |
|---|---|---|---|
| Number | Structure | Name | Data |
| 53a | 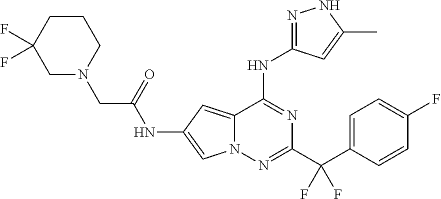 | N-[2-[Difluoro- (4-fluoro- phenyl)- methyl]-4-(5- methyl-1H- pyrazol-3- ylamino)- pyrrolo[2,1- f][1,2,4]triazin- 6-yl]-2-(3,3- difluoro- piperidin-1-yl)- acetamide | ¹H NMR (300 MHz, DMSO- d₆) δ 1.69 (m, 2H), 1.9 (m, 2H), 2.21 (s, 3H), 2.60 (m, 2H), 2.88 (t, 2H), 3.31 (s, 2H), 6.21 (S, 1H), 7.32 (s, 1H), 7.36 (t, 2H), 7.70 (dd, 2H), 8.05 (d, 1H), 10.24 (s, 1H), 10.85 (s, 1H), 12.20 (s, 1H); LC-MS (ESI) m/z 535 (M + H)⁺; 12.98 min. |
| 53b | 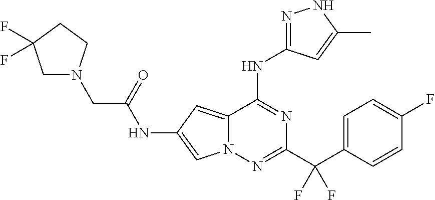 | N-[2-[Difluoro- (4-fluoro- phenyl)- methyl]-4-(5- methyl-1H- pyrazol-3- ylamino)- pyrrolo[2,1- f][1,2,4]triazin- 6-yl]-2-(3,3- difluoro- pyrrolidin-1-yl)- acetamide | ¹H NMR (300 MHz, DMSO- d₆) δ 2.14 (s, 3H), 2.26 (m, 2H), 2.87 (t, 2H), 3.08 (t, 2H), 3.35 (s, 2H), 6.20 (s, 1H), 7.36 (t and s, 3H), 7.70 (dd, 2H), 8.04 (d, 1H), 10.33 (s, 1H), 10.83 (s, 1H), 12.19 (s, 1H); LC-MS (ESI) m/z 521 (M + H)⁺; 12.64 min. |
| 53c | 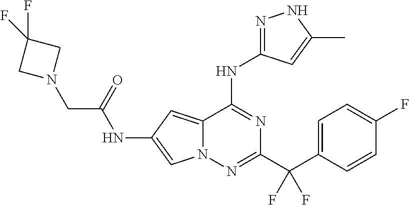 | 2-(3,3-Difluoro- azetidin-1-yl)- N-[2-[difluoro- (4-fluoro- phenyl)- methyl]-4-(5- methyl-1H- pyrazol-3- ylamino)- pyrrolo[2,1- f][1,2,4]triazin- 6-yl]-acetamide | ¹H NMR (300 MHz, DMSO- d₆) δ 2.21 (s, 3H), 3.44 (s, 2H), 3.78 (t, 4H), 6.20 (s, 1H), 7.36 (t and s, 3H), 7.69 (dd, 2H), 8.02 (d, 1H), 10.31 (s, 1H), 10.83 (s, 1H), 12.19 (s, 1H); LC-MS (ESI) m/z 507 (M + H)⁺; 12.26 min. |
| 53d | 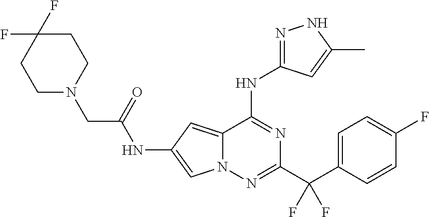 | N-[2-[Difluoro- (4-fluoro- phenyl)- methyl]-4-(5- methyl-1H- pyrazol-3- ylamino)- pyrrolo[2,1- f][1,2,4]triazin- 6-yl]-2-(4,4- difluoro- piperidin-1-yl)- acetamide | ¹H NMR (300 MHz, DMSO- d₆) δ 2.02 (m, 4H), 2.21 (s, 3H), 2.66 (t, 4H), 2.26 (s, 2H), 6.21 (s, 1H), 7.36 (t and s, 3H), 7.70 (dd, 2H), 8.04 (s, 1H), 10.24 (s, 1H), 10.83 (s, 1H), 12.19 (s, 1H); LC-MS (ESI) m/z 535 (M + H)⁺; 12.23 min. |
| 53e | 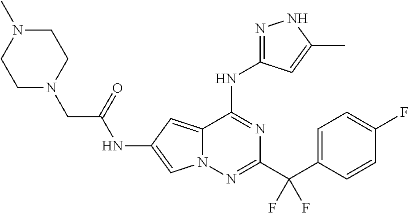 | N-[2-[Difluoro- (4-fluoro- phenyl)- methyl]-4-(5- methyl-1H- pyrazol-3- ylamino)- pyrrolo[2,1- f][1,2,4]triazin- 6-yl]-2-(4- piperazin-1-yl)- acetamide | ¹H NMR (300 MHz, DMSO- d₆) δ 2.17 (s, 3H), 2.21 (s, 3H), 2.38 (m, 8H), 3.13 (s, 2H), 6.20 (s, 1H), 7.35 (t and s, 3H), 7.69 (dd, 2H), 8.04 (d, 1H), 10.17 (s, 1H), 10.82 (s, 1H), 12.19 (s, 1H); LC-MS (ESI) m/z 514 (M + H)⁺; 8.59 min. |

### Example 54

### Preparation of 2-(difluoro(4-fluoronhenyl)methyl)-N-(5-methyl-1H-pyrazol-3-yl)-6-(morpholinomethyl)pyrrolo[1,2-f][1,2,4]triazin-4-amine

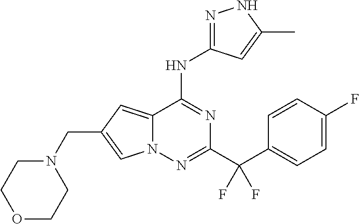

Step A: To a solution of (2-(difluoro(4-fluorophenyl)methyl)-4-(5-methyl-1H-pyrazol-3-ylamino)pyrrolo[1,2-f][1,2,4]triazin-6-yl)methanol from Example 47 (513.0 mg, 1.32 mmol) in DCM (11 mL) at 0° C. was added Dess-Martin periodinane (671.83 mg, 1.58 mmol). The mixture was stirred at rt overnight, then the reaction was quenched with sodium thiosulfate and 10% aq. NaHCO₃. The resulting mixture was extracted with a EtOAc/MeOH (9:1) and the separated organic layer was concentrated. The residue was sonicated with ether, then the solid was collected by filtration and dried to give 2-(difluoro(4-fluorophenyl)methyl)-4-(5-methyl-1H-pyrazol-3-ylamino)pyrrolo[1,2-f][1,2,4]triazine-6-carbaldehyde (453 mg, 90%) as white solid. ¹H NMR (300 MHz, DMSO-d₆) δ 12.45 (s, 1H), 11.35 (s, 1H), 9.97 (s, 1H), 8.49 (s, 1H), 7.78-7.72 (m 3H), 7.40-7.28 (m, 2H), 6.25 (s, 1H), 2.25 (s, 3H); LC-MS (ESI) m/z 387 (M+H)⁺.

Step B: To a solution of 2-(difluoro(4-fluorophenyl)methyl)-4-(5-methyl-1H-pyrazol-3-ylamino)pyrrolo[1,2-f][1,2,4]triazine-6-carbaldehyde (150 mg, 0.388 mmol) and morpholine (67.6 uL, 0.776 mmol) in dichloroethane (5 mL) was added sodium triacetoxy borohydride (131.57 mg, 0.621 mmol). The mixture was stirred at rt for 90 min, then 10% NaHCO₃ was added. The resulting mixture was extracted with ethyl acetate, then the organic layer was washed with brine, dried and concentrated. The residue was purified by silica gel chromatography using DCM/MeOH as eluent, to give 2-(difluoro(4-fluorophenyl)methyl)-N-(5-methyl-1H-pyrazol-3-yl)-6-(morpholinomethyl)pyrrolo[1,2-f][1,2,4]triazin-4-amine (17 mg, 10%) as white solid. ¹H NMR (300 MHz, DMSO-d₆) δ 12.20 (s, 1H), 10.79 (s, 1H), 7.75-7.67 (m, 3H), 7.39-7.33 (m, 2H), 7.26 (s, 1H), 6.22 (s, 1H), 3.57 (s, 4H), 3.51 (s, 2H), 2.27 (s, 4H), 2.21 (s, 3H); LC-MS (ESI) m/z 458 (M+H)⁺.

The following compounds were made using a similar procedure but replacing the morpholine with an appropriately substituted amine:

| Example | | | |
|---|---|---|---|
| Number | Structure | Name | Analytical Data |
| 54a | 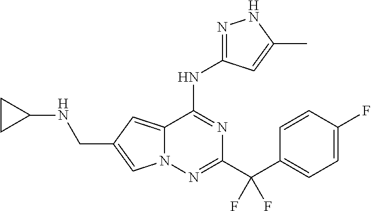 | 6-((cyclopropylamino)methyl)- 2-(difluoro(4- fluorophenyl)methyl)-N-(5- methyl-1H-pyrazol-3- yl)pyrrolo[1,2-f][1,2,4]triazin- 4-amine | LC-MS (ESI) m/z 428 (M + H)⁺; 8.63 min |
| 54b | 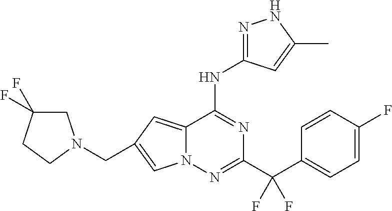 | 2-(difluoro(4- fluorophenyl)methyl)-6-((3,3- difluoropyrrolidin-1- yl)methyl)-N-(5-methyl-1H- pyrazol-3-yl)pyrrolo[1,2- f][1,2,4]triazin-4-amine | LC-MS (ESI) m/z 478 (M + H)⁺; 11.48 min |
| 54c | 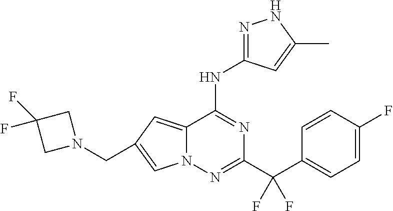 | 2-(difluoro(4- fluorophenyl)methyl)-6-((3,3- difluoroazetidin-1-yl)methyl)- N-(5-methyl-1H-pyrazol-3- yl)pyrrolo[1,2-f][1,2,4]triazin- 4-amine | LC-MS (ESI) m/z 464 (M + H)⁺; 11.49 min |

### Example 55

### Preparation of (Z)-1-(2-(difluoro(4-fluorophenyl)methyl)-4-(5-methyl-1H-pyrazol-3-ylamino)pyrrolo[1,2-f][1,2,4]triazin-6-yl)ethanone oxime

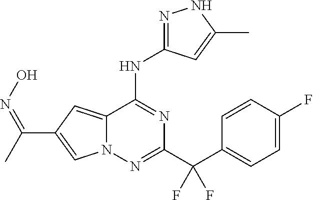

Step A: To a solution of 1-(2-(difluoro(4-fluorophenyl)methyl)-4-(5-methyl-1H-pyrazol-3-ylamino)pyrrolo[1,2-f][1,2,4]triazin-6-yl)ethanone from Example 50 (200 mg, 0.499 mmol) in EtOH (3 mL) were added pyridine (80.7 μL, 0.998 mmol) and hydroxylamine hydrochloride (52.01 mg, 0.748 mmol) and the reaction mixture was heated at 40° C. for 2 h. The mixture was diluted with DCM and washed with aq. cupric sulfate. The emulsion formed was removed by filtering through a Celite pad. The filtrate was concentrated and the residue was purified by silica gel chromatography using DCM/MeOH as eluent, followed by preparative TLC to afford (Z)-1-(2-(difluoro(4-fluorophenyl)methyl)-4-(5-methyl-1H-pyrazol-3-ylamino)pyrrolo[1,2-f][1,2,4]triazin-6-yl)ethanone oxime (13.36 mg, 7%) as a 1:1 mixture isomers. LC-MS (ESI) m/z 414 (M−H)⁺.

### Example 56

### Preparation of 2-(difluoro(4-fluorophenyl)methyl)-4-(5-methyl-1H-pyrazol-3-ylamino)pyrrolo[1,2-f][1,2,4]triazine-6-carbaldehyde oxime

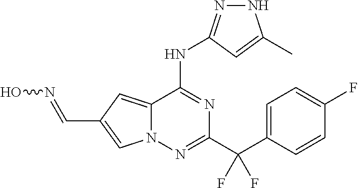

Step A: To a stirred solution of (2-(difluoro(4-fluorophenyl)methyl)-4-(5-methyl-1H-pyrazol-3-ylamino)pyrrolo[1,2-f][1,2,4]triazin-6-yl)methanol from Example 47 (2.0 g, 5.15 mmol) in DCM (15 mL) and DMA (15 mL) at 0° C. was added Dess-Martin periodane (2.2 g, 5.15 mmol). The reaction mixture was stirred at rt for 4 h. Additional Dess-Martin reagent (700 mg, 1.75 mmol) was added, and stirring was continued for 1 h, whereupon LC-MS indicated that the reaction was complete. To the reaction mixture were added 10% MeOH in EtOAc (20 mL), saturated Na₂SO₃ (20 mL), and aq. NaHCO₃ (20 mL), and the mixture was stirred at rt for 20 min. The separated aqueous layer was extracted with 10% MeOH in EtOAc (3×100 mL) and the combined organic phases were dried over MgSO₄, filtered, and evaporated in vacuo to give 2-(difluoro(4-fluorophenyl)methyl)-4-(5-methyl-1H-pyrazol-3-ylamino)pyrrolo[1,2-f][1,2,4]triazine-6-carbaldehyde (1.31 g) as a brown oil which solidified slowly over time. ¹H NMR (300 MHz, DMSO-d₆) δ 11.36 (s, 1H), 9.98 (s, 1H), 8.54 (s, 1H), 7.73 (m, 4H), 7.38 (t, 3H), 6.25 (s, 1H), 2.23 (s, 3H); LC-MS (ESI) m/z 387 (M+H)⁺.

Step B: To a stirred solution of 2-(difluoro(4-fluorophenyl)methyl)-4-(5-methyl-1H-pyrazol-3-ylamino)pyrrolo[1,2-f][1,2,4]triazine-6-carbaldehyde (100 mg, 0.26 mmol) in EtOH (4 mL) was added hydroxylamine hydrochloride (20 mg, 0.28 mmol) and pyridine (22 μL, 0.28 mmol). The resulting mixture was stirred under argon at 40° C. for 1 h, whereupon LC-MS indicated that the reaction was complete. The solvent was removed in vacuo and the residue was purified by reverse-phase preparative HPLC (eluting with 20-80% acetonitrile). Lyophilization of the pure fractions gave 2-(difluoro(4-fluorophenyl)methyl)-4-(5-methyl-1H-pyrazol-3-ylamino)pyrrolo[1,2-f][1,2,4]triazine-6-carbaldehyde oxime. ¹H NMR (300 MHz, DMSO-d₆) δ 12.24 (br s, 1H), 11.5 (s, 1H), 11.2 (br s, 1H), 8.24 (s, 1H), 7.72 (m, 3H), 7.49 (s, 1H), 7.37 (m, 2H), 6.21 (s, 1H), 2.21 (s, 3H); LC-MS (ESI) m/z 402 (M+H)⁺.

### Example 57

### Preparation of (4-fluorophenyl)(6-(hydroxymethyl)-4-(5-methyl-1H-pyrazol-3-ylamino)pyrrolo[1,2-f][1,2,4]triazin-2-yl)methanol

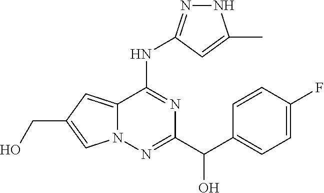

Step A. To a solution of methyl 1-amino-5-carbamoyl-1H-pyrrole-3-carboxylate from Example 44, step B (500 mg, 2.72 mmol) in THF (8 mL) was added 5-(4-fluorophenyl)-1,3-dioxolane-2,4-dione (693.5 mmol, 3.53 mmol) and the reaction mixture was heated at reflux for 12 h. The solvent was evaporated and the residue was sonicated with ethyl acetate. The solid was collected by filtration and dried to give methyl 5-carbamoyl-1-(2-hydroxy-2-phenylacetamido)-1H-pyrrole-3-carboxylate (650 mg, 75%) as a solid. ¹H NMR: (300 MHz, DMSO-d₆) δ 11.65 (s, 1H), 7.69 (s, 1H), 7.56-7.48 (m, 3H), 7.26-7.13 (m, 4H), 6.50 (d, 1H), 5.17 (d, 1H), 3.72 (s, 3H); LC-MS (ESI) m/z 336 (M+H)⁺.

Step B. To a solution of methyl 5-carbamoyl-1-(2-hydroxy-2-phenylacetamido)-1H-pyrrole-3-carboxylate (1 g, 2.98 mmol) in dioxane:DMF (12 mL) was added PTSA (850 mg, 4.47 mmol) and the mixture was heated at 180° C. in a microwave for 25 min. The reaction mixture was diluted with water and the precipitate was collected and dried to give methyl 2-((4-fluorophenyl)(hydroxy)methyl)-4-hydroxypyrrolo[1,2-f][1,2,4]triazine-6-carboxylate (600 mg, 64%) as a solid. ¹H NMR: (300 MHz, DMSO-d₆) δ 11.88 (s, 1H), 8.08 (s, 1H), 7.61-7.56 (m, 2H), 7.23-7.16 (m, 3H), 6.59 (s, 1H), 5.54 (s, 1H), 3.78 (s, 3H); LC-MS (ESI) m/z 318 (M+H)⁺.

Step C. To a solution of methyl 2-((4-fluorophenyl)(hydroxy)methyl)-4-hydroxypyrrolo[1,2-f][1,2,4]triazine-6-carboxylate (558 mg, 1.75 mmol) in 2:1 DCM:DMA (9 mL) was added Dess-Martin periodinane (742.26 mg, 1.75 mmol) at 0° C. and the mixture was stirred at rt for 2 h. To the mixture were added EtOAc/MeOH, aq. sodium thiosulfate and 10% aq. NaHCO₃. The emulsion formed was removed by filtering through a Celite pad. The filtrate was concentrated to give methyl 2-(4-fluorobenzoyl)-4-hydroxypyrrolo[1,2-f][1,2,4]triazine-6-carboxylate (450 mg, 82%) as a white solid. LC-MS (ESI) m/z 316 (M+H)⁺.

Step D. To a solution of methyl 2-(4-fluorobenzoyl)-4-hydroxypyrrolo[1,2-f][1,2,4]triazine-6-carboxylate (356 mg, 1.12 mmol) in DMF (5 mL) were added PyBrop (631 mg, 1.35 mmol) and triethylamine (0.472 mL, 3.38 mmol). The reaction mixture was stirred at rt for 2 h, then diluted with water. The precipitate was collected by filtration and dried to give methyl 2-(4-fluorobenzoyl)-4-(5-methyl-1H-pyrazol-3-ylamino)pyrrolo[1,2-f][1,2,4]triazine-6-carboxylate (310 mg, 89%). LC-MS (ESI) m/z 395 (M+H)⁺.

Step E: To a solution of methyl 2-(4-fluorobenzoyl)-4-(5-methyl-1H-pyrazol-3-ylamino)pyrrolo[1,2-f][1,2,4]triazine-6-carboxylate (125 mg, 0.316 mmol) in THF (3 mL) at 0° C. was added 1M DIBAL-H in THF (1.58 mL, 1.58 mmol). The reaction mixture was stirred at rt for 1 h, then MeOH was added and the mixture was stirred for an additional 30 min at rt. The mixture was filtered through Celite to resolve the emulsion, and the filtrate was concentrated. The residue was purified by preparative HPLC (Phenomenex phenylhexyl reverse phase column eluted with gradient of solvent A=0.05% HOAc/H₂O and solvent B=0.05% HOAc/CH₃CN) to afford (4-fluorophenyl)(6-(hydroxymethyl)-4-(5-methyl-1H-pyrazol-3-ylamino)pyrrolo[1,2-f][1,2,4]triazin-2-yl) MeOH (6 mg, 1%) as a white solid. ¹H NMR (300 MHz, DMSO-d₆) δ 12.10 (brs, 1H), 10.44 (s, 1H), 7.59-7.51 (m, 3H), 7.18-7.09 (m, 3H), 6.39 (s, 1H), 5.92 (s, 1H), 5.52 (s, 1H), 5.00 (brs, 1H), 4.51 (s, 2H), 2.22 (s, 3H); LC-MS (ESI) m/z 369 (M+H)⁺.

### Example 58

### Preparation of 2-(2-((4-fluorophenyl)(hydroxy)methyl)-4-(5-methyl-1H-pyrazol-3-ylamino)pyrrolo[1,2-f][1,2,4]triazin-6-yl)propan-2-ol

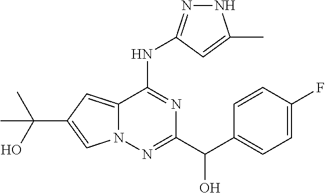

Step A: To a solution of methyl 2-(4-fluorobenzoyl)-4-(5-methyl-1H-pyrazol-3-ylamino)pyrrolo[1,2-f][1,2,4]triazine-6-carboxylate from Example 57, Step D (115 mg, 0.29 mmol) in 2:1 MeOH/THF (4.5 mL) at 0° C. was added NaBH₄ (22.06 mg, 0.583 mmol) and the mixture was stirred at rt for 1 h. The reaction mixture was quenched with aq ammonium chloride and extracted with a mixture of DCM/MeOH. The organic layer was washed with brine, concentrated, and dried to give methyl 2-((4-fluorophenyl)(hydroxy)methyl)-4-(5-methyl-1H-pyrazol-3-ylamino)pyrrolo[1,2-f][1,2,4]triazine-6-carboxylate (40 mg, 35%). LC-MS (ESI) m/z 397 (M+H)⁺.

Step B: To a solution of methyl 2-((4-fluorophenyl)(hydroxy)methyl)-4-(5-methyl-1H-pyrazol-3-ylamino)pyrrolo[1,2-f][1,2,4]triazine-6-carboxylate (45 mg, 0.114 mmol) in THF (3 mL) at 0° C. was added methylmagnesium bromide (3M in ether, 0.189 mL, 0.568 mmol) and the reaction mixture was stirred at rt for 1 h. Additional methyl magnesium bromide solution (0.378 mL, 1.13 mmol) was added and the reaction mixture was stirred at rt for 1 h. The reaction was quenched with aq. ammonium chloride and extracted with a mixture of DCM/MeOH. The organic layer was concentrated and the residue was purified by preparative TLC to afford 2-(2-((4-fluorophenyl)(hydroxy)methyl)-4-(5-methyl-1H-pyrazol-3-ylamino)pyrrolo[1,2-f][1,2,4]triazin-6-yl)propan-2-ol (3.49 mg, 7%) as a white solid. ¹H NMR (300 MHz, DMSO-d₆) δ 12.07 (s, 1H), 10.35 (s, 1H), 7.55-7.50 (m, 3H), 7.17-7.11 (m, 3H), 6.43 (s, 1H), 5.86 (s, 1H), 5.52 (s, 1H), 4.90 (s, 1H), 2.22 (s, 3H), 1.45 (s, 6H); LC-MS (ESI) m/z 397 (M+H)⁺.

### Example 59

### Preparation of 2-(2-(1-(4-fluorophenyl)-1-hydroxyethyl)-4-(5-methyl-1H-pyrazol-3-ylamino)pyrrolo[1,2-f][1,2,4]triazin-6-yl)propan-2-ol

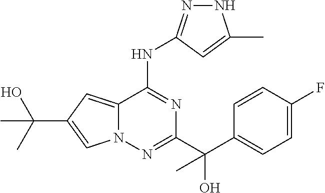

Step A: To a solution of methyl 2-(4-fluorobenzoyl)-4-(5-methyl-1H-pyrazol-3-ylamino)pyrrolo[1,2-f][1,2,4]triazine-6-carboxylate from Example 57, Step D (150 mg, 0.38 mmol) in THF (3 mL) at 0° C. was added methylmagnesium bromide (3M in ether, 0.633 mL, 1.9 mmol) and the mixture was stirred at rt for 30 min. The reaction was quenched with aq. ammonium chloride and extracted with DCM. The organic layer was concentrated and the residue was purified by preparative HPLC (Phenomenex phenylhexyl reverse phase column eluted with gradient of solvent A=0.05% HOAc/H₂O and solvent B=0.05% HOAc/CH₃CN) to afford 2-(2-(1-(4-fluorophenyl)-1-hydroxyethyl)-4-(5-methyl-1H-pyrazol-3-ylamino)pyrrolo[1,2-f][1,2,4]triazin-6-yl)propan-2-ol (25.15 mg, 16%) as a white solid. ¹H NMR (300 MHz, DMSO-d₆) δ 12.18 (s, 1H), 10.82 (s, 1H), 8.14 (s, 1H), 7.68 (s, 1H), 7.58-7.53 (m, 2H), 7.15-7.09 (m, 2H), 6.21 (s, 1H), 5.72 (s, 1H), 3.80 (s, 3H), 3.33 (s, 3H), 3.48 (s, 3H), 1.85 (s, 3H); LC-MS (ESI) m/z 411 (M+H)⁺.

### Example 60

### Preparation of 2-(difluoro(4-fluorophenyl)methyl)-4-(5-methyl-1H-pyrazol-3-ylamino)pyrrolo[1,2-f][1,2,4]triazin-6-ol

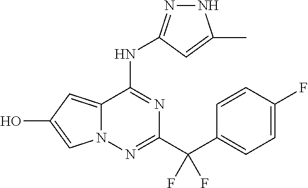

Step A: To BF₃.O(Et)₂ (10 mL) at 0° C. was added H₂O₂ (0.66 mL) and the mixture was stirred at 0° C. for 30 min. The mixture was cooled to −20° C. and 2-(2-(difluoro(4-fluorophenyl)methyl)-4-(5-methyl-1H-pyrazol-3-ylamino)pyrrolo[1,2-f][1,2,4]triazin-6-yl)propan-2-ol (500 mg, 1.20 mmol) was added. After stirring at −20° C. for 1 h, the mixture was cooled to −40° C. and diluted with DCM. Saturated aq Na₂SO₃ (2 mL) was added and the mixture was allowed to warm slowly to rt. After addition of more DCM, the organic layer was separated, dried, and concentrated to give 2-(difluoro(4-fluorophenyl)methyl)-4-(5-methyl-1H-pyrazol-3-ylamino)pyrrolo[1,2-f][1,2,4]triazin-6-ol (280 mg, 62%). ¹H NMR (300 MHz, DMSO-d₆) δ 12.20 (s, 1H), 10.50 (s, 1H), 9.46 (s, 1H), 7.69-7.65 (m, 2H), 7.38-7.34 (m, 3H), 6.79 (s, 1H), 6.19 (s, 1H), 2.20 (s, 3H); LC-MS (ESI)m/z 375 (M+H)⁺.

### Example 61

### Preparation of 2-(difluoro(4-fluorophenyl)methyl)-N-(5-methyl-1H-pyrazol-3-yl)-6-(3-morpholinopropoxy)pyrrolo[1,2-f][1,2,4]triazin-4-amine

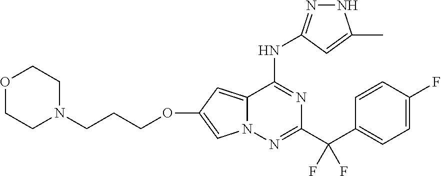

Step A: To a solution of 2-(difluoro(4-fluorophenyl)methyl)-4-(5-methyl-1H-pyrazol-3-ylamino)pyrrolo[1,2-f][1,2,4]triazin-6-ol from Example 60 (350 mg, 0.93 mmol) in DMF (3 mL) were added Cs₂CO₃ (304.6 mg, 0.935 mmol) and 1-bromo-3-chloropropane (191.3 mg, 1.21 mmol) and the mixture was heated at 50° C. for 8 h. Additional 1-bromo-3-chloropropane (73.6 mg, 0.46 mmol) was added, and the mixture was heated for 6 h at 50° C. Ethyl acetate was added, and the solution was washed with water and brine. The organic layer was dried and concentrated, and the residue was purified by silica gel chromatography using DCM/MeOH as eluent to give 6-(3-chloropropoxy)-2-(difluoro(4-fluorophenyl)methyl)-N-(5-methyl-1H-pyrazol-3-yl)pyrrolo[1,2-f][1,2,4]triazin-4-amine (102 mg, 25%). LC-MS (ESI) m/z 451 (M+H)⁺.

Step B: To a solution of 6-(3-chloropropoxy)-2-(difluoro(4-fluorophenyl)methyl)-N-(5-methyl-1H-pyrazol-3-yl)pyrrolo[1,2-f][1,2,4]triazin-4-amine (101 mg, 0.22 mmol) in DMF (3 mL) were added morpholine (58.54 mg, 0.672 mmol), NaI (33.5 mg, 0.224 mmol) and DIPEA (0.117 mL, 0.672 mmol), and the mixture was heated at 80° C. for 18 h. The resulting mixture was purified by preparative HPLC (Phenomenex phenylhexyl reverse phase column eluted with gradient of solvent A=0.05% HOAc/H₂O and solvent B=0.05% HOAc/CH₃CN) to afford 2-(difluoro(4-fluorophenyl)methyl)-N-(5-methyl-1H-pyrazol-3-yl)-6-(3-morpholinopropoxy)pyrrolo[1,2-f][1,2,4]triazin-4-amine (25.75 mg, 23%) as a white solid. ¹H NMR (300 MHz, DMSO-d₆) δ 12.20 (s, 1H), 10.62 (s, 1H), 7.70-7.64 (m, 3H), 7.38-7.33 (m, 2H), 6.95 (s, 1H), 6.22 (s, 1H), 4.01 (t, 2H), 3.58-3.55 (m, 4H), 2.43-2.36 (m, 6H), 2.20 (s, 3H), 1.98 (t, 2H); LC-MS (ESI) m/z 502 (M+H)⁺.

### Example 62

### Preparation of 2-(2-((4-fluoronhenyl)(methoxy)methyl)-4-(5-methyl-1H-pyrazol-3-ylamino)pyrrolo[1,2-f][1,2,4]triazin-6-yl)propan-2-ol

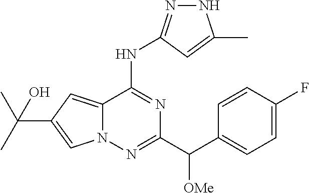

Step A: To a solution of methyl 2-((4-fluorophenyl)(hydroxy)methyl)-4-(5-methyl-1H-pyrazol-3-ylamino)pyrrolo[1,2-f][1,2,4]triazine-6-carboxylate from Example 58, Step A (500 mg, 1.26 mmol) in DCM at 0° C. was added phosphorous tribromide (0.238 mL, 2.52 mmol). The mixture was stirred at rt for 1 h and then 10% NaHCO₃ was added. The mixture was extracted with DCM and the organic layer was washed with brine, dried, and concentrated to give methyl 2-(bromo(4-fluorophenyl)methyl)-4-(5-methyl-1H-pyrazol-3-ylamino)pyrrolo[1,2-f][1,2,4]triazine-6-carboxylate (560 mg, 96%) as a white solid. ¹H NMR (300 MHz, DMSO-d₆) δ 12.20 (s, 1H), 11.08 (s, 1H), 8.18 (s, 1H), 7.87-7.75 (m, 3H), 7.24-7.18 (m, 2H), 6.63 (s, 1H), 6.23 (s, 1H), 3.83 (s, 3H), 2.27 (s, 3H); LC-MS (ESI) m/z, 459 (M+H)⁺, 461(M+2H)⁺.

Step B: A solution of methyl 2-(bromo(4-fluorophenyl)methyl)-4-(5-methyl-1H-pyrazol-3-ylamino)pyrrolo[1,2-f][1,2,4]triazine-6-carboxylate (300 mg, 0.65 mmol) in MeOH was heated in a sealed tube at 90° C. for 1 h. The solvent were evaporated to give methyl 2-((4-fluorophenyl)(methoxy)methyl)-4-(5-methyl-1H-pyrazol-3-ylamino)pyrrolo[1,2-f][1,2,4]triazine-6-carboxylate (266 mg, 100%). ¹H NMR (300 MHz, DMSO-d₆) δ 11.50 (s, 1H), 8.20 (s, 1H), 7.82-7.73 (s, 1H), 7.59-7.54 (m, 2H), 7.23-7.17 (m, 2H), 6.47 (s, 1H), 5.30 (s, 1H), 3.81 (s, 3H), 3.37 (s, 3H), 2.29 (s, 3H); LC-MS (ESI) m/z 411 (M+H)⁺.

Step C: To a solution of methyl 2-((4-fluorophenyl)(methoxy)methyl)-4-(5-methyl-1H-pyrazol-3-ylamino)pyrrolo[1,2-f][1,2,4]triazine-6-carboxylate (150 mg, 0.365 mmol) in THF (3 mL) at 0° C. was added 3M methylmagnesium bromide in ether (0.609 mL, 1.827 mmol). The mixture was stirred at rt for 1 h then additional 3M methyl magnesium bromide in ether (0.609 mL, 1.827 mmol) was added, and the mixture was stirred for an additional 1 h at rt. Aq ammonium chloride was added, and the mixture was extracted with ethyl acetate. The organic layer was dried and concentrated, and the residue was purified by preparative HPLC (Phenomenex phenylhexyl reverse phase column eluted with gradient of solvent A=0.05% HOAc/H₂O and solvent B=0.05% HOAc/CH₃CN) to afford 2-(2-((4-fluorophenyl)(methoxy)methyl)-4-(5-methyl-1H-pyrazol-3-ylamino)pyrrolo[1,2-f][1,2,4]triazin-6-yl)propan-2-ol (24.66 mg, 17%) as a white solid. ¹H NMR (300 MHz, DMSO-d₆) δ 12.10 (s, 1H), 10.41 (s, 1H), 7.95-7.51 (m, 3H), 7.19-7.14 (s, 3H), 6.45 (s, 1H), 5.17 (s, 1H), 4.91 (s, 1H), 3.33 (s, 3H), 2.25 (s, 3H), 1.45 (s, 6H); LC-MS (ESI) m/z 411 (M+H)⁺.

### Example 63

### Preparation of methyl (4-fluorophenyl)(6-(2-hydroxypropan-2-yl)-4-(5-methyl-1H-pyrazol-3-ylamino)pyrrolo[1,2-f][1,2,4]triazin-2-yl)methylcarbamate

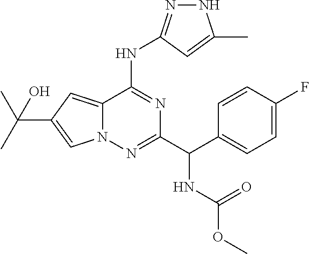

Step A: To a solution of methyl 2-(bromo(4-fluorophenyl)methyl)-4-(5-methyl-1H-pyrazol-3-ylamino)pyrrolo[1,2-f][1,2,4]triazine-6-carboxylate from Example 62, Step A (980 mg, 2.133 mmol) in DMF (10 mL) was added NaN₃ (207.9 mg, 3.19 mmol) and the mixture was stirred at rt for 2 h. The mixture was diluted with ethyl acetate, washed with water and brine, and concentrated to give methyl 2-(azido(4-fluorophenyl)methyl)-4-(5-methyl-1H-pyrazol-3-ylamino)pyrrolo[1,2-f][1,2,4]triazine-6-carboxylate (870 mg, 96%). LC-MS (ESI) m/z 422 (M+H)⁺.

Step B: To a solution of methyl 2-(azido(4-fluorophenyl)methyl)-4-(5-methyl-1H-pyrazol-3-ylamino)pyrrolo[1,2-f][1,2,4]triazine-6-carboxylate (900 mg, 2.13 mmol) in 4:3 THF/MeOH (35 mL) was added 10% Pd/C (180 mg) and the mixture was stirred under an atmosphere of H₂ for 3 h. The mixture was filtered and the filtrate was concentrated to dryness to give methyl 2-(amino(4-fluorophenyl)methyl)-4-(5-methyl-1H-pyrazol-3-ylamino)pyrrolo[1,2-f][1,2,4]triazine-6-carboxylate (703 mg, 84%) as a solid. ¹H NMR (300 MHz, DMSO-d₆) δ 12.28 (s, 1H), 10.89 (s, 1H), 8.20 (s, 1H), 7.75 (s, 1H), 7.60-7.56 (m, 2H), 7.23-7.17 (m, 2H), 6.48 (s, 1H), 4.96 (s, 1H), 3.87 (s, 3H), 2.31 (s, 3H); LC-MS (ESI) m/z 396 (M+H)⁺.

Step C: To a solution of methyl 2-(amino(4-fluorophenyl)methyl)-4-(5-methyl-1H-pyrazol-3-ylamino)pyrrolo[1,2-f][1,2,4]triazine-6-carboxylate (300 mg, 0.758 mmol) and DIPEA (0.198 mL, 1.137 mmol) in THF (15 mL) at 0° C. was added methyl chloroformate (46.6 uL, 0.604 mmol), and the mixture was stirred at 0° C. for 2 h. The mixture was diluted with ethyl acetate and washed with brine. The organic layer was concentrated and the residue was purified by silica gel chromatography using DCM/MeOH as eluent, to give methyl 2-((4-fluorophenyl)(methoxycarbonylamino)methyl)-4-(5-methyl-1H-pyrazol-3-ylamino)pyrrolo[1,2-f][1,2,4]triazine-6-carboxylate (125 mg, 37%) as a solid. LC-MS (ESI) m/z 454 (M+H)⁺.

Step D: To a solution of methyl 2-((4-fluorophenyl)(methoxycarbonylamino)methyl)-4-(5-methyl-1H-pyrazol-3-ylamino)pyrrolo[1,2-f][1,2,4]triazine-6-carboxylate (75 mg, 0.165 mmol) in THF (15 mL) at 0° C. was added 3M methylmagnesium bromide in ether (0.165 mL, 0.495 mmol). The reaction mixture was stirred at rt for 15 min, after which the mixture was cooled to 0° C. and additional 3M methylmagnesium bromide in ether (0.33 ml, 0.99 mmol) was added. The mixture was stirred at rt for 1 h, quenched with aq ammonium chloride, and extracted with ethyl acetate. The organic layer was separated and concentrated, and the residue was purified by preparative HPLC (Phenomenex phenylhexyl reverse phase column eluted with gradient of solvent A=0.05% HOAc/H₂O and solvent B=0.05% HOAc/CH₃CN) to give methyl (4-fluorophenyl)(6-(2-hydroxypropan-2-yl)-4-(5-methyl-1H-pyrazol-3-ylamino)pyrrolo[1,2-f][1,2,4]triazin-2-yl)methylcarbamate (6.5 mg, 9%) as a white solid. ¹H NMR (300 MHz, DMSO-d₆) δ 12.21 (s, 1H), 10.88 (s, 1H), 8.11 (m, 2H), 7.69 (s, 1H), 7.54-7.43 (m, 2H), 7.19-7.13 (m, 2H), 6.38 (s, 1H), 5.72-5.69 (m 1H), 3.79 (s, 3H), 3.57 (s, 3H), 3.33 (s, 3H), 1.86 (s, 3H); LC-MS (ESI) m/z 454 (M+H)⁺.

### Example 64

### Preparation of 2-(difluoro(4-fluorophenyl)methyl)-N-(5-methyl-1H-pyrazol-3-yl)-6-((2-(piperidin-1-yl)ethoxy)methyl)pyrrolo[1,2-f][1,2,4]triazin-4-amine acetate salt

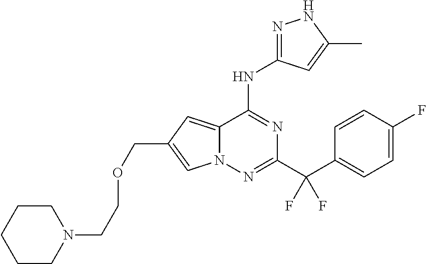

To a solution of 2-(piperidin-1-yl)ethanol (60 mg, 4.66 mmol) in anhydrous DMF (4 mL) was added NaH (60% in mineral oil). After stirring for 10 min, 6-(bromomethyl)-2-(difluoro(4-fluorophenyl)methyl)-N-(5-methyl-1H-pyrazol-3-yl)pyrrolo[1,2-f][1,2,4]triazin-4-amine from Example 52, Step A (350 mg, 0.77 mmol) was added, and the mixture was stirred at rt overnight. The solvent was removed under reduced pressure and the residue was suspended in water. The solid was collected by filtration, then purified on reverse phase HPLC to afford 2-(difluoro(4-fluorophenyl)methyl)-N-(5-methyl-1H-pyrazol-3-yl)-6-((2-(piperidin-1-yl)ethoxy)methyl)pyrrolo[1,2-f][1,2,4]triazin-4-amine as a white solid (25 mg, 5%). ¹H NMR (DMSO-d₆): δ 11.90 (bs, 1H), 7.79 (s, 1H), 7.69 (m, 2H), 7.34 (t, 2H), 7.07 (bs, 1H), 5.97 (s, 1H), 4.65 (s, 2H), 3.96 (s, 2H), 3.4-3.2 (m, 6H), 2.13 (s, 3H), 1.9-1.7 (m, 4H), 1.77 (s, 3H), 1.57 (m, 2H); LC-MS (ESI) m/z 500 (M+H)⁺.

The following compounds were prepared using a similar procedure, replacing the 2-(piperidin-1-yl)ethanol with the appropriately substituted alcohol:

| Example | | | |
|---|---|---|---|
| Number | Structure | Name | Analytical Data |
| 64a | 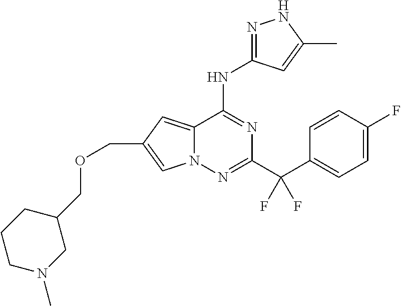 | 2-(difluoro(4- fluorophenyl)methyl)- N-(5-methyl-1H- pyrazol-3-yl)-6-(((1- methylpiperidin-3- yl)methoxy)methyl) pyrrolo[1,2- f][1,2,4]triazin-4- amine | LC-MS (ESI) m/z 502 (M + H)⁺ |
| 64b | 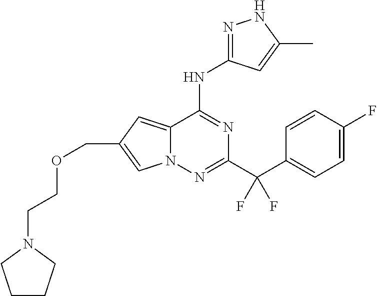 | 2-(difluoro(4- fluorophenyl)methyl)- N-(5-methyl-1H- pyrazol-3-yl)-6-((2- (pyrrolidin-1- yl)ethoxy)methyl) pyrrolo[1,2- f][1,2,4]triazin-4- amine | ¹HNMR (DMSO- d6): δ 8.01 (d, 1H), 7.70 (m, 2H), 7.28- 7.19 (m, 3H), 6.38 (s, 1H), 4.72 (s, 2H), 4.11 (s, 2H), 3.7-3.6 (m, 4H), 3.41 (m, 2H), 2.30 (s, 3H), 2.25 (m, 4H), 1.91 (s, 3H). LC-MS (ESI) m/z 486 (M + H)⁺. |
| 64c | 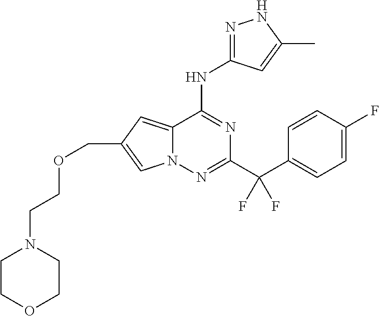 | 2-(difluoro(4- fluorophenyl)methyl)- N-(5-methyl-1H- pyrazol-3-yl)-6-((2- morpholinoethoxy) methyl)pyrrolo[1,2- f][1,2,4]triazin-4- amine | 1HNMR (dmso- d6); δ7.85 (s, 1H), 7.69 (m, 2H), 7.36 (t, 2H), 7.25 (bs, 1H), 6.23 (s, 1H), 4.82 (s, 2H), 3.9- 4.1 (m, 6H), 3.5-3.4 (m, 6H), 2.14 (s, 3H), 1.80 (s, 3H). LC-MS (ESI) m/z 502 (M + H)⁺ |
| 64d | 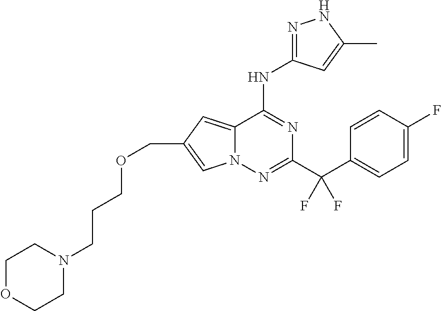 | 2-(difluoro(4- fluorophenyl)methyl)- N-(5-methyl-1H- pyrazol-3-yl)-6-((3- morpholinopropoxy) methyl)pyrrolo[1,2- f][1,2,4]triazin-4- amine | LC-MS (ESI) m/z 516 (M + H)⁺. |

### Example 65

### Preparation of (4-fluorophenyl)(4-(5-methyl-1H-pyrazol-3-ylamino)pyrrolo[1,2-f][1,2,4]triazin-2-yl)methanone oxime

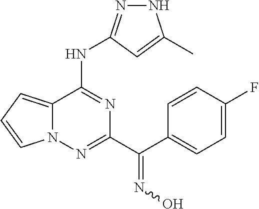

To a solution of (4-fluorophenyl)(4-(5-methyl-1H-pyrazol-3-ylamino)pyrrolo[1,2-f][1,2,4]triazin-2-yl)methanone from Example 1 (30 mg, 0.089 mmol) in EtOH (1 mL) was added a 50% solution of hydroxylamine in water (0.026 mL, 0.356 mmol). The reaction mixture was heated at 70° C., overnight, then the mixture was purified on reverse phase HPLC to yield (4-fluorophenyl)(4-(5-methyl-1H-pyrazol-3-ylamino)pyrrolo[1,2-f][1,2,4]triazin-2-yl)methanone oxime as a white solid (14 mg, 44%). ¹H NMR (DMSO-d₆): δ 11.78 (bs, 1H), 1.1.58 (s, 1H), 10.64 (bs, 1H), 7.75 (s, 1H), 7.49 (m, 2H), 7.3-7.2 (m, 3H), 6.70 (m, 1H), 6.22 (s, 1H), 2.17 (s, 3H); LC-MS (ESI) m/z 352 (M+H)⁺.

### Example 66

### Preparation of 1-(4-fluorophenyl)-1-(4-(5-methyl-1H-pyrazol-3-ylamino)pyrrolo[1,2-f][1,2,4]triazin-2-yl)ethanol

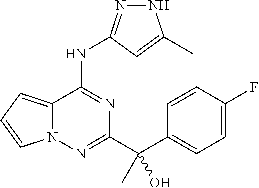

To a solution of (4-fluorophenyl)(4-(5-methyl-1H-pyrazol-3-ylamino)pyrrolo[1,2-f][1,2,4]triazin-2-yl)methanone from Example 1 (100 mg, 0.297 mmol) in THF (3 mL) at room temperature was added a 3M solution of methylmagnesium bromide in THF (0.396 mL, 1.19 mmol). The reaction mixture was stirred overnight, then the reaction was quenched by addition of 0.5 N HCl and extracting with EtOAc (2×10 mL). The combined organic layers were washed with brine and dried over MgSO₄. After evaporation of the solvent, the crude product was purified on HPLC to yield 1-(4-fluorophenyl)-1-(4-(5-methyl-1H-pyrazol-3-ylamino)pyrrolo[1,2-f][1,2,4]triazin-2-yl)ethanol as a white solid (49 mg, 47%). 1H NMR (DMSO-d₆): δ 12.12 (s, 1H), 10.50 (s, 1H), 7.80 (s, 1H), 7.70 (s, 2H), 7.12 (m, 3H), 6.66 (s, 1H), 6.24 (s, 1H), 5.63 (s, 1H), 2.20 (s, 3H), 1.85 (s, 3H); LC-MS (ESI) m/z 353 (M+H)⁺.

The following compounds were prepared in a similar manner, replacing the methylmagnesium bromide with the appropriate organometallic reagent or by replacing the (4-fluorophenyl)(4-(5-methyl-1H-pyrazol-3-ylamino)pyrrolo[1,2-f][1,2,4]triazin-2-yl)methanone with (4-(1H-pyrazol-3-ylamino)-6-fluoropyrrolo[1,2-f][1,2,4]triazin-2-yl)(4-fluorophenyl)methanone from Example 94.

| Example | | | |
|---|---|---|---|
| Number | Structure | Name | Analytical Data |
| 66a | 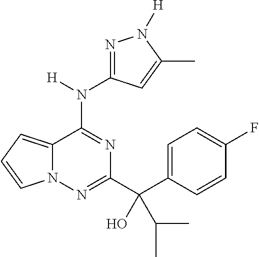 | 1-(4-Fluoro-phenyl)-2- methyl-1-[4-(5-methyl-1H- pyrazol-3-ylamino)- pyrrolo[2,1-f][1,2,4]triazin-2- yl]-propan-1-ol | LC-MS (ESI) m/z 381 (M + H)⁺; 14.0 min |
| 66b | 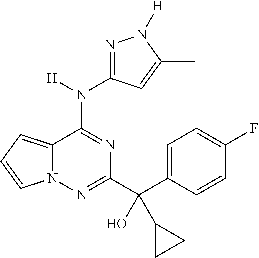 | Cyclopropyl-(4-fluoro- phenyl)-[4-(5-methyl-1H- pyrazol-3-ylamino)- pyrrolo[2,1-f][1,2,4]triazin-2- yl]-methanol | LC-MS (ESI) m/z 379 (M + H)⁺; 12.8 min |
| 66c | 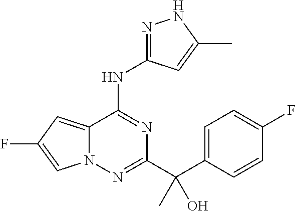 | 1-[6-Fluoro-4-(5-methyl-1H- pyrazol-3-ylamino)- pyrrolo[2,1-f][1,2,4]triazin-2- yl]-1-(4-fluoro-phenyl)- ethanol | LC-MS (ESI) m/z 371 (M + H)⁺; 12.5 min |

### Example 67

### Preparation of 3-(4-(1H-pyrazol-3-ylamino)pyrrolo[1,2-f][1,2,4]triazin-2-yl)-3-(4-fluorophenyl)propan-1-ol

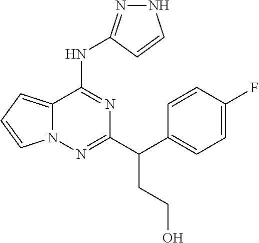

Step A: To a solution of ethyl 3-(4-(1H-pyrazol-3-ylamino)pyrrolo[1,2-f][1,2,4]triazin-2-yl)-3-(4-fluorophenyl)acrylate prepared as described in Example 40 (3.92 g, 0.01 mol) in MeOH (50 mL) was added 10% of Pd/C (800 mg). The mixture was stirred under 1 atm. of H₂ for 2 days. The catalyst was separated by filtration through a short plug of Celite and the solvent was removed under vacuum to yield ethyl 3-(4-(1H-pyrazol-3-ylamino)pyrrolo[1,2-f][1,2,4]triazin-2-yl)-3-(4-fluorophenyl)propanoate (3.5 g, 89%). LC-MS (ESI) m/z 395 (M+H)⁺.

Step B: To a suspension of LAH in anhydrous THF at 0° C. was added a solution of ethyl 3-(4-(1H-pyrazol-3-ylamino)pyrrolo[1,2-f][1,2,4]triazin-2-yl)-3-(4-fluorophenyl)propanoate (3.5 g, 8.9 mmol) in THF. After stirring overnight at rt, a solution of 2N of NaOH was added at ° C. The resulting mixture was stirred 15 min at After adding MgSO₄, the mixture was filtered and the solvent evaporated to dryness. The crude product was purified on silica gel column to yield 3-(4-(1H-pyrazol-3-ylamino)pyrrolo[1,2-f][1,2,4]triazin-2-yl)-3-(4-fluorophenyl)propan-1-ol as an off-white solid (1.5 g, 48%). ¹H NMR (DMSO-d₆): δ 12.50 (s, 1H), 10.58 (s, 1H), 7.73 (s, 1H), 7.66 (s, 1H), 7.45 (m, 2H), 7.20 (s, 1H), 7.11 (2H, t), 6.86 (s, 1H), 6.63 (d, 1H), 4.51 (m, 1H), 4.08 (m 1H), 2.40 (m, 2H), 2.08 (m, 2H); LC-MS (ESI) m/z 353 (M+H)⁺.

### Example 68

### Preparation of 2-(1-(4-fluorophenyl)-3-morpholinopropyl)-N-(1H-pyrazol-3-yl)pyrrolo[1,2-f][1,2,4]triazin-4-amine

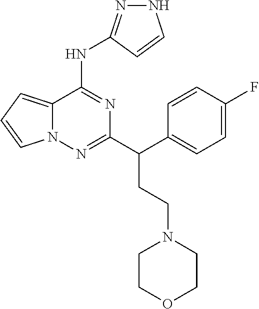

Step A: To a solution of 3-(4-(1H-pyrazol-3-ylamino)pyrrolo[1,2-f][1,2,4]triazin-2-yl)-3-(4-fluorophenyl)propan-1-ol from Example 67 (176 mg, 0.5 mmol) in DCM (5 mL) was added Dess-Martin periodinane (340 mg, 0.8 mmol). The mixture was stirred at rt for 3 h, then diluted with DCM (10 mL) then water was added. The aqueous later was separated and extracted with DCM, and the combined organic extracts were dried over MgSO₄ and concentrated to give crude 3-(4-(1H-pyrazol-3-ylamino)pyrrolo[1,2-f][1,2,4]triazin-2-yl)-3-(4-fluorophenyl)propanal (182 mg, 100%). TLC(Rf: 0.5, DCM/MeOH, 10/0.5).

Step B: To a solution of 3-(4-(1H-pyrazol-3-ylamino)pyrrolo[1,2-f][1,2,4]triazin-2-yl)-3-(4-fluorophenyl)propanal (175 mg, 0.50 mmol), morpholine (87 μL, 1 mmol) and catalytic amount of acetic acid in DCM (5 mL) was added NaBH₃CN (189 mg, 3 mmol). The mixture was stirred at rt overnight, then washed with saturated aq. NH₄Cl and brine, then dried over MgSO₄. The solvent was evaporated under vacuum and the crude product was purified on silica gel to yield 2-(1-(4-fluorophenyl)-3-morpholinopropyl)-N-(1H-pyrazol-3-yl)pyrrolo[1,2-f][1,2,4]triazin-4-amine as a white solid (34 mg, 16%). ¹H NMR (DMSO-d₆): δ 12.52 (s, 1H), 10.59 (s, 1H), 7.74 (s, 1H), 7.67 (s, 1H), 7.47 (t, 2H), 7.20 (s, 1H), 7.11 (t, 2H), 6.86 (s, 1H), 6.63 (s, 1H), 3.99 (m, 1H), 3.50 (s, 4H), 2.44 (m, 1H), 2.3-2.2 (m, 6H), 2.03 (m, 1H); LC-MS (ESI) m/z 422 (M+H)⁺.

The following compounds were prepared using a similar procedure, replacing the morpholine with an appropriate amine:

| Example | | | Analytical |
|---|---|---|---|
| Number | Structure | Name | Data |
| 68a | 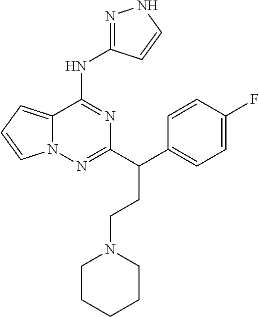 | {2-[1-(4-Fluoro-phenyl)-3-piperidin-1- yl-propyl]-pyrrolo[2,1-f][1,2,4]triazin- 4-yl}-(1H-pyrazol-3-yl)-amine | LC-MS (ESI) m/z 420 (M + H)⁺; 8.3 min |
| 68b | 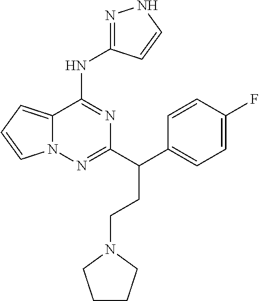 | {2-[1-(4-Fluoro-phenyl)-3-pyrrolidin- 1-yl-propyl]-pyrrolo[2,1- f][1,2,4]triazin-4-yl}-(1H-pyrazol-3- yl)-amine | LC-MS (ESI) m/z 406 (M + H)⁺; 8.1 min |
| 68c | 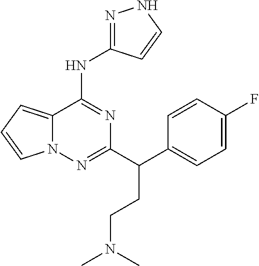 | {2-[3-Dimethylamino-1-(4-fluoro- phenyl)-propyl]-pyrrolo[2,1- f][1,2,4]triazin-4-yl}-(1H-pyrazol-3- yl)-amine | LC-MS (ESI) m/z 380 (M + H)⁺; 7.9 min |

### Example 69

### Preparation of 2-(amino(4-fluorophenyl)methyl)-N-(5-methyl-1H-pyrazol-3-yl)pyrrolo[1,2-f][1,2,4]triazin-4-amine

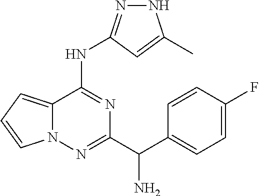

Step A: To (4-fluorophenyl)(4-(5-methyl-1H-pyrazol-3-ylamino)pyrrolo[1,2-f][1,2,4]triazin-2-yl)methanol prepared according to Example 2 (206 mg, 0.61 mmol) in DCM (5.0 mL) was added phosphorous tribromide (0.17 mL, 1.82 mmol). The solution was heated to 60° C. for 15 min, cooled to rt, then diluted with DCM and extracted with a sodium bicarbonate solution. The DCM layer was dried with sodium sulfate and then evaporated to give crude 2-(bromo(4-fluorophenyl)methyl)-N-(5-methyl-1H-pyrazol-3-yl)pyrrolo[1,2-f][1,2,4]triazin-4-amine (249 mg, quantitative) which was used without further purification. LC-MS (ESI) m/z 401, 403 (M+H)⁺.

Step B: To a solution of sodium azide (81 mg, 1.25 mmol) in DMF (2 mL) was added crude 2-(bromo(4-fluorophenyl)methyl)-N-(5-methyl-1H-pyrazol-3-yl)pyrrolo[1,2-f][1,2,4]triazin-4-amine (100 mg, 0.25 mmol), and the solution was stirred at rt for 30 min. Water was then added and the solution was extracted with ethyl acetate, followed by evaporation of the organic layer. The crude material was dissolved in EtOH (10 mL) and ammonium sulfide (0.5 mL, minimum 20% solution) was added. The solution was stirred at room temperature for 2 h, and then an additional 0.5 mL of the ammonium sulfide solution was added. The solution was stirred at room temperature overnight, followed by heating to 60° C. for 2 h, addition of another 0.5 mL of ammonium sulfide solution, and further heating at 45° C. overnight. The crude mixture was purified by preparative HPLC (Varian diphenyl reverse phase column, eluted with gradient of solvent B=0.05% HOAC/CH₃CN and solvent A=0.05% HOAc/H₂O) to afford 2-(amino(4-fluorophenyl)methyl)-N-(5-methyl-1H-pyrazol-3-yl)pyrrolo[1,2-f][1,2,4]triazin-4-amine as its acetate salt (26 mg, 26%). ¹H NMR (300 MHz, DMSO-d₆) δ 1.91 (s, 3H), 2.24 (s, 3H), 5.00 (s, 1H), 6.39 (s, 1H), 6.66 (m, 1H), 7.15-7.25 (m, 3H), 7.54 (m, 2H), 7.69 (s, 1H), 10.68 (s, 1H); LC-MS (ESI) m/z 338 (M+H)⁺.

### Example 70

### Preparation of N-((4-fluorophenyl)(4-(5-methyl-1H-pyrazol-3-ylamino)pyrrolo[1,2-f][1,2,4]triazin-2-yl)methyl)-2-morpholinoacetamide

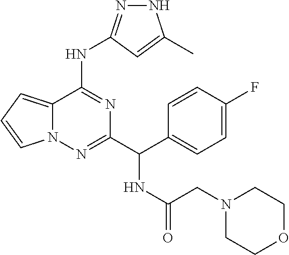

Step A: To 2-(bromo(4-fluorophenyl)methyl)-N-(5-methyl-1H-pyrazol-3-yl)pyrrolo[1,2-f][1,2,4]triazin-4-amine from Example 69 Step A (410 mg, 1.02 mmol) in DMF (20 mL) was added sodium azide (80 mg, 1.22 mmol). The solution was heated at 90° C. for 30 min, cooled to rt, then diluted with water and extracted with ethyl acetate. The ethyl acetate layer was evaporated to give crude 2-(azido(4-fluorophenyl)methyl)-N-(5-methyl-1H-pyrazol-3-yl)pyrrolo[1,2-f][1,2,4]triazin-4-amine (320 mg, 86%) which was used in the next step without further purification. LC-MS (ESI) m/z 364 (M+H)⁺.

Step B: To 2-(azido(4-fluorophenyl)methyl)-N-(5-methyl-1H-pyrazol-3-yl)pyrrolo[1,2-f][1,2,4]triazin-4-amine (230 mg, 0.63 mmol) in THF (1.89 mL) at 0° C. was added lithium aluminum hydride (1M in THF, 0.66 mL, 0.66 mmol). After 10 min, additional THF (1 mL) was added, and the solution was stirred at 0° C. for 1 h. 1 N NaOH (0.9 mL) was added and the solution was brought to room temperature. Ethyl acetate (30 mL) was added and the mixture was dried over sodium sulfate. The mixture was filtered through Celite washing with ethyl acetate, and the filtrate was concentrated to afford crude 2-(amino(4-fluorophenyl)methyl)-N-(5-methyl-1H-pyrazol-3-yl)pyrrolo[1,2-f][1,2,4]triazin-4-amine (90 mg, 41%), which was used without further purification. LC-MS (ESI) m/z 338 (M+H)⁺.

Step C: To 2-(amino(4-fluorophenyl)methyl)-N-(5-methyl-1H-pyrazol-3-yl)pyrrolo[1,2-f][1,2,4]triazin-4-amine (90 mg, 0.27 mmol) in THF (2.5 mL) was added 2-chloroacetyl chloride (0.042 mL, 0.54 mmol), followed by diisopropylethylamine (0.057 mL, 0.32 mmol) and the mixture was stirred at rt for 1 h. Then 1 N NaOH (2.5 mL) and ethyl acetate (2.5 mL) were added and the mixture was stirred for 20 min. The organic layer was separated and washed with 10% NaOH, then concentrated to give 180 mg of a crude mixture containing 2-chloro-N-((4-fluorophenyl)(4-(5-methyl-1H-pyrazol-3-ylamino)pyrrolo[1,2-f][1,2,4]triazin-2-yl)methyl)acetamide, which was used without further purification. LC-MS (ESI) m/z 414 (M+H)⁺.

Step D: To the crude mixture containing 2-chloro-N-((4-fluorophenyl)(4-(5-methyl-1H-pyrazol-3-ylamino)pyrrolo[1,2-f][1,2,4]triazin-2-yl)methyl)acetamide (180 mg) was added DMF (3.5 mL), followed by potassium iodide (85 mg, 0.43 mmol) and morpholine (1.5 mL), and the solution was heated to 70° C. overnight. The mixture was purified directly by preparative HPLC (Varian diphenyl reverse phase column, eluted with gradient of solvent B=0.05% HOAC/CH₃CN and solvent A=0.05% HOAc/H₂O) to afford N-((4-fluorophenyl)(4-(5-methyl-1H-pyrazol-3-ylamino)pyrrolo[1,2-f][1,2,4]triazin-2-yl)methyl)-2-morpholinoacetamide (52 mg, 26% over 2 steps). ¹H NMR (300 MHz, DMSO-d₆) δ 2.26 (s, 3H), 2.46 (m, 4H), 2.96 (d, 1H), 3.12 (d, 1H), 3.55 (t, 4H), 5.86 (d, 1H), 6.46 (s, 1H), 6.68 (t, 1H), 7.15-7.25 (m, 3H), 7.47 (m, 2H), 7.70 (s, 1H), 8.65 (d, 1H), 10.66 (s, 1H), 12.24 (s, 1H); LC-MS (ESI) m/z 465 (M+H)⁺.

The following compounds were prepared using a similar procedure, replacing the morpholine in Step D with an appropriately substituted amine:

| Example | | | Analytical |
|---|---|---|---|
| Number | Structure | Name | Data |
| 70a | 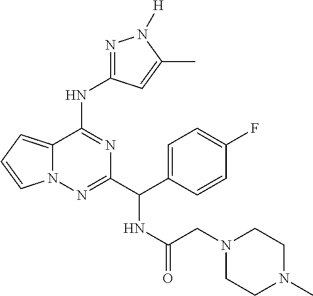 | N-{(4-Fluoro- phenyl)-[4-(5-methyl- 1H-pyrazol-3- ylamino)-pyrrolo[2,1- f][1,2,4]triazin-2-yl]- methyl}-2-(4-methyl- piperazin-1-yl)- acetamide | LC-MS (ESI) m/z (M + H)⁺; 8.11 min |
| 70b | 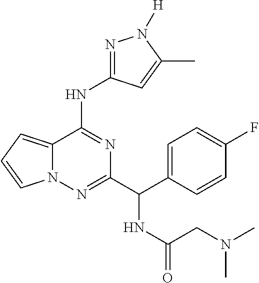 | 2-Dimethylamino-N- {(4-fluoro-phenyl)- [4-(5-methyl-1H- pyrazol-3-ylamino)- pyrrolo[2,1- f][1,2,4]triazin-2-yl]- methyl}-acetamide | LC-MS (ESI) m/z 423 (M + H)⁺; 8.06 min |
| 70c | 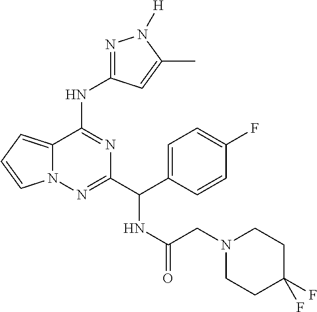 | 2-(Difluoro- piperidin-1-yl)-N- {(4-fluoro-phenyl)- [4-(5-methyl-1H- pyrazol-3-ylamino)- pyrrolo[2,1- f][1,2,4]triazin-2-yl]- methyl}-acetamide | LC-MS (ESI) m/z 499 (M + H)⁺; 11.67 min |
| 70d | 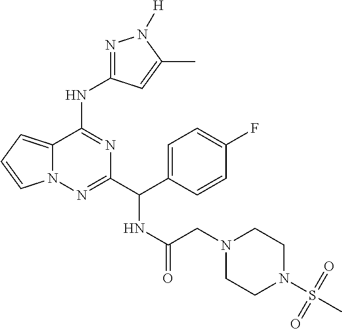 | N-{(4-Fluoro- phenyl)-[4-(5-methyl- 1H-pyrazol-3- ylamino)-pyrrolo[2,1- f][1,2,4]triazin-2-yl]- methyl}-2-(4- methanesulfonyl- piperazin-1-yl)- acetamide | LC-MS (ESI) m/z 542 (M + H)⁺; 10.68 min |
| 70e | 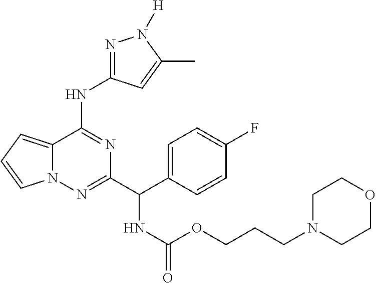 | {(4-Fluoro-phenyl)- [4-(5-methyl-1H- pyrazol-3-ylamino)- pyrrolo[2,1- f][1,2,4]triazin-2-yl]- methyl}-carbamic acid 3-morpholin-4- yl-propyl ester | LC-MS (ESI) m/z 509 (M + H)⁺; 8.16 min |
| 70f | 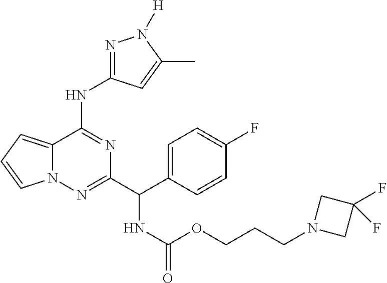 | {(4-Fluoro-phenyl)- [4-(5-methyl-1H- pyrazol-3-ylamino)- pyrrolo[2,1- f][1,2,4]triazin-2-yl]- methyl}-carbamic acid 3-(3,3-difluoro- azetidin-1-yl)-propyl ester | LC-MS (ESI) m/z 515 (M + H)⁺; 9.55 min |
| 70g | 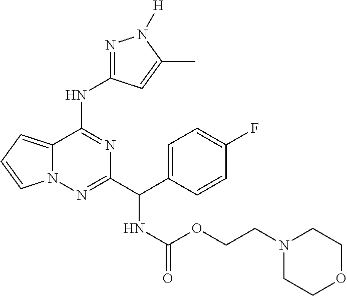 | {(4-Fluoro-phenyl)- [4-(5-methyl-1H- pyrazol-3-ylamino)- pyrrolo[2,1- f][1,2,4]triazin-2-yl]- methyl}-carbamic acid 2-morpholin-4- yl-ethyl ester | LC-MS (ESI) m/z 495 (M + H)⁺; 8.20 min |

### Example 71

### Preparation of N((4-fluorophenyl)(4-(5-methyl-1H-pyrazol-3-ylamino)pyrrolo[1,2-f][1,2,4]triazin-2-yl)methyl)cyclopropanecarboxamide

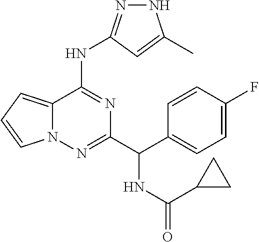

To crude 2-(amino(4-fluorophenyl)methyl)-N-(5-methyl-1H-pyrazol-3-yl)pyrrolo[1,2-f][1,2,4]triazin-4-amine (140 mg, 0.41 mmol) in DCM (5 mL) were added 0-(7-Azabenzotriazol-1-yl)-N,N,N′,N′-tetramethyluronium hexafluorophosphate (157 mg, 0.41 mmol), cyclopropanecarboxylic acid (0.032 mL, 0.41 mmol), and diisopropylethylamine (0.14 mL, 0.82 mmol). The solution was stirred for 30 min, then the solution was extracted with water, and the separated aqueous phase was back-extracted with DCM. The combined organic layers were concentrated and then EtOH (5 mL) was added, followed by 25% sodium methoxide in MeOH (1 mL). The solution was stirred for 5 min and then evaporated. The crude mixture was partitioned between DCM and water, and the DCM layer was concentrated. The crude mixture was purified by preparative HPLC (Varian diphenyl reverse phase column, eluted with gradient of solvent B=0.05% HOAC/CH₃CN and solvent A=0.05% HOAc/H₂O) to afford N4(4-fluorophenyl)(4-(5-methyl-1H-pyrazol-3-ylamino)pyrrolo[1,2-f][1,2,4]triazin-2-yl)methyl)cyclopropanecarboxamide (28 mg, 17%). ¹H NMR (300 MHz, DMSO-d₆) δ 0.69 (s, 4H), 1.87 (m, 1H), 2.22 (s, 3H), 5.98 (d, 1H), 6.44 (s, 1H), 6.66 (s, 1H), 7.15-7.25 (m, 3H), 7.49 (m, 2H), 7.69 (s, 1H), 8.97 (d, 1H), 10.56 (s, 1H), 12.13 (s, 1H); LC-MS (ESI) m/z 406 (M+H)⁺.

### Example 72

### Preparation of 2-((4-fluorophenyl)(2-morpholinoethoxy)methyl)-N-(5-methyl-1H-pyrazol-3-yl)pyrrolo[1,2-f][1,2,4]triazin-4-amine

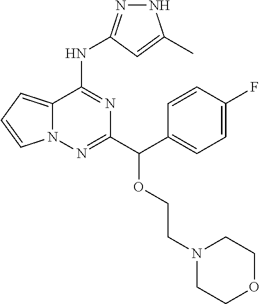

Step A: To (4-fluorophenyl)(4-(5-methyl-1H-pyrazol-3-ylamino)pyrrolo[1,2-f][1,2,4]triazin-2-yl)methanol (300 mg, 0.88 mmol), in 2-chloroethanol (2 mL) was added methanesulfonic acid (0.1 mL). The solution was heated to 120° C. under microwave irradation for 20 min., followed by additional irradiation at 140° C. for 20 min. The crude solution was purified by silica gel chromatography (DCM/methanol 0-10%) to afford 2-((2-chloroethoxy)(4-fluorophenyl)methyl)-N-(5-methyl-1H-pyrazol-3-yl)pyrrolo[1,2-f][1,2,4]triazin-4-amine (210 mg, 59%). LC-MS (ESI) m/z 401 (M+H)⁺.

Step B: To 24(2-chloroethoxy)(4-fluorophenyl)methyl)-N-(5-methyl-1H-pyrazol-3-yl)pyrrolo[1,2-f][1,2,4]triazin-4-amine (105 mg, 0.26 mmol) in DMF (2.5 mL) were added morpholine (0.068 mL, 0.78 mmol), diisopropylethylamine (0.137 mL, 0.78 mmol) and potassium iodide (43 mg, 0.26 mmol) and the solution was heated to 60° C. for 36 h. An additional 0.07 mL of morpholine was then added and the solution was heated to 70° C. for 6 h and then 60° C. for an additional 12 h. The crude mixture was then purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(2-morpholinoethoxy)methyl)-N-(5-methyl-1H-pyrazol-3-yl)pyrrolo[1,2-f][1,2,4]triazin-4-amine (23 mg, 20%). ¹H NMR (300 MHz, DMSO-d₆) δ 2.26 (s, 3H), 2.3-2.6 (m, 6H), 3.48 (t, 4H), 3.61 (t, 2H), 5.32 (s, 1H), 6.53 (s, 1H), 6.67 (t, 1H), 7.1-7.3 (m, 3H), 7.57 (t, 1H), 7.71 (s, 1H), 10.56 (s, 1H), 12.08 (s, 1H); LC-MS (ESI) m/z 452 (M+H)⁺.

The following compounds were prepared using a similar procedure, replacing the morpholine in Step B with an appropriate cyclic amine:

| Example | | | Analytical |
|---|---|---|---|
| Number | Structure | Name | Data |
| 72a | 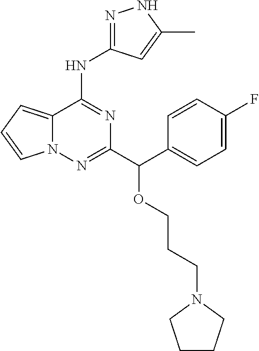 | {2-[(4-Fluoro-phenyl)- (3-pyrrolidin-1-yl- propoxy)-methyl]- pyrrolo[2,1-f][1,2,4]triazin- 4-yl}-(5-methyl-1H- pyrazol-3-yl)-amine | LC-MS (ESI) m/z 450 (M + H)⁺; 8.43 min |
| 72b | 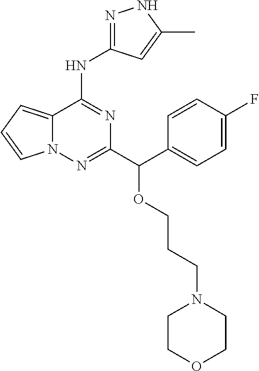 | {2-[(4-Fluoro-phenyl)- (3-morpholin-4-yl- propoxy)-methyl]- pyrrolo[2,1-f][1,2,4]triazin- 4-yl}-(5-methyl-1H- pyrazol-3-yl)-amine | LC-MS (ESI) m/z 466 (M + H)⁺; 8.32 min |
| 72c | 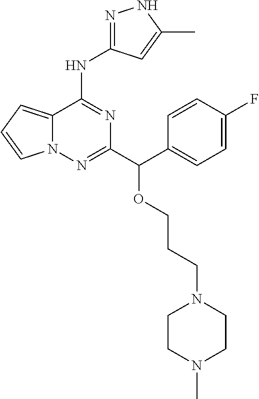 | (2-{(4-Fluoro-phenyl)- [3-(4-methyl-piperazin- 1-yl)-propoxy]- methyl}-pyrrolo[2,1- f][1,2,4]triazin-4-yl)- (5-methyl-1H-pyrazol- 3-yl)-amine | LC-MS (ESI) m/z 479 (M + H)⁺; 8.21 min |
| 72d | 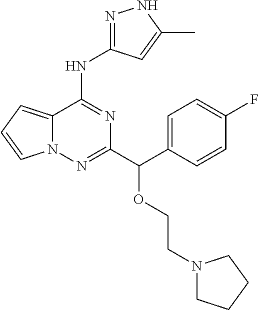 | {2-[(4-Fluoro-phenyl)- (2-pyrrolidin-1-yl- ethoxy)-methyl]- pyrrolo[2,1-f][1,2,4] triazin-4-yl}-(5-methyl- 1H-pyrazol-3-yl)-amine | LC-MS (ESI) m/z 436 (M + H)⁺; 8.29 min |
| 72e | 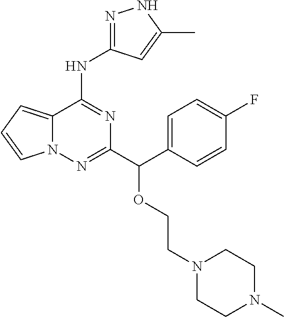 | (2-{(4-Fluoro-phenyl)- [2-(4-methyl-piperazin- 1-yl)-ethoxy]-methyl}- pyrrolo[2,1-f][1,2,4] triazin-4-yl)-(5-methyl- 1H-pyrazol-3-yl)-amine | LC-MS (ESI) m/z 465 (M + H)⁺; 8.10 min |
| 72f | 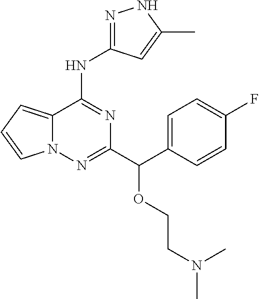 | {2-[(2-Dimethylamino- ethoxy)-(4-fluoro- phenyl)-methyl]- pyrrolo[2,1-f][1,2,4] triazin-4-yl}-(5-methyl- 1H-pyrazol-3-yl)-amine | LC-MS (ESI) m/z 410 (M + H)⁺; 8.14 min |

### Example 73

### Preparation of 2-((4-fluorophenyl)(methylamino)methyl)-N-(5-methyl-1H-pyrazol-3-yl)pyrrolo[1,2-f][1,2,4]triazin-4-amine

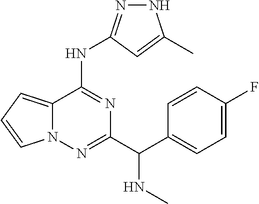

To 2-(bromo(4-fluorophenyl)methyl)-N-(5-methyl-1H-pyrazol-3-yl)pyrrolo[1,2-f][1,2,4]triazin-4-amine from Example 69, Step A (107 mg, 0.27 mmol) was added methylamine (4 mL, 2M in THF) and the solution was heated at 70° C. for 10 min then stirred at room temperature overnight. The mixture was concentrated, diluted with DMSO (3 mL), and purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(methylamino)methyl)-N-(5-methyl-1H-pyrazol-3-yl)pyrrolo[1,2-f][1,2,4]triazin-4-amine as its acetate salt (50 mg, 44%). ¹H NMR (300 MHz, DMSO-d₆) δ 1.91 (s, 3H), 2.25 (s, 3H), 2.32 (s, 3H), 4.65 (s, 1H), 6.24 (s, 1H), 6.65 (t, 1H), 7.1-7.3 (m, 3H), 7.52 (t, 1H), 7.70 (s, 1H), 10.53 (s, 1H); LC-MS (ESI) m/z 352 (M+H)⁺.

The following compounds were synthesized using a similar procedure using the appropriate amine, thiol or alcohol, to substitute the methylamine. In certain preparations, an enantiomeric form of the amine or alcohol was reacted with a racemic mixture of 2-(bromo(4-fluorophenyl)methyl)-N-(5-methyl-1H-pyrazol-3-yl)pyrrolo[1,2-f][1,2,4]triazin-4-amine] to produce a mixture of diastereomers.

The following compounds were synthesized using a similar procedure using the appropriate amine, thiol or alcohol in place of the methylamine. In certain preparations, an enantiomerically pure form of the amine or alcohol was used, producing a mixture of two diastereomers.

| Example | | | Analytical |
|---|---|---|---|
| Number | Structure | Name | Data |
| 73a | 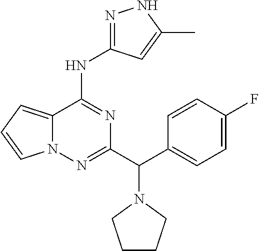 | {2-[(4-Fluoro-phenyl)- pyrrolidin-1-yl- methyl]-pyrrolo[2,1- f][1,2,4]triazin-4-yl}- (5-methyl-1H-pyrazol- 3-yl)-amine | LC-MS (ESI) m/z 392 (M + H)⁺; 8.29 min |
| 73b | 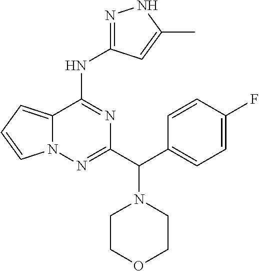 | {2-[(4-Fluoro-phenyl)- morpholin-4-yl- methyl]-pyrrolo[2,1- f][1,2,4]triazin-4-yl}- (5-methyl-1H-pyrazol- 3-yl)-amine | LC-MS (ESI) m/z 408 (M + H)⁺; 9.18 min |
| 73c | 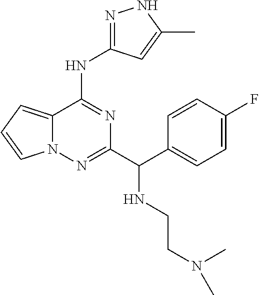 | N¹-((4-fluorophenyl)(4- (5-methyl-1H-pyrazol- 3-ylamino)pyrrolo[1,2- f][1,2,4]triazin-2- yl)methyl)-N²,N²- dimethylethane-1,2-diamine | LC-MS (ESI) m/z 409 (M + H)⁺; 8.03 min |
| 73d | 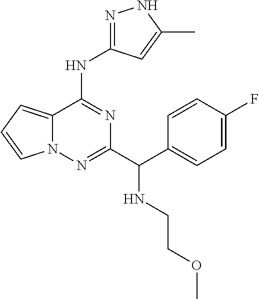 | {2-[(4-Fluoro-phenyl)- (2-methoxy-ethylamino)- methyl]-pyrrolo[2,1- f][1,2,4]triazin-4-yl}- (5-methyl-1H-pyrazol- 3-yl)-amine | LC-MS (ESI) m/z 396 (M + H)⁺; 8.17 min |
| 73e | 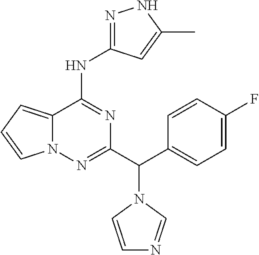 | {2-[(4-Fluoro-phenyl)- imidazol-1-yl-methyl]- pyrrolo[2,1-f][1,2,4] triazin-4-yl}-(5-methyl- 1H-pyrazol-3-yl)-amine | LC-MS (ESI) m/z 396 (M + H)⁺; 8.36 min |
| 73f | 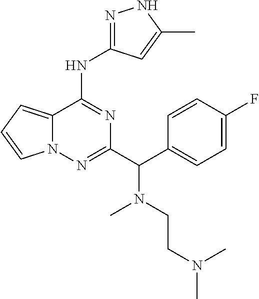 | N-{(4-Fluoro-phenyl)- [4-(5-methyl-1H- pyrazol-3-ylamino)- pyrrolo[2,1-f][1,2,4] triazin-2-yl]-methyl}- N,N′,N′-trimethyl- ethane-1,2-diamine | LC-MS (ESI) m/z 423 (M + H)⁺; 8.44 min |
| 73g | 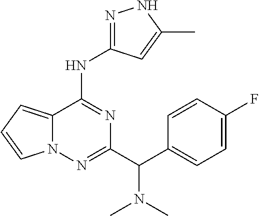 | (2-[Dimethylamino-(4- fluoro-phenyl)-methyl]- pyrrolo[2,1-f][1,2,4] triazin-4-yl}-(5-methyl- 1H-pyrazol-3-yl)-amine | LC-MS (ESI) m/z 366 (M + H)⁺; 8.16 min |
| 73h | 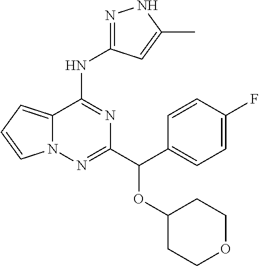 | {2-[(4-Fluoro-phenyl)- (tetrahydro-pyran-4- yloxy)-methyl]- pyrrolo[2,1-f][1,2,4] triazin-4-yl}-(5-methyl- 1H-pyrazol-3-yl)-amine | LC-MS (ESI) m/z 423 (M + H)⁺; 11.84 min |
| 73i | 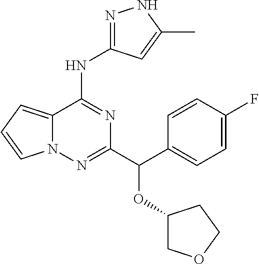 | 2-((R,S)-(4-fluorophenyl) ((R)-tetrahydrofuran-3- yloxy)methyl)-N-(5- methyl-1H-pyrazol-3- yl)pyrrolo[1,2-f] [1,2,4]triazin-4-amine {2-[(4-Fluoro-phenyl)- (tetrahydro-furan-3- yloxy)-methyl]- pyrrolo [2,1-f][1,2,4] triazin-4-yl}- (5-methyl-1H-pyrazol- 3-yl)-amine | LC-MS (ESI) m/z 409 (M + H)⁺; 11.61 min |
| 73j | 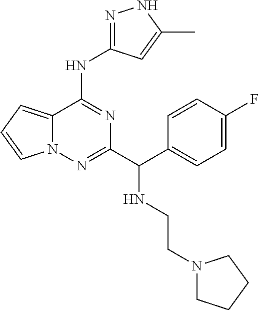 | {2-[(4-Fluoro-phenyl)- (2-pyrrolidin-1-yl- ethylamino)-methyl]- pyrrolo[2,1-f][1,2,4] triazin-4-yl}-(5-methyl- 1H-pyrazol-3-yl)-amine | LC-MS (ESI) m/z 435 (M + H)⁺; 8.13 min |
| 73k | 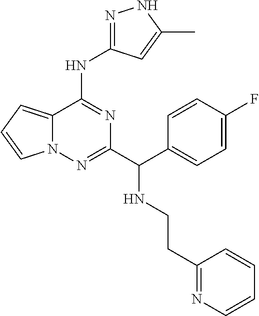 | {2-[(4-Fluoro-phenyl)- (2-pyridin-2-yl- ethylamino)-methyl]- pyrrolo[2,1-f][1,2,4] triazin-4-yl}- (5-methyl-1H-pyrazol- 3-yl)-amine | LC-MS (ESI) m/z 443 (M + H)⁺; 8.47 min |
| 73l | 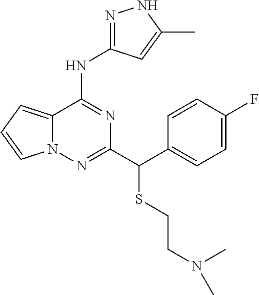 | {2-[(2-Dimethylamino- ethylsulfanyl)-(4- fluoro-phenyl)-methyl]- pyrrolo[2,1-f][1,2,4] triazin-4-yl}-(5-methyl- 1H-pyrazol-3-yl)-amine | LC-MS (ESI) m/z 426 (M + H)⁺; 8.31 min |
| 73m | 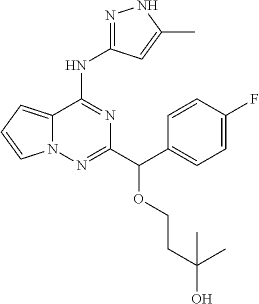 | 4-{(4-Fluoro-phenyl)- [4-(5-methyl-1H- pyrazol-3-ylamino)- pyrrolo[2,1- f][1,2,4]triazin-2-yl]- methoxy}-2-methyl- butan-2-ol | LC-MS (ESI) m/z 425 (M + H)⁺; 11.80 min |
| 73n | 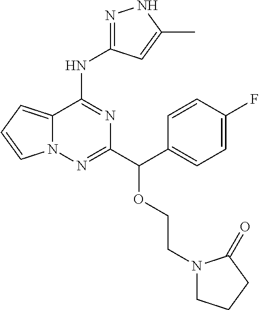 | 1-(2-{(4-Fluoro- phenyl)-[4-(5-methyl- 1H-pyrazol-3- ylamino)-pyrrolo[2,1- f][1,2,4]triazin-2-yl]- methoxy}-ethyl)- pyrrolidin-2-one | LC-MS (ESI) m/z 450 (M + H)⁺; 10.76 min |
| 73o | 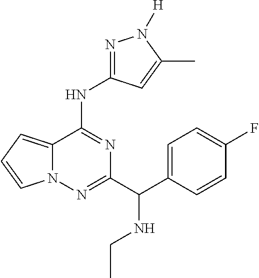 | {2-[Ethylamino-(4- fluoro-phenyl)-methyl]- pyrrolo[2,1- f][1,2,4]triazin-4-yl}- (5-methyl-1H-pyrazol- 3-yl)-amine | LC-MS (ESI) m/z 366 (M + H)⁺; 8.02 min |
| 73p | 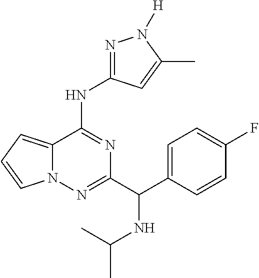 | {2-[(4-Fluoro-phenyl)- isopropylamino- methyl]-pyrrolo[2,1- f][1,2,4]triazin-4-yl}- (5-methyl-1H-pyrazol- 3-yl)-amine | LC-MS (ESI) m/z 380 (M + H)⁺; 8.08 min |
| 73q | 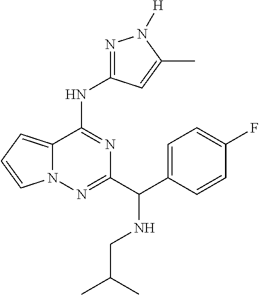 | {2-[(4-Fluoro-phenyl)- isobutylamino-methyl]- pyrrolo[2,1- f][1,2,4]triazin-4-yl}- (5-methyl-1H-pyrazol- 3-yl)-amine | LC-MS (ESI) m/z 394 (M + H)⁺; 8.52 min |
| 73r | 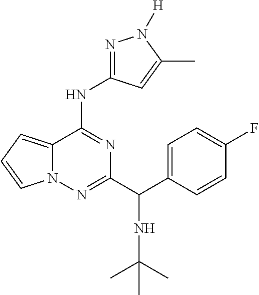 | {2-[tert-Butylamino-(4- fluoro-phenyl)-methyl]- pyrrolo[2,1- f][1,2,4]triazin-4-yl}- (5-methyl-1H-pyrazol- 3-yl)-amine | LC-MS (ESI) m/z 394 (M + H)⁺; 8.29 min |
| 73s | 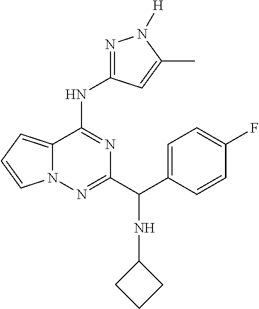 | {2-[Cyclobutylamino- (4-fluoro-phenyl)- methyl]-pyrrolo[2,1- f][1,2,4]triazin-4-yl}- (5-methyl-1H-pyrazol- 3-yl)-amine | LC-MS (ESI) m/z 392 (M + H)⁺; 8.33 min |
| 73t | 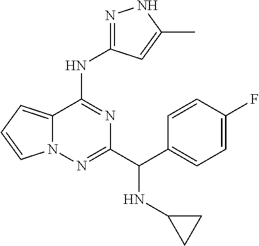 | {2-[Cyclopropylamino- (4-fluoro-phenyl)- methyl]-pyrrolo[2,1- f][1,2,4]triazin-4-yl}- (5-methyl-1H-pyrazol- 3-yl)-amine | LC-MS (ESI) m/z 378 (M + H)⁺; 8.45 min |
| 73u | 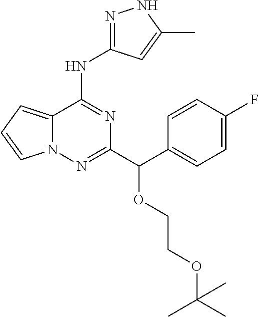 | {2-[(2-tert-Butoxy- ethoxy)-(4-fluoro- phenyl)-methyl]- pyrrolo[2,1-f][1,2,4] triazin-4-yl}- (5-methyl-1H-pyrazol- 3-yl)-amine | LC-MS (ESI) m/z 439 (M + H)⁺; 13.07 min |
| 73v | 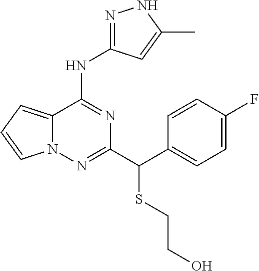 | 2-{(4-Fluoro-phenyl)- [4-(5-methyl-1H- pyrazol-3-ylamino)- pyrrolo[2,1-f][1,2,4] triazin-2-yl]- methylsulfanyl}-ethanol | LC-MS (ESI) m/z 399 (M + H)⁺; 11.21 min |
| 73w | 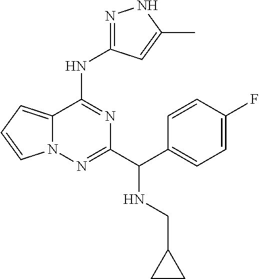 | {2-[(Cyclopropylmethyl- amino)-(4-fluoro- phenyl)-methyl]- pyrrolo[2,1-f][1,2,4] triazin-4-yl}-(5-methyl- 1H-pyrazol-3-yl)-amine | LC-MS (ESI) m/z 392 (M + H)⁺; 8.28 min |
| 73x | 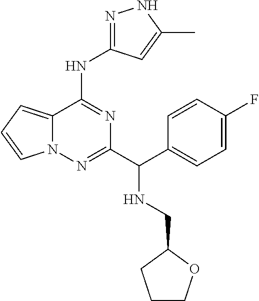 | 2-((R,S)-(4-fluorophenyl) (((S)-tetrahydrofuran-2- yl)methylamino)methyl)- N-(5-methyl-1H- pyrazol-3-yl)pyrrolo[1,2-f] [1,2,4]triazin-4-amine | LC-MS (ESI) m/z 422 (M + H)⁺; 8.33 min |
| 73y | 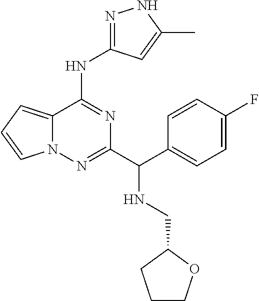 | 2-((R,S)-(4-fluorophenyl) (((R)-tetrahydrofuran-2- yl)methylamino)methyl)- N-(5-methyl-1H- pyrazol-3-yl)pyrrolo[1,2- f][1,2,4]triazin-4-amine | LC-MS (ESI) m/z 422 (M + H)⁺; 8.37 min |
| 73z | 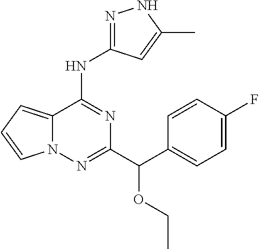 | {2-[Ethoxy-(4-fluoro- phenyl)-methyl]- pyrrolo[2,1-f][1,2,4] triazin-4-yl}-(5-methyl- 1H-pyrazol-3-yl)-amine | LC-MS (ESI) m/z 367 (M + H)⁺; 12.74 min |
| 73aa | 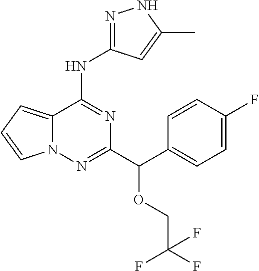 | {2-[(4-Fluoro-phenyl)- (2,2,2-trifluoro- ethoxy)-methyl]- pyrrolo[2,1-f][1,2,4] triazin-4-yl}-(5-methyl- 1H-pyrazol-3-yl)-amine | LC-MS (ESI) m/z 421 (M + H)⁺; 13.31 min |
| 73bb | 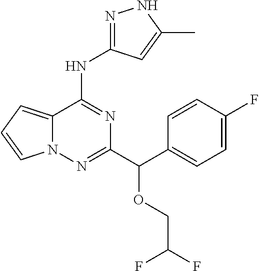 | {2-[(2,2-Difluoro- ethoxy)-(4-fluoro- phenyl)-methyl]- pyrrolo[2,1-f][1,2,4] triazin-4-yl}-(5-methyl- 1H-pyrazol-3-yl)-amine | LC-MS (ESI) m/z 403 (M + H)⁺; 12.67 min |
| 73cc | 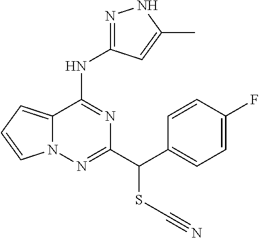 | {2-[(4-Fluoro-phenyl)- thiocyanato-methyl]- pyrrolo[2,1-f][1,2,4] triazin-4-yl}-(5-methyl- 1H-pyrazol-3-yl)-amine | LC-MS (ESI) m/z 380 (M + H)⁺; 11.83 min |
| 73dd | 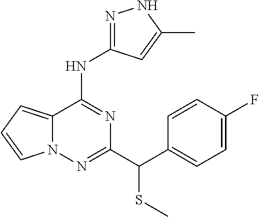 | {2-[(4-Fluoro-phenyl)- methylsulfanyl- methyl]-pyrrolo[2,1- f][1,2,4]triazin-4-yl}- (5-methyl-1H-pyrazol- 3-yl)-amine | LC-MS (ESI) m/z 369 (M + H)⁺; 12.10 min |

### Example 74

### Preparation of 2-(difluoro(4-fluorophenyl)methyl)-6-((2-methoxyethoxy)methyl)-N-(5-methyl-1H-pyrazol-3-yl)pyrrolo[1,2-f][1,2,4]triazin-4-amine

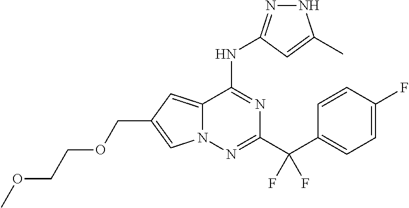

To a solution of 2-methoxyethanol (0.56 mL, 7.1 mmol) in THF (6 mL) was added a 60% suspension of NaH in mineral oil at 0° C. After stirring for 15 min, 6-(bromomethyl)-2-(difluoro(4-fluorophenyl)methyl)-N-(5-methyl-1H-pyrazol-3-yl)pyrrolo[1,2-f][1,2,4]triazin-4-amine from Example 52, Step A (400 mg, 0.88 mol) was added at room temperature. The reaction mixture was stirred at room temperature for 2 h, then diluted with EtOAc (5 mL) and water. The organic phase was separated and washed with brine. After drying over MgSO₄, the solvent was evaporated under vacuum and the crude product was purified by preparative HPLC to yield 2-(difluoro(4-fluorophenyl)methyl)-6-((2-methoxyethoxy)methyl)-N-(5-methyl-1H-pyrazol-3-yl)pyrrolo[1,2-f][1,2,4]triazin-4-amine as a white solid (18 mg, 5%). ¹H NMR (DMSO-d₆): δ 12.21 (s, 1H), 10.88 (s, 1H), 7.82 (s, 1H), 7.70 (t, 2H), 7.36 (t, 2H), 7.30 (s, 1H), 6.23 (s, 1H), 4.52 (s, 2H), 3.54 (m, 2H), 3.47 (m, 2H), 3.25 (s, 3H), 2.21 (s, 3H); LC-MS (ESI) m/z 447 (M+H)⁺.

### Example 75

### Preparation of 3-(2-(4-fluorobenzoyl)pyrrolo[1,2-f][1,2,4]triazin-4-ylamino)-1H-pyrazole-5-carbonitrile

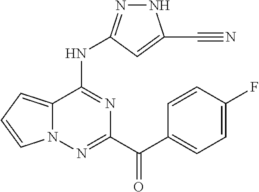

Step A. To a solution of 5-nitro-3-pyrazolecarboxylic acid (6.28 g, 40 mmol) in DMF (30 mL) was added carbonyldiimidazole (12.96 g, 80 mmol), and the mixture was allowed to stir for 30 min, followed by the addition of 2M ammonia in MeOH (60 mL). The mixture was stirred at rt overnight, then concentrated under reduced pressure to afford a residue, which was triturated with ether to afford 3-nitro-1H-pyrazole-5-carboxamide (3.0 g, 48% yield). LC-MS (ESI) m/z 155 (M−H)⁻.

Step B. To 3-nitro-1H-pyrazole-5-carboxamide (3 g, 19.2 mmol) in pyridine (30 mL) was added phosphorous oxychloride (5.9 g, 38.4 mmol), and the resulting solution was stirred for 3 h at rt. The mixture was diluted with ice, then extracted with DCM (100 mL). The organic phase was separated, dried over sodium sulfate, filtered and concentrated under reduced pressure to afford crude 3-nitro-1H-pyrazole-5-carbonitrile (1 g, 38%). LC-MS (ESI) m/z 137 (M−H)⁻.

Step C. To a solution of 3-nitro-1H-pyrazole-5-carbonitrile (1 g, 7.24 mmol) in acetic acid (10 mL) and water (2 mL) was added zinc powder (2.35 g, 36.24 mmol) at 0° C. The mixture was stirred at room temperature. for 3 h, then filtered. To the filtrate was added ammonium hydroxide to give pH ˜8, followed by extraction with ethyl acetate (30 mL). The separated organic phase was dried over sodium sulfate, filtered and concentrated under reduced pressure to afford crude 3-amino-1H-pyrazole-5-carbonitrile (200 mg, 28%), which was used without further purification. LC-MS (ESI) m/z 107 (M−H)⁻.

Step D. A mixture of 3-amino-1H-pyrazole-5-carbonitrile (51 mg, 0.47 mmol) and (4-chloropyrrolo[1,2-f][1,2,4]triazin-2-yl)(4-fluorophenyl)methanone from General Procedure B (100 mg, 0.36 mmol) in. DMF (2 mL) was stirred at room temperature overnight. The solution was then concentrated under reduced pressure to afford a residue, which was purified by preparative HPLC to afford 3-(2-(4-fluorobenzoyl)pyrrolo[1,2-f][1,2,4]triazin-4-ylamino)-1H-pyrazole-5-carbonitrile (35 mg, 28%) as yellow solid. ¹H NMR (300 MHz, DMSO-d₆) δ 6.95 (s, 1H), 6.97-7.42 (m, 4H), 8.0 (s, 1H), 8.12-8.16 (m, 2H), 11.46 (bs, 1H), 13.95 (bs, 1H); LC-MS (ESI) m/z 348 (M+H)⁺.

The following compound was prepared from 3-(2-(4-fluorobenzoyl)pyrrolo[1,2-f][1,2,4]triazin-4-ylamino)-1H-pyrazole-5-carbonitrile, using General Procedure D in Example 2:

| Example | | | Analytical |
|---|---|---|---|
| Number | Structure | Name | Data |
| 75a | 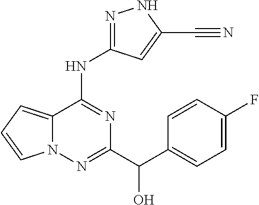 | 5-{2-[(4-Fluoro-phenyl)- hydroxy-methyl]- pyrrolo[2,1-f][1,2,4]triazin- 4-ylamino}-1H-pyrazole- 5-carbonitrile | LC-MS (ESI) m/z 350 (M + H)⁺; 11.90 min |

### Example 76

### Preparation of (6-chloro-4-(5-methyl-1H-pyrazol-3-ylamino)pyrrolo[1,2-f][1,2,4]triazin-2-yl)(4-fluorophenyl)methanone

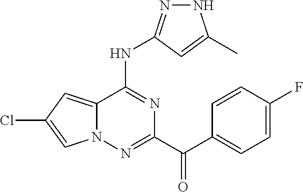

Step A: To a stirred solution of 2,2,2-trichloro-1-(1H-pyrrol-2-yl)ethanone (12.7 g, 60 mmol) in DCM (60 mL) at 0° C. was slowly added sulfuryl chloride (9.5 g, 70 mmol), stirring was continued at room temperature overnight. The mixture was diluted with DCM (300 mL) and poured onto ice (150 g). The separated organic layer was washed with saturated NaHCO₃ and brine, dried over NaSO₄, filtered and concentrated in vacuo. The residue was purified by silica gel chromatography eluting with diethyl ether-hexane (1:10) to give 2,2,2-trichloro-1-(4-chloro-1H-pyrrol-2-yl)ethanone as a white solid (9.1 g, 61%). ¹H NMR (300 MHz, CDCl₃) δ 7.12 (dd, 1H), 7.28 (dd, 1H), 9.48 (br s, 1H).

Step B: To a stirred solution of 2,2,2-trichloro-1-(4-chloro-1H-pyrrol-2-yl)ethanone (9.1 g, 37 mmol) in MeOH (40 mL) at rt was slowly added sodium methoxide (2.39 g, 44 mmol), and the solution was stirred at rt for 1 h. The solution was diluted with DCM (400 mL), washed with saturated NaHCO₃ and brine. The organic phase was dried over Na₂SO₄, filtered, and concentrated in vacuo to give methyl 4-chloro-1H-pyrrole-2-carboxylate as a white solid (5.7 g, 94%). NMR (300 MHz, CDCl₃) δ 3.86 (s, 3H), 6.81 (dd, 1H), 6.90 (dd, 1H), 9.31 (br s, 1H); LC-MS (ESI) m/z 160 (M+H)⁺.

Step C: To a suspension of ammonium chloride (18 g, 340 mmol) in Et₂O (330 mL) at −10° C. was added ammonium hydroxide (28.2 g, 806 mmol). Then 5-7% aqueous sodium hypochlorite (432 mL) was added slowly, after which the mixture was stirred at −5° C. for 1 h. The separated organic layer was washed with brine and dried over anhydrous K₂CO₃ at −78° C. for 1 h to afford a ca. 0.3 M solution of chloroamine in Et₂O which was used directly in next step.

Step D: To a solution of methyl 4-chloro-1H-pyrrole-2-carboxylate (5.7 g, 36 mmol) in 30 mL of DMF at 0° C. was added NaH (2.03 g, 46 mmol). After stirring at 0° C. for 1 h, the chloroamine solution from above (168 mL, 50.4 mmol) was added. The solution was stirred at rt overnight, then diluted with EtOAc (300 mL) and washed with saturated aq NaHCO₃ and brine. The resulting organic phase was dried over Na₂SO₄, filtered and concentrated in vacuo to provide methyl 1-amino-4-chloro-1H-pyrrole-2-carboxylate as a white solid (6.08 g, 97%). ¹H NMR (300 MHz, CDCl₃) δ 3.83 (s, 3H), 5.55 (br s, 2H), 6.73 (d, 1H), 6.91 (d, 1H); LC-MS (ESI) m/z 175 (M+H)⁺.

Step E: Methyl 1-amino-4-chloro-1H-pyrrole-2-carboxylate (9.9 g, 56.7 mmol) was dissolved in ca. 10 M ammonia/methanol (300 mL). The solution was stirred at 90° C. under 9 atm of pressure overnight, then concentrated under reduced pressure. The residue was triturated with petroleum ether and filtered, providing 1-amino-4-chloro-1H-pyrrole-2-carboxamide (8.6 g, 94.5%). ¹H NMR (300 MHz, DMSO-d₆) δ 6.65 (s, 3H), 6.96 (s, 1H), 7.26 (bs, 1H), 7.91 (bs, 1H).

Step F: To a solution of 1-amino-4-chloro-1H-pyrrole-2-carboxamide (4.0 g, 25.1 mmol) and pyridine (2 g, 25.1 mmol) in THF (30 mL) was added dropwise ethyl choroglyoxylate (3.42 g, 25.1 mmol). The mixture was stirred at room temperature, then concentrated under reduce pressure. DCM (50 mL) was added and the suspended solid was collected by filtration, providing ethyl 2-(2-carbamoyl-4-chloro-1H-pyrrol-1-ylamino)-2-oxoacetate (4 g, 61.5%). ¹H NMR (300 MHz, DMSO-d₆) δ 1.31 (t, 3H), 4.33 (q, 2H), 6.88 (s, 1H), 7.1 (bs, 1H), 7.19 (s, 1H), 7.59 (bs, 1H), 12.16 (s, 1H).

Step G: To a solution of ethyl 2-(2-carbamoyl-4-chloro-1H-pyrrol-1-ylamino)-2-oxoacetate (10 g, 38.5 mmol) and triethylamine (89.7 g, 0.88 mol) in 1,2-dichloroethane (350 mL) was added dropwise chlorotrimethylsilane (83.7 g, 088 mmol). The mixture was stirred overnight, then cooled and poured into ice water. The resulting mixture was extracted with DCM, and the combined organic phases were washed with water and brine, dried over magnesium sulfate, filtered, and evaporated under reduced pressure to give a residue, which was purified by silica gel chromatography to provide ethyl 6-chloro-4-hydroxypyrrolo[1,2-f][1,2,4]triazine-2-carboxylate (6 g, 64.5%). LC-MS (ESI) m/z 240 (M−H)⁻.

Step H: A mixture of ethyl 6-chloro-4-hydroxypyrrolo[1,2-f][1,2,4]triazine-2-carboxylate (3.0 g, 12.4 mmol) and phosphorous oxychloride (30 mL) was stirred for 5 min, after which N,N-diethylaniline (3.7 g) was added dropwise. This mixture was stirred at 85° C. for 2 h, then added to ice water. The suspended solid was collected by filtration, washed with water and petroleum ether, then dried under vacuum to yield ethyl 4,6-dichloropyrrolo[1,2-f][1,2,4]triazine-2-carboxylate (2.0 g, 62%). ¹H NMR (300 MHz, DMSO-d₆) δ 1.36 (t, 3H), 4.40 (q, 2H), 7.42 (s, 1H), 8.74 (s, 1H).

Step I: To 4,6-dichloropyrrolo[1,2-f][1,2,4]triazine-2-carboxylate (0.05 g, 0.19 mmol) in THF (0.6 mL) at −40° C. was added 1 M 4-fluorophenylmagnesium bromide in THF (0.044 mL), and the solution stirred for 2 days at −40° C. Then 1 N HCl (0.6 mL) and ethyl acetate (6 mL) were added and the organic layer was separated, dried over sodium sulfate, filtered and evaporated to give a residue (0.07 g). To the residue in DMF (2 mL), were added 5-methyl-1H-pyrazol-3-amine (0.044 g, 0.45 mmol), potassium iodide (0.037 g, 0.23 mmol), and diisopropylethylamine (0.04 mL, 0.23 mmol), and the solution was stirred at rt for 30 min. Water was added and the mixture was extracted with ethyl acetate. The organic layer was dried over sodium sulfate, filtered, and evaporated. The residue was purified by preparative HPLC (Varian diphenyl reverse phase column, eluted with gradient of solvent B=0.05% HOAC/CH₃CN and solvent A=0.05% HOAc/H₂O) to afford (6-chloro-4-(5-methyl-1H-pyrazol-3-ylamino)pyrrolo[1,2-f][1,2,4]triazin-2-yl)(4-fluorophenyl)methanone (3 mg, 4%). ¹H NMR (300 MHz, DMSO-d₆) δ 2.08 (s, 3H), 6.52 (s, 1H), 7.37-7.45 (m, 3H), 8.13-8.15 (m, 3H), 10.97 (bs, 1H), 12.29 (bs, 1H); LC-MS (ESI) m/z 371 (M+H)⁺.

The following compound was prepared from (6-chloro-4-(5-methyl-1H-pyrazol-3-ylamino)pyrrolo[1,2-f][1,2,4]triazin-2-yl)(4-fluorophenyl)methanone using General Procedure D in Example 2.

| Example | | | Analytical |
|---|---|---|---|
| Number | Structure | Name | Data |
| 76a | 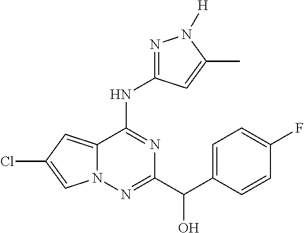 | [6-Chloro-4-(5-methyl-1H- pyrazol-3-ylamino)- pyrrolo[2,1-f][1,2,4]triazin- 2-yl]-(4-fluoro-phenyl)- methanol | LC-MS (ESI) m/z 373 (M + H)⁺; 12.07 min |

### Example 77

### Preparation of 2-((4-fluorophenyl)(((R)-tetrahydrofuran-2-yl)methoxy)methyl)-N-(5-methyl-1H-pyrazol-3-yl)pyrrolo[1,2-f][1,2,4]triazin-4-amine

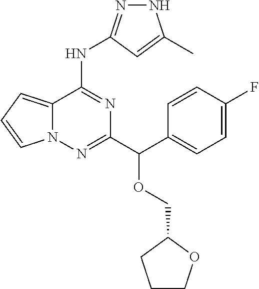

To a suspension of 2-(bromo(4-fluorophenyl)methyl)-N-(5-methyl-1H-pyrazol-3-yl)pyrrolo[1,2-f][1,2,4]triazin-4-amine from Example 69, Step A (0.069 g, 0.17 mmol) in DCM (2 mL) were added (R)-(tetrahydrofuran-2-yl)methanol (0.05 mL, 0.51 mmol), 2,6-di-tert-butylpyridine (0.077 mL, 0.34 mmol) silver trifluoromethansulfonate (0.065 g, 0.26 mmol). The solution was stirred for 10 min, filtered rinsing with MeOH, and the filtrate was concentrated. The residue was purified by preparative HPLC (Phenomenex C-18 reverse phase column, eluted with gradient of solvent B=0.05% HOAC/CH₃CN and solvent A=0.05% HOAc/H₂O) to afford 2-((4-fluorophenyl)(((R)-tetrahydrofuran-2-yl)methoxy)methyl)-N-(5-methyl-1H-pyrazol-3-yl)pyrrolo[1,2-f][1,2,4]triazin-4-amine (4.6 mg, 6% yield).). ¹H NMR (300 MHz, DMSO-d₆) δ 1.66-1.91 (m, 4H), 2.26 (s, 3H), 3.4-3.7 (m, 4H), 4.02 (m, 1H), 5.35 (s, 1H), 6.5-6.7 (m, 2H), 7.15-7.2 (m, 3H), 7.56 (t, 2H), 7.72 (s, 1H), 10.6 (s, 1H), 12.2 (bs, 1H); LC-MS (ESI) m/z 423 (M+H)⁺.

### Example 78

### Preparation of 2-((2-(tert-butylamino)ethoxy)(4-fluorophenyl)methyl)-N-(5-methyl-1H-pyrazol-3-yl)pyrrolo[1,2-f][1,2,4]triazin-4-amine

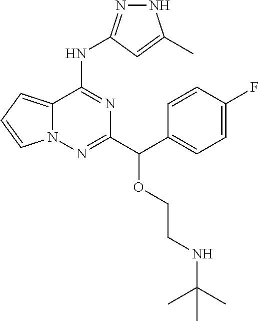

Step A: To a suspension of (4-fluorophenyl)(4-(5-methyl-1H-pyrazol-3-ylamino)pyrrolo[1,2-f][1,2,4]triazin-2-yl)methanol from Example 2 (0.050 g, 0.15 mmol) in 2-chloroethanol (0.7 mL) was added methanesulfonic acid (0.05 mL) and the solution was heated to 140° C. in under microwave irradiation for 40 min. The mixture was made basic with triethylamine and then purified by silica gel chromatography eluting with 0-20% MeOH/DCM to afford 2-((2-chloroethoxy)(4-fluorophenyl)methyl)-N-(5-methyl-1H-pyrazol-3-yl)pyrrolo[1,2-f][1,2,4]triazin-4-amine (40 mg, 69% yield). LC-MS (ESI) m/z 401 (M+H)⁺.

Step B: To 24(2-chloroethoxy)(4-fluorophenyl)methyl)-N-(5-methyl-1H-pyrazol-3-yl)pyrrolo[1,2-f][1,2,4]triazin-4-amine (0.18 g, 0.45 mmol) in 1-methylpyrrolidin-2-one (5 mL) were added potassium iodide (90 mg, 0.54 mmol) and 2-methylpropan-2-amine (1.5 mL) and the solution was heated at 70° C. for 3 days. The mixture was purified by preparative HPLC (Varian diphenyl reverse phase column, eluted with gradient of solvent B=0.05% HOAC/CH₃CN and solvent A=0.05% HOAc/H₂O) to afford 2-((2-(tert-butylamino)ethoxy)(4-fluorophenyl)methyl)-N-(5-methyl-1H-pyrazol-3-yl)pyrrolo[1,2-f][1,2,4]triazin-4-amine as the acetate salt (22 mg, 10%). NMR (300 MHz, DMSO-d₆) δ 0.95 (s, 9H), 1.88 (s, 3H), 2.25 (s, 3H), 2.68 (m, 2H), 3.54 (m, 2H), 5.30 (s, 1H), 6.55 (s, 1H), 6.66 (m, 1H), 7.15-7.21 (m, 3H), 7.57 (m, 2H), 7.71 (m, 1H), 10.61 (bs, 1H); LC-MS (ESI) m/z 438 (M+H)⁺.

### Example 79

### Preparation of 2-((4-fluorophenyl)(2-morpholinoethylthio)methyl)-N-(5-methyl-1H-pyrazol-3-yl)pyrrolo[1,2-f][1,2,4]triazin-4-amine

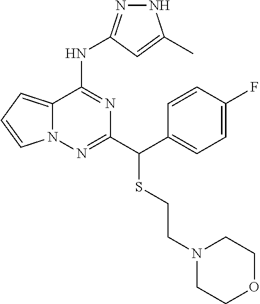

To potassium thioacetate (0.0.085 g, 0.75 mmol) in DMF (1 mL) was added 2-(bromo(4-fluorophenyl)methyl)-N-(5-methyl-1H-pyrazol-3-yl)pyrrolo[1,2-f][1,2,4]triazin-4-amine from Example 69, Step A (0.1 g, 0.25 mmol) and the solution was stirred at rt for 20 min. Then 25% sodium methoxide in MeOH (0.1 mL) was added and the mixture was stirred at rt for 5 min. Then 4-(2-chloroethyl)morpholine hydrochloride (0.093 g, 0.5 mmol) was added, followed by 25% sodium methoxide in MeOH (0.1 mL). The mixture was stirred at rt for 2 h, sodium borohydride (20 mg) was added, and the solution was stirred overnight. Acetic acid (0.4 mL) was added and the resulting mixture was purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(2-morpholinoethylthio)methyl)-N-(5-methyl-1H-pyrazol-3-yl)pyrrolo[1,2-f][1,2,4]triazin-4-amine (25 mg, 21%). ¹H NMR (300 MHz, DMSO-d₆) δ 2.25-2.35 (m, 5H), 2.4-2.6 (m, 6H), 3.51 (m, 4H), 5.16 (s, 1H), 6.64-6.66 (m, 2H), 7.13-7.19 (m, 3H), 7.65-7.70 (m, 3H), 10.61 (s, 1H), 12.16 (bs, 1H); LC-MS (ESI) m/z 468 (M+H)⁺.

### Example 80

### Preparation of 2-((4-fluorophenyl)(3-(methylthio)propylamino)methyl)-N-(5-methyl-1H-pyrazol-3-yl)pyrrolo[1,2-f][1,2,4]triazin-4-amine

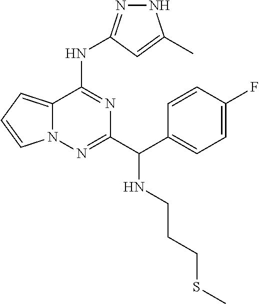

To 2-(bromo(4-fluorophenyl)methyl)-N-(5-methyl-1H-pyrazol-3-yl)pyrrolo[1,2-f][1,2,4]triazin-4-amine from Example 69, Step A (0.15 g, 0.37 mmol) in THF (1 mL) was added 3-(methylthio)propan-1-amine (0.1 mL, 0.93 mmol), and the solution was heated at 50° C. overnight. An aliquot (0.4 mL) of this mixture was purified by preparative HPLC (Varian diphenyl reverse phase column, eluted with a gradient of solvent B=0.05% HOAC/CH₃CN and solvent A=0.05% HOAc/H₂O) to afford 2-((4-fluorophenyl)(3-(methylthio)propylamino)methyl)-N-(5-methyl-1H-pyrazol-3-yl)pyrrolo[1,2-f][1,2,4]triazin-4-amine (24 mg, 45%). ¹H NMR (300 MHz, DMSO-d₆) δ 1.71 (m, 2H), 1.99 (s, 3H), 2.26 (s, 3H), 2.5-2.6 (m, 4H), 4.67 (s, 1H), 6.46 (s, 1H), 6.63 (m, 1H), 7.10-7.18 (m, 3H), 7.52 (m, 2H), 7.68 (s, 1H), 10.52 (bs, 1H), 12.2 (bs, 1H); LC-MS (ESI) m/z 426 (M+H)⁺.

### Example 81

### Preparation of N-((2-(difluoro(4-fluorophenyl)methyl)-4-(5-methyl-1H-pyrazol-3-ylamino)pyrrolo[1,2-f][1,2,4]triazin-6-yl)methyl)-2-morpholinoacetamide

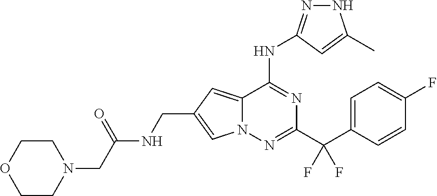

To a stirred solution of 6-(aminomethyl)-2-(difluoro(4-fluorophenyl)methyl)-N-(5-methyl-1H-pyrazol-3-yl)pyrrolo[1,2-f][1,2,4]triazin-4-amine from Example 48 (72 mg, 0.186 mmol) in 1:1 THF/DMF (6 mL) was added DIPEA (65 μL, 0.372 mmol). The mixture was cooled to −10° C. and chloroacetyl chloride (5 drops) was added slowly. When LC-MS indicated the reaction was complete, morpholine (0.372 mmol) was added and the mixture was heated at 50° C. overnight. The mixture was then cooled to rt and purified by preparative HPLC to give N-((2-(difluoro(4-fluorophenyl)methyl)-4-(5-methyl-1H-pyrazol-3-ylamino)pyrrolo[1,2-f][1,2,4]triazin-6-yl)methyl)-2-morpholinoacetamide. ¹H NMR (300 MHz, DMSO-d₆) δ 12.21 (s, 1H), 10.85 (s, 1H), 8.23 (t, 1H), 7.70 (m, 3H), 7.36 (t, 2H), 7.22 (s, 1H), 6.22 (s, 1H), 4.33 (d, 2H), 3.61 (s, 4H), 2.97 (s, 2H), 2.44 (s, 4H), 2.22 (s, 3H); LC-MS (ESI) m/z 515 (M+H)⁺.

### Example 82

### Preparation of 1-(2-(difluoro(4-fluorophenyl)methyl)-4-(5-methyl-1H-pyrazol-3-ylamino)pyrrolo[1,2-f][1,2,4]triazin-6-yl)-2,2,2-trifluoroethanol

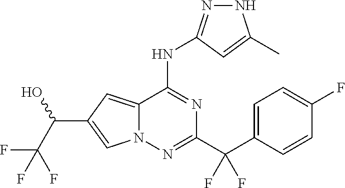

To a stirred solution of 2-(difluoro(4-fluorophenyl)methyl)-4-(5-methyl-1H-pyrazol-3-ylamino)pyrrolo[1,2-f][1,2,4]triazine-6-carbaldehyde from Example 56, Step A (140 mg, 0.36 mmol) in THF (2 mL) was added CF₃-TMS (600 μL, 4 mmol). The mixture was flushed with argon, and 1.0 M tetrabutylammonium fluoride in THF (1 mL, 1 mmol) was added dropwise at rt. After 30 min, the crude product was purified by reverse phase HPLC eluting with 10-80% AcCN to give 1-(2-(difluoro(4-fluorophenyl)methyl)-4-(5-methyl-1H-pyrazol-3-ylamino)pyrrolo[1,2-f][1,2,4]triazin-6-yl)-2,2,2-trifluoroethanol. ¹H NMR (300 MHz, MeOH-d₄) δ 7.80 (s; 1H), 7.71 (m, 2H), 7.22 (m, 3H), 6.33 (s, 1H), 5.22 (q, 1H), 2.29 (s, 3H); LC-MS (ESI) m/z 457 (M+H)⁺.

### Example 83

### Preparation of 2-amino-1-(2-(difluoro(4-fluorophenyl)methyl)-4-(5-methyl-1H-pyrazol-3-ylamino)pyrrolo[1,2-f][1,2,4]triazin-6-yl)ethanol

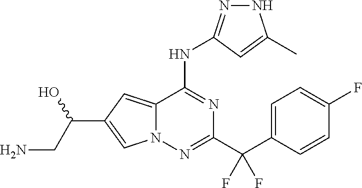

Step A: To a stirred solution of 2-(difluoro(4-fluorophenyl)methyl)-4-(5-methyl-1H-pyrazol-3-ylamino)pyrrolo[1,2-f][1,2,4]triazine-6-carbaldehyde from Example 56, Step A (2.62 g, 6.78 mmol) in THF (30 mL) were added nitromethane (6 mL, 100 mmol) and Amberlyst-A-21 (3 g). The mixture was stirred at rt for 8 h, then filtered through Celite washing with MeOH. The filtrate was concentrated in vacuo and the residue was dried under high vacuum to give 1-(2-(difluoro(4-fluorophenyl)methyl)-4-(5-methyl-1H-pyrazol-3-ylamino)pyrrolo[1,2-f][1,2,4]triazin-6-yl)-2-nitroethanol.

Step B: To 1-(2-(difluoro(4-fluorophenyl)methyl)-4-(5-methyl-1H-pyrazol-3-ylamino)pyrrolo[1,2-f][1,2,4]triazin-6-yl)-2-nitroethanol (1.25 g) in a round-bottom flask was flushed with argon were added Raney nickel (1 g) followed by MeOH (60 mL). The reaction flask was evacuated and filled with hydrogen, then stirred at rt for 2 h. The mixture was filtered through Celite and the filtrate was concentrated in vacuo. The residue was purifited by preparative HPLC to afford 2-amino-1-(2-(difluoro(4-fluorophenyl)methyl)-4-(5-methyl-1H-pyrazol-3-ylamino)pyrrolo[1,2-f][1,2,4]triazin-6-yl)ethanol as the acetate salt. ¹H NMR (300 MHz, DMSO-d₆) δ 7.74 (s, 1H), 7.70 (m, 2H), 7.36 (t, 2H), 7.27 (s, 1H), 6.23 (s, 1H), 4.62 (m, 1H), 2.76 (m, 2H), 2.21 (s, 3H), 1.86 (s, 3H); LC-MS (ESI) m/z 418 (M+H)⁺.

### Example 84

### Preparation of N-(2-(2-(difluoro(4-fluorophenyl)methyl)-4-(5-methyl-1H-pyrazol-3-ylamino)pyrrolo[1,2-f][1,2,4]triazin-6-yl)-2-hydroxyethyl)acetamide

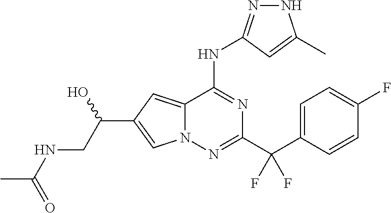

To a stirred solution of 2-amino-1-(2-(difluoro(4-fluorophenyl)methyl)-4-(5-methyl-1H-pyrazol-3-ylamino)pyrrolo[1,2-f][1,2,4]triazin-6-yl)ethanol from Example 83 (49 mg, 0.12 mmol) in DMF (2 mL) was added DIPEA (30 uL, 0.18 mmol), followed by dropwise addition of acetyl chloride. Upon confirming complete reaction by LC-MS, the mixture was purified by reverse phase HPLC eluting with 10-80% AcCN to give N-(2-(2-(difluoro(4-fluorophenyl)methyl)-4-(5-methyl-1H-pyrazol-3-ylamino)pyrrolo[1,2-f][1,2,4]triazin-6-yl)-2-hydroxyethyl)acetamide. ¹H NMR (300 MHz, MeOH-d₄) δ 7.70 (m, 3H), 7.22 (t, 2H), 7.02 (s, 1H), 6.32 (s, 1H), 3.55 (m, 1H), 3.42 (m, 1H), 2.28 (s, 3H), 1.95 (s, 3H); LC-MS (ESI) m/z 460 (M+H)⁺.

### Example 85

### 1-(2-(difluoro(4-fluorophenyl)methyl)-4-(5-methyl-1H-pyrazol-3-ylamino)pyrrolo[1,2-f][1,2,4]triazin-6-yl)-2-morpholinoethanol

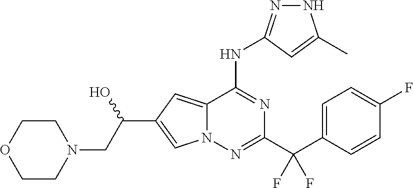

To a stirred solution of 2-amino-1-(2-(difluoro(4-fluorophenyl)methyl)-4-(5-methyl-1H-pyrazol-3-ylamino)pyrrolo[1,2-f][1,2,4]triazin-6-yl)ethanol from Example 83 (69 mg, 0.165 mmol) in THF (3 mL) were added TEA (58 μL, 0.41 mmol) and bis(2-bromoethyl)ether (20 mL, 0.165 mmol). The mixture was heated at 80° C. for 2.5 days, then purified by preparative HPLC eluting with 10-80% AcCN to give 1-(2-(difluoro(4-fluorophenyl)methyl)-4-(5-methyl-1H-pyrazol-3-ylamino)pyrrolo[1,2-f][1,2,4]triazin-6-yl)-2-morpholinoethanol. ¹H NMR (300 MHz, MeOH-d₄) δ 7.72 (s, 1H), 7.68 (m, 2H), 7.22 (t, 2H), 7.03 (s, 1H), 6.31 (s, 1H), 5.01 (m, 1H), 3.72 (m, 4H), 2.66 (m, 6H), 2.28 (s, 3H); LC-MS (ESI) m/z 488 (M+H)⁺.

### Example 86

### 5-(2-(difluoro(4-fluorophenyl)methyl)-4-(5-methyl-1H-pyrazol-3-ylamino)pyrrolo[1,2-f][1,2,4]triazin-6-yl)oxazolidin-2-one

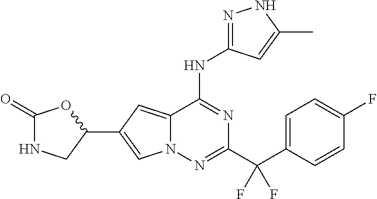

To a stirred solution of 2-amino-1-(2-(difluoro(4-fluorophenyl)methyl)-4-(5-methyl-1H-pyrazol-3-ylamino)pyrrolo[1,2-f][1,2,4]triazin-6-yl)ethanol from Example 83 (69 mg, 0.165 mmol) and DIPEA (72 μL, 0.413 mmol) in THF (3 mL) at 0° C. was added diphosgene (4 drops). The mixture was then stirred at rt for 2.5 days, then purified by preparative HPLC eluting with 10-80% AcCN to give 5-(2-(difluoro(4-fluorophenyl)methyl)-4-(5-methyl-1H-pyrazol-3-ylamino)pyrrolo[1,2-f][1,2,4]triazin-6-yl)oxazolidin-2-one. ¹H NMR (300 MHz, MeOD-d₄) δ 7.83 (s, 1H), 7.70 (t, 2H), 7.22 (t, 2H), 7.14 (s, 1H), 6.34 (s, 1H), 5.81 (t, 1H), 3.99 (t, 1H), 3.65 (t, 1H), 2.29 (t, 3H); LC-MS (ESI) m/z 444 (M+H)⁺.

### Example 87

### Preparation of 2-(1-(4-fluorophenyl)ethyl)-N-(1H-pyrazol-3-yl)pyrrolo[1,2-f][1,2,4]triazin-4-amine

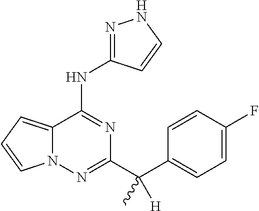

To a solution of 1-(4-(1H-pyrazol-3-ylamino)pyrrolo[1,2-f][1,2,4]triazin-2-yl)-1-(4-fluorophenyl)ethanol from Example 66 (90 mg, 0.26 mmol) in TFA (3 mL) were added triethylsilane (2 mL) and methanesulfonic acid (2 drops). The mixture was stirred in a sealed tube at rt overnight, then concentrated; The residue was purified on silica gel eluting with MeOH/DCM. The mixture of products obtained (95 mg) was dissolved in MeOH (4 mL) and hydrogenated at 1 atm of H₂ in the presence 10% Pd/C for 20 h. The catalyst was removed by filtration through a short pad of Celite, the filtrate was concentrated to dryness, and the crude mixture was purified by reverse phase HPLC to yield 2-(1-(4-fluorophenyl)ethyl)-N-(1H-pyrazol-3-yl)pyrrolo[1,2-f][1,2,4]triazin-4-amine as a white solid (15 mg, 18%). ¹H NMR (DMSO-d₆): δ 12.45 (s, 1H), 10.56 (s, 1H), 7.67 (s, 2H), 7.42 (dd, 2H), 7.1 (s, 1H), 7.14-7.07 (m, 2H), 6.77 (s, 1H), 6.63 (m, 1H), 4.10 (d, 1H), 1.61 (d, 3H). LC-MS (ESI) m/z 323 (M+H)⁺.

### Example 88

### Preparation of 4-(4-(1H-pyrazol-3-ylamino)pyrrolo[1,2-f][1,2,4]triazin-2-yl)-4-(4-fluorophenyl)butan-1-ol

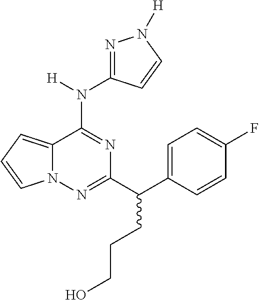

Step A: To a solution of ((2-bromoethoxy)methyl)benzene (1.34 mL, 7.6 mmol) in diethyl ether (20 mL) were added magnesium turnings (0.185 g, 7.6 mmol) and the mixture was heated at reflux for 30 min. The mixture was cooled to −20° C., and a solution of (4-(1H-pyrazol-5-ylamino)pyrrolo[1,2-f][1,2,4]triazin-2-yl)(4-fluorophenyl)methanone (700 mg, 2.17 mmol) in THF (20 mL) was added. The mixture was stirred at −20° C. for 1 hour, then aqueous ammonium chloride was added, and the precipitated solid was collected by filtration, washing with water. The crude solid was purified using chromatography on silica gel eluting with 2-8% MeOH/DCM to afford 1-(4-(1H-pyrazol-5-ylamino)pyrrolo[1,2-f][1,2,4]triazin-2-yl)-4-(benzyloxy)-1-(4-fluorophenyl)butan-1-ol (625 mg, 85%).

Step B: A solution of 1-(4-(1H-pyrazol-5-ylamino)pyrrolo[1,2-f][1,2,4]triazin-2-yl)-1-(4-fluorophenyl)butan-1-ol (625 mg, 1.45 mmol) in trifluoroacetic acid (4 mL) and trifluoroacetic anhydride (4 mL) in a sealed flash under Ar was heated at 60° C. for 60 h. Aqueous sodium bicarbonate was added to pH 7, and the mixture was extracted with ethyl acetate (3×100 mL) and the combined organic layers were dried over sodium sulfate, then filtered and concentrated to yield a brown solid (360 mg). The residue was chromatographed (silica, 20-60% ethyl acetate/hexanes) to afford to afford 220 mg of material containing 1-(4-(1H-pyrazol-5-ylamino)pyrrolo[1,2-f][1,2,4]triazin-2-yl)-1-(4-fluorophenyl)butane-1,4-diol as a significant component. LC-MS (ESI) m/z 383 (M+H)⁺.

Step C: A solution of crude 1-(4-(1H-pyrazol-5-ylamino)pyrrolo[1,2-f][1,2,4]triazin-2-yl)-1-(4-fluorophenyl)butane-1,4-diol (190 mg, 0.50 mmol) in trifluoroacetic acid (2 ml) and trifluoroacetic anhydride (2 ml) in a sealed reaction flask under argon was stirred at 80° C. for 20 h and 60° C. for 50 hours, then heated under microwave irradiation for 15 min at 110° C. and 30 min at 130° C. respectively. The brown reaction solution was concentrated and the residue was partitioned between DCM and water, and the separated organic phase was washed with brine and concentrated. The solid was triturated with DCM to yield a peach solid (140 mg). LC-MS (ESI) m/z 365 (M+H)⁺. To a solution of the above solid (ca. 0.3 mmol) in 3:1 MeOH/H₂O was added LiOH (26.3 mg, 1.1 mmol). After 3.5 h, the mixture was partitioned between DCM and water, then the separated organic phase was washed with brine and concentrated. LCMS LC-MS (ESI) m/z 365 (M+H)⁺. The residue in anhydrous EtOH (5 mL) was stirred at 25° C. overnight under an H₂ atmosphere in the presence of Pd/C (29 mg). Then MeOH (10 mL) and Pd black (30 mg) were added, and hydrogenation was continued at 58 psi for 20 hours. Following filtration, the filtrate was evaporated and the residue was triturated with DCM. The triturates were purified on silica gel eluting with 3-6% MeOH/DCM. Pure fractions were combined and the residue was lyophilized from 1:4 acetonitrile/H₂O and dried under vacuum to afford 4-(4-(1H-pyrazol-5-ylamino)pyrrolo[1,2-f][1,2,4]triazin-2-yl)-4-(4-fluorophenyl)butan-1-ol. LC-MS (ESI) m/z 367 (M+H)⁺; ¹HNMR (300 MHz, DMSO-d₆). δ 12.50 (s, 1H), 10.59 (s, 1H), 7.73 (m, 2H), 7.66 (m, 2H), 7.20 (br, 1H), 7.10 (t, 2H), 6.86 (br s, 1H), 6.63 (dd, 1H), 4.39 (t, 1H), 3.88 (t, 1H), 3.8 (m), 2.21 (m, 1H), 1.95 (m, 1H), 1.37 (m, 2H).

### Example 89

### Preparation of 2-(2-(difluoro(4-fluorophenyl)methyl)-4-(5-methyl-1H-pyrazol-3-ylamino)pyrrolo[1,2-f][1,2,4]triazin-6-yl)propan-2-ol

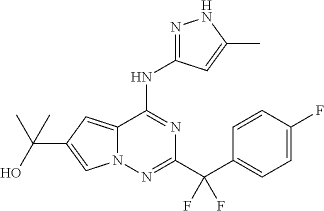

To a solution of methyl 2-(difluoro(4-fluorophenyl)methyl)-4-(5-methyl-1H-pyrazol-3-ylamino)pyrrolo[1,2-f][1,2,4]triazine-6-carboxylate from Example 44 (200 mg, 0.515 mmol) in dry THF (5 mL) at room temperature was added 3 M methylmagnesium bromide in THF (1.7 mL, 5.15 mmol). The mixture was stirred overnight, then the reaction was quenched by adding acetone (0.5 mL). The resulting mixture was diluted with ethyl acetate and 1N HCl (5 mL) was added. The separated aqueous phase was extracted with ethyl acetate (2×15 mL) and the combined organic layers were washed with brine solution and dried over MgSO₄. The solvent was evaporated under vacuum and the crude product was purified on reverse phase HPLC to yield of 2-(2-(difluoro(4-fluorophenyl)methyl)-4-(5-methyl-1H-pyrazol-3-ylamino) pyrrolo[1,2-f][1,2,4]triazin-6-yl)propan-2-ol as a white solid (50 mg, 23%). ¹H NMR (DMSO-_{d6}): δ 12.16 (s, 1H), 10.76 (s, 1H), 7.71 (s, 1H), 7.68 (dd, 2H), 7.32 (t, 2H), 7.20 (s, 1H), 6.23 (s, 1H), 4.98 (s, 1H), 2.21 (s, 3H), 1.46 (s, 6H). LC-MS (ESI) m/z 417 (M+H)⁺.

### Example 90

### Preparation of [2-(4-Fluoro-benzyl)-pyrrolo[2,1-f][1,2,4]triazin-4-yl]-(5-methyl-1H-pyrazol-3-yl)-amine

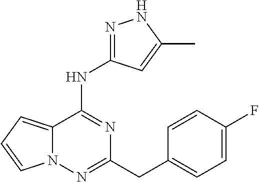

To a solution of (4-fluorophenyl)(4-(5-methyl-1H-pyrazol-3-ylamino)pyrrolo[1,2-f][1,2,4]triazin-2-yl)methanol from Example 2 (100 mg, 0.29 mmol) in TFA (3 mL) at 0° C. were added triethylsilane (2 mL) and methanesulfonic acid (2 drops), and the mixture was heated at 40° C. for 20 h. After cooling to rt, the mixture was concentrated to dryness and the residue was purified on reverse phase HPLC to yield (4-fluorophenyl)(4-(5-methyl-1H-pyrazol-3-ylamino)pyrrolo[1,2-f][1,2,4]triazin-2-yl)methanol as a white solid (67 mg, 72%). ¹H NMR (DMSO-d₆): δ 12.10 (s, 1H), 10.41 (s, 1H), 7.65 (s, 1H), 7.40 (dd, 2H), 7.26-7.10 (m, 3H), 6.61 (d, 1H), 6.39 (s, 1H), 3.91 (s, 2H), 2.22 (s, 3H). LC-MS (ESI) m/z 323 (M+FI)⁺.

### Example 91

### Preparation of (6-fluoro-4-(5-methyl-1H-pyrazol-3-ylamino)pyrrolo[1,2-f][1,2,4]triazin-2-yl)(4-fluorophenyl)methanone

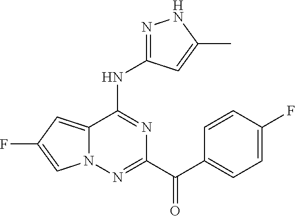

Step A: Methyl 4-fluoro-1H-pyrrole-2-carboxylate was prepared in six steps from (2S,4R)-4-hydroxypyrrolidine-2-carboxylic acid according to Demange et. al. Tetrahedron Lett, 1998, 39, 1169 and Leroy et. al. Tetrahedron 2002, 58, 6713. Methyl 1-amino-4-fluoro-1H-pyrrole-2-carboxylate was synthesized from methyl 4-fluoro-1H-pyrrole-2-carboxylate following the procedure described in Hynes et. al., J. Org. Chem. 2004, 69, 1370. Methyl 1-amino-4-fluoro-1H-pyrrole-2-carboxylate was dissolved in 7N ammonia in MeOH and heated at 120° C. in a sealed tube for 24 h. The solution was concentrated and the residue was triturated with 5:1 petroleum ether/diethylether 1-to afford 1-amino-4-fluoro-1H-pyrrole-2-carboxamide. ¹H NMR (DMSO-d₆): δ 7.97 (bs, 1H), 7.24 (bs, 1H), 6.83 (t, 1H), 6.59 (s, 2H), 6.45 (d, 1H).

Step B: (4-Chloro-6-fluoropyrrolo[1,2-f][1,2,4]triazin-2-yl)(4-fluorophenyl)methanone was obtained from 1-amino-4-fluoro-1H-pyrrole-2-carboxamide following the methods described in General Procedure A (Steps B, C and D) and General Procedure B. Subsequently, General Procedure C was followed using (4-chloro-6-fluoropyrrolo[1,2-f][1,2,4]triazin-2-yl)(4-fluorophenyl)methanone in place of (4-chloropyrrolo[1,2-f][1,2,4]triazin-2-yl)(4-fluorophenyl)methanone. The reaction mixture was diluted with water and the solid was collected by filtration. The crude product was purified by reverse phase HPLC (yield 12%). ¹H NMR (DMSO-d₆): δ 12.24 (bs, 1H), 10.89 (bs, 1H), 8.12 (dd, 2H), 8.00 (m, 1H), 7.39 (t, 2H), 7.26 (s, 1H), 6.51 (s, 1H), 2.18 (s, 3H); LC-MS (ESI) m/z 355 (M+H)⁺.

### Example 92

### Preparation of (6-fluoro-4-(5-methyl-1H-pyrazol-3-ylamino)pyrrolo[1,2-f][1,2,4]triazin-2-yl)(4-fluoronhenyl)methanol

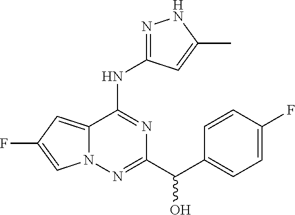

(6-Fluoro-4-(5-methyl-1H-pyrazol-3-ylamino)pyrrolo[1,2-f][1,2,4]triazin-2-yl)(4-fluorophenyl)methanol was obtained following the procedure of Example 2, replacing the (4-fluorophenyl)(4-(5-methyl-1H-pyrazol-3-ylamino)pyrrolo[1,2-f][1,2,4]triazin-2-yl)methanone in Example 2 with (6-fluoro-4-(5-methyl-1H-pyrazol-3-ylamino)pyrrolo[1,2-f][1,2,4]triazin-2-yl)(4-fluorophenyl)methanone from Example 91. Yield 19%. ¹H NMR (DMSO-d₆): δ 11.50 (bs, 1H), 7.94 (s, 1H), 7.55 (dd, 2H), 7.25 (s, 1H), 7.17 (t, 2H), 6.40 (s, 1H), 5.63 (s, 1H), 5.10 (bs, 1H), 2.27 (s, 3H); LC-MS (ESI) m/z 357 (M+H)⁺.

### Example 93

### Preparation of 6-fluoro-2-((4-fluorophenyl)(methoxy)methyl)-N-(5-methyl-1H-pyrazol-3-yl)pyrrolo[1,2-f][1,2,4]triazin-4-amine

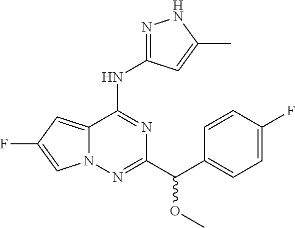

A suspension of (6-fluoro-4-(5-methyl-1H-pyrazol-3-ylamino)pyrrolo[1,2-f][1,2,4]triazin-2-yl)(4-fluorophenyl)methanol from Example 92 (200 mg, 0.56 mmol) and PBr₃ (0.116 mL, 1.12 mmol) in DCM (5 mL) was heated in a sealed tube at 60° C. for 10 min. After cooling to rt, the mixture was diluted with DCM (20 mL) and treated with saturated aq NaHCO₃ (4 mL) and stirred for 0.25 h. The organic phase separated and the aqueous phase was extracted with DCM (10 mL). The combined organic layers were washed with brine, dried over MgSO₄, and concentrated to dryness to yield 2-(bromo(4-fluorophenyl)methyl)-6-fluoro-N-(5-methyl-1H-pyrazol-3-yl)pyrrolo[1,2-f][1,2,4]triazin-4-amine as an off white solid (105 mg, 45%). LC-MS (ESI) m/z 419 (M+H)⁺. A mixture of 2-(bromo(4-fluorophenyl)methyl)-6-fluoro-N-(5-methyl-1H-pyrazol-3-yl)pyrrolo[1,2-f][1,2,4]triazin-4-amine (70 mg, 0.167 mmol) and Ag₂CO₃ (46 mg, 0.167 mmol) in MeOH (10 mL) was heated at 50° C. for 2 h. The mixture was filtered and the filtrate was evaporated to dryness. The crude product was purified by reverse phase HPLC to yield 6-fluoro-2-((4-fluorophenyl)(methoxy)methyl)-N-(5-methyl-1H-pyrazol-3-yl)pyrrolo[1,2-f][1,2,4]triazin-4-amine as a white solid

(14 mg, 10% over two steps). ¹H NMR (DMSO-d₆): δ 12.17 (s, 1H), 10.57 (s, 1H), 7.85 (m, 1H), 7.54 (dd, 2H), 7.18 (t, 2H), 7.07 (s, 1H), 6.46 (s, 1H); LC-MS (ESI) m/z 371 (M+H)⁺.

### Example 94

### Preparation of (441H-pyrazol-3-ylamino)-6-fluoropyrrolo[1,2-f][1,2,4]triazin-2-yl)(4-fluorophenyl)methanone

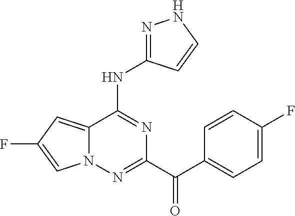

The procedure of Example 5 was followed using (4-chloro-6-fluoropyrrolo[1,2-f][1,2,4]triazin-2-yl)(4-fluorophenyl)methanone from Example 91 in place of (4-chloropyrrolo[1,2-f][1,2,4]triazin-2-yl)(4-fluorophenyl)methanone. The resulting mixture was diluted with water and the solid was collected by filtration. The crude product was purified by reverse phase HPLC to afford (4-(1H-pyrazol-3-ylamino)-6-fluoropyrrolo[1,2-f][1,2,4]triazin-2-yl)(4-fluorophenyl)methanone in 63% yield. ¹H NMR (DMSO-d₆): δ 12.57 (s, 1H), 11.02 (s, 1H), 8.13 (dd, 2H), 8.02 (s, 1H), 7.66 (s, 1H), 7.39 (t, 2H), 7.27 (s, 1H), 6.75 (s, 1H); LC-MS (ESI) m/z 341 (M+H)⁺.

### Example 95

### Preparation of 2-(difluoro(4-fluorophenyl)methyl)-6-fluoro-N-(5-methyl-1H-pyrazol-3-yl)pyrrolo[1,2-f][1,2,4]triazin-4-amine

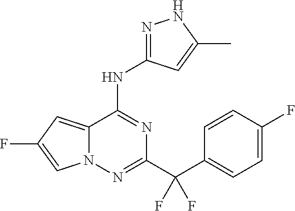

4-Chloro-2-(difluoro(4-fluorophenyl)methyl)-6-fluoropyrrolo[1,2-f][1,2,4]triazine was obtained following the procedure of Example 4, substituting the 1-amino-1H-pyrrole-2-carboxamide in Example 4 with 4-fluoro-1H-pyrrole-2-carboxamide from Example 91. Then General Procedure C was followed, using 4-chloro-2-(difluoro(4-fluorophenyl)methyl)-6-fluoropyrrolo[1,2-f][1,2,4]triazine in place of (4-chloropyrrolo[1,2-f][1,2,4]triazin-2-yl)(4-fluorophenyl)methanone. The resulting mixture was diluted with water and the solid was collected by filtration. The crude product was purified by reverse phase HPLC to afford 2-(difluoro(4-fluorophenyl)methyl)-6-fluoro-N-(5-methyl-1H-pyrazol-3-yl)pyrrolo[1,2-f][1,2,4]triazin-4-amine in 13% yield. ¹H NMR (DMSO-d₆): δ 12.25 (s, 1H), 10.92 (s, 1H), 8.00 (m, 1H), 7.70 (dd, 2H), 7.37 (t, 2H), 7.20 (s, 1H), 6.23 (s, 1H), 2.21 (s, 3H); LC-MS (ESI) m/z 377 (M+H)⁺.

### Example 96

### Preparation of 2-(difluoro(4-fluorophenyl)methyl)-6-fluoro-N-(1H-pyrazol-3-yl)pyrrolo[1,2-f][1,2,4]triazin-4-amine

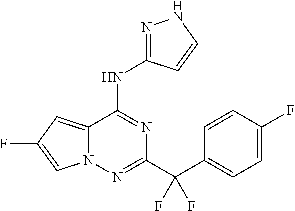

The procedure of Example 5 was followed using 4-chloro-2-(difluoro(4-fluorophenyl)methyl)-6-fluoropyrrolo[1,2-f][1,2,4]triazine from Example 95 in place of the (4-chloropyrrolo[1,2-f][1,2,4]triazin-2-yl)(4-fluorophenyl)methanone in Example 5. The resulting mixture was diluted with water and the solid was collected by filtration. The crude product was purified by reverse phase HPLC to afford 2-(difluoro(4-fluorophenyl)methyl)-6-fluoro-N-(1H-pyrazol-3-yl)pyrrolo[1,2-f][1,2,4]triazin-4-amine in 11% yield. ¹H NMR (DMSO-d₆): δ 12.59 (s, 1H), 11.05 (s, 1H), 8.01 (m, 1H), 7.69 (m, 3H), 7.35 (t, 2H), 7.20 (s, 1H), 6.63 (s, 1H). LC-MS (ESI) m/z 363 (M+H)⁺.

### Example 97

### Preparation of (2-(difluoro(4-fluorophenyl)methyl)-4-(5-methyl-1H-pyrazol-3-ylamino)pyrrolo[1,2-f][1,2,4]triazin-6-yl)(morpholino) methanethione

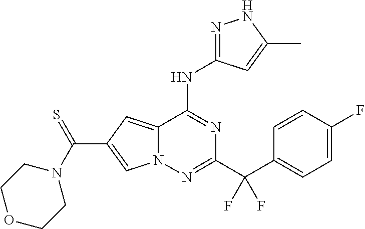

A mixture of (2-(difluoro(4-fluorophenyl)methyl)-4-(5-methyl-1H-pyrazol-3-ylamino)pyrrolo[1,2-f][1,2,4]triazin-6-yl)(morpholino)methanone (21 mg, 0.05 mmol) and Lawesson reagent (40 mg, 0.1 mmol) in anhydrous xylene (2 mL) was heated at 150° C. for 24 h. The solvent was evaporated under vacuum and the residue was purified on silica gel eluting with DCM/MeOH to afford 6-fluoro-2-((4-fluorophenyl) (methoxy)methyl)-N-(5-methyl-1H-pyrazol-3-yl)pyrrolo[1,2-f][1,2,4]triazin-4-amine as a yellow solid (10 mg, 48%). ¹H NMR (DMSO-d₆): δ 12.26 (s, 1H), 11.08 (s, 1H), 8.04 (s, 1H), 7.71 (dd, 2H), 7.48 (s, 1H), 7.38 (t, 2H), 6.26 (s, 1H), 4.33 (s, 2H), 3.93 (s, 2H), 3.80 (m, 2H), 3.68 (s, 2H), 2.23 (s, 3H); LC-MS (ESI) m/z 488 (M+H)⁺.

### Example 98

### Preparation of 2-(difluoro(4-fluorophenyl)methyl)-N-(5-methyl-1H-pyrazol-3-yl)-6-(methylsulfonylmethyl)pyrrolo[1,2-f][1,2,4]triazin-4-amine

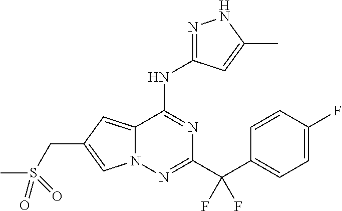

To a solution of 6-(bromomethyl)-2-(difluoro(4-fluorophenyl)methyl)-N-(5-methyl-1H-pyrazol-3-yl)pyrrolo[1,2-f][1,2,4]triazin-4-amine from Example 52, Step A (400 mg, 0.9 mmol) in DMF (10 mL) was added sodium methanethiolate (252 mg, 3.6 mmol) at room temperature. The reaction mixture was heated at 50° C. overnight, then the mixture was diluted with water (100 mL) and extracted with EtOAc (3×100 mL). The combined organic layers were dried over MgSO₄, filtered, and concentrated. The residue was dissolved in 3/1 MeOH/THF (40 mL), and OXONE (3.0 g, 4.8 mmol) in water (8 mL) was added at room temperature, and the mixture was stirred overnight. The mixture was concentrated to a small volume and the residue was diluted with water (20 mL). The resulting mixture was extracted with EtOAc (3×80 mL), and the combined organic layers were washed with brine and dried over Na₂SO₄. The solvent was evaporated under vacuum and the residue purified by reverse phase HPLC to afford 2-(difluoro(4-fluorophenyl)methyl)-N-(5-methyl-1H-pyrazol-3-yl)-6-(methylsulfonylmethyl)pyrrolo[1,2-f][1,2,4]triazin-4-amine as a white solid (8 mg, 2%). ¹H NMR (DMSO-d₆): δ 12.22 (s, 1H), 11.01 (s, 1H), 7.87 (d, 1H), 7.73-768 (m, 2H), 7.42-7.33 (m, 3H), 6.23 (s, 1H), 4.52 (s, 2H), 2.93 (s, 3H), 2.22 (s, 3H); LC-MS (ESI) m/z 451 (M+H)⁺.

### Example 99

### Preparation of N-(2-(difluoro(4-fluorophenyl)methyl)-4-(5-methyl-1H-pyrazol-3-ylamino)pyrrolo[1,2-f][1,2,4]triazin-6-yl)-3-morpholinopropanamide

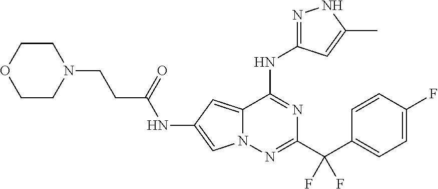

A mixture of 2-(difluoro(4-fluorophenyl)methyl)-N-(5-methyl-1H-pyrazol-3-yl)-6-nitropyrrolo[1,2-f][1,2,4]triazin-4-amine from Example 46 Step F (300 mg, 0.74 mmol), 10% Pd/C (50 mg), and 10% HCl (5 mL) in MeOH (15 mL) was stirred under a hydrogen atmosphere for 1.5 h. The mixture was filtered through Celite washing with MeOH. The filtration was concentrated and ethyl ether was added to the residue to form a precipitate, which was collected by filtration and dried under vacuum to afford a solid (292 mg).

To a solution of 3-morpholinopropanoic acid (159 mg, 1 mmol) in DMF (8 mL) was added HATU (456 mg, 1.2 mmol), followed by addition of the above solid and iPr₂NEt (1 mL). The mixture was stirred at room temperature overnight, then quenched with water and extracted with DCM. Extracts were dried over MgSO₄ and concentrated, and the residue was purified by silica gel chromatography using a mixture of EtOAc-hexanes as eluent to afford N-(2-(difluoro(4-fluorophenyl)methyl)-4-(5-methyl-1H-pyrazol-3-ylamino)pyrrolo[1,2-f][1,2,4]triazin-6-yl)-3-morpholinopropanamide (0.39 mg, 10%) as a solid. ¹H NMR (300 MHz, DMSO-d₆) δ 12.20 (s, 1H), 10.80 (s, 1H), 10.50 (s, 1H), 8.04 (d, 1H), 7.69 (dd, 2H), 7.36 (t, 2H), 7.22 (s, 1H), 6.20 (s, 1H), 3.56 (t, 4H), 2.63 (t, 2H), 2.50 (overlapping with solvent, 2H), 2.41 (t, 4H), 2.21 (s, 3H); LC-MS (ESI) m/z 515 (M+H)⁺.

### Example 100

### Preparation of N-(5-chloro-2-(difluoro(4-fluorophenyl)methyl)-4-(5-methyl-1H-pyrazol-3-ylamino)pyrrolo[1,2-f][1,2,4]triazin-6-yl)-3-morpholinopropanamide

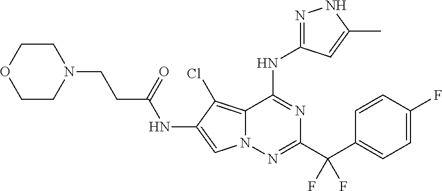

The silica gel chromatography of Example 99 also afforded N-(5-chloro-2-(difluoro(4-fluorophenyl)methyl)-4-(5-methyl-1H-pyrazol-3-ylamino)pyrrolo[1,2-f][1,2,4]triazin-6-yl)-3-morpholinopropanamide (0.19 mg, 5%) as a solid. ¹H NMR (300 MHz, DMSO-d₆) δ 12.21 (s, 1H), 11.01 (s, 1H), 10.33 (s, 1H), 7.90 (s, 1H), 7.71 (dd, 2H), 7.37 (t, 2H), 6.16 (s, 1H), 3.63 (t, 4H), 2.64 (t, 2H), 2.56 (t, 2H), 2.50 (overlapping with solvent, 4H), 2.27 (s, 3H); LC-MS (ESI) m/z 549 (M+H)⁺.

### Example 101

### Preparation of N-(2-(difluoro(4-fluorophenyl)methyl)-4-(5-methyl-1H-pyrazol-3-ylamino)pyrrolo[1,2-f][1,2,4]triazin-6-yl)acrylamide

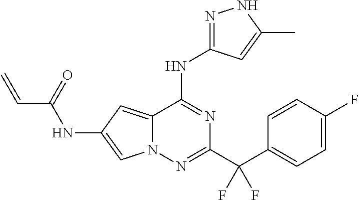

To a suspension of 2-(difluoro(4-fluorophenyl)methyl)-N⁴-(5-methyl-1H-pyrazol-3-yl)pyrrolo[1,2-f][1,2,4]triazine-4,6-diamine from Example 46 Step G (200 mg, 0.49 mmol) in DCM under argon was added 3-chloropropanoyl chloride (89 mg, 0.7 mmol), followed by addition of saturated NaHCO₃ solution (5 mL). The mixture was stirred at rt for 6 h, then quenched with water and extracted with DCM. The organic extracts were dried over MgSO₄ and concentrated. To the residue was added EtOAc (10 mL) and 10% NaOH solution (5 mL) and the mixture was stirred at rt for 30 min. The mixture was diluted with water and extracted with EtOAc, and the extracts were dried over MgSO₄ and concentrated to afford a solid (195 mg).

To the above solid dissolved in DMF (10 mL), were added KI (90 mg), 3,3-difluoropyrrolidine hydrochloride (90 mg, 0.63 mmol), and DIPEA (0.6 mL) and the mixture was heated at 60° C. for 7 h. Additional 3,3-difluoropyrrolidine hydrochloride (60 mg, 0.31 mmol) and iPr₂NEt (0.5 mL) were added and the mixture was heated at 80° C. overnight. The mixture was concentrated, diluted with water, and extracted with DCM. The combined extracts were dried over MgSO₄ and concentrated, and the residue was purified by silica gel chromatography using EtOAc-hexanes as eluent to afford N-(2-(difluoro(4-fluorophenyl)methyl)-4-(5-methyl-1H-pyrazol-3-ylamino)pyrrolo[1,2-f][1,2,4]triazin-6-yl)acrylamide (0.44 mg, 21%) as a solid. ¹H NMR: (300 MHz, DMSO-d₆) δ 12.21 (s, 1H), 10.86 (s, 1H), 10.63 (s, 1H), 8.13 (d, 1H), 7.71 (dd, 2H), 7.36 (t, 2H), 7.28 (s, 1H), 6.44 (dd, 1H), 6.24-6.30 (m, 2H), 5.75 (dd, 1H), 2.22 (s, 3H); LC-MS (ESI) m/z 428 (M+H)⁺.

### Example 102

### Preparation of N-(2-(difluoro(4-fluorophenyl)methyl)-4-(5-methyl-1H-pyrazol-3-ylamino)pyrrolo[1,2-f][1,2,4]triazin-6-yl)-2-(4,4-difluoropiperidin-1-yl)ethanesulfonamide

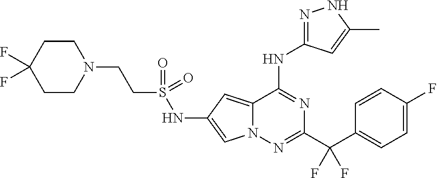

Step A: To a suspension of 2-(difluoro(4-fluorophenyl)methyl)-N⁴-(5-methyl-1H-pyrazol-3-yl)pyrrolo[1,2-f][1,2,4]triazine-4,6-diamine (738 mg, 1.8 mmol) in DCM (15 mL) under argon was added 2-chloroethanesulfonyl chloride (326 mg, 2 mmol), followed by addition of saturated aq NaHCO₃ (10 mL). The mixture was stirred at rt for 6 h, then additional 2-chloroethanesulfonyl chloride (100 mg, 0.6 mmol) was added, and stirring was continued at rt overnight. The reaction was quenched by addition of saturated aq NaHCO₃, and the resulting mixture was extracted with DCM. The combined extracts were dried over MgSO₄ and concentrated, and the residue was purified by silica gel chromatography eluting with EtOAc-hexanes to afford N-(2-(difluoro(4-fluorophenyl)methyl)-4-(5-methyl-1H-pyrazol-3-ylamino)pyrrolo[1,2-f][1,2,4]triazin-6-yl)ethenesulfonamide (0.75 mg, 9%) as a solid. ¹H NMR (300 MHz, DMSO-d₆) δ 9.7 (br, 1H), 9.1 (br, 1H), 7.71 (dd, 2H), 7.60 (d, 1H), 7.14 (t, 2H), 6.76 (s, 1H), 6.51-6.62 (m, 3H), 6.25 (d, 1H), 5.99 (d, 1H), 2.35 (s, 3H); LC-MS (ESI) m/z 464 (M+H)⁺.

Step B: A mixture of N-(2-(difluoro(4-fluorophenyl)methyl)-4-(5-methyl-1H-pyrazol-3-ylamino)pyrrolo[1,2-f][1,2,4]triazin-6-yl)ethenesulfonamide (75 mg, 0.16 mmol), 4,4-difluoropiperidine hydrochloride (79 mg, 0.5 mmol), and iPr₂NEt (0.5 mL) in CH₃CN (10 mL) was heated at 90° C. for 8 h. The reaction was quenched by addition of saturated aq NaHCO₃ and the resulting mixture was extracted with DCM. The extracts were dried over MgSO₄ and concentrated, and the residue was purified by silica gel chromatography eluting with EtOAc-hexanes to afford N-(2-(difluoro(4-fluorophenyl)methyl)-4-(5-methyl-1H-pyrazol-3-ylamino)pyrrolo[1,2-f][1,2,4]triazin-6-yl)-2-(4,4-difluoropiperidin-1-yl)ethanesulfonamide (0.60 mg, 64%) as a solid. ¹H NMR (300 MHz, DMSO-d₆) δ 12.20 (s, 1H), 10.89 (s, 1H), 9.87 (s, 1H), 7.69 (dd, 2H), 7.61 (d, 1H), 7.36 (t, 2H), 7.22 (s, 1H), 6.22 (s, 1H), 3.29 (t, 2H), 2.77 (t, 2H), 2.47 (m, 4H), 2.21 (s, 3H), 1.87 (m, 4H); LC-MS (ESI) m/z 585 (M+H)⁺.

### Example 103

### Preparation of (7-bromo-4-(5-methyl-1H-pyrazol-3-ylamino)pyrrolo[1,2-f][1,2,4]triazin-2-yl)(4-fluorophenyl)methanone

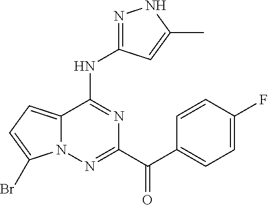

Step A: To a mixture of ethyl 4-chloropyrrolo[1,2-f][1,2,4]triazine-2-carboxylate from General Procedure A Step D (940 mg, 4.2 mmol) in THF (12 mL) was added NBS (890 mg, 5 mmol). The mixture was stirred at rt for 5 h, then the reaction was quenched by addition of saturated aq NaHCO₃, and the resulting mixture was extracted with DCM, the combined extracts were dried over MgSO₄, filtered, and concentrated, and the residue was purified by silica gel chromatography eluting with EtOAc-hexanes to afford ethyl 7-bromo-4-chloropyrrolo[1,2-f][1,2,4]triazine-2-carboxylate (1.154 g, 91%) as a solid. ¹H NMR (300 MHz, CDCl₃) δ 7.26 (s, 1H), 7.17 (s, 1H), 4.55 (q, 2H), 1.58 (t, 3H); LC-MS (ESI) m/z 304 (M+H)⁺.

Step B: To a solution of ethyl 7-bromo-4-chloropyrrolo[1,2-f][1,2,4]triazine-2-carboxylate (152 mg, 0.5 mmol) in THF (10 mL) at −40° C. was added a 1.0 M solution of (4-fluorophenyl)magnesium bromide in THF (0.6 mL, 0.6 mmol). The mixture was stirred for 4 h at −40° C., then the reaction was quenched with water and the resulting mixture was extracted with EtOAc. The organic layer was separated, dried, and concentrated, and the residue was purified by silica gel chromatography eluting with EtOAc-hexanes to afford (7-bromo-4-chloropyrrolo[1,2-f][1,2,4]triazin-2-yl)(4-fluorophenyl)methanone (61 mg, 34%) as a solid. ¹H NMR (300 MHz, CDCl₃) δ 8.28 (d, 1H), 8.25 (d, 1H), 7.18-7.24 (m, 4H); LC-MS (ESI) m/z 354 (M+H)⁺.

Step C: (7-Bromo-4-(5-methyl-1H-pyrazol-3-ylamino)pyrrolo[1,2-f][1,2,4]triazin-2-yl)(4-fluorophenyl)methanone was obtained following the procedure described in Example 44 Step G, substituting methyl 4-chloro-2-(difluoro(4-fluorophenyl)methyl)pyrrolo[1,2-f][1,2,4]triazine-6-carboxylate in Example 44 with (7-bromo-4-chloropyrrolo[1,2-f][1,2,4]triazin-2-yl)(4-fluorophenyl)methanone (91 mg, 44% yield). ¹H NMR (300 MHz, DMSO-d₆) δ 12.25 (s, 1H), 11.00 (s, 1H), 8.17 (m, 2H), 7.36-7.44 (m, 3H), 7.01 (d, 1H), 6.52 (s, 1H), 1.99 (s, 3H); LC-MS (ESI) m/z 415 (M+H)⁺.

### Example 104

### Preparation of 2-(4-fluorophenyl)-2-(4-(5-methyl-1H-pyrazol-3-ylamino)pyrrolo[1,2-f][1,2,4]triazin-2-yl)ethanol

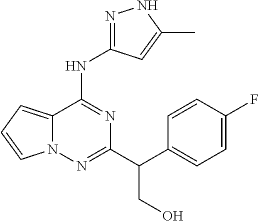

Step A. To trimethylsulfoxonium chloride (1 g, 11.9 mmol) in DMSO (20 mL) was added 60% sodium hydride in mineral oil (476 mg, 11.9 mmol) and the solution was stirred under argon at room temperature for 30 min. Then (4-fluorophenyl)(4-(5-methyl-1H-pyrazol-3-ylamino)pyrrolo[1,2-f][1,2,4]triazin-2-yl)methanone (1.52 g, 2.9 mmol) in DMSO (5 mL) was added, and the solution was stirred at rt for 3 h. The reaction was quenched with aq ammonium chloride, and then the mixture was extracted with ethyl acetate. The organic phase was dried over sodium sulfate and evaporated. Purification of the residue by silica gel chromatography (DCM/methanol 0-10%) afforded 2-(2-(4-fluorophenyl)oxiran-2-yl)-N-(5-methyl-1H-pyrazol-3-yl)pyrrolo[1,2-f][1,2,4]triazin-4-amine (685 mg, 67%). LC-MS (ESI) m/z 351 (M+H)⁺.

Step B. To a solution of 2-(2-(4-fluorophenyl)oxiran-2-yl)-N-(5-methyl-1H-pyrazol-3-yl)pyrrolo[1,2-f][1,2,4]triazin-4-amine (70 mg, 0.2 mmol) in MeOH (5 mL) was added 10% palladium on carbon (70 mg) and the mixture stirred under an atmosphere of H₂ overnight. The mixture was filtered and the filtrate was purified by preparative HPLC (Varian diphenyl reverse phase column, eluted with gradient of solvent B=0.05% HOAC/CH₃CN and solvent A=0.05% HOAc/H₂O) to afford 2-(4-fluorophenyl)-2-(4-(5-methyl-1H-pyrazol-3-ylamino)pyrrolo[1,2-f][1,2,4]triazin-2-yl)ethanol (18 mg, 25%). ¹H NMR: (300 MHz, DMSO-d₆) δ 2.27 (s, 3H), 3.85 (m, 1H), 4.03 (t, 1H), 4.2 (m, 1H), 4.81 (t, 1H), 6.53 (s, 1H), 6.62 (m, 1H), 7.08-7.17 (m, 3H), 7.45 (m, 2H), 7.65 (m, 1H), 10.4 (s, 1H), 12.03 (bs, 1H); LC-MS (ESI) m/z 353 (M+H)⁺.

### Example 105

### Preparation of 1-(4-fluorophenyl)-1-(4-(5-methyl-1H-pyrazol-3-ylamino)pyrrolo[1,2-f][1,2,4]triazin-2-yl)ethane-1,2-diol

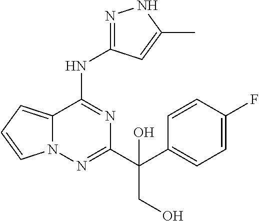

Step A. To 2-(2-(4-fluorophenyl)oxiran-2-yl)-N-(5-methyl-1H-pyrazol-3-yl)pyrrolo[1,2-f][1,2,4]triazin-4-amine from Example 104, Step A (78 mg, 0.22 mmol) in DCM (1 mL) were added triethylsilane (0.11 mL, 0.67 mmol) followed by trifluoroacetic acid (1 mL). The solution was kept at room temperature for 25 min, then methansulfonic acid (0.05 mL) was added, and the solution was stirred at rt for 3 h. Then borane trifluoride diethyl etherate (2 drops) was added and the solution was stirred at rt overnight. Then additional triethylsilane (0.3 mL) and acetonitrile (1 mL) were added, and the solution was heated at 50° C. for 1 h. Then 10% aq potassium carbonate was added, and the mixture was extracted with ethyl acetate. The organic layer was evaporated and the residue was purified by preparative HPLC (Varian diphenyl reverse phase column, eluted with gradient of solvent B=0.05% HOAC/CH₃CN and solvent A=0.05% HOAc/H₂O) to afford 1-(4-fluorophenyl)-1-(4-(5-methyl-1H-pyrazol-3-ylamino)pyrrolo[1,2-f][1,2,4]triazin-2-yl)ethane-1,2-diol (13 mg, 16%) ¹H NMR: (300 MHz, DMSO-d₆) δ 2.25 (s, 3H), 3.85 (dd, 1H), 4.18 (dd, 1H), 4.80 (t, 1H), 5.47 (s, 1H), 6.34 (s, 1H), 6.68 (m, 1H), 7.05-7.20 (m, 3H), 7.64 (m, 2H), 7.72 (s, 1H), 10.38 (s, 1H), 12.30 (bs, 1H); LC-MS (ESI) m/z 369 (M+H)⁺.

### Example 106

### Preparation of 2-fluoro-2-(4-fluorophenyl)-2-(4-(5-methyl-1H-pyrazol-3-ylamino)pyrrolo[1,2-f][1,2,4]triazin-2-yl)ethanol

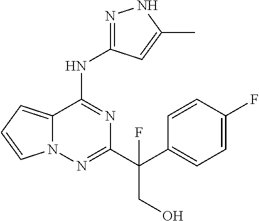

Step A. To 2-(2-(4-fluorophenyl)oxiran-2-yl)-N-(5-methyl-1H-pyrazol-3-yl)pyrrolo[1,2-f][1,2,4]triazin-4-amine, Example 104, Step A, (67 mg, 0.19 mmol) in DCM (5 mL) at 0° C. was added 70% hydrogen fluoride/pyridine (0.03 mL, 0.95 mmol). The solution was allowed to warm to rt and stir for 1.5 h, then additional 70% hydrogen fluoride/pyridine (0.1 mL) was added, and the mixture was stirred for 15 min. Then triethylamine (0.4 mL) was added and the solution was concentrated. The residue was purified by preparative HPLC (Varian diphenyl reverse phase column, eluted with gradient of solvent B=0.05% HOAC/CH₃CN and, solvent A=0.05% HOAc/H₂O) to afford 2-fluoro-2-(4-fluorophenyl)-2-(4-(5-methyl-1H-pyrazol-3-ylamino)pyrrolo[1,2-f][1,2,4]triazin-2-yl)ethanol (14 mg, 19%) ¹H NMR: (300 MHz, DMSO-d₆) δ 2.24 (s, 3H), 4.1-4.4 (m, 2H), 5.25 (m, 1H), 6.33 (s, 1H), 6.70 (m, 1H), 7.18-7.24 (m, 3H), 7.54 (m, 2H), 7.75 (s, 1H), 10.62 (s, 1H), 12.15 (bs, 1H); LC-MS (ESI) m/z 371 (M+H)⁺.

### Example 107

### Preparation of 2-(1-fluoro-1-(4-fluoronhenyl)-2-methoxyethyl)-N-(5-methyl-1H-pyrazol-3-yl)pyrrolo[1,2-f][1,2,4]triazin amine

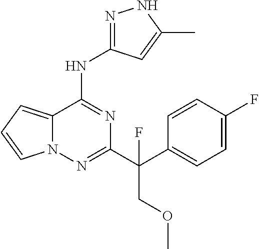

Step A: To 2-(2-(4-fluorophenyl)oxiran-2-yl)-N-(5-methyl-1H-pyrazol-3-yl)pyrrolo[1,2-f][1,2,4]triazin-4-amine from Example 104 Step A (200 mg, 0.57 mmol) in MeOH (8 mL) was added 25% NaOMe/MeOH (1.6 mL) and the mixture was stirred at 60° C. overnight in a sealed vial. Aq ammonium chloride was added and the resulting mixture was extracted with EtOAc. The organic layer was dried over sodium sulfate and concentrated, and the residue was purified by silica gel chromatography eluting with DCM/MeOH (0-10%) to afford 1-(4-fluorophenyl)-2-methoxy-1-(4-(5-methyl-1H-pyrazol-3-ylamino)pyrrolo[1,2-f][1,2,4]triazin-2-yl)ethanol (180 mg, 82%). LC-MS (ESI) m/z 383 (M+H)⁺.

Step B: To 1-(4-fluorophenyl)-2-methoxy-1-(4-(5-methyl-1H-pyrazol-3-ylamino)pyrrolo[1,2-f][1,2,4]triazin-2-yl)ethanol (82 mg, 0.21 mmol) in DCM (5 mL) at −70° C. under argon was added diethylaminosulfur trifluoride (0.2 mL, 1.51 mmol). The mixture was stirred for 15 min and then aq sodium bicarbonate was added. The resulting mixture was extracted with DCM and the organic layer was dried over sodium sulfate and concentrated. The residue was purified by preparative HPLC (Varian diphenyl reverse phase column, eluted with gradient of solvent B=0.05% HOAC/CH₃CN and solvent A=0.05% HOAc/H₂O) to afford 2-fluoro-2-(4-fluorophenyl)-2-(4-(5-methyl-1H-pyrazol-3-ylamino)pyrrolo[1,2-f][1,2,4]triazin-2-yl)ethanol (7 mg, 9%) ¹H NMR: (300 MHz, DMSO-d₆) δ 2.23 (s, 3H), 3.33 (s, 3H), 4.15-4.36 (m, 2H), 6.28 (s, 1H), 6.70 (m, 1H), 7.19-7.25 (m, 3H), 7.51 (m, 2H), 7.76 (m, 1H), 10.66 (s, 1H), 12.18 (bs, 1H); LC-MS (ESI) m/z 385 (M+H)⁺.

### Example 108

### Preparation of 2-(2-amino-1-fluoro-1-(4-fluorophenyl)ethyl)-N-(5-methyl-1H-pyrazol-3-yl)pyrrolo[1,2-f][1,2,4]triazin-4-amine

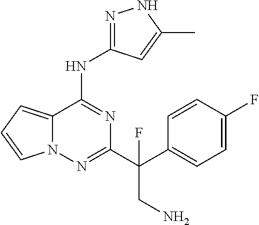

To 2-(2-(4-fluorophenyl)oxiran-2-yl)-N-(5-methyl-1H-pyrazol-3-yl)pyrrolo[1,2-f][1,2,4]triazin-4-amine from Example 104 Step A (100 mg, 0.28 mmol) in DMF (2 mL) was added sodium azide (120 mg) and the mixture was heated at 85° C. for 2 h. Water was added and the resulting mixture was extracted with EtOAc. The organic layer was dried over sodium sulfate and concentrated. To the residue in DCM (5 mL) at 0° C. was added diethylaminosulfur trifluoride (0.12 mL, 0.85 mmol), and the mixture was stirred for 45 min. The resulting mixture was filtered through a silica plug washing with DCM and MeOH. The filtrate was concentrated, and the residue was dissolved in MeOH (5 mL) and acetic acid (0.1 mL), then 10% palladium on carbon (100 mg) was added and the mixture was stirred under an atmosphere of hydrogen overnight. The mixture was filtered, the filtrate was concentrated, and the residue was purified by silica gel chromatography eluting with 0-10% DCM/MeOH. The isolated material (90 mg) was dissolved in MeOH (10 mL), 10% palladium on carbon (90 mg) was added, and the mixture was stirred under an atmosphere of hydrogen for 2 h. The resulting mixture was filtered, and the filtrate was purified by preparative HPLC (Varian diphenyl reverse phase column, eluted with gradient of solvent B=0.05% HOAC/CH₃CN and solvent A=0.05% HOAc/H₂O) to afford 2-(2-amino-1-fluoro-1-(4-fluorophenyl)ethyl)-N-(5-methyl-1H-pyrazol-3-yl)pyrrolo[1,2-f][1,2,4]triazin-4-amine as the acetate salt (4 mg, 4%) ¹H NMR: (300 MHz, DMSO-d₆) δ 1.89 (s, 3H), 2.24 (s, 3H), 3.5 (m, 2H), 6.35 (s, 1H), 6.69 (m, 1H), 7.18-7.24 (m, 3H), 7.52 (m, 2H), 7.75 (s, 1H), 10.67 (bs, 1H); LC-MS (ESI) m/z 370 (M+H)⁺.

### Example 109

### Preparation of 5-(4-fluorophenyl)-5-(4-(5-methyl-1H-pyrazol-3-ylamino)pyrrolo[1,2-f][1,2,4]triazin-2-yl)oxazolidin-2-one

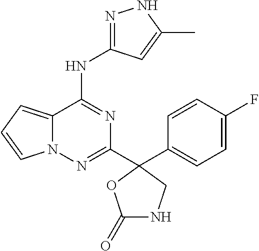

To 2-(2-(4-fluorophenyl)oxiran-2-yl)-N-(5-methyl-1H-pyrazol-3-yl)pyrrolo[1,2-f][1,2,4]triazin-4-amine from Example 104 Step A (220 mg, 0.62 mmol) in DMF (2 mL) was added sodium azide (204 mg, 3.14 mmol) and the mixture was heated at 85° C. for 3 h. Water was added and the resulting mixture was extracted with EtOAc. The organic layer was dried over sodium sulfate and concentrated. The residue was dissolved in MeOH (10 mL), then 10% palladium on carbon (50 mg) was added and the mixture was stirred under an atmosphere of hydrogen for 1 h. The resulting mixture was filtered and concentrated, then THF (10 mL) was added and the solution was cooled to 0° C. Carbonyldiimidazole (100 mg, 0.62 mmol) was added and the mixture was allowed to warm to rt and stir overnight. The solution was then heated at 65° C. for 2.5 h before additional carbonyldiimidazole (250 mg) was added. Heating at 65° C. was continued for 1 h, then the mixture was purified by preparative HPLC (Varian diphenyl reverse phase column, eluted with gradient of solvent B=0.05% HOAC/CH₃CN and solvent A=0.05% HOAc/H₂O) to afford 544-fluorophenyl)-5-(4-(5-methyl-1H-pyrazol-3-ylamino)pyrrolo[1,2-f][1,2,4]triazin-2-yl)oxazolidin-2-one (85 mg, 35%) ¹H NMR: (300 MHz, DMSO-d₆) δ 2.3 (s, 3H), 3.88 (d, 1H), 4.58 (d, 1H), 6.26 (s, 1H), 6.71 (m, 1H), 7.22-7.28 (m, 3H), 7.53 (m, 2H), 7.75 (m, 1H), 7.94 (s, 1H), 10.70 (s, 1H), 12.17 (bs, 1H); LC-MS (ESI) m/z 394 (M+H)⁺.

### Example 110

### Preparation of 2-((4-fluorophenyl)(methoxyamino)methyl)-N-(5-methyl-1H-pyrazol-3-yl)pyrrolo[1,2-f][1,2,4]triazin-4-amine

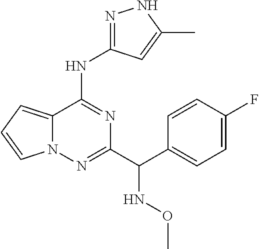

Step A: To (4-fluorophenyl)(4-(5-methyl-1H-pyrazol-3-ylamino)pyrrolo[1,2-f][1,2,4]triazin-2-yl)methanone from Example 1 (500 mg, 1.48 mmol) in EtOH (4 mL) was added O-methylhydroxylamine hydrochloride (248 mg, 2.97 mmol) and the solution heated at 110° C. in a microwave reactor for 15 min. Water was added and the resulting suspension was filtered. The collected solid was dried to afford (4-fluorophenyl)(4-(5-methyl-1H-pyrazol-3-ylamino)pyrrolo[1,2-f][1,2,4]triazin-2-yl)methanone O-methyl oxime as a mixture of isomers (540 mg, quantitative) LC-MS (ESI) m/z 366 (M+H)⁺.

Step B: To (4-fluorophenyl)(4-(5-methyl-1H-pyrazol-3-ylamino)pyrrolo[1,2-f][1,2,4]triazin-2-yl)methanone O-methyl oxime (50 mg, 0.14 mmol) in THF (1 mL) was added lithium aluminum hydride (20 mg) and the mixture was stirred for 20 min at rt. Methanol (3 mL) was added and the mixture was filtered. To the filtrate was added acetic acid (0.2 mL) and the resulting solution was purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(methoxyamino)methyl)-N-(5-methyl-1H-pyrazol-3-yl)pyrrolo[1,2-f][1,2,4]triazin-4-amine (7 mg, 14%) ¹H NMR: (300 MHz, DMSO-d₆) δ 2.27 (s, 3H), 3.44 (s, 3H), 5.08 (d, 1H), 6.56 (bs, 1H), 6.65 (m, 1H), 7.15-7.22 (m, 3H), 7.54 (m, 2H), 7.68 (s, 1H), 10.56 (bs, 1H), 12.17 (bs, 1H); LC-MS (ESI) m/z 368 (M+H)⁺.

### Example 111

### Preparation of 2-(2-(4-fluorophenyl)-1,3-dioxolan-2-yl)-N-(5-methyl-1H-pyrazol-3-yl)pyrrolo[1,2-f][1,2,4]triazin-4-amine

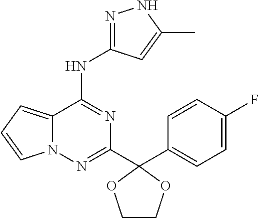

To (4-fluorophenyl)(4-(5-methyl-1H-pyrazol-3-ylamino)pyrrolo[1,2-f][1,2,4]triazin-2-yl)methanone from Example 1 (180 mg, 0.53 mmol) in toluene (2 mL) were added ethylene glycol (1 mL) and methanesulfonic acid (0.1 mL) and the solution was heated at 110° C. for 1.5 h, followed by heating at 50° C. for 3 days. The resulting mixture was concentrated, then triethylamine (0.3 mL) was added and the mixture was purified by preparative HPLC (Varian diphenyl reverse phase column, eluted with gradient of solvent B=0.05% HOAC/CH₃CN and solvent A=0.05% HOAc/H₂O) to afford 2-(2-(4-fluorophenyl)-1,3-dioxolan-2-yl)-N-(5-methyl-1H-pyrazol-3-yl)pyrrolo[1,2-f][1,2,4]triazin-4-amine (46 mg, 23%) ¹H NMR: (300 MHz, DMSO-d₆) δ 2.21 (s, 3H), 3.98-4.2 (m, 4H), 6.24 (s, 1H), 6.68 (m, 1H), 7.17-7.27 (m, 3H), 7.58 (m, 2H), 7.73 (s, 1H), 10.54 (bs, 1H), 12.11 (bs, 1H); LC-MS (ESI) m/z 381 (M+H)⁺.

### Example 112

### Preparation of 2-(2-(4-fluorophenyl)-1,3-oxathiolan-2-yl)-N-(5-methyl-1H-pyrazol-3-yl)pyrrolo[1,2-f][1,2,4]triazin-4-amine

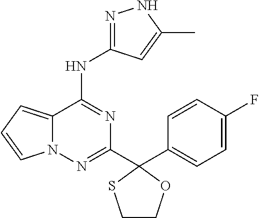

To (4-fluorophenyl)(4-(5-methyl-1H-pyrazol-3-ylamino)pyrrolo[1,2-f][1,2,4]triazin-2-yl)methanone from Example 1 (100 mg, 0.29 mmol) in 1,2-dichloroethane (1.5 mL) were added 2-mercaptoethanol (1 mL) and chlorotrimethylsilane (0.4 mL) and the solution heated at 60° C. overnight. The solution was allowed to cool and DCM was added followed by 10% aq potassium carbonate. The organic layer was concentrated and the residue was purified by preparative HPLC (Varian diphenyl reverse phase column, eluted with gradient of solvent B=0.05% HOAC/CH₃CN and solvent A=0.05% HoAc/H₂O) to afford 2-(2-(4-fluorophenyl)-1,3-oxathiolan-2-yl)-N-(5-methyl-1H-pyrazol-3-yl)pyrrolo[1,2-f][1,2,4]triazin-4-amine (32 mg, 28%). ¹H NMR: (300 MHz, DMSO-d₆) δ 2.25 (s, 3H), 3.1-3.27 (m, 2H), 4.34 (t, 2H), 6.43 (s, 1H), 6.66 (m, 1H), 7.13-7.25 (m, 3H), 7.67 (m, 1H), 7.74 (m, 1H), 10.59 (bs, 1H), 12.16 (bs, 1H); LC-MS (ESI) m/z 397 (M+H)⁺.

### Example 113

### Preparation of 3-(4-fluorophenyl)-3-(4-(5-methyl-1H-pyrazol-3-ylamino)pyrrolo[1,2-f][1,2,4]triazin-2-yl)propan-1-ol

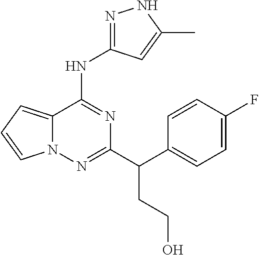

To a suspension of sodium hydride (600 mg, 15 mmol) in dry THF (20 mL) at −10° C. was added dropwise a solution of triethyl phosphonoacetate (3.36 g, 15 mmol). The mixture was stirred at room temperature for 45 min, then cooled to −10° C., whereafter a solution of (4-fluorophenyl)(4-(5-methyl-1H-pyrazol-3-ylamino)pyrrolo[1,2-f][1,2,4]triazin-2-yl)methanone (1.0 g, 3 mmol) in THF was added dropwise and the mixture was stirred at rt overnight. To resulting mixture was diluted with water (100 mL) and extracted with ethyl acetate (150 mL). The organic layer was washed with brine, dried over magnesium sulfate, and concentrated, and the residue was purified by silica gel chromatography to afford ethyl 3-(4-fluorophenyl)-3-(4-(5-methyl-1H-pyrazol-3-ylamino)pyrrolo[1,2-f][1,2,4]triazin-2-yl)acrylate (860 mg, 70%). LC-MS (ESI) m/z 407 (M+H)⁺.

### Example 114

### Preparation of 1-((4-fluorophenyl)(4-(5-methyl-1H-pyrazol-3-ylamino)pyrrolo[1,2-f][1,2,4]triazin-2-yl)methyl)-3-methylurea

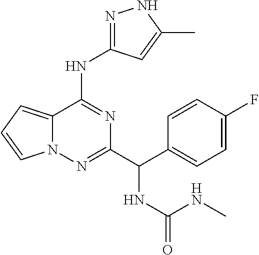

To 2-(amino(4-fluorophenyl)methyl)-N-(5-methyl-1H-pyrazol-3-yl)pyrrolo[1,2-f][1,2,4]triazin-4-amine from Example 69 Step B (170 mg, 0.5 mmol) in THF (5 mL) at 0° C. was added dropwise phenyl chlorformate (0.063 mL, 0.5 mmol), followed by diisopropylethyl amine (0.087 mL, 0.5 mmol). The mixture was stirred for 10 min and then concentrate. THF (2 mL) was added followed by 1M methylamine/THF (5 mL) and the reaction stirred for 3 h at rt. The solvent was evaporated, water was added, and the mixture was extracted with ethyl acetate. The organic phase was concentrated and the residue was purified by preparative HPLC (Varian diphenyl reverse phase column, eluted with gradient of solvent B=0.05% HOAC/CH₃CN and solvent A=0.05% HOAc/H₂O) to afford 1-((4-fluorophenyl)(4-(5-methyl-1H-pyrazol-3-ylamino)pyrrolo[1,2-f][1,2,4]triazin-2-yl)methyl)-3-methylurea (35 mg, 16%). ¹H NMR: (300 MHz, DMSO-d₆) δ 2.25 (s, 3H), 2.56 (d, 3H), 5.77 (d, 1H), 6.19 (m, 1H), 6.46 (s, 1H), 6.66 (t, 1H), 6.8 (m, 1H), 7.06-7.28 (m, 3H), 7.42 (m, 2H), 7.68 (s, 1H), 10.55 (s, 1H), 12.15 (bs, 1H); LC-MS (ESI) m/z 395 (M+H)⁺.

### Example 115

### Preparation of 2-(1-amino-1-(4-fluorophenyl)ethyl)-N-(5-methyl-1H-pyrazol-3-yl)pyrrolo[1,2-f][1,2,4]triazin-4-amine

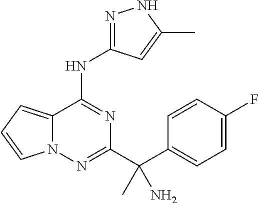

Step A: To (4-fluorophenyl)(4-(5-methyl-1H-pyrazol-3-ylamino)pyrrolo[1,2-f][1,2,4]triazin-2-yl)methanone from Example 1 (1 g, 2.7 mmol) in THF (10 mL) at 0° C. was added dropwise 1.4 M methylmagnesium bromide/THF (2 mL). The solution was allowed to warm to room temperature for 1 h. Then additional amounts of 1.4 M methylmagnesium bromide/THF followed by periods of stirring at rt as follows: 3 mL, 1 h; 5 mL, 0.75 h; 3 mL, 0.5 h; 5 mL, 0.25 h. Then 1 M aq HCl was added and the mixture was extracted with ethyl acetate. The organic layer was dried over sodium sulfate and concentrated to a residue (1.1 g). To this residue was added sodium azide (1.98 g, 30.5 mmol) and the mixture was cooled to 0° C. Trifluoroacetic acid (20 mL) was then added slowly, and the resulting solution was allowed to warm to room temperature overnight. EtOAc was added followed by slow addition of aq NaHCO₃. The organic layer was dried over sodium sulfate and concentrated, and the residue was purified by silica gel chromatography (MeOH/DCM 0-8%) to afford 2-(1-azido-1-(4-fluorophenyl)ethyl)-N-(5-methyl-1H-pyrazol-3-yl)pyrrolo[1,2-f][1,2,4]triazin-4-amine (740 mg, 73%). LC-MS (ESI) m/z 378 (M+H)⁺.

Step B: To 2-(1-azido-1-(4-fluorophenyl)ethyl)-N-(5-methyl-1H-pyrazol-3-yl)pyrrolo[1,2-f][1,2,4]triazin-4-amine (100 mg, 0.26 mmol), and 20% palladium hydroxide on carbon (49 mg) was added MeOH (3.5 mL) and the mixture was stirred under an atmosphere of hydrogen overnight. The mixture was filtered and the filtrate was concentrated. The residue was purified by preparative HPLC (Phenomenex C-18 reverse phase column, eluted with gradient of solvent B=0.05% HOAC/CH₃CN and solvent A=0.05% HOAc/H₂O) to afford 2-(1-amino-1-(4-fluorophenyl)ethyl)-N-(5-methyl-1H-pyrazol-3-yl)pyrrolo[1,2-f][1,2,4]triazin-4-amine as its acetate salt (51 mg, 48%). ¹H NMR: (300 MHz, DMSO-d₆) δ 1.72 (s, 3H), 1.91 (s, 3H), 2.18 (s, 3H), 6.13 (s, 1H), 6.65 (d, 1H), 7.09-7.17 (m, 3H), 7.52 (m, 2H), 7.71 (s, 1H), 10.42 (s, 1H); LC-MS (ESI) m/z 352 (M+H)⁺.

### Example 116

### Preparation of 2-((4-fluorophenyl)(methylsulfinyl)methyl)-N-(5-methyl-1H-pyrazol-3-yl)pyrrolo[1,2-f][1,2,4]triazin-4-amine

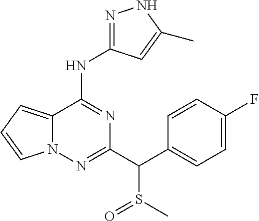

To 2-(bromo(4-fluorophenyl)methyl)-N-(5-methyl-1H-pyrazol-3-yl)pyrrolo[1,2-f][1,2,4]triazin-4-amine from Example 58 step A (220 mg, 0.55 mmol) in DMF (3 mL) was added sodium methanethiolate (93 mg, 1.32 mmol). The solution was stirred for 10 min at rt. To a 2 mL aliquot of this solution were added ethyl acetate and water, and the separated organic layer was concentrated. To the residue in DCM (5 mL) at 0° C. was added 3-chlorobenzoperoxoic acid (130 mg, 0.55 mmol), and the mixture was stirred at 0° C. for 30 min. DMSO (4 mL) was added and DCM was evaporated under reduced pressure. The mixture was purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(methylsulfinyl)methyl)-N-(5-methyl-1H-pyrazol-3-yl)pyrrolo[1,2-f][1,2,4]triazin-4-amine (19 mg, 14%). ¹H NMR (300 MHz, DMSO-d₆) δ 2.30 (s, 3H), 2.43 (s, 3H), 5.23 (s, 1H), 6.68-6.8 (m, 3H), 7.2-7.35 (m, 3H), 7.7-7.8 (m, 3H), 10.75 (s, 1H), 12.28 (s, 1H); LC-MS (ESI) m/z 385 (M+H)⁺.

### Example 117

### Preparation of 2-((4-fluorophenyl)(methylsulfonyl)methyl)-N-(5-methyl-1H-pyrazol-3-yl)pyrrolo[1,2-f][1,2,4]triazin-4-amine

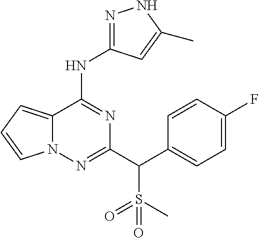

Also isolated from the chromatography of Example 116 was 2-((4-fluorophenyl)(methylsulfonyl)methyl)-N-(5-methyl-1H-pyrazol-3-yl)pyrrolo[1,2-f][1,2,4]triazin-4-amine (32 mg, 22%). ¹HNMR (300 MHz, DMSO-d₆) δ 2.26 (s, 3H), 3.02 (s, 3H), 5.82 (s, 1H), 6.72 (m, 2H), 7.23-7.28 (m, 3H), 7.52 (m, 1H), 7.89 (m, 2H), 10.76 (bs, 1H), 12.31 (bs, 1H); LC-MS (ESI) m/z 401 (M+H)⁺.

### Example 118

### Preparation of (4-(1H-pyrazol-3-ylamino)pyrrolo[1,2-f][1,2,4]triazin-2-yl)(2-(methylthio)phenyl)methanone

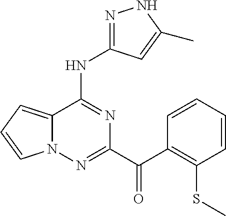

Treatment of ethyl 4-chloropyrrolo[1,2-f][1,2,4]triazine-2-carboxylate with (2-(methylthio)phenyl)magnesium bromide as described in General Procedure B, followed by treatment of the intermediate (4-chloropyrrolo[1,2-f][1,2,4]triazin-2-yl)(2-(methylthio)phenyl)methanone with 1H-pyrazol-3-amine according to Example 5 afforded (4-(1H-pyrazol-3-ylamino)pyrrolo[1,2-f][1,2,4]triazin-2-yl)(2-(methylthio) phenyl)methanone. ¹HNMR (DMSO-d₆): δ 12.52 (s, 1H), 10.97 (s, 1H), 7.94 (s, 1H), 7.67-7.53 (m, 4H), 7.40 (s, 1H), 7.30 (s, 1H), 6.83 (s, 1H), 6.70 (s, 1H), 2.40 (s, 3H); LC-MS (ESI) m/z 351 (M+H)⁺.

### Example 119

### Preparation of (4-chlorophenyl)(4-(5-methyl-1H-pyrazol-3-ylamino)pyrrolo[1,2-f][1,2,4]triazin-2-yl)methanone

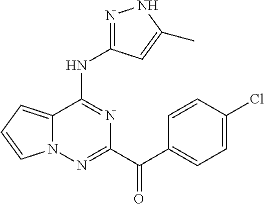

Step A: To a solution of ethyl 4-chloropyrrolo[1,2-f][1,2,4]triazine-2-carboxylate from General Procedure A Step D (4.05 g, 18 mmol) in DMF (40 mL) was added 5-methyl-1H-pyrazol-3-amine (2.1 g, 21.6 mmol) followed by triethylamine (2.75 mL, 19.8 mmol) and potassium iodide (3 g, 18 mmol). The mixture was stirred at rt for 2 h, then diluted with water. The precipitate was collected by filtration and dried to give ethyl 4-(5-methyl-1H-pyrazol-3-ylamino)pyrrolo[1,2-f][1,2,4]triazine-2-carboxylate (4.98 g, 97%) as a solid. LC-MS (ESI) m/z 287 (M+H)⁺.

Step B: To ethyl 4-(5-methyl-1H-pyrazol-3-ylamino)pyrrolo[1,2-f][1,2,4]triazine-2-carboxylate (300 mg, 1.04 mmol) in 1:1 MeOH:THF (4 mL) at 0° C. was added slowly 3N NaOH (2 mL) and the mixture was stirred at rt overnight. The mixture was concentrated, and the residue was diluted with water and acidified with 3N HCl. The precipitated solid was collected by filtration and dried to give 4-(5-methyl-1H-pyrazol-3-ylamino)pyrrolo[1,2-f][1,2,4]triazine-2-carboxylic acid (240 mg, 89%). LC-MS (ESI) m/z 259 (M+H)⁺.

Step C: To 4-(5-methyl-1H-pyrazol-3-ylamino)pyrrolo[1,2-f][1,2,4]triazine-2-carboxylic acid (210 mg, 0.813 mmol) in DMF (3 mL) was added HATU (340.03 mg, 0.894 mmol) and the mixture was stirred at rt for 10 min. Then N,O-dimethylhydroxylamine hydrochloride (79.3 mg, 0.813 mmol) was added followed by DIPEA (0.283 uL, 1.626 mmol). The mixture was stirred at rt for 1 h, diluted with 1:9 MeOH/EtOAc, and washed with brine. The organic layer was concentrated and the residue was dried to give N-methoxy-N-methyl-4-(5-methyl-1H-pyrazol-3-ylamino)pyrrolo[1,2-f][1,2,4]triazine-2-carboxamide (243 mg, 99%) as a white solid. LC-MS (ESI) m/z 302 (M+H)⁺.

Step D: To methyl N-methoxy-N-methyl-4-(5-methyl-1H-pyrazol-3-ylamino)pyrrolo[1,2-f][1,2,4]triazine-2-carboxamide (243 mg, 0.806 mmol) in THF (4 mL) at 0° C. was added 1M p-chlorophenylmagnesium bromide/THF (2.4 mL). The reaction mixture was stirred at rt for 1 h, then the reaction was quenched by addition of aq ammonium chloride. DCM/MeOH was added and the organic layer was separated and concentrated. The residue was sonicated with MeOH and the solid was collected by filtration and dried to afford (4-chlorophenyl)(4-(5-methyl-1H-pyrazol-3-ylamino)pyrrolo[1,2-f][1,2,4]triazin-2-yl)methanone (174 mg, 61%). ¹H NMR (300 MHz, DMSO-d₆) δ 12.22 (s, 1H), 10.866 (s, 1H), 8.03 (d, 2H), 7.86 (s, 1H), 7.64 (d, 2H), 7.40 (s, 1H), 6.83 (s, 1H), 6.53 (s, 1H), 2.18 (s, 3H); LC-MS (ESI) m/z 369 (M+H)⁺.

The following compounds were made using a similar procedure, but replacing in Step D the p-chlorophenylmagnesium bromide with the appropriately substituted organometallic reagent:

| Example | | | Analytical |
|---|---|---|---|
| Number | Structure | Name | Data |
| 119a | 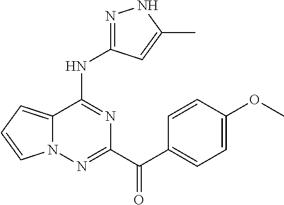 | (4-Methoxy-phenyl)-[4- (5-methyl-1H-pyrazol-3- ylamino)-pyrrolo[2,1- f][1,2,4]triazin-2-yl]- methanone | LC-MS (ESI) m/z 349 (M + H)⁺; 11.55 min |
| 119b | 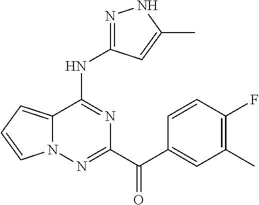 | (4-Fluoro-3-methyl- phenyl)-[4-(5-methyl- 1H-pyrazol-3-ylamino)- pyrrolo[2,1-f][1,2,4] triazin-2-yl]-methanone | LC-MS (ESI) m/z 351 (M + H)⁺; 12.65 min |
| 119c | 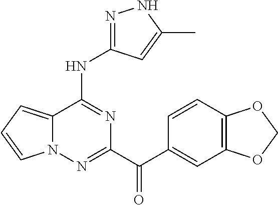 | Benzo[1,3]dioxol-5-yl- [4-(5-methyl-1H-pyrazol- 3-ylamino)-pyrrolo[2,1- f][1,2,4]triazin-2-yl]- methanone | LC-MS (ESI) m/z 363 (M + H)⁺; 11.35 min |
| 119d | 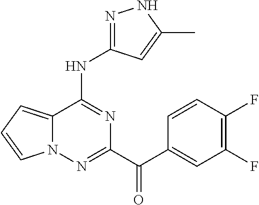 | (3,4-Difluoro-phenyl)-[4- (5-methyl-1H-pyrazol-3- ylamino)-pyrrolo[2,1- f][1,2,4]triazin-2-yl]- methanone | LC-MS (ESI) m/z 355 (M + H)⁺; 12.49 min |

The following compounds were made using the compounds above, using General Procedure D described in Example 2.

| Example | | | Analytical |
|---|---|---|---|
| Number | Structure | Name | Data |
| 119e | 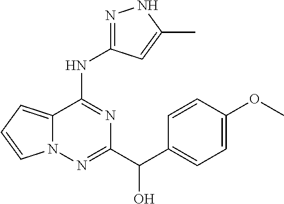 | (4-Methoxy-phenyl)-[4- (5-methyl-1H-pyrazol-3- ylamino)-pyrrolo[2,1- f][1,2,4]triazin-2-yl]- methanol | LC-MS (ESI) m/z 351 (M + H)⁺; 10.09 min |
| 119f | 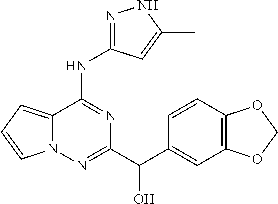 | Benzo[1,3]dioxol-5-yl- [4-(5-methyl-1H-pyrazol- 3-ylamino)-pyrrolo[2,1- f][1,2,4]triazin-2-yl]- methanol | LC-MS (ESI) m/z 365 (M + H)⁺; 9.92 min |
| 119g | 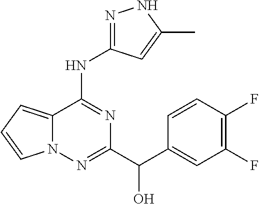 | (3,4-Difluoro-phenyl)-[4- (5-methyl-1H-pyrazol-3- ylamino)-pyrrolo[2,1- f][1,2,4]triazin-2-yl]- methanol | LC-MS (ESI) m/z 357 (M + H)⁺; 11.11 min |
| 119h | 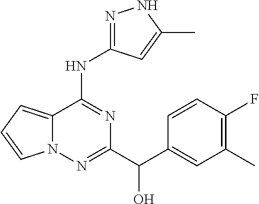 | (4-Fluoro-3-methyl- phenyl)-[4-(5-methyl- 1H-pyrazol-3-ylamino)- pyrrolo[2,1-f][1,2,4] triazin-2-yl]-methanol | LC-MS (ESI) m/z 353 (M + H)⁺; 10.95 min |
| 119i | 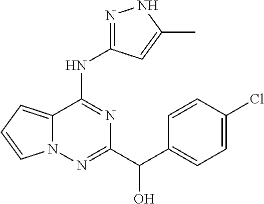 | (4-Chloro-phenyl)-[4-(5- methyl-1H-pyrazol-3- ylamino)-pyrrolo[2,1- f][1,2,4]triazin-2-yl]- methanol | LC-MS (ESI) m/z 355 (M + H)⁺; 11.27 min |

### Example 120

### Preparation of methyl (4-fluorophenyl)(4-(5-methyl-1H-pyrazol-3-ylamino)pyrrolo[1,2-f][1,2,4]triazin-2-yl)methylcarbamate

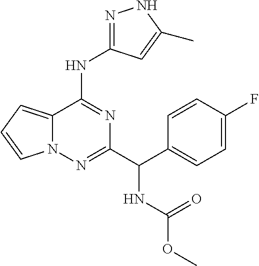

2-(amino(4-fluorophenyl)methyl)-N-(5-methyl-1H-pyrazol-3-yl)pyrrolo[1,2-f][1,2,4]triazin-4-amine from Example 69 step B was converted to the free base. To the free base (300 mg, 0.88 mmol) in THF (3 mL) was added diisopropylethylamine (0.2 mL) followed by addition of methyl chloroformate (6 drops). The solution was stirred for 5 min and then 10% aq sodium hydroxide was added and the mixture was stirred for 30 min. The mixture was extracted with ethyl acetate, and the extracts were dried over sodium sulfate and concentrated. The residue was purified by preparative HPLC (Varian diphenyl reverse phase column, eluted with gradient of solvent B=0.05% HOAC/CH₃CN and solvent A=0.05% HOAc/H₂O) to afford methyl (4-fluorophenyl)(4-(5-methyl-1H-pyrazol-3-ylamino)pyrrolo[1,2-f][1,2,4]triazin-2-yl)methylcarbamate (50 mg, 14%). ¹H NMR: (300 MHz, DMSO-d₆) δ 2.23 (s, 3), 3.57 (s, 3H), 5.71 (d, 1H), 6.42 (s, 1H), 6.65 (m, 1H), 7.13-7.2 (m, 3H), 7.52 (m, 2H), 7.68 (s, 1H), 8.03 (d, 1H), 10.54 (s, 1H), 12.13 (s, 1H); LC-MS (ESI) m/z 396 (M+H)⁺.

The following compounds were prepared using a similar procedure, but replacing methyl chloroformate with an appropriately substituted chloroformate, carbamoyl chloride, sulfonyl chloride or an activated carboxylic acid optionally followed by a boc-deprotection, when boc is present.

| Example | | | Analytical |
|---|---|---|---|
| Number | Structure | Name | Data |
| 120a | 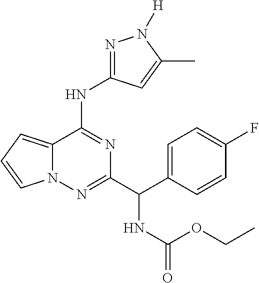 | {(4-Fluoro-phenyl)-[4-(5- methyl-1H-pyrazol-3-ylamino)- pyrrolo[2,1-f][1,2,4]triazin-2- yl]-methyl}-carbamic acid ethyl ester | LC-MS (ESI) m/z 410 (M + H)⁺; 12.26 min |
| 120b | 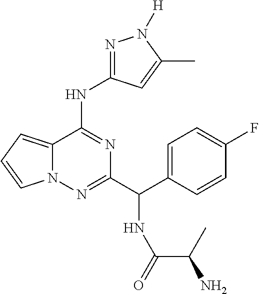 | 2-((R,S)-(4-fluorophenyl)((R)- tetrahydrofuran-3- yloxy)methyl)-N-(5-methyl- 1H-pyrazol-3-yl)pyrrolo[1,2- f][1,2,4]triazin-4-amine 2-Amino-N-{(4-fluoro-phenyl)- [4-(5-methyl-1H-pyrazol-3- ylamino)-pyrrolo[2,1- f][1,2,4]triazin-2-yl]-methyl}- propionamide | LC-MS (ESI) m/z 409 (M + H)⁺; 7.98 min |
| 120c | 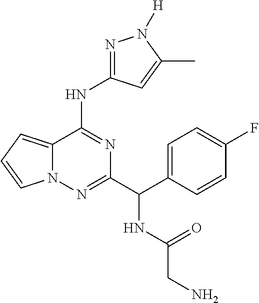 | 2-Amino-N-{(4-fluoro-phenyl)- [4-(5-methyl-1H-pyrazol-3- ylamino)-pyrrolo[2,1- f][1,2,4]triazin-2-yl]-methyl}- acetamide | LC-MS (ESI) m/z 395 (M + H)⁺; 7.89 min |
| 120d | 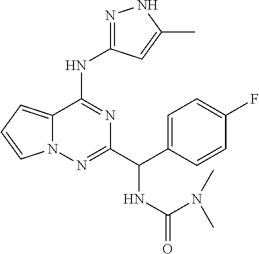 | 3-{(4-Fluoro-phenyl)-[4-(5- methyl-1H-pyrazol-3-ylamino)- pyrrolo[2,1-f][1,2,4]triazin-2- yl]-methyl}-1,1-dimethyl-urea | LC-MS (ESI) m/z 409 (M + H)⁺; 10.83 min |
| 120e | 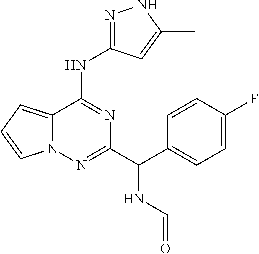 | N-{(4-Fluoro-phenyl)-[4-(5- methyl-1H-pyrazol-3-ylamino)- pyrrolo[2,1-f][1,2,4]triazin-2- yl]-methyl}-formamide | LC-MS (ESI) m/z 366 (M + H)⁺; 10.55 min |
| 120f | 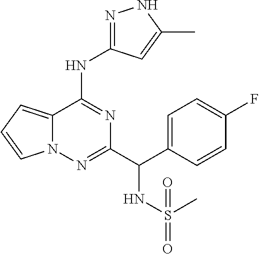 | N-{(4-Fluoro-phenyl)-[4-(5- methyl-1H-pyrazol-3-ylamino)- pyrrolo[2,1-f][1,2,4]triazin-2- yl]-methyl}-methanesulfonamide | LC-MS (ESI) m/z 416 (M + H)⁺; 11.25 min |
| 120g | 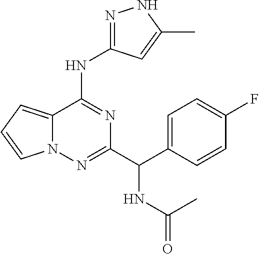 | N-{(4-Fluoro-phenyl)-[4-(5- methyl-1H-pyrazol-3-ylamino)- pyrrolo[2,1-f][1,2,4]triazin-2- yl]-methyl}-acetamide | LC-MS (ESI) m/z 380 (M + H)⁺; 10.37 min |
| 120h | 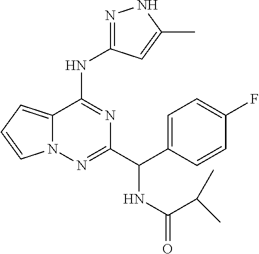 | N-{(4-Fluoro-phenyl)-[4-(5- methyl-1H-pyrazol-3-ylamino)- pyrrolo[2,1-f][1,2,4]triazin-2- yl]-methyl}-isobutyramide | LC-MS (ESI) m/z 408 (M + H)⁺; 11.50 min |
| 120i | 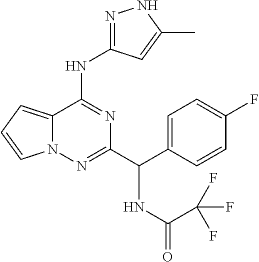 | 2,2,2-Trifluoro-N-{(4-fluoro- phenyl)-[4-(5-methyl-1H- pyrazol-3-ylamino)- pyrrolo[2,1-f][1,2,4]triazin-2- yl]-methyl}-acetamide | LC-MS (ESI) m/z 434 (M + H)⁺; 12.58 min |

### Example 121

### Preparation of 2-((4-fluorophenyl)(2-methoxyethoxy)methyl)-N-(5-methyl-1H-pyrazol-3-yl)pyrrolo[1,2-f][1,2,4]triazin-4-amine

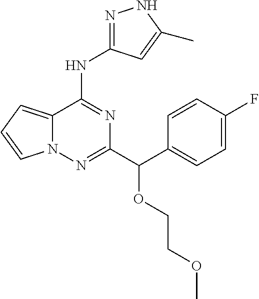

To (4-fluorophenyl)(4-(5-methyl-1H-pyrazol-3-ylamino)pyrrolo[1,2-f][1,2,4]triazin-2-yl)methanol from Example 2 (100 mg, 0.3 mmol) in 2-methoxyethanol (1.5 mL) was added methanesulfonic acid (0.05 mL). The solution was heated in a microwave reactor at 140° C. for 30 min. Triethylamine (0.15 mL) was then added and the mixture was purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(2-methoxyethoxy)methyl)-N-(5-methyl-1H-pyrazol-3-yl)pyrrolo[1,2-f][1,2,4]triazin-4-amine (49 mg, 40%). ¹H NMR: (300 MHz, DMSO-d₆) δ 2.26 (s, 3H), 3.25 (s, 3H), 3.51-3.67 (m, 4H), 5.34 (s, 1H), 6.52 (s, 1H), 6.66 (m, 1H), 7.15-7.26 (m, 3H), 7.57 (m, 2H), 7.72 (s, 1H) 10.58 (s, 1H), 12.12 (bs, 1H); LC-MS (ESI) m/z 419 (M+Na)⁺.

The following compounds were prepared using a similar procedure, replacing the 2-methoxyethanol with the appropriate alcohol:

| Example | | | Analytical |
|---|---|---|---|
| Number | Structure | Name | Data |
| 121a | 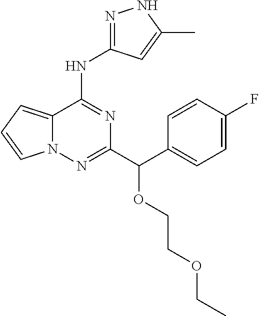 | {2-[(2-Ethoxy-ethoxy)- (4-fluoro-phenyl)-methyl]- pyrrolo[2,1-f][1,2,4]triazin- 4-yl}-(5-methyl-1H-pyrazol- 3-yl)-amine | LC-MS (ESI) m/z 411 (M + H)⁺; 12.54 min |
| 121b | 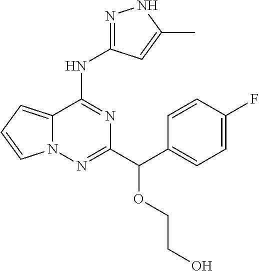 | 2-{(4-Fluoro-phenyl)-[4- (5-methyl-1H-pyrazol-3- ylamino)-pyrrolo[2,1-f][1,2,4] triazin-2-yl]-methoxy}-ethanol | LC-MS (ESI) m/z 383 (M + H)⁺; 10.53 min |
| 121c | 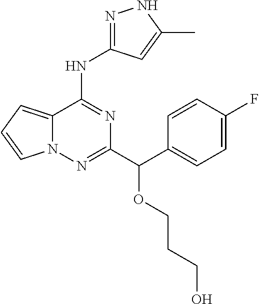 | 3-{(4-Fluoro-phenyl)-[4- (5-methyl-1H-pyrazol-3- ylamino)-pyrrolo[2,1-f][1,2,4] triazin-2-yl]-methoxy}- propan-1-ol | LC-MS (ESI) m/z 397 (M + H)⁺; 10.67 min |

### Example 122

### Preparation of 2-((4-fluorophenyl)(3-morpholinopropylthio)methyl)-N-(5-methyl-1H-pyrazol-3-yl)pyrrolo[1,2-f][1,2,4]triazin-4-amine

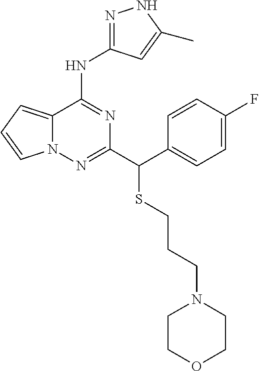

To potassium thioacetate (37 mg, 0.32 mmol) in DMF (2 mL) was added 2-(bromo(4-fluorophenyl)methyl)-N-(5-methyl-1H-pyrazol-3-yl)pyrrolo[1,2-f][1,2,4]triazin-4-amine from Example 69 Step A (100 mg, 0.25 mmol) and the solution stirred at rt for 20 min. Then 25% NaOMe/MeOH (0.1 mL) was added followed by addition of 1-bromo-3-chloropropane (0.074 mL, 0.75 mmol) and sodium borohydride (20 mg). The solution was stirred at rt for 1.5 h and then quenched with 2N HCl. The resulting mixture was extracted with EtOAc, and the organic layer was dried over sodium sulfate and concentrated. To the residue was added 1-methylpyrrolidin-2-one (2 mL) and morpholine (0.2 mL) and the mixture was heated to 60° C. overnight. Potassium iodide (100 mg) and triethylamine (0.1 mL) were added and heating was continued at 80° C. overnight. Acetic acid (0.4 mL) was added and the resulting mixture was purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(3-morpholinopropylthio)methyl)-N-(5-methyl-1H-pyrazol-3-yl)pyrrolo[1,2-f][1,2,4]triazin-4-amine (49 mg, 40%). ¹H NMR: (300 MHz, DMSO-d₆) δ 1.67 (m, 2H), 2.1-2.3 (m, 7H), 3.49 (m, 4H), 5.12 (s, 1H), 6.65-6.68 (m, 2H), 7.14-7.2 (m, 3H), 7.67-7.71 (m, 3H), 10.62 (s, 1H), 12.15 (bs, 1H); LC-MS (ESI) m/z 482 (M+H)⁺.

The following compounds were prepared using a similar procedure, replacing 1-bromo-3-chloropropane with 1-bromo-2-chloroethane for certain compounds, and replacing the morpholine with an appropriate amine:

| Example | | | Analytical |
|---|---|---|---|
| Number | Structure | Name | Data |
| 122a | 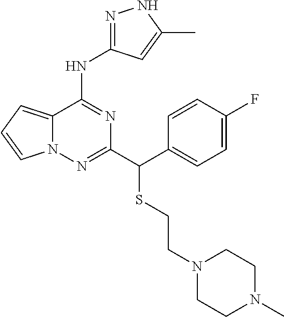 | (2-{(4-Fluoro-phenyl)-[2-(4- methyl-piperazin-1-yl)- ethylsulfanyl]-methyl}- pyrrolo[2,1-f][1,2,4]triazin- 4-yl)-(5-methyl-1H-pyrazol- 3-yl)-amine | LC-MS (ESI) m/z 481 (M + H)⁺; 8.38 min |
| 122b | 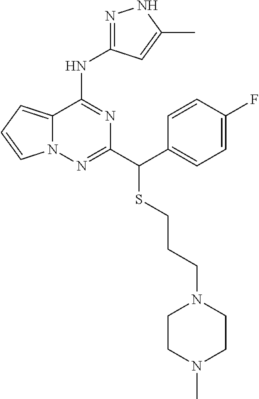 | (2-{(4-Fluoro-phenyl)-[3-(4- methyl-piperazin-1-yl)- propylsulfanyl]-methyl}- pyrrolo[2,1-f][1,2,4]triazin- 4-yl)-(5-methyl-1H-pyrazol- 3-yl)-amine | LC-MS (ESI) m/z 495 (M + H)⁺; 8.38 min |
| 122c | 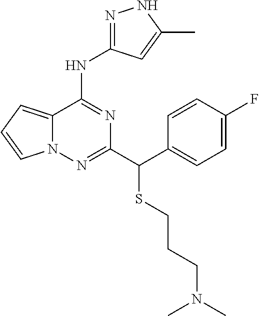 | {2-[(3-Dimethylamino- propylsulfanyl)-(4-fluoro- phenyl)-methyl]-pyrrolo[2,1- f][1,2,4]triazin-4-yl}-(5- methyl-1H-pyrazol-3-yl)- amine | LC-MS (ESI) m/z 440 (M + H)⁺; 8.39 min |
| 122d | 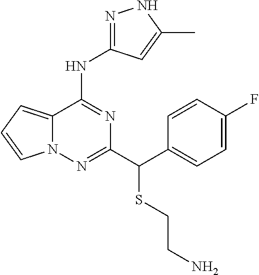 | {2-[(2-Amino-ethylsulfanyl)- (4-fluoro-phenyl)-methyl]- pyrrolo[2,1-f][1,2,4]triazin- 4-yl}-(5-methyl-1H-pyrazol- 3-yl)-amine | LC-MS (ESI) m/z 398 (M + H)⁺; 8.05 min |
| 122e | 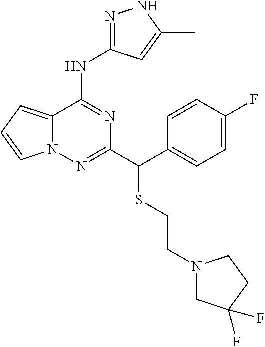 | {2-[[2-(3,3-Difluoro- pyrrolidin-1-yl)-ethyl- sulfanyl]-(4-fluoro-phenyl)- methyl]-pyrrolo[2,1-f][1,2,4] triazin-4-yl}-(5-methyl-1H- pyrazol-3-yl)-amine | LC-MS (ESI) m/z 488 (M + H)⁺; 12.38 min |

### Example 123

### Preparation of (2S)-((R,S)(4-fluorophenyl)(4-(5-methyl-1H-pyrazol-3-ylamino)pyrrolo[1,2-f][1,2,4]triazin-2-yl)methyl) 2-aminopropanoate

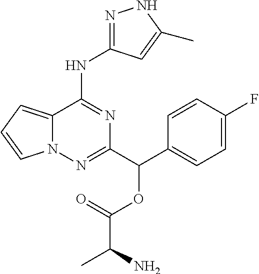

To (S)-2-(tert-butoxycarbonylamino)propanoic acid (141 mg, 0.74 mmol) was added cesium carbonate (98 mg, 0.3 mmol) in DMF (2 mL) and the mixture was stirred for 15 min. Then 2-(bromo(4-fluorophenyl)methyl)-N-(5-methyl-1H-pyrazol-3-yl)pyrrolo[1,2-f][1,2,4]triazin-4-amine from Example 69 Step A (50 mg, 0.12 mmol) was added and the mixture was stirred at rt overnight. Aq sodium bicarbonate was added and the resulting mixture was extracted with EtOAc. The organic extract was concentrated, then DCM (10 mL) was added, and the mixture was filtered. To the filtrate was added 4N HCl/dioxane (0.5 mL), and the mixture was stirred for 2 h and then concentrated. The residue was purified by preparative HPLC (Phenomonex C-18 reverse phase column, eluted with gradient of solvent B=0.05% HOAC/CH₃CN and solvent A=0.05% HOAc/H₂O) to afford (2S)-(4-fluorophenyl)(4-(5-methyl-1H-pyrazol-3-ylamino)pyrrolo[1,2-f][1,2,4]triazin-2-yl)methyl 2-aminopropanoate (23 mg, 47%). ¹H NMR: (300 MHz, DMSO-d₆) δ 1.40 (m, 3H), 2.25 (s, 314), 3.93 (m, 1H), 6.49-6.68 (m, 4H), 7.22-7.28 (m, 3H), 7.62-7.71 (m, 3H), 10.68 (s, 1H); LC-MS (ESI) m/z 410 (M+H)⁺.

The following compounds were prepared using a similar procedure, replacing (S)-2-(tert-butoxycarbonylamino)propanoic acid with the appropriate amino acid. In the case of Example 123c, no boc deprotection step was required.

| Example | | | Analytical |
|---|---|---|---|
| Number | Structure | Name | Data |
| 123a | 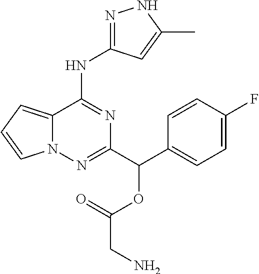 | Amino-acetic acid (4- fluoro-phenyl)-[4-(5- methyl-1H-pyrazol-3- ylamino)-pyrrolo[2,1- f][1,2,4]triazin-2-yl]- methyl ester | LC-MS (ESI) m/z 396 (M + H)⁺; 8.03 min |
| 123b | 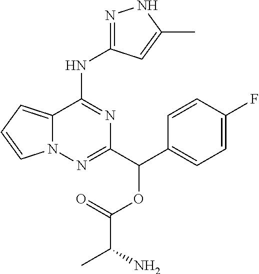 | (R)-((R,S)-(4-fluoro- phenyl)(4-(5-methyl- 1H-pyrazol-3-ylamino) pyrrolo[1,2-f][1,2,4] triazin-2-yl)methyl) 2- aminopropanoate | LC-MS (ESI) m/z 410 (M + H)⁺; 8.09 min |
| 123c | 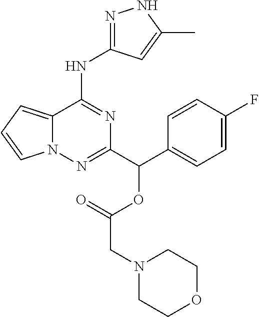 | Morpholin-4-yl-acetic acid (4-fluoro-phenyl)-[4-(5- methyl-1H-pyrazol-3- ylamino)-pyrrolo[2,1- f][1,2,4]triazin-2-yl]- methyl ester | LC-MS (ESI) m/z 466 (M + H)⁺; 10.21 min |
| 123d | 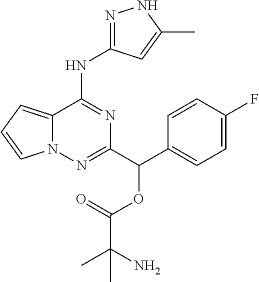 | 2-Amino-2-methyl- propionic acid (4-fluoro- phenyl)-[4-(5-methyl-1H- pyrazol-3-ylamino)- pyrrolo[2,1-f][1,2,4]triazin- 2-yl]-methyl ester | LC-MS (ESI) m/z 424 (M + H)⁺; 8.33 min |
| 123e | 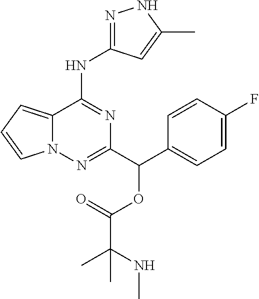 | 2-Methyl-2-methylamino- propionic acid (4-fluoro- phenyl)-[4-(5-methyl-1H- pyrazol-3-ylamino)- pyrrolo[2,1-f][1,2,4]triazin- 2-yl]-methyl ester | LC-MS (ESI) m/z 438 (M + H)⁺; 8.37 min |

### Example 124

### Preparation of methyl 2-((4-fluorophenyl)(methoxy)methyl)-N-(5-methyl-1H-pyrazol-3-yl)pyrrolo[1,2-f][1,2,4]triazin-4-amine

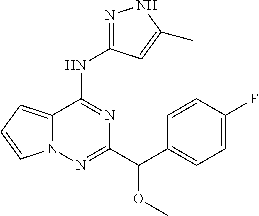

2-(bromo(4-fluorophenyl)methyl)-N-(5-methyl-1H-pyrazol-3-yl)pyrrolo[1,2-f][1,2,4]triazin-4-amine from Example 69 Step A, (80 mg, 0.2 mmol) was added to a solution of silver carbonate (55 mg, 0.2 mmol) in MeOH (2 mL). The mixture was stirred for 3 h at rt and then purified by preparative HPLC (Varian diphenyl reverse phase column, eluted with gradient of solvent B=0.05% HOAC/CH₃CN and solvent A=0.05% HOAc/H₂O) to afford 2-((4-fluorophenyl)(methoxy)methyl)-N-(5-methyl-1H-pyrazol-3-yl)pyrrolo[1,2-f][1,2,4]triazin-4-amine (31 mg, 44%). ¹H NMR: (300 MHz, DMSO-d₆) δ 2.26 (s, 3), 3.35 (s, 3H), 5.20 (s, 1H), 6.48 (s, 1H), 6.66 (m, 1H), 7.15-7.21 (m, 3H), 7.55 (m, 2H), 7.73 (m, 1H), 10.57 (bs, 1H), 12.15 (bs, 1H); LC-MS (ESI) m/z 353 (M+H)⁺.

### Example 125

### Preparation of N-(4-bromo-5-methyl-1H-pyrazol-3-yl)-2-(difluoro(4-fluorophenyl)methyl)-6-(methylsulfonylmethyl)pyrrolo[1,2-f][1,2,4]triazin-4-amine

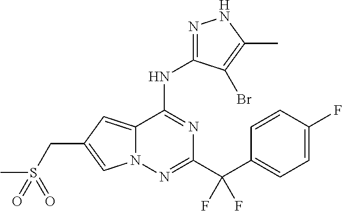

To a solution of 6-(bromomethyl)-2-(difluoro(4-fluorophenyl)methyl)-N-(5-methyl-1H-pyrazol-3-yl)pyrrolo[1,2-f][1,2,4]triazin-4-amine (320 mg, 0.71 mmol) in THF (2 mL), sodium methanethiolate (99 mg, 1.42 mmol) was added at room temperature. After stirring the reaction mixture at room temperature for 1 h, methanol (25 mL) and an aqueous solution of OXONE (4 mL, 1.3 g, 2.13 mmol) were added. The heterogeneous mixture was stirred for 3 days at room temperature. The solid was separated by filtration and the filtrate was concentrated to dryness. The residue was taken in ethyl acetate and washed with water and brine solution. After drying over MgSO₄, the solvent was evaporated under vacuum and the crude product purified on HPLC. N-(4-bromo-5-methyl-1H-pyrazol-3-yl)-2-(difluoro(4-fluorophenyl)methyl)-6-(methylsulfonylmethyl)pyrrolo[1,2-f][1,2,4]triazin-4-amine was isolated as a white solid (6.5 mg, 1.7%). ¹HNMR (DMSO-d₆): δ 2.25 (s, 3H), 3.49 (s, 3H), 4.53 (s, 2H), 7.29-7.24 (m, 3H), 7.57 (m, 2H), 7.88 (s, 1H), 10.38 (s, 1H), 12.97 (s, 1H); LC-MS (ESI) m/z 531 (M+H)⁺; 12.00 min.

### Example 126

### Competition Binding Assay to Determine Binding Constants (K_{d}) of the Compounds Against JAK Kinases

Competition binding assays used herein were developed, validated and performed as described in Fabian et al., Nature Biotechnology 2005, 23, 329-336. Kinases were produced as fusions to T7 phage (See, Fabian et al. or WO04/015142) or alternatively, the kinases were expressed in HEK-293 cells and subsequently tagged with DNA for PCR detection (See, WO08/005,310). For the binding assays, streptavidin-coated magnetic beads were treated with biotinylated affinity ligands for 30 min at rt to generate affinity resins. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinase, liganded affinity beads, and test compounds in 1× binding buffer (20% SeaBlock, 0.17×PBS, 0.05% Tween 20, 6 mM DTT). Test compounds were prepared as 100× stocks in DMSO and rapidly diluted into the aqueous environment. DMSO was added to control assays lacking a test compound. Primary screen interactions were performed in polypropylene 384-well plates in a final volume of 34 μL, while K_{d} determinations were performed in polystyrene 96-well plates in a final volume of 135 μL. The assay plates were incubated at room temperature with shaking for 1 hour, long enough for binding reactions to reach equilibrium, and the affinity beads were washed extensively with wash buffer (1×PBS, 0.05% Tween 20) to remove unbound protein. The beads were then resuspended in elution buffer (1×PBS, 0.05% Tween 20, 2 μM non-biotinylated affinity ligand) and incubated at room temperature with shaking for 30 min. The kinase concentration in the eluates was measured by quantitative PCR. Each kinase was tested individually against each compound. K_{ds} were determined using eleven serial threefold dilutions. A selectivity score, which is a quantitative measure of selectivity of a compound against a panel of enzymes, may be calculated for a compound by dividing the number of enzymes for which a compound meets a set criteria, (for example, a binding constant of 100 nM or less), by the total number of enzymes tested. A kinase selectivity score, S35, for example, is calculated for each compound by dividing the number of kinases for which a compound displayed inhibition of 65% or greater compared to negative control lacking inhibitors (DMSO only), divided by the 321 distinct kinases tested excluding mutant variants.

In one embodiment, the compounds provided herein were found to have Kds of less than about 1 μM against JAK2. In another embodiment, the compounds provided herein were found to have Kds of less than about 200 nM against JAK2. In another embodiment, the compounds provided herein were found to have Kds of less than about 100 nM against JAK2.

In another embodiment, the compounds provided herein were found to have Kds of less than about 5 μM against JAK3. In another embodiment, the compounds provided herein were found to have Kds of less than about 1 μM against JAK3. In another embodiment, the compounds provided herein were found to have Kds of less than about 200 nM against JAK3. In another embodiment, the compounds provided herein were found to have Kds of less than about 100 nM against JAK3.

### Example 127

### csTF-1 Cell-Based Reporter Assay

csTF-1 cells are derived from the human erythroleukemia cell line that is growth dependent on GM-CSF and has an intact GM-CSFR/JAK2/STAT5 pathway. The cell line contains stably integrated beta-lactamase reporter gene under the control of the regulatory factor 1 (irf 1) response element recognized by the activated transcription factor STAT5. csTF-1 cells (Invitrogen K₁₂₁₉) were washed with assay media (97% OPTIMEM/0.5% dialyzed FBS/0.1 mM NEAR/1 mM Na pyr/P/S) and seeded in the same media at 5×10⁵ cell/mL in T150 flask. After 16 hour incubation, cells were seeded at 2×10⁵ cell/well in 50 p. 1 volume, into Costar, clear bottom, 96-well assay plates. Serial dilutions of compounds were added to the plates with final DMSO concentration at 0.5% and GM-CSF at 2 ng/mL and the plates were then incubated at 30° C. and 5% CO₂ for 4 hours. The plates were brought to room temperature before adding Substrate Mixture according to manufacturer's protocol (Invitrogen, Catalog #K₁₀₈₅). The assay plates containing the substrate mixture were incubated in the dark at room temperature for 2 hours. Blue and green fluorescence was measured with excitation at 409 nm and emission at 460 nm (for blue) and excitation at 409 nm and emission at 530 nm (for green) using Spectra Max Gemini EM. The compounds provided herein were found to have IC₅₀ of less than about 5 μM. In another embodiment, the compounds provided herein were found to have activity IC₅₀ of less than about 500 nM.

### Example 128

### STAT5A Phosphorylation ELISA

A STAT5A phosphorylation ELISA (Biosource, STAT5A [pY693] kit, Catalog #KH00761) was used to measure the inhibition of STAT5a phosphorylation in the HEL-2 human leukemia cell line in the presence of the compounds provided herein. The HEL-2 cell line contains constitutively activated JAK2 carrying the V617F mutation. A total STAT5A ELISA was also run in parallel (Biosource, tSTAT5A kit, Catalog #KHO0751).

HEL cells were plated at 200,000 cells per well in RPMI complete with 0.5% serum into a 96 well plate and incubated overnight in a 37° C. incubator with 10% CO₂. The compound plate was set up by aliquoting into row B the positive control and titrating the test compounds in serial dilutions (using RPMI media with 0.5% FBS) into rows C-G for a total of 6 compounds per plate. The first well of each row contained the DMSO control. An aliquot from each well of the compound plate was transferred to the plated cells and then incubated for two hours at 37° C. The compound/media solution was aspirated off and the cells washed with cold PBS. The cells were then lysed for 30 minutes at 4° C. with Cell Extraction Buffer containing Phosphastase Inhibitor (catalog #FNN0011 from Invitrogen) and protease inhibitors (catalog #1836 170 001). The plate was centrifuged for 30 minutes at 2500 rpm to pellet out the cell debris. The cleared lysates were transferred to a 96-well Nunc plate and used in the ELISA protocol as described in Catalog #KH00761 and KH00751.

Since modifications will be apparent to those of skill in the art, it is intended that the claimed subject matter be limited only by the scope of the appended claims.

1. A compound having formula (I): 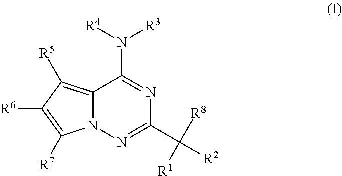
or a pharmaceutically acceptable salt thereof, wherein:
R¹ and R² are selected from (i), (ii), (iii) and (iv) as follows:
(i) R¹ and R² together form ═O, ═S, ═NR⁹ or ═CR¹⁰R¹¹;
(ii) R¹ and R² together with the carbon atom to which they are attached, form optionally substituted cycloalkyl, oxacycloalkyl or heterocyclyl, wherein the substituents, when present, are one, two or three groups selected from oxo, alkyl, and halo;
(iii) R¹ and R² are each independently hydrogen, halo, haloalkyl, alkyl, alkenyl, alkynyl, cycloalkyl, hydroxyalkyl, alkoxyalkyl, aminoalkyl, heterocyclylalkyl, heteroaryl, —OR¹² or —S(O)ₚR¹²; or
(iv) R¹ is —OR¹², —NR¹³R¹⁴, —S(O)ₚR¹², —N(R¹⁵)R¹⁶OR¹², —N(R¹⁵)R¹⁶NR¹³R¹⁴, —N(R¹⁵)R¹⁶S(O)ₚR¹², —NR¹⁵S(O)ₚR¹², —OR¹⁶OR¹², —OR¹⁶S(O)pR¹², —OR¹⁶NR¹³R¹⁴, —S(O)ₚR¹⁶OR¹², —S(O)ₚR¹⁶NR¹³R¹⁴, S(O)ₚNR¹³R¹⁴, —S(CN), —OC(O)R¹², —NR¹⁵C(O)NR¹³R¹⁴, —NR¹⁵C(O)R¹² or —R¹⁸C(O)OR¹², and R² is hydrogen, cyano, haloalkyl, alkyl, alkenyl, alkynyl, cycloalkyl, hydroxyalkyl or alkoxyalkyl;
R³ is cycloalkyl, aryl, heterocyclyl or heteroaryl;
R⁴ is hydrogen or alkyl;
R⁵, R⁶ and R⁷ are each independently selected from hydrogen, halo, nitro, hydroxy, alkyl, alkenyl, alkynyl, cycloalkyl, heterocyclyl, heterocyclylalkyl, —OR^{x}, —NR¹⁵C(O)R¹⁷, —NR¹⁵C(O)OR¹⁷, —NR¹⁵C(O)NR¹⁹R²⁹, —NR¹⁵S(O)ₚR¹⁷, —C(O)NR¹⁹R²⁰—S(O)ₚNR¹⁹R²⁰, —NR¹⁵C(O)OR¹⁷ or —NR¹⁵C(O)NR¹⁹R²⁰—C(S)NR¹⁹R²⁹, —C(O)OR¹⁷, —C(S)OR¹⁷ and —C(═NOR¹⁵)R²¹ wherein the alkyl, alkenyl, alkynyl, cycloalkyl, heterocyclyl and heterocyclylalkyl may each be optionally substituted with one, two, three or four groups selected from halo, alkyl, haloalkyl, hydroxyalkyl, cyano, oxo, —R^{w}—OR^{x}, —R^{w}—OR^{w}NR^{y}r^{z}, —R^{w}—NR^{y}R^{z}, —R^{w}—NR^{u}C(O)R^{v}, —R^{w}—NR^{u}S(O)ₚR^{v} and —S(O)ₚR^{v};
R⁸ is cycloalkyl, aryl, heteroaryl or heterocyclyl;
R⁹ is alkyl, —OR¹² or —NR¹³R¹⁴;
R¹⁰ and R¹¹ are each independently hydrogen, alkyl or —C(O)OR¹²;
R¹² is hydrogen, alkyl, cycloalkyl, cycloalkylalkyl, heterocyclyl or heterocyclylalkyl, wherein the alkyl, cycloalkyl, cycloalkylalkyl, heterocyclyl and heterocyclylalkyl may each be optionally substituted with one, two or three groups selected from halo, alkyl, hydroxy, alkoxy, amino, alkylamino, alkylthio, alkylsulfinyl and alkylsulfonyl;
each R¹³ and R¹⁴ is independently selected from (i) and (ii) below:
(i) R¹³ and R¹⁴ are each independently hydrogen, alkyl, cycloalkyl, cycloalkylalkyl, heterocyclyl or heterocyclylalkyl, wherein the alkyl, cycloalkyl, cycloalkylalkyl, heterocyclyl and heterocyclylalkyl may each be optionally substituted with one, two or three groups independently selected from halo, oxo, alkyl, hydroxy, alkoxy and amino, or
(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;
each R¹⁵ is independently hydrogen or alkyl;
each R¹⁶ is independently alkylene or alkenylene;
each R¹⁷ is alkyl, alkenyl, alkynyl, cycloalkyl, cycloalkylalkyl, alkoxy, heterocyclyl, heterocyclylalkyl, aryl, aralkyl, heteroaryl or heteroaralkyl, any of which may be optionally substituted with one, two or three groups selected from halo, alkyl, hydroxy, alkoxy, amino, alkylthio and alkylsulfonyl;
each R¹⁸ is independently alkylene or a direct bond;
each R¹⁹ and R²⁰ is independently selected from (i) and (ii) below:
(i) R¹⁹ and R²⁰ are each independently hydrogen, alkyl, cycloalkyl, cycloalkylalkyl or heterocyclylalkyl; or
(ii) R¹⁹ and R²⁰, together with the nitrogen atom to which they are attached, form a heterocyclyl;
R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³, and —R¹⁸NR¹⁵C(O)R²¹; wherein the alkyl, alkenyl, alkynyl, cycloalkyl, cycloalkylalkyl, heterocyclyl and heterocyclylalkyl may each be optionally substituted with one to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z}, and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};
R²¹ is hydrogen, alkyl, haloalkyl or cycloalkyl;
R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, and —R¹⁸NR²²R²³; wherein the alkyl, alkenyl, alkynyl, cycloalkyl, cycloalkylalkyl, heterocyclyl and heterocyclylalkyl may each be optionally substituted with one to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z}, and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z}; or two adjacent Q² groups on R⁸ may, together with the carbon atoms to which they are attached, form alkylenedioxy;
R²² and R²³ are independently selected from (i) or (ii) below:
(i) each R²² and R²³ is independently hydrogen or alkyl wherein each alkyl may each be optionally substituted by one or more substituents independently selected from the group consisting of halo, heterocyclyl, —OR^{x}, —NR^{y}R^{z} and —C(O)OR^{x}, or
(ii) R²² and R²³, together with the nitrogen atom to which they are attached, form a heterocyclyl which may optionally be substituted with one or more substituents independently selected from the group consisting of halo, oxo, alkyl, haloalkyl, hydroxyalkyl, —OR^{x}, —NR^{y}R^{z} and —C(O)OR^{x};
each R^{u} is independently hydrogen or alkyl;
each R^{v} is independently alkyl, haloalkyl, alkenyl, alkynyl or cycloalkyl;
each R^{w} is independently a direct bond or alkylene;
each R^{x} is independently hydrogen, alkyl, alkenyl, alkynyl or cycloalkyl;
each R^{y} and R^{z} is independently selected from (i) and (ii) below:
(i) R^{y} and R^{z} are each independently hydrogen, alkyl, cycloalkyl or cycloalkylalkyl, or
(ii) R^{y} and R^{z}, together with the nitrogen atom to which they are attached, form a heterocyclyl which may be optionally substituted with one or more substituents independently selected from the group consisting of halo, oxo, alkyl, haloalkyl, hydroxyalkyl, hydroxy and alkoxy; and
p is an integer selected from 0, 1 and 2.

2. The compound of claim 1 having formula: 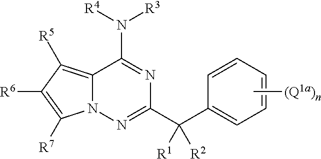
or a pharmaceutically acceptable salt thereof, wherein R³ is azolyl or azinyl optionally substituted with one to three substituents selected from the group consisting of halo, amino, alkyl and cycloalkyl.

3. The compound of claim 1, wherein R³ is azolyl optionally substituted with one to three substituents selected from the group consisting of halo, amino, alkyl and cycloalkyl.

4. The compound of claim 3, wherein R³ is pyrazolyl, optionally substituted with one to three substituents selected from fluoro, amino, methyl, cyclopropyl and cyclobutyl.

5. The compound of claim 3, wherein R³ is thiazolyl, optionally substituted with halo.

6. The compound of claim 1, wherein R⁸ is cycloalkyl, aryl, heterocyclyl or heteroaryl, optionally substituted with one, two or three substituents selected from halo, cyano, alkyl and alkoxy.

7. The compound of claim 1, wherein R⁸ is phenyl optionally substituted with one, two or three substituents selected from fluoro, chloro, cyano, methyl, —OR²¹ and —SR²¹ wherein R²¹ is methyl.

8. The compound of claim 1, wherein R⁸ is thienyl optionally substituted with one, two or three substituents selected from halo, cyano, alkyl, —OR²¹ and —SR²¹, wherein R²¹ is alkyl.

9. The compound of claim 1, wherein R⁵, R⁶ and R⁷ are each independently hydrogen, halo or alkyl.

10. The compound of claim 1, wherein R⁵, R⁶ and R⁷ are each independently hydrogen, chloro or fluoro.

11. The compound of claim 1, wherein the compound has formula: 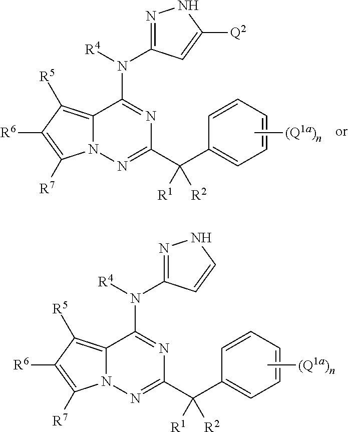
or a pharmaceutically acceptable salt thereof, where n is 0-5; Q² is selected from halo, amino, alkyl, alkenyl, alkynyl, cycloalkyl, and cycloalkylalkyl; and Q^{1a} is selected from halo, cyano, alkyl and alkoxy.

12. The compound of claim 11, wherein R¹ and R² are selected from (i), (ii) and (iii) as follows:
(i) R¹ and R² together form ═O;
(ii) R¹ and R² are each independently hydrogen or halo; and
(iii) R¹ is —OR¹², —NR¹³R¹⁴ or —N(R¹⁵)C(O)OR¹², and R² is hydrogen, cyano, alkyl, haloalkyl, hydroxyalkyl, alkoxyalkyl or cycloalkyl.

13. The compound of claim 1 selected from
(4-fluorophenyl)(4-(5-methyl-1H-pyrazol-3-ylamino)pyrrolo[1,2-f][1,2,4]triazin-2-yl)methanone;
(4-fluorophenyl)(4-(5-methyl-1H-pyrazol-3-ylamino)pyrrolo[1,2-f][1,2,4]triazin-2-yl)methanol;
2-(fluoro(4-fluorophenyl)methyl)-N-(5-methyl-1H-pyrazol-3-yl)pyrrolo[1,2-f][1,2,4]triazin-4-amine;
2-(difluoro(4-fluorophenyl)methyl)-N-(5-methyl-1H-pyrazol-3-yl)pyrrolo[1,2-f][1,2,4]triazin-4-amine;
(4-(1H-pyrazol-3-ylamino)pyrrolo[1,2-f][1,2,4]triazin-2-yl)(4-fluorophenyl)methanone;
(4-(1H-pyrazol-3-ylamino)pyrrolo[1,2-f][1,2,4]triazin-2-yl) (4-fluorophenyl)methanol;
2-(fluoro(4-fluorophenyl)methyl)-N-(1H-pyrazol-3-yl)pyrrolo[1,2-f][1,2,4]triazin-4-amine;
2-(difluoro(4-fluorophenyl)methyl)-N-(1H-pyrazol-3-yl)pyrrolo[1,2-f][1,2,4]triazin-4-amine;
(4-(1H-pyrazol-3-ylamino)-7-chloropyrrolo[1,2-f][1,2,4]triazin-2-yl)(4-fluorophenyl)methanone;
2-(4-fluorobenzyl)-N-(1H-pyrazol-3-yl)pyrrolo[1,2-f][1,2,4]triazin-4-amine;
(3-fluorophenyl)(4-(5-methyl-1H-pyrazol-3-ylamino)pyrrolo[1,2-f][1,2,4]triazin-2-yl)methanone;
(3-fluorophenyl)(4-(5-methyl-1H-pyrazol-3-ylamino)pyrrolo[1,2-f][1,2,4]triazin-2-yl)methanol;
(4-(1H-pyrazol-3-ylamino)pyrrolo[1,2-f][1,2,4]triazin-2-yl)(3-fluorophenyl)methanone;
(4-(1H-pyrazol-3-ylamino)pyrrolo[1,2-f][1,2,4]triazin-2-yl)(3-fluorophenyl)methanol;
(4-(1H-pyrazol-3-ylamino)pyrrolo[1,2-f][1,2,4]triazin-2-yl)(2-methoxyphenyl)methanone;
(4-(1H-pyrazol-3-ylamino)pyrrolo[1,2-f][1,2,4]triazin-2-yl)(2-methoxyphenyl)methanol;
(5-fluoro-2-methoxyphenyl)(4-(5-methyl-1H-pyrazol-3-ylamino)pyrrolo-[1,2-f][1,2,4]triazin-2-yl)methanone;
(5-fluoro-2-methoxyphenyl)(4-(5-methyl-1H-pyrazol-3-ylamino)pyrrolo[1,2-f][1,2,4]triazin-2-yl)methanol;
(4-(1H-pyrazol-3-ylamino)pyrrolo[1,2-f][1,2,4]triazin-2-yl)(5-fluoro-2-methoxyphenyl)methanone;
4-(4-(1H-pyrazol-3-ylamino)pyrrolo[1,2-f][1,2,4]triazine-2-carbonyl)benzonitrile;
4-((4-(1H-pyrazol-3-ylamino)pyrrolo[1,2-f][1,2,4]triazin-2-yl)(hydroxy)methyl)benzonitrile;
(4-(1H-pyrazol-3-ylamino)pyrrolo[1,2-f][1,2,4]triazin-2-yl)(2,4-difluorophenyl)methanone;
5-(4-(1H-pyrazol-3-ylamino)pyrrolo[1,2-f][1,2,4]triazine-2-carbonyl)-2-fluorobenzonitrile;
3-(4-(1H-pyrazol-3-ylamino)pyrrolo[1,2-f][1,2,4]triazine-2-carbonyl)benzonitrile;
(4-(5-cyclopropyl-1H-pyrazol-3-ylamino)pyrrolo[1,2-f][1,2,4]triazin-2-yl)(4-fluorophenyl)methanone;
(4-(5-cyclopropyl-1H-pyrazol-3-ylamino)pyrrolo[1,2-f][1,2,4]triazin-2-yl)(4-fluorophenyl)methanol;
(4(5-cyclobutyl-1H-pyrazol-3-ylamino)pyrrolo[1,2-f][1,2,4]triazin-2-yl)(4-fluorophenyl)methanone;
(4-(5-cyclobutyl-1H-pyrazol-3-ylamino)pyrrolo[1,2-f][1,2,4]triazin-2-yl)(4-fluorophenyl)methanol;
(4-fluorophenyl)(4-(thiazol-2-ylamino)pyrrolo[1,2-f][1,2,4]triazin-2-yl)methanone;
(4-fluorophenyl)(4-(thiazol-2-ylamino)pyrrolo[1,2-f][1,2,4]triazin-2-yl)methanol;
(4-fluorophenyl)(4-(5-fluorothiazol-2-ylamino)pyrrolo[1,2-f][1,2,4]triazin-2-yl)methanone;
(4-fluorophenyl)(4-(pyrazin-2-ylamino)pyrrolo[1,2-f][1,2,4]triazin-2-yl)methanone;
(4-(1H-pyrazol-3-ylamino)pyrrolo[1,2-f][1,2,4]triazin-2-yl)(cyclopropyl)methanone;
(4-(1H-pyrazol-3-ylamino)pyrrolo[1,2-f][1,2,4]triazin-2-yl)(cyclohexyl)methanone;
(4-(5-methyl-1H-pyrazol-3-ylamino)pyrrolo[1,2-f][1,2,4]triazin-2-yl) (thiophen-2-yl)methanone;
(4-(1H-pyrazol-3-ylamino)pyrrolo[1,2-f][1,2,4]triazin-2-yl)(thiophen-2-yl)methasone;
(4-(1H-pyrazol-3-ylamino)pyrrolo[1,2-f][1,2,4]triazin-2-yl) (thiophen-2-yl)methanol;
N-((4-(1H-pyrazol-3-ylamino)pyrrolo[1,2-f][1,2,4]triazin-2-yl)(4-fluorophenyl)methyl)-2-hydroxyethanamonium acetate;
2-(1-(4-fluorophenyl)vinyl)-N-(1H-pyrazol-5-yl)pyrrolo[1,2-f][1,2,4]triazin-4-amine;
ethyl 3-(4-(1H-pyrazol-3-ylamino)pyrrolo[1,2-f][1,2,4]triazin-2-yl)-3-(4-fluorophenyl)acrylate;
ethyl 3-(4-(1H-pyrazol-3-ylamino)pyrrolo[1,2-f][1,2,4]triazin-2-yl)-3-(4-fluorophenyl)propanoate;
(4-(5-methyl-1H-pyrazol-3-ylamino)pyrrolo[1,2-f][1,2,4]triazin-2-yl)(quinolin-8-yl)methanone;
(4-(5-methyl-1H-pyrazol-3-ylamino)pyrrolo[1,2-f][1,2,4]triazin-2-yl)(quinolin-8-yl)methanol;
2-((4-(1H-pyrazol-3-ylamino)pyrrolo[1,2-f][1,2,4]triazin-2-yl)(4-fluorophenyl)methylamino)ethanol;
2-(difluoro(4-fluorophenyl)methyl)-4-(5-methyl-1H-pyrazol-3-ylamino)-N-(2-morpholinoethyl)pyrrolo[1,2-f][1,2,4]triazine-6-carboxamide;
2-[difluoro-(4-fluoro-phenyl)-methyl]-4-(5-methyl-1H-pyrazol-3-ylamino)-pyrrolo[2,1-f][1,2,4]triazine-6-carboxylic acid (2-dimethylamino-ethyl)-amide;
2-[difluoro-(4-fluoro-phenyl)-methyl]-4-(5-methyl-1H-pyrazol-3-ylamino)-pyrrolo[2,1-f][1,2,4]triazine-6-carboxylic acid (3-pyrrolidin-1-yl-propyl)-amide;
[2-[difluoro-(4-fluoro-phenyl)-methyl]-4-(5-methyl-1H-pyrazol-3-ylamino)-pyrrolo[2,1-f][1,2,4]triazin-6-yl]-(4-methyl-piperazin-1-yl)-methanone;
[1,4′]Bipiperidinyl-1′-yl-[2-[difluoro-(4-fluoro-phenyl)-methyl]-4-(5-methyl-1H-pyrazol-3-ylamino)-pyrrolo[2,1-f][1,2,4]triazin-6-yl]-methanone;
2-[Difluoro-(4-fluoro-phenyl)-methyl]-4-(5-methyl-1H-pyrazol-3-ylamino)-pyrrolo[2,1-f][1,2,4]triazine-6-carboxylic acid (3-morpholin-4-yl-propyl)-amide;
2-[Difluoro-(4-fluoro-phenyl)-methyl]-4-(5-methyl-1H-pyrazol-3-ylamino)-pyrrolo[2,1-f][1,2,4]triazine-6-carboxylic acid (3-piperidin-1-yl-propyl)-amide;
2-[Difluoro-(4-fluoro-phenyl)-methyl]-4-(5-methyl-1H-pyrazol-3-ylamino)-pyrrolo[2,1-f][1,2,4]triazine-6-carboxylic acid [2-(4-methyl-piperazin-1-yl)-ethyl]-amide;
3-(Diethylamino)-N-(2-(difluoro(4-fluorophenyl)methyl)-4-(5-methyl-1H-pyrazol-3-ylamino)pyrrolo[1,2-f][1,2,4]triazin-6-yl)propanamide;
N[2-[Difluoro-(4-fluoro-phenyl)-methyl]-4-(5-methyl-1H-pyrazol-3-ylamino)-pyrrolo[2,1-f][1,2,4]triazin-6-yl]-3-(4,4-difluoro-piperidin-1-yl)-propionamide;
N-[2-[Difluoro-(4-fluoro-phenyl)-methyl]-4-(5-methyl-1H-pyrazol-3-ylamino)-pyrrolo[2,1-f][1,2,4]triazin-6-yl]-3-(4-methyl-piperazin-1-yl)-propionamide;
(2-(difluoro(4-fluorophenyl)methyl)-4-(5-methyl-1H-pyrazol-3-ylamino)pyrrolo[1,2-f][1,2,4]triazin-6-yl)methanol;
6-(aminomethyl)-2-(difluoro(4-fluorophenyl)methyl)-N-(5-methyl-1H-pyrazol-3-yl)pyrrolo[1,2-f][1,2,4]triazin-4-amine;
N((2-(difluoro(4-fluorophenyl)methyl)-4-(5-methyl-1H-pyrazol-3-ylamino)pyrrolo[1,2-f][1,2,4]triazin-6-yl)methyl)acetamide;
[2-[Difluoro-(4-fluoro-phenyl)-methyl]-4-(5-methyl-1H-pyrazol-3-ylamino)-pyrrolo[2,1-f][1,2,4]triazin-6-ylmethyl]-carbamic acid methyl ester;
N-[2-[Difluoro-(4-fluoro-phenyl)-methyl]-4-(5-methyl-1H-pyrazol-3-ylamino)-pyrrolo[2,1-f][1,2,4]triazin-6-ylmethyl]-methanesulfonamide;
N-[2-[Difluoro-(4-fluoro-phenyl)-methyl]-4-(5-methyl-1H-pyrazol-3-ylamino)-pyrrolo[2,1-f][1,2,4]triazin-6-ylmethyl]-isobutyramide;
N-[2-[Difluoro-(4-fluoro-phenyl)-methyl]-4-(5-methyl-1H-pyrazol-3-ylamino)-pyrrolo[2,1-f][1,2,4]triazin-6-ylmethyl]-2,2-dimethyl-propionamide;
1-(2-(difluoro(4-fluorophenyl)methyl)-4-(5-methyl-1H-pyrazol-3-ylamino)pyrrolo[1,2-f][1,2,4]triazin-6-yl)ethanone;
1-(2-(difluoro(4-fluorophenyl)methyl)-4-(5-methyl-1H-pyrazol-3-ylamino)pyrrolo[1,2-f][1,2,4]triazin-6-yl)ethanol;
2-(difluoro(4-fluorophenyl)methyl)-6-(ethoxymethyl)-N-(5-methyl-1H-pyrazol-3-yl)pyrrolo[1,2-f][1,2,4]triazin-4-amine;
2-(difluoro(4-fluorophenyl)methyl)-6-(isopropoxymethyl)-N-(5-methyl-1H-pyrazol-3-yl)pyrrolo[1,2-f][1,2,4]triazin-4-amine;
N-(2-(Difluoro(4-fluorophenyl)methyl)-4-(5-methyl-1H-pyrazol-3-ylamino)pyrrolo[1,2-f][1,2,4]triazin-6-yl)-2-morpholinoacetamide;
N-[2-[Difluoro-(4-fluoro-phenyl)-methyl]-4-(5-methyl-1H-pyrazol-3-ylamino)-pyrrolo[2,1-f][1,2,4]triazin-6-yl]-2-(3,3-difluoro-piperidin-1-yl)-acetamide;
N-[2-[Difluoro-(4-fluoro-phenyl)-methyl]-4-(5-methyl-1H-pyrazol-3-ylamino)-pyrrolo[2,1-f][1,2,4]triazin-6-yl]-2-(3,3-difluoro-pyrrolidin-1-yl)-acetamide;
2-(3,3-Difluoro-azetidin-1-yl)-N-[2-[difluoro-(4-fluoro-phenyl)-methyl]-4-(5-methyl-1H-pyrazol-3-ylamino)-pyrrolo[2,1-f][1,2,4]triazin-6-yl]-acetamide;
N-[2-[Difluoro-(4-fluoro-phenyl)-methyl]-4-(5-methyl-1H-pyrazol-3-ylamino)-pyrrolo[2,1-f][1,2,4]triazin-6-yl]-2-(4,4-difluoro-piperidin-1-yl)-acetamide;
N-[2-[Difluoro-(4-fluoro-phenyl)-methyl]-4-(5-methyl-1H-pyrazol-3-ylamino)-pyrrolo[2,1-f][1,2,4]triazin-6-yl]-2-(4-methyl-piperazin-1-yl)-acetamide;
2-(difluoro(4-fluorophenyl)methyl)-N-(5-methyl-1H-pyrazol-3-yl)-6-(morpholinomethyl)pyrrolo[1,2-f][1,2,4]triazin-4-amine;
6-((cyclopropylamino)methyl)-2-(difluoro(4-fluorophenyl)methyl)-N-(5-methyl-1H-pyrazol-3-yl)pyrrolo[1,2-f][1,2,4]triazin-4-amine;
2-(difluoro(4-fluorophenyl)methyl)-6-((3,3-difluoropyrrolidin-1-yl)methyl)-N-(5-methyl-1H-pyrazol-3-yl)pyrrolo[1,2-f][1,2,4]triazin-4-amine;
2-(difluoro(4-fluorophenyl)methyl)-6-((3,3-difluoroazetidin-1-yl)methyl)-N-(5-methyl-1H-pyrazol-3-yl)pyrrolo[1,2-f][1,2,4]triazin-4-amine;
(Z)-1-(2-(difluoro(4-fluorophenyl)methyl)-4-(5-methyl-1H-pyrazol-3-ylamino)pyrrolo[1,2-f][1,2,4]triazin-6-yl)ethanone oxime;
2-(difluoro(4-fluorophenyl)methyl)-4-(5-methyl-1H-pyrazol-3-ylamino)pyrrolo[1,2f][1,2,4]triazine-6-carbaldehyde oxime;
(4-fluorophenyl)(6-(hydroxymethyl)-4-(5-methyl-1H-pyrazol-3-ylamino)pyrrolo[1,2-f][1,2,4]triazin-2-yl)methanol;
2-(2-((4-fluorophenyl)(hydroxy)methyl)-4-(5-methyl-1H-pyrazol-3-ylamino)pyrrolo[1,2-f][1,2,4]triazin-6-yl)propan-2-ol;
2-(2-(1-(4-fluorophenyl)-1-hydroxyethyl)-4-(5-methyl-1H-pyrazol-3-ylamino)pyrrolo[1,2-f][1,2,4]triazin-6-yl)propan-2-ol;
2-(difluoro(4-fluorophenyl)methyl)-4-(5-methyl-1H-pyrazol-3-ylamino)pyrrolo[1,2-f][1,2,4]triazin-6-ol;
2-(difluoro(4-fluorophenyl)methyl)-N-(5-methyl-1H-pyrazol-3-yl)-6-(3-morpholinopropoxy)pyrrolo[1,2-f][1,2,4]triazin-4-amine;
2-(2-44-fluorophenyl)(methoxy)methyl)-4-(5-methyl-1H-pyrazol-3-ylamino)pyrrolo[1,2-f][1,2,4]triazin-6-yl)propan-2-ol;
methyl (4-fluorophenyl)(6-(2-hydroxypropan-2-yl)-4-(5-methyl-1H-pyrazol-3-ylamino)pyrrolo[1,2-f][1,2,4]triazin-2-yl)methylcarbamate;
2-(difluoro(4-fluorophenyl)methyl)-N-(5-methyl-1H-pyrazol-3-yl)-6-((2-(piperidin-1-yl)ethoxy)methyl)pyrrolo[1,2-f][1,2,4]triazin-4-amine acetate;
2-(difluoro(4-fluorophenyl)methyl)-N-(5-methyl-1H-pyrazol-3-yl)-6-(((1-methylpiperidin-3-yl)methoxy)methyl)pyrrolo[1,2-f][1,2,4]triazin-4-amine;
2-(difluoro(4-fluorophenyl)methyl)-N-(5-methyl-1H-pyrazol-3-yl)-6-((2-(pyrrolidin-1-yl)ethoxy)methyl) pyrrolo[1,2-f][1,2,4]triazin-4-amine;
2-(difluoro(4-fluorophenyl)methyl)-N-(5-methyl-1H-pyrazol-3-yl)-6-((2-morpholinoethoxy)methyl)pyrrolo[1,2-f][1,2,4]triazin-4-amine;
2-(difluoro(4-fluorophenyl)methyl)-N-(5-methyl-1H-pyrazol-3-yl)-6-((3-morpholinopropoxy)methyl)pyrrolo[1,2-f][1,2,4]triazin-4-amine;
(4-fluorophenyl)(4-(5-methyl-1H-pyrazol-3-ylamino)pyrrolo[1,2-f][1,2,4]triazin-2-yl)methanone oxime;
1-(4-fluorophenyl)-1-(4-(5-methyl-1H-pyrazol-3-ylamino)pyrrolo[1,2-f][1,2,4]triazin-2-yl)ethanol;
1-(4-Fluoro-phenyl)-2-methyl-1-[4-(5-methyl-1H-pyrazol-3-ylamino)-pyrrolo[2,1-f][1,2,4]triazin-2-yl]-propan-1-ol;
Cyclopropyl-(4-fluoro-phenyl)-[4-(5-methyl-1H-pyrazol-3-ylamino)-pyrrolo[2,1-f][1,2,4]triazin-2-yl]-methanol;
1-[6-Fluoro-4-(5-methyl-1H-pyrazol-3-ylamino)-pyrrolo[2,1-f][1,2,4]triazin-2-yl]-1-(4-fluoro-phenyl)-ethanol;
3-(4-(1H-pyrazol-3-ylamino)pyrrolo[1,2-f][1,2,4]triazin-2-yl)-3-(4-fluorophenyl)propan-1-ol;
2-(1-(4-fluorophenyl)-3-morpholinopropyl)-N-(1H-pyrazol-3-yl)pyrrolo[1,2-f][1,2,4]triazin-4-amine;
{2-[1-(4-Fluoro-phenyl)-3-piperidin-1-yl-propyl]-pyrrolo[2,1-f][1,2,4]triazin-4-yl}-(1H-pyrazol-3-yl)-amine;
{2-[1-(4-Fluoro-phenyl)-3-pyrrolidin-1-yl-propyl]-pyrrolo[2,1-f][1,2,4]triazin-4-yl}-(1H-pyrazol-3-yl)-amine;
{2-[3-Dimethylamino-1-(4-fluoro-phenyl)-propyl]-pyrrolo[2,1-f][1,2,4]triazin-4-yl}-(1H-pyrazol-3-yl)-amine;
2-(amino(4-fluorophenyl)methyl)-N-(5-methyl-1H-pyrazol-3-yl)pyrrolo[1,2-f][1,2,4]triazin-4-amine;
N-((4-fluorophenyl)(4-(5-methyl-1H-pyrazol-3-ylamino)pyrrolo[1,2-f][1,2,4]triazin-2-yl)methyl)-2-morpholinoacetamide;
N-{(4-Fluoro-phenyl)-[4-(5-methyl-1H-pyrazol-3-ylamino)-pyrrolo[2,1-f][1,2,4]triazin-2-yl]-methyl}-2-(4-methyl-piperazin-1-yl)-acetamide;
2-Dimethylamino-N-{(4-fluoro-phenyl)-[4-(5-methyl-1H-pyrazol-3-ylamino)-pyrrolo[2,1-f][1,2,4]triazin-2-yl]-methyl}-acetamide;
2-(Difluoro-piperidin-1-yl)-N-{(4-fluoro-phenyl)-[4-(5-methyl-1H-pyrazol-3-ylamino)pyrrolo[2,1-f][1,2,4]triazin-2-yl]-methyl}-acetamide;
N-{(4-Fluoro-phenyl)-[4-(5-methyl-1H-pyrazol-3-ylamino)-pyrrolo[2,1-f][1,2,4]triazin-2-yl]-methyl}-2-(4-methanesulfonyl-piperazin-1-yl)-acetamide;
{(4-Fluoro-phenyl)-[4-(5-methyl-1H-pyrazol-3-ylamino)-pyrrolo[2,1-f][1,2,4]triazin-2-yl]-methyl}-carbamic acid 3-morpholin-4-yl-propyl ester;
{(4-Fluoro-phenyl)-[4-(5-methyl-1H-pyrazol-3-ylamino)-pyrrolo[2,1-f][1,2,4]triazin-2-yl]-methyl}-carbamic acid 3-(3,3-difluoro-azetidin-1-yl)-propyl ester;
{(4-Fluoro-phenyl)-[4-(5-methyl-1H-pyrazol-3-ylamino)-pyrrolo[2, 14][1,2,4]triazin-2-yl]-methyl}-carbamic acid 2-morpholin-4-yl-ethyl ester;
N-((4-fluorophenyl)(4-(5-methyl-1H-pyrazol-3-ylamino)pyrrolo[1,2-f][1,2,4]triazin-2-yl)methyl)cyclopropanecarboxamide;
2-((4-fluorophenyl)(2-morpholinoethoxy)methyl)-N-(5-methyl-1H-pyrazol-3-yl)pyrrolo[1,2-f][1,2,4]triazin-4-amine;
{2-[(4-Fluoro-phenyl)-(3-pyrrolidin-1-yl-propoxy)-methyl]-pyrrolo[2,1-f][1,2,4]triazin-4-yl}-(5-methyl-1H-pyrazol-3-yl)-amine;
{2-[(4-Fluoro-phenyl)-(3-morpholin-4-yl-propoxy)-methyl]-pyrrolo[2,1-f][1,2,4]triazin-4-yl}-(5-methyl-1H-pyrazol-3-yl)-amine;
(2-{(4-Fluoro-phenyl)-[3-(4-methyl-piperazin-1-yl)-propoxy]-methyl}-pyrrolo[2,1-f][1,2,4]triazin-4-yl)-(5-methyl-1H-pyrazol-3-yl)-amine;
{2-[(4-Fluoro-phenyl)-(2-pyrrolidin-1-yl-ethoxy)-methyl]-pyrrolo[2,1-f][1,2,4]triazin-4-yl}-(5-methyl-1H-pyrazol-3-yl)-amine;
(2-{(4-Fluoro-phenyl)-[2-(4-methyl-piperazin-1-yl)-ethoxy]-methyl}-pyrrolo[2,1-f][1,2,4]triazin-4-yl)-(5-methyl-1H-pyrazol-3-yl)-amine;
{2-[(2-Dimethylamino-ethoxy)-(4-fluoro-phenyl)-methyl]-pyrrolo[2,1-f][1,2,4]triazin-4-yl}-(5-methyl-1H-pyrazol-3-yl)-amine;
2((4-fluorophenyl)(methylamino)methyl)-N-(5-methyl-1H-pyrazol-3-yl)pyrrolo[1,2-f][1,2,4]triazin-4-amine;
{2-[(4-Fluoro-phenyl)-pyrrolidin-1-yl-methyl]-pyrrolo[2,1-f][1,2,4]triazin-4-yl}-(5-methyl-1H-pyrazol-3-yl)-amine;
{2-[(4-Fluoro-phenyl)-morpholin-4-yl-methyl]-pyrrolo[2,1-f][1,2,4]triazin-4-yl}-(5-methyl-1H-pyrazol-3-yl)-amine;
N¹-((4-fluorophenyl)(4-(5-methyl-1H-pyrazol-3-ylamino)pyrrolo[1,2-f][1,2,4]triazin-2-yl)methyl)-N²,N²-dimethylethane-1,2-diamine;
{2-[(4-Fluoro-phenyl)-(2-methoxy-ethylamino)-methyl]-pyrrolo[2,1-f][1,2,4]triazin-4-yl}-(5-methyl-1H-pyrazol-3-yl)-amine;
{2-[(4-Fluoro-phenyl)-imidazol-1-yl-methyl]-pyrrolo[2,1-f][1,2,4]triazin-4-yl}-(5-methyl-1H-pyrazol-3-yl)-amine;
N-{(4-Fluoro-phenyl)-[4-(5-methyl-1H-pyrazol-3-ylamino)-pyrrolo[2,1-f][1,2,4]triazin-2-yl}methyl]-N,N′,N-trimethyl-ethane-1,2-diamine;
{2-[Dimethylamino-(4-fluoro-phenyl)-methyl]-pyrrolo[2,1-f][1,2,4]triazin-4-yl}-(5-methyl-1H-pyrazol-3-yl)-amine;
{2-[(4-Fluoro-phenyl)-(tetrahydro-pyran-4-yloxy)-methyl]-pyrrolo[2,1-f][1,2,4]triazin-4-yl}-(5-methyl-1H-pyrazol-3-yl)-amine;
{2-[(4-Fluoro-phenyl)-(tetrahydro-furan-3-yloxy)-methyl]-pyrrolo[2,1-f][1,2,4]triazin-4-yl}-(5-methyl-1H-pyrazol-3-yl)-amine;
2-((R,S)-(4-fluorophenyl)((R)-tetrahydrofuran-3-yloxy)methyl)-N-(5-methyl-1H-pyrazol-3-yl)pyrrolo[1,2-f][1,2,4]triazin-4-amine
{2-[(4-Fluoro-phenyl)-(2-pyrrolidin-1-yl-ethylamino)-methyl]-pyrrolo[2,1-f][1,2,4]triazin-4-yl}-(5-methyl-1H-pyrazol-3-yl)-amine;
{2-[(4-Fluoro-phenyl)-(2-pyridin-2-yl-ethylamino)-methyl]-pyrrolo[2,1-f][1,2,4]triazin-4-yl}-(5-methyl-1H-pyrazol-3-yl)-amine;
{2-[(2-Dimethylamino-ethylsulfanyl)-(4-fluoro-phenyl)-methyl]-pyrrolo[2,1-f][1,2,4]triazin-4-yl}-(5-methyl-1H-pyrazol-3-yl)-amine;
4-{(4-Fluoro-phenyl)-[4-(5-methyl-1H-pyrazol-3-ylamino)-pyrrolo[2,1-f][1,2,4]triazin-2-yl]-methoxy}-2-methyl-butan-2-ol;
1-(2-{(4-Fluoro-phenyl)-[4-(5-methyl-1H-pyrazol-3-ylamino)-pyrrolo[2,1-f][1,2,4]triazin-2-yl]-methoxy}-ethyl)-pyrrolidin-2-one;
{2-[Ethylamino-(4-fluoro-phenyl)-methyl]-pyrrolo[2,1-f][1,2,4]triazin-4-yl}-(5-methyl-1H-pyrazol-3-yl)-amine;
{2-[(4-Fluoro-phenyl)-isopropylamino-methyl]-pyrrolo[2,1-f][1,2,4]triazin-4-yl}-(5-methyl-1H-pyrazol-3-yl)-amine;
{2-[(4-Fluoro-phenyl)-isobutylamino-methyl]-pyrrolo[2,1-f][1,2,4]triazin-4-yl}-(5-methyl-1H-pyrazol-3-yl)-amine;
{2-[tert-Butylamino-(4-fluoro-phenyl)-methyl]-pyrrolo[2,1-f][1,2,4]triazin-4-yl}-(5-methyl-1H-pyrazol-3-yl)-amine;
{2-[Cyclobutylamino-(4-fluoro-phenyl)-methyl]-pyrrolo[2,1-f][1,2,4]triazin-4-yl}-(5-methyl-1H-pyrazol-3-yl)-amine;
{2-[Cyclopropylamino-(4-fluoro-phenyl)-methyl]-pyrrolo[2,1-f][1,2,4]triazin-4-yl}-(5-methyl-1H-pyrazol-3-yl)-amine;
{2-[(2-tert-Butoxy-ethoxy)-(4-fluoro-phenyl)-methyl]-pyrrolo[2,1-f][1,2,4]triazin-4-yl}-(5-methyl-1H-pyrazol-3-yl)-amine;
2-{(4-Fluoro-phenyl)-[4-(5-methyl-1H-pyrazol-3-ylamino)-pyrrolo[2,1-f][1,2,4]triazin-2-yl]-methylsulfanyl}-ethanol;
{2-[(Cyclopropylmethyl-amino)-(4-fluoro-phenyl)-methyl]-pyrrolo[2,1-f][1,2,4]triazin-4-yl}-(5-methyl-1H-pyrazol-3-yl)-amine;
2-((R,S)-(4-fluorophenyl)(((S)-tetrahydrofuran-2-yl)methylamino)methyl)-N-(5-methyl-1H-pyrazol-3-yl)pyrrolo[1,2-f][1,2,4]triazin-4-amine;
2-((R,S)-(4-fluorophenyl)(((R)-tetrahydrofuran-2-yl)methylamino)methyl)-N-(5-methyl-1H-pyrazol-3-yl)pyrrolo[1,2-f][1,2,4]triazin-4-amine;
{2-[Ethoxy-(4-fluoro-phenyl)-methyl]-pyrrolo[2,1-f][1,2,4]triazin-4-yl}-(5-methyl-1H-pyrazol-3-yl)-amine;
{2-[(4-Fluoro-phenyl)-(2,2,2-trifluoro-ethoxy)-methyl]-pyrrolo[2,1-f][1,2,4]triazin-4-yl}-(5-methyl-1H-pyrazol-3-yl)-amine;
{2-[(2,2-Difluoro-ethoxy)-(4-fluoro-phenyl)-methyl]-pyrrolo[2,1-f][1,2,4]triazin-4-yl}-(5-methyl-1H-pyrazol-3-yl)-amine;
{2-[(4-Fluoro-phenyl)-thiocyanato-methyl]-pyrrolo[2,1-f][1,2,4]triazin-4-yl}-(5-methyl-1,4-pyrazol-3-yl)-amine;
{2-[(4-Fluoro-phenyl)-methylsulfanyl-methyl]-pyrrolo[2,1-f][1,2,4]triazin-4-yl}-(5-methyl-1H-pyrazol-3-yl)-amine;
2-(difluoro(4-fluorophenyl)methyl)-6-((2-methoxyethoxy)methyl)-N-(5-methyl-1H-pyrazol-3-yl)pyrrolo[1,2-f][1,2,4]triazin-4-amine;
3-(2-(4-fluorobenzoyl)pyrrolo[1,2-f][1,2,4]triazin-4-ylamino)-1H-pyrazole-5-carbonitrile;
5-{2-[(4-Fluoro-phenyl)-hydroxy-methyl]-pyrrolo[2,1-f][1,2,4]triazin-4-ylamino}-21H-pyrazole-35-carbonitrile;
(6-chloro-4-(5-methyl-1H-pyrazol-3-ylamino)pyrrolo[1,2-f][1,2,4]triazin-2-yl)(4-fluorophenyl)methanone;
[6-Chloro-4-(5-methyl-1H-pyrazol-3-ylamino)-pyrrolo[2,1-f][1,2,4]triazin-2-yl]-(4-fluorophenyl)-methanol;
2-((4-fluorophenyl)(((R)-tetrahydrofuran-2-yl)methoxy)methyl)-N-(5-methyl-1H-pyrazol-3-yl)pyrrolo[1,2-f][1,2,4]triazin-4-amine;
2-((2-(tert-butylamino)ethoxy)(4-fluorophenyl)methyl)-N-(5-methyl-1H-pyrazol-3-yl)pyrrolo[1,2-f][1,2,4]triazin-4-amine;
2((4-fluorophenyl)(2-morpholinoethylthio)methyl)-N-(5-methyl-1H-pyrazol-3-yl)pyrrolo[1,2-f][1,2,4]triazin-4-amine;
2-((4-fluorophenyl)(3-(methylthio)propylamino)methyl)-N-(5-methyl-1H-pyrazol-3-yl)pyrrolo[1,2-f][1,2,4]triazin-4-amine;
N-((2-(difluoro(4-fluorophenyl)methyl)-4-(5-methyl-1H-pyrazol-3-ylamino)pyrrolo[1,2-f][1,2,4]triazin-6-yl)methyl)-2-morpholinoacetamide;
1-(2-(difluoro(4-fluorophenyl)methyl)-4-(5-methyl-1H-pyrazol-3-ylamino)pyrrolo[1,2-f][1,2,4]triazin-6-yl)-2,2,2-trifluoroethanol;
2-amino-1-(2-(difluoro(4-fluorophenyl)methyl)-4-(5-methyl-1H-pyrazol-3-ylamino)pyrrolo[1,2-f][1,2,4]triazin-6-yl)ethanol;
N-(2-(2-(difluoro(4-fluorophenyl)methyl)-4-(5-methyl-1H-pyrazol-3-ylamino)pyrrolo[1,2-f][1,2,4]triazin-6-yl)-2-hydroxyethyl)acetamide;
1-(2-(difluoro(4-fluorophenyl)methyl)-4-(5-methyl-1H-pyrazol-3-ylamino)pyrrolo[1,2-f][1,2,4]triazin-6-yl)-2-morpholinoethanol;
5-(2-(difluoro(4-fluorophenyl)methyl)-4-(5-methyl-1H-pyrazol-3-ylamino)pyrrolo[1,2-f][1,2,4]triazin-6-yl)oxazolidin-2-one;
2-(1-(4-fluorophenyl)ethyl)-N-(1H-pyrazol-3-yl)pyrrolo[1,2-f][1,2,4]triazin-4-amine;
4-(4-(1H-pyrazol-3-ylamino)pyrrolo[1,2-f][1,2,4]triazin-2-yl)-4-(4-fluorophenyl)butan-1-ol;
2-(2-(difluoro(4-fluorophenyl)methyl)-4-(5-methyl-1H-pyrazol-3-ylamino)pyrrolo[1,2-f][1,2,4]triazin-6-yl)propan-2-ol;
[2-(4-Fluoro-benzyl)-pyrrolo[2,1-f][1,2,4]triazin-4-yl]-(5-methyl-1H-pyrazol-3-yl)-amine;
(6-fluoro-4-(5-methyl-1H-pyrazol-3-ylamino)pyrrolo[1,2-f][1,2,4]triazin-2-yl)(4-fluorophenyl)methanone;
(6-fluoro-4-(5-methyl-1H-pyrazol-3-ylamino)pyrrolo[1,2-f][1,2,4]triazin-2-yl)(4-fluorophenyl)methanol;
6-fluoro-2((4-fluorophenyl)(methoxy)methyl)-N-(5-methyl-1H-pyrazol-3-yl)pyrrolo[1,2-f][1,2,4]triazin-4-amine;
(4-(1H-pyrazol-3-ylamino)-6-fluoropyrrolo[1,2-f][1,2,4]triazin-2-yl)(4-fluorophenyl)methanone;
2-(difluoro(4-fluorophenyl)methyl)-6-fluoro-N-(5-methyl-1H-pyrazol-3-yl)pyrrolo[1,2-f][1,2,4]triazin-4-amine;
2-(difluoro(4-fluorophenyl)methyl)-6-fluoro-N-(1H-pyrazol-3-yl)pyrrolo[1,2-f][1,2,4]triazin-4-amine;
(2-(difluoro(4-fluorophenyl)methyl)-4-(5-methyl-1H-pyrazol-3-ylamino)pyrrolo[1,2-f][1,2,4]triazin-6-yl)(morpholino) methanethione;
2-(difluoro(4-fluorophenyl)methyl)-N-(5-methyl-1H-pyrazol-3-yl)-6-(methylsulfonylmethyl)pyrrolo[1,2-f][1,2,4]triazin-4-amine;
N-(2-(difluoro(4-fluorophenyl)methyl)-4(5-methyl-1H-pyrazol-3-ylamino)pyrrolo[1,2-f][1,2,4]triazin-6-yl)-3-morpholinopropanamide;
N-(5-chloro-2-(difluoro(4-fluorophenyl)methyl)-4-(5-methyl-1H-pyrazol-3-ylamino)pyrrolo[1,2-f][1,2,4]triazin-6-yl)-3-morpholinopropanamide;
N-(2-(difluoro(4-fluorophenyl)methyl)-4-(5-methyl-1H-pyrazol-3-ylamino)pyrrolo[1,2-f][1,2,4]triazin-6-yl)acrylamide;
N(2-(difluoro(4-fluorophenyl)methyl)-4-(5-methyl-1H-pyrazol-3-ylamino)pyrrolo[1,2-f][1,2,4]triazin-6-yl)-2-(4,4-difluoropiperidin-1-yl)ethanesulfonamide;
(7-bromo-4-(5-methyl-1H-pyrazol-3-ylamino)pyrrolo[1,2-f][1,2,4]triazin-2-yl)(4-fluorophenyl)methanone;
2-(4-fluorophenyl)-2-(4-(5-methyl-1H-pyrazol-3-ylamino)pyrrolo[1,2-f][1,2,4]triazin-2-yl)ethanol;
1-(4-fluorophenyl)-1-(4-(5-methyl-1H-pyrazol-3-ylamino)pyrrolo[1,2-f][1,2,4]triazin-2-yl)ethane-1,2-diol;
2-fluoro-2-((4-fluorophenyl)-2-(4-(5-methyl-1H-pyrazol-3-ylamino)pyrrolo[1,2-f][1,2,4]triazin-2-yl)ethanol;
2-(1-fluoro-1-(4-fluorophenyl)-2-methoxyethyl)-N-(5-methyl-1H-pyrazol-3-yl)pyrrolo[1,2-f][1,2,4]triazin-4-amine;
2-(2-amino-1-fluoro-1-(4-fluorophenyl)ethyl)-N-(5-methyl-1H-pyrazol-3-yl)pyrrolo[1,2-f][1,2,4]triazin-4-amine;
5-(4-fluorophenyl)-5-(4-(5-methyl-1H-pyrazol-3-ylamino)pyrrolo[1,2-f][1,2,4]triazin-2-yl)oxazolidin-2-one;
2-((4-fluorophenyl)(methoxyamino)methyl)-N-(5-methyl-1H-pyrazol-3-yl)pyrrolo[1,2-f][1,2,4]triazin-4-amine;
2-(2-(4-fluorophenyl)-1,3-dioxolan-2-yl)-N-(5-methyl-1H-pyrazol-3-yl)pyrrolo[1,2-f][1,2,4]triazin-4-amine;
2-(2-(4-fluorophenyl)-1,3-oxathiolan-2-yl)-N-(5-methyl-1H-pyrazol-3-yl)pyrrolo[1,2-f][1,2,4]triazin-4-amine;
3-(4-fluorophenyl)-3-(4-(5-methyl-1H-pyrazol-3-ylamino)pyrrolo[1,2-f][1,2,4]triazin-2-yl)propan-1-ol;
1-((4-fluorophenyl)(4-(5-methyl-1H-pyrazol-3-ylamino)pyrrolo[1,2-f][1,2,4]triazin-2-yl)methyl)-3-methylurea;
2-(1-amino-1-(4-fluorophenyl)ethyl)-N-(5-methyl-1H-pyrazol-3-yl)pyrrolo[1,2-f][1,2,4]triazin-4-amine;
2-((4-fluorophenyl)(methylsulfinyl)methyl)-N-(5-methyl-1H-pyrazol-3-yl)pyrrolo[1,2-f][1,2,4]triazin-4-amine;
2-((4-fluorophenyl)(methylsulfonyl)methyl)-N-(5-methyl-1H-pyrazol-3-yl)pyrrolo[1,2-f][1,2,4]triazin-4-amine;
(4-(1H-pyrazol-3-ylamino)pyrrolo[1,2-f][1,2,4]triazin-2-yl)(2-(methylthio)phenyl)methanone;
(4-chlorophenyl)(4-(5-methyl-1H-pyrazol-3-ylamino)pyrrolo[1,2-f][1,2,4]triazin-2-yl)methanone;
(4-Methoxy-phenyl)-[4-(5-methyl-1H-pyrazol-3-ylamino)-pyrrolo[2,1-f][1,2,4]triazin-2-yl]-methanone;
(4-Fluoro-3-methyl-phenyl)-[4-(5-methyl-1H-pyrazol-3-ylamino)-pyrrolo[2,1-f][1,2,4]triazin-2-yl]-methanone;
Benzo[1,3]dioxol-5-yl-[4-(5-methyl-1H-pyrazol-3-ylamino)-pyrrolo[2,1-f][1,2,4]triazin-2-yl]-methanone;
(3,4-Difluoro-phenyl)-[4-(5-methyl-1H-pyrazol-3-ylamino)-pyrrolo[2,1-f][1,2,4]triazin-2-yl]-methanone;
(4-Methoxy-phenyl)-[4-(5-methyl-1H-pyrazol-3-ylamino)-pyrrolo[2,1-f][1,2,4]triazin-2-yl]-methanol;
Benzo[1,3]dioxol-5-yl-[4-(5-methyl-1H-pyrazol-3-ylamino)-pyrrolo[2, 14][1,2,4]triazin-2-yl]-methanol;
(3,4-Difluoro-phenyl)-[4-(5-methyl-1H-pyrazol-3-ylamino)-pyrrolo[2,1-f][1,2,4]triazin-2-yl]-methanol;
(4-Fluoro-3-methyl-phenyl)-[4-(5-methyl-1H-pyrazol-3-ylamino)-pyrrolo[2,1-f][1,2,4]triazin-2-yl]-methanol;
(4-Chloro-phenyl)-[4-(5-methyl-1H-pyrazol-3-ylamino)-pyrrolo[2,1-f][1,2,4]triazin-2-yl]-methanol;
methyl (4-fluorophenyl)(4-(5-methyl-1H-pyrazol-3-ylamino)pyrrolo[1,2-f][1,2,4]triazin-2-yl)methylcarbamate;
{(4-Fluoro-phenyl)-[4-(5-methyl-1H-pyrazol-3-ylamino)-pyrrolo[2,1-f][1,2,4]triazin-2-yl]-methyl}-carbamic acid ethyl ester;
2-Amino-N-{(4-fluoro-phenyl)-[4-(5-methyl-1H-pyrazol-3-ylamino)-pyrrolo[2,1-f][1,2,4]triazin-2-yl]-methyl}-propionamide;
(R)-2-amino-N—((R,S)-(4-fluorophenyl)(4-(5-methyl-1H-pyrazol-3-ylamino)pyrrolo[1,2-f][1,2,4]triazin-2-yl)methyl)propanamide;
2-Amino-N-{(4-fluoro-phenyl)-[4-(5-methyl-1H-pyrazol-3-ylamino)-pyrrolo[2,1-f][1,2,4]triazin-2-yl]-methyl}-acetamide;
3-{(4-Fluoro-phenyl)-[4-(5-methyl-1H-pyrazol-3-ylamino)-pyrrolo[2,1-f][1,2,4]triazin-2-yl]-methyl}-1,1-dimethyl-urea;
N-{(4-Fluoro-phenyl)-[4-(5-methyl-1H-pyrazol-3-ylamino)-pyrrolo[2,1-f][1,2,4]triazin-2-yl]-methyl}-formamide;
N-{(4-Fluoro-phenyl)-[4-(5-methyl-1H-pyrazol-3-ylamino)-pyrrolo[2,1-f][1,2,4]triazin-2-yl]-methyl}-methanesulfonamide;
N-{(4-Fluoro-phenyl)-[4-(5-methyl-1-pyrazol-3-ylamino)-pyrrolo[2,1-f][1,2,4]triazin-2-yl]-methyl}-acetamide;
N-{(4-Fluoro-phenyl)-[4-(5-methyl-1H-pyrazol-3-ylamino)-pyrrolo[2,1-f][1,2,4]triazin-2-yl]-methyl}-isobutyramide;
2,2,2-Trifluoro-N-{(4-fluoro-phenyl)-[4-(5-methyl-1H-pyrazol-3-ylamino)-pyrrolo[2,1-f][1,2,4]triazin-2-yl]-methyl}-acetamide;
2-((4-fluorophenyl)(2-methoxyethoxy)methyl)-N-(5-methyl-1H-pyrazol-3-yl)pyrrolo[1,2-f][1,2,4]triazin-4-amine;
{2-[(2-Ethoxy-ethoxy)-(4-fluoro-phenyl)-methyl]-pyrrolo[2,1-f][1,2,4]triazin-4-yl}-(5-methyl-1H-pyrazol-3-yl)-amine;
2-{(4-Fluoro-phenyl)-[4-(5-methyl-1H-pyrazol-3-ylamino)-pyrrolo[2,1-f][1,2,4]triazin-2-yl]-methoxy}-ethanol;
3-{(4-Fluoro-phenyl)-[4-(5-methyl-1H-pyrazol-3-ylamino)-pyrrolo[2,1-f][1,2,4]triazin-2-yl]-methoxy}-propan-1-ol;
2-((4-fluorophenyl)(3-morpholinopropylthio)methyl)-N-(5-methyl-1H-pyrazol-3-yl)pyrrolo[1,2-f][1,2,4]triazin-4-amine;
(2-{(4-Fluoro-phenyl)-[2-(4-methyl-piperazin-1-yl)-ethyl sulfanyl]-methyl}-pyrrolo[2,1-f][1,2,4]triazin-4-yl)-(5-methyl-1H-pyrazol-3-yl)-amine;
(2-{(4-Fluoro-phenyl)-[3-(4-methyl-piperazin-1-yl)-propyl sulfanyl]methyl}-pyrrolo[2,1-f][1,2,4]triazin-4-yl)-(5-methyl-1H-pyrazol-3-yl)-amine;
{2-[(3-Dimethylamino-propylsulfanyl)-(4-fluoro-phenyl)-methyl]-pyrrolo[2,1-f][1,2,4]triazin-4-yl}-(5-methyl-1H-pyrazol-3-yl)-amine;
{2-[(2-Amino-ethylsulfanyl)-(4-fluoro-phenyl)-methyl]-pyrrolo[2,1-f][1,2,4]triazin-4-yl}-(5-methyl-1H-pyrazol-3-yl)-amine;
{2-[[2-(3,3-Difluoro-pyrrolidin-1-yl)-ethylsulfanyl]-(4-fluoro-phenyl)-methyl]-pyrrolo[2,1f][1,2,4]triazin-4-yl}-(5-methyl-1H-pyrazol-3-yl)-amine;
(2S)-((R,S)(4-fluorophenyl)(4-(5-methyl-1H-pyrazol-3-ylamino)pyrrolo[1,2-f][1,2,4]triazin-2-yl)methyl) 2-aminopropanoate;
Amino-acetic acid (4-fluoro-phenyl)-[4-(5-methyl-1H-pyrazol-3-ylamino)-pyrrolo[2,1-f][1,2,4]triazin-2-yl]-methyl ester;
(R)—((R,S)-(4-fluorophenyl)(4-(5-methyl-1H-pyrazol-3-ylamino)pyrrolo[1,2-f][1,2,4]triazin-2-yl)methyl) 2-aminopropanoate;
Morpholin-4-yl-acetic acid (4-fluoro-phenyl)-[4-(5-methyl-1H-pyrazol-3-ylamino)pyrrolo[2,1-f][1,2,4]triazin-2-yl]-methyl ester;
2-Amino-2-methyl-propionic acid (4-fluoro-phenyl)-[4-(5-methyl-1H-pyrazol-3-ylamino)pyrrolo[2,1-f][1,2,4]triazin-2-yl]-methyl ester;
2-Methyl-2-methylamino-propionic acid (4-fluoro-phenyl)-[4-(5-methyl-1H-pyrazol-3-ylamino)-pyrrolo[2,1-f][1,2,4]triazin-2-yl]-methyl ester;
methyl 2-((4-fluorophenyl)(methoxy)methyl)-N-(5-methyl-1H-pyrazol-3-yl)pyrrolo[1,2-f][1,2,4]triazin-4-amine; and
N-(4-bromo-5-methyl-1H-pyrazol-3-yl)-2-(difluoro(4-fluorophenyl)methyl)-6-(methylsulfonylmethyl)pyrrolo[1,2-f][1,2,4]triazin-4-amine.

14. A pharmaceutical composition comprising a compound of claim 1 and a pharmaceutically acceptable carrier.

15. A method for treatment of a disease selected from an inflammatory disease, an inflammatory condition, an autoimmune disease and cancer comprising administering a therapeutically effective amount of a compound of claim 1.

16. The method of claim 15, wherein the disease is modulated by JAK.

17. The method of claim 16, wherein the disease is modulated by wild type or mutant JAK2.

18. A method for the treatment of a disease comprising administering a therapeutically effective amount of a compound of claim 1, wherein the disease is selected from myeloproliferative disorder, polycythemia vera (PCV), essential thrombocythemia (ET), primary myelofibrosis (PMF), chronic eosinophilic leukemia (CEL), chronic myelomonocytic leukemia (CMML), systemic mastocytosis (SM), idiopathic myelofibrosis (IMF), myeloid leukemia, chronic myeloid leukemia (CML), imatinib-resistant CML, acute myeloid leukemia (AML), acute megakaryoblastic leukemia (AMKL), myeloma, cancer of the head and neck, prostate cancer, breast cancer, ovarian cancer, melanoma, lung cancer, brain cancer, pancreatic cancer, renal cancer, immunodeficiency, autoimmune diseases, tissue transplant rejection, graft-versus-host disease, wound, kidney disease, multiple sclerosis, thyroiditis, type 1 diabetes, sarcoidosis, psoriasis, allergic rhinitis, inflammatory bowel disease including Crohn's disease and ulcerative colitis (UC), systemic lupus erythematosis (SLE), arthritis, osteoarthritis, rheumatoid arthritis, osteoporosis, asthma and chronic obstructive pulmonary disease (COPD).

19. The method of claim 18 further comprising administering a second pharmaceutical agent selected from anti-proliferative agent, anti-inflammatory agent, immunomodulatory agent and immunosuppressive agent.

20. A method of modulating JAK by administering a compound of claim 1.

21. A compound having formula (I): 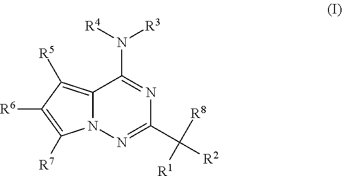
or a pharmaceutically acceptable salt thereof, wherein:
R¹ and R² are selected from (i), (ii), (iii) and (iv) as follows:
(i) R¹ and R² together form ═O, ═S, ═NR⁹ or ═CR¹⁰R¹¹;
(ii) R¹ and R² together with the carbon atom to which they are attached, form cycloalkyl or oxacycloalkyl;
(iii) R¹ and R² are each independently hydrogen, halo, haloalkyl, alkyl, alkenyl, alkynyl, cycloalkyl, hydroxyalkyl, alkoxyalkyl, —OR¹² or —SR¹²; or
(iv) R¹ is —OR¹², —NR¹³R¹⁴, —SR¹², —N(R¹⁵)R¹⁶OR¹² or —R¹⁸C(O)OR¹², and R² is hydrogen, cyano, haloalkyl, alkyl, alkenyl, alkynyl, cycloalkyl, hydroxyalkyl or alkoxyalkyl;
R³ is cycloalkyl, aryl, heterocyclyl or heteroaryl;
R⁴ is hydrogen or alkyl;
R⁵, R⁶ and R⁷ are each independently hydrogen, halo, nitro, alkyl, alkenyl, alkynyl or cycloalkyl, —OR^{x}, —NR¹⁵C(O)R¹⁷, —C(O)NR¹⁹R²⁰ wherein the alkyl, alkenyl, alkynyl and cycloalkyl may each be optionally substituted with one, two or three groups selected from halo, alkyl, haloalkyl, hydroxyalkyl, cyano, —R^{w}—OR^{x} and —R^{w}—NR^{y}R^{z};
R⁸ is cycloalkyl, aryl, heteroaryl or heterocyclyl;
R⁹ is alkyl, —OR¹² or —NR¹³R¹⁴;
R¹⁰ and R¹¹ are each independently hydrogen, alkyl or —C(O)OR¹²;
R¹² is hydrogen, alkyl, cycloalkyl, cycloalkylalkyl, heterocyclyl or heterocyclylalkyl wherein the alkyl, cycloalkyl or cycloalkylalkyl, heterocyclyl or heterocyclylalkyl may each be optionally substituted with one, two or three groups selected from halo, alkyl, hydroxy, alkoxy and amino;
each R¹³ and R¹⁴ is independently selected from (i) or (ii) below:
(i) R¹³ and R¹⁴ are each independently hydrogen, alkyl, cycloalkyl, cycloalkylalkyl, heterocyclyl or heterocyclylalkyl wherein the alkyl, cycloalkyl, cycloalkylalkyl, heterocyclyl or heterocyclylalkyl may each be optionally substituted with one, two or three groups independently selected from halo, oxo, alkyl, hydroxy, alkoxy and amino, or
(ii) R¹³ and R¹⁴, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;
each R¹⁵ is independently hydrogen or alkyl;
each R¹⁶ is independently alkylene or alkenylene;
each R¹⁷ is alkyl, alkenyl, alkynyl, cycloalkyl, cycloalkylalkyl, heterocyclyl, heterocyclylalkyl, aryl, aralkyl, heteroaryl or heteroaralkyl;
each R¹⁸ is independently alkylene or a direct bond;
each R¹⁹ and R²⁰ is independently selected from (i) or (ii) below:
(i) R¹⁹ and R²⁰ are each independently hydrogen, alkyl, cycloalkyl or cycloalkylalkyl wherein the alkyl, cycloalkyl or cycloalkylalkyl may each be optionally substituted with one, two or three groups independently selected from halo, oxo, alkyl, haloalkyl, hydroxyalkyl, hydroxy, alkoxy and amino, or
(ii) R¹⁹ and R²⁰, together with the nitrogen atom to which they are attached, form a heterocyclyl which may be optionally substituted with one, two or three groups independently selected from halo, oxo, alkyl, haloalkyl, hydroxyalkyl, hydroxy, alkoxy and amino;
R¹⁷, R¹⁹ and R²⁰ may optionally be substituted by one or more substituents independently selected from the group Q¹ consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸NR²²R²³, —R¹⁸NR¹⁵C(O)R²¹; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};
R³ and R⁸ may optionally be substituted by one or more substituents independently selected from the group Q² consisting of nitro, halo, cyano, oxo, thioxo, imino, amino, alkyl, alkenyl, alkynyl, cycloalkyl, cycloalkylalkyl, heterocyclyl, heterocyclylalkyl, aryl, aralkyl, heteroaryl, heteroaralkyl, —R¹⁸OR²¹, —R¹⁸SR²¹, —R¹⁸NR²²R²³; wherein the alkyl, alkenyl, alkynyl, cycloalkyl, cycloalkylalkyl, heterocyclyl and heterocyclylalkyl may each be optionally substituted with one and wherein the to five groups independently selected from halo, cyano, oxo, alkyl, haloalkyl, hydroxyalkyl, alkoxyalkyl, cyanoalkyl, —R^{w}—OR^{x} and —R^{w}—NR^{y}R^{z} and wherein the aryl, aralkyl, heteroaryl and heteroaralkyl may each be optionally substituted with one to five groups independently selected from halo, cyano, alkyl, haloalkyl, hydroxyalkyl, alkoxyalkyl, cyanoalkyl, —R^{w}—OR^{x} and —R^{w}—NR^{y}R^{z};
R²² and R²³ are independently selected from (i) or (ii) below:
(i) each R²² and R²³ is independently hydrogen or alkyl wherein the alkyl may each be optionally substituted by one or more substituents independently selected from the group consisting of halo, heterocyclyl, —OR^{x}, —NR^{y}R^{z} and —C(O)OR^{x}, or
(ii) R²² and R²³, together with the nitrogen atom to which they are attached, form a heterocyclyl which may optionally be substituted with one or more substituents independently selected from the group consisting of halo, oxo, alkyl, haloalkyl, hydroxyalkyl, —OR^{x}, —NR^{y}R^{z} and —C(O)OR^{x};
each R^{w} is independently a direct bond or alkylene;
each R^{x} is independently hydrogen, alkyl, alkenyl, alkynyl or cycloalkyl; and
each R^{y} and R^{z} is independently selected from (i) or (ii) below:
(i) R^{y} and R^{z} are each independently hydrogen, alkyl, cycloalkyl or cycloalkylalkyl, or
(ii) R^{y} and R^{z}, together with the nitrogen atom to which they are attached, form a heterocyclyl which may be optionally substituted with one or more substituents independently selected from the group consisting of halo, oxo, alkyl, haloalkyl, hydroxyalkyl, hydroxy and alkoxy.
